(19) United States
(12) Patent Application Publication
(10) Pub. No.: US 2024/0260466 A1
(43) Pub. Date: Aug. 1, 2024

(54) PLURALITY OF HOST MATERIALS AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Su-Hyun LEE, Gyeonggi-do (KR); So-Young JUNG, Gyeonggi-do (KR); Dong-Gil KIM, Gyeonggi-do (KR); Sang-Hee CHO, Gyeonggi-do (KR); Jin-Ri HONG, Gyeonggi-do (KR); Doo-Hyeon MOON, Gyeonggi-do (KR)

(21) Appl. No.: 18/431,116

(22) Filed: Feb. 2, 2024

Related U.S. Application Data
(63) Continuation of application No. 17/113,992, filed on Dec. 7, 2020, now abandoned.

(30) Foreign Application Priority Data
Dec. 20, 2019 (KR) 10-2019-0171955
Oct. 29, 2020 (KR) 10-2020-0141706

(51) Int. Cl. H10K 85/60; H10K 50/11; H10K 101/00; H10K 101/10

(52) U.S. Cl. CPC H10K 85/6574; H10K 85/615; H10K 85/633; H10K 85/636; H10K 85/654; H10K 50/11; H10K 2101/10; H10K 2101/90

(57) ABSTRACT

The present disclosure relates to a plurality of host materials comprising a first host material comprising a compound represented by formula 1, and a second host material comprising a compound represented by formula 2, and an organic electroluminescent device comprising the same. By comprising a specific combination of compounds of the present disclosure as host materials, it is possible to provide an organic electroluminescent device having improved lifetime properties as compared with a conventional organic electroluminescent device.

### CLAIM OF BENEFIT OF PRIOR APPLICATION

This application claims priority under 35 U.S.C. § 120 from U.S. patent application Ser. No. 17/113,992, filed Dec. 7, 2020, which is incorporated by reference herein in their entirety.

### TECHNICAL FIELD

The present disclosure relates to a plurality of host materials and an organic electroluminescent device comprising the same.

### BACKGROUND ART

In 1987, Tang et al. of Eastman Kodak first developed a small molecule green organic electroluminescent device (OLED) of TPD/Alq3 bilayer consisting of a light-emitting layer and a charge transport layer. Since then, the research on an OLED has been rapidly carried out, and it has been commercialized. At present, phosphorescent materials, which provide excellent luminous efficiency in realizing panels, are mainly used in organic electroluminescent devices. Thus, an OLED which has high luminous efficiency and/or long lifetime is required for long time uses and high resolution of displays.

Korean Patent Appl. Laid-Open No. 2017-0043439 discloses a new organic electroluminescent compound. However, the aforementioned reference does not specifically disclose a specific combination of host materials claimed in the present disclosure. In addition, there is a need for the development of a light-emitting material having improved performances, for example, improved lifetime properties, by combining the compound disclosed in the aforementioned reference with a specific compound.

### DISCLOSURE OF INVENTION

### Technical Problem

The objective of the present disclosure is to provide an organic electroluminescent device having improved lifetime properties, by comprising a specific combination of compounds as host materials.

### Solution to Problem

The present inventors found that the above objective can be achieved by a plurality of host materials comprising a first host material comprising a compound represented by the following formula 1, and a second host material comprising a compound represented by the following formula 2:

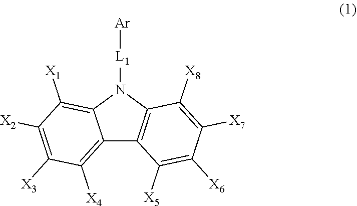

wherein,
Ar represents a substituted or unsubstituted (C6-C30)aryl, or a substituted or unsubstituted (3- to 30-membered)heteroaryl containing at least one of nitrogen(s), oxygen(s) and sulfur(s), or —NX₉X₁₀;
L₁ represents a single bond, a substituted or unsubstituted (C6-C30)arylene, or a substituted or unsubstituted (3- to 30-membered)heteroarylene;
X₁ to X₃, each independently, represent hydrogen, deuterium, a halogen, a cyano, a carboxyl, a nitro, a hydroxyl, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NX₁₁X₁₂, or —SiX₁₃X₁₄X₁₅; or two or more adjacent ones of X₁ to X₃ may be linked to each other to form a substituted or unsubstituted, monocyclic ring(s) or polycyclic ring(s) having 2 to 5 rings, in which at least one ring must be formed;
X₉ and X₁₀, each independently, represent a substituted or unsubstituted (C1-C30)alkyl, a substituted or unsubstituted (C3-C30)cycloalkyl, a substituted or unsubstituted (C6-C30)aryl, or a substituted or unsubstituted (3- to 30-membered)heteroaryl; and
X₁₁ to X₁₅, each independently, represent hydrogen, deuterium, a halogen, a cyano, a carboxyl, a nitro, a hydroxyl, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl; or adjacent ones of X₁₁ to X₁₅ may be linked to each other to form a ring(s);

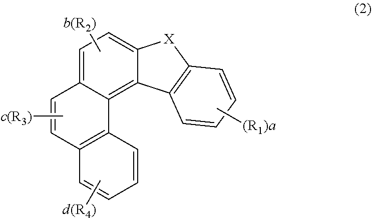

wherein,
X represents O, S, or CR₁₁R₁₂;
R₁ to R₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, or a substituted or unsubstituted (C6-C30)aryl(3- to 30-membered)heteroarylamino,
in which, at least one of R₁ to R₄, each independently, represent a substituted or unsubstituted (C6-C30)aryl, a substituted or unsubstituted (3- to 30-membered)heteroaryl, a substituted or unsubstituted mono- or di-(C6-C30)arylamino, a substituted or unsubstituted mono- or di-(3- to 30-membered)heteroarylamino, or a substituted or unsubstituted (C6-C30)aryl(3- to 30-membered)heteroarylamino; with the proviso that any one of R₁ to R₄ does not represent a triphenylene;
R₁₁ and R₁₂, each independently, represent a substituted or unsubstituted (C1-C30)alkyl, a substituted or unsubstituted (C6-C30)aryl, or a substituted or unsubstituted (3- to 30-membered)heteroaryl; or R₁₁ and R₁₂ may be linked to each other to form a ring(s);
a and d, each independently, represent an integer of 1 to 4; b and c, each independently, represent an integer of 1 or 2; in which if a to d, each independently, are an integer of 2 or more, each of R₁, each of R₂, each of R₃, and each of R₄ may be the same or different; and
the heteroaryl contains at least one heteroatom selected from B, N, O, S, Si, and P.

### Advantageous Effects of Invention

By comprising a specific combination of compounds of the present disclosure as host materials, it is possible to provide an organic electroluminescent device having improved lifetime properties as compared with the conventional organic electroluminescent device, and manufacture a display system or a light system using the same.

### MODE FOR THE INVENTION

Hereinafter, the present disclosure will be described in detail. However, the following description is intended to explain the disclosure, and is not meant in any way to restrict the scope of the disclosure.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (containing host and dopant materials), an electron buffer material, a hole blocking material, an electron transport material, an electron injection material, etc.

The term “a plurality of organic electroluminescent materials” in the present disclosure means an organic electroluminescent material(s) comprising a combination of at least two compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of two or more compounds which may be comprised in at least one of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. The two or more compounds may be comprised in the same layer or different layers, and may be mixture-evaporated or co-evaporated, or may be individually evaporated.

The term “a plurality of host materials” in the present disclosure means an organic electroluminescent material comprising a combination of at least two host materials. It may mean both a material before being comprised in an organic electroluminescent device (for example, before vapor deposition) and a material after being comprised in an organic electroluminescent device (for example, after vapor deposition). The plurality of host materials of the present disclosure may be comprised in any light-emitting layer constituting an organic electroluminescent device. The two or more compounds comprised in the plurality of host materials of the present disclosure may be included in one light-emitting layer or may be respectively included in different light-emitting layers. For example, the two or more host materials may be mixture-evaporated to form a layer, or separately co-evaporated at the same time to form a layer.

Herein, the term “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, sec-butyl, tert-butyl, etc. The term “(C2-C30)alkenyl” is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. The term “(C2-C30)alkynyl” is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. The term “(3- to 7-membered)heterocycloalkyl” is meant to be a cycloalkyl having 3 to 7, preferably 5 to 7, ring backbone atoms, and including at least one heteroatom selected from the group consisting of B, N, O, S, Si, and P, and preferably the group consisting of O, S, and N. The above heterocycloalkyl may include tetrahydrofuran, pyrrolidine, thiolan, tetrahydropyran, etc. The term “(C6-C30)aryl(ene)” is meant to be a monocyclic or fused ring radical derived from an aromatic hydrocarbon having 6 to 30 ring backbone carbon atoms. The above aryl(ene) may be partially saturated, and may comprise a spiro structure. The number of ring backbone carbon atoms is preferably 6 to 20, and more preferably 6 to 15. The above aryl may include phenyl, biphenyl, terphenyl, naphthyl, binaphthyl, phenylnaphthyl, naphthylphenyl, fluorenyl, phenylfluorenyl, diphenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-benzofluorene]yl, azulenyl, tetramethyldihydrophenanthrenyl, etc. More specifically, the aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenylyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl(ene)” is an aryl(ene) having 3 to 30 ring backbone atoms, preferably 3 to 25 ring backbone atoms, more preferably 5 to 20 ring backbone atoms, and including at least one, preferably 1 to 4 heteroatoms selected from the group consisting of B, N, O, S, Si, and P. The above heteroaryl(ene) may be a monocyclic ring, or a fused ring condensed with at least one benzene ring; may be partially saturated; may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, benzonaphthofuranyl, dibenzothiophenyl, dibenzoselenophenyl, benzonaphthothiophenyl, benzofuroquinolinyl, benzofuroquinazolinyl, benzofuronaphthyridinyl, benzofuropyrimidinyl, naphthofuropyrimidinyl, benzothienoquinolinyl, benzothienoquinazolinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, naphthyridinyl, carbazolyl, benzocarbazolyl, phenoxazinyl, phenanthridinyl, phenanthroxazolyl, benzodioxolyl, dihydroacridinyl, benzotriazolphenazinyl, imidazopyridyl, chromenoquinazolinyl, thiochromenoquinazolinyl, dimethylbenzoperimidinyl, indolocarbazolyl, indenocarbazolyl, etc. More specifically, the heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. “Halogen” includes F, Cl, Br, and I.

In addition, “ortho (o-),” “meta (m-),” and “para (p-)” are prefixes, which represent the relative positions of substituents, respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

In the formulas of the present disclosure, a ring formed by a linkage of adjacent substituents means that at least two adjacent substituents are linked to or fused with each other to form a substituted or unsubstituted mono- or polycyclic (3- to 30-membered) alicyclic or aromatic ring, or the combination thereof; preferably, a substituted or unsubstituted mono- or polycyclic (3- to 26-membered) alicyclic or aromatic ring, or the combination thereof; more preferably, an unsubstituted mono- or polycyclic (5- to 20-membered) aromatic ring. Also, the ring may contain at least one heteroatom selected from B, N, O, S, Si, and P, preferably at least one heteroatom selected from N, O, and S. For example, the ring may be a substituted or unsubstituted, benzene ring, indene ring, indole ring, benzoindole ring, benzofuran ring, benzothiophene ring, etc.

Herein, “substituted” in the expression “substituted or unsubstituted” means that a hydrogen atom in a certain functional group is replaced with another atom or another functional group, i.e., a substituent, and also includes that the hydrogen atom is replaced with a group formed by a linkage of two or more substituents. For example, the group formed by a linkage of two or more substituents may be pyridine-triazine. That is, pyridine-triazine may be interpreted as a heteroaryl substituent, or as a substituent in which two heteroaryls are linked. In the present disclosure, the substituents of the substituted alkyl, the substituted aryl, the substituted arylene, the substituted heteroaryl, the substituted heteroarylene, the substituted cycloalkyl, the substituted cycloalkenyl, the substituted heterocycloalkyl, the substituted alkoxy, the substituted trialkylsilyl, the substituted dialkylarylsilyl, the substituted alkyldiarylsilyl, the substituted triarylsilyl, the substituted mono- or di-alkylamino, the substituted mono- or di-arylamino, the substituted alkylarylamino, the substituted mono- or di-heteroarylamino, the substituted arylheteroarylamino, the substituted monocyclic ring, and the substituted polycyclic ring, each independently, are at least one selected from the group consisting of deuterium; a halogen; a cyano; a carboxyl; a nitro; a hydroxyl; a phosphineoxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of a (C1-C30)alkyl(s), a (C6-C30)aryl(s) and a (3- to 30-membered)heteroaryl(s); a (C6-C30)aryl unsubstituted or substituted with at least one of a cyano(s), a (C1-C30)alkyl(s) and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C6-C30)arylamino unsubstituted or substituted with a (C1-C30)alkyl(s); a (C1-C30)alkyl(C6-C30)arylamino; a mono- or di-(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituents, each independently, are at least one selected from the group consisting of a cyano; a (C1-C20)alkyl; a (5- to 25-membered)heteroaryl unsubstituted or substituted with at least one of a (C6-C25)aryl(s) and a (5- to 25-membered)heteroaryl(s); a (C6-C25)aryl unsubstituted or substituted with at least one of a cyano(s) and a (C1-C20)alkyl(s); a mono- or di-(C6-C25)arylamino unsubstituted or substituted with a (C1-C10)alkyl(s); a mono- or di-(5- to 25-membered)heteroarylamino; and a (C6-C25)aryl(5- to 25-membered)heteroarylamino. According to another embodiment of the present disclosure, the substituents, each independently, are at least one selected from the group consisting of a cyano; a (C1-C10)alkyl; a (5- to 20-membered)heteroaryl unsubstituted or substituted with at least one of a (C6-C18)aryl(s) and a (5- to 20-membered)heteroaryl(s); a (C6-C25)aryl unsubstituted or substituted with at least one of a cyano(s) and a (C1-C10)alkyl(s); a di(C6-C18)arylamino unsubstituted or substituted with a (C1-C6)alkyl(s); and a (C6-C18)aryl(5- to 20-membered)heteroarylamino. For example, the substituents, each independently, may be at least one selected from the group consisting of a cyano; a methyl; a phenyl unsubstituted or substituted with a cyano(s); a naphthyl; a biphenyl; a dimethylfluorenyl; a diphenylfluorenyl; a phenanthrenyl; a naphthylphenyl; a phenylnaphthyl; a terphenyl; a triazinyl substituted with at least one selected from the group consisting of a phenyl, a naphthyl, and a pyridyl; a dibenzothiophenyl; a dibenzofuranyl; a diphenylamino; a dibiphenylamino; a phenylbiphenylamino; a dimethylfluorenylphenylamino; a phenyldibenzothiofuranylamino; and a phenyldibenzothiophenylamino.

Herein, the heteroaryl, the heteroarylene, and the heterocycloalkyl, each independently, may contain at least one heteroatom selected from B, N, O, S, Si, and P. Also, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, and a substituted or unsubstituted (C1-C30)alkyl(C6-C30)arylamino.

In formula 1, Ar represents a substituted or unsubstituted (C6-C30)aryl, or a substituted or unsubstituted (3- to 30-membered)heteroaryl containing at least one of nitrogen(s), oxygen(s) and sulfur(s), or —NX₉X₁₀. According to one embodiment, Ar represents a substituted or unsubstituted (C6-C25)aryl, or a substituted or unsubstituted (5- to 20-membered)heteroaryl containing at least one of nitrogen(s), oxygen(s) and sulfur(s), or —NX₉X₁₀. According to another embodiment, Ar represents a (C6-C25)aryl unsubstituted or substituted with a (C1-C6)alkyl(s); or a (5- to 20-membered)heteroaryl unsubstituted or substituted with at least one of a (C6-C18)aryl(s) and a (5- to 20-membered)heteroaryl(s), containing at least one of nitrogen(s), oxygen(s) and sulfur(s); or —NX₉X₁₀. Specifically, Ar may represent a substituted or unsubstituted phenyl, a substituted or unsubstituted naphthyl, a substituted or unsubstituted biphenyl, a substituted or unsubstituted terphenyl, a substituted or unsubstituted fluorenyl, a substituted or unsubstituted benzofluorenyl, a substituted or unsubstituted triphenylenyl, a substituted or unsubstituted spirobifluorenyl, a substituted or unsubstituted pyridyl, a substituted or unsubstituted triazinyl, a substituted or unsubstituted pyrimidinyl, a substituted or unsubstituted quinolyl, a substituted or unsubstituted quinazolinyl, a substituted or unsubstituted quinoxalinyl, a substituted or unsubstituted benzoquinazolinyl, a substituted or unsubstituted benzoquinoxalinyl, a substituted or unsubstituted benzofuropyrimidinyl, a substituted or unsubstituted carbazolyl, a substituted or unsubstituted dibenzothiophenyl, a substituted or unsubstituted benzothiophenyl, a substituted or unsubstituted dibenzofuranyl, a substituted or unsubstituted benzofuranyl, a substituted or unsubstituted naphthyridinyl, a substituted or unsubstituted benzonaphthofuranyl, a substituted or unsubstituted benzonaphthothiophenyl, or —NX₉X₁₀. For example, Ar may represent a phenyl, a naphthyl, a biphenyl, a terphenyl, a dimethylfluorenyl, a dimethylbenzofluorenyl, a spirobifluorenyl, a substituted pyridyl, a substituted pyrimidinyl, a substituted triazinyl, a substituted quinolyl, a substituted quinazolinyl, a substituted quinoxalinyl, a substituted naphthyridinyl, a dibenzofuranyl, a dibenzothiophenyl, a substituted carbazolyl, a substituted benzofuropyrimidinyl, a substituted benzoquinoxalinyl, a substituted benzoquinazolinyl, or —NX₉X₁₀; and the substituent(s) of the substituted pyridyl, the substituted pyrimidinyl, the substituted triazinyl, the substituted quinolyl, the substituted quinazolinyl, the substituted quinoxalinyl, the substituted naphthyridinyl, the substituted carbazolyl, the substituted benzofuropyrimidinyl, the substituted benzoquinoxalinyl, and the substituted benzoquinazolinyl, each independently, may be at least one selected from the group consisting of a phenyl, a naphthyl, a biphenyl, a dibenzofuranyl, a dibenzothiophenyl, and a dimethylfluorenyl.

X₉ and X₁₀, each independently, represent a substituted or unsubstituted (C1-C30)alkyl, a substituted or unsubstituted (C3-C30)cycloalkyl, a substituted or unsubstituted (C6-C30)aryl, or a substituted or unsubstituted (3- to 30-membered)heteroaryl. According to one embodiment, X₉ and X₁₀, each independently, represent a substituted or unsubstituted (C6-C25)aryl, or a substituted or unsubstituted (5- to 25-membered)heteroaryl. According to another embodiment, X₉ and X₁₀, each independently, represent an unsubstituted (C6-C18)aryl. For example, X₉ and X₁₀, each independently, represent a phenyl, a naphthyl, a biphenyl, or a naphthylphenyl, etc.

In formula 1, L₁ represents a single bond, a substituted or unsubstituted (C6-C30)arylene, or a substituted or unsubstituted (3- to 30-membered)heteroarylene. According to one embodiment, L₁ represents a single bond, a substituted or unsubstituted (C6-C25)arylene, or a substituted or unsubstituted (5- to 25-membered)heteroarylene. According to another embodiment, L₁ represents a single bond, an unsubstituted (C6-C20)arylene, or a (5- to 20-membered)heteroarylene unsubstituted or substituted with a (C6-C18)aryl(s). For example, L₁ may represent a single bond, a phenylene, a naphthylene, a biphenylene, a triazinylene substituted with a phenyl(s), a pyrimidinylene substituted with a phenyl(s), a quinolylene, a quinazolinylene unsubstituted or substituted with a phenyl(s), a quinoxalinylene unsubstituted or substituted with a phenyl(s), a naphthyridinylene, a carbazolylene, a benzofuropyrimidinylene, a benzoquinoxalinylene, or a benzoquinazolinylene, etc.

In formula 1, X₁ to X₃, each independently, represent hydrogen, deuterium, a halogen, a cyano, a carboxyl, a nitro, a hydroxyl, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NX₁₁X₁₂, or —SiX₁₃X₁₄X₁₅; or two or more adjacent ones of X₁ to X₃ may be linked to each other to form a substituted or unsubstituted, monocyclic ring(s) or polycyclic ring(s) having 2 to 5 rings, in which at least one ring must be formed. That is, X₁ and X₂ may be linked to each other to form a ring(s), and/or X₂ and X₃ may be linked to each other to form a ring(s), and/or X₃ and X₄ may be linked to each other to form a ring(s), and/or X₄ and X₅ may be linked to each other to form a ring(s), and/or X₅ and X₆ may be linked to each other to form a ring(s), and/or X₆ and X₇ may be linked to each other to form a ring(s), and/or X₇ and X₈ may be linked to each other to form a ring(s), and one or more rings are necessarily formed in a structure according to formula 1. Also, it may be excluded that any one of X₁ to X₈ contains a carbazole ring. According to one embodiment, X₁ to X₈, each independently, represent hydrogen, a substituted or unsubstituted (C1-C20)alkyl, a substituted or unsubstituted (C6-C25)aryl, a substituted or unsubstituted (5- to 25-membered)heteroaryl, or —NX₁₁X₁₂; or two or more adjacent ones of X₁ to X₈ may be linked to each other to form a substituted or unsubstituted, monocyclic ring(s) or polycyclic ring(s) having 2 to 5 rings, in which at least one ring must be formed. According to another embodiment, X₁ to X₈, each independently, represent hydrogen; or two or more adjacent ones of X₁ to X₈ may be linked to each other to form a substituted or unsubstituted, monocyclic ring(s) or polycyclic ring(s) having 2 to 5 rings, in which at least one ring must be formed. For example, X₁ to X₈, each independently, represent hydrogen; or two or more adjacent ones of X₁ to X₈ may be linked to each other to form a benzene ring; an indole ring substituted with a phenyl(s), a naphthyl(s), a biphenyl(s), or a terphenyl(s); or a benzoindole ring substituted with a phenyl(s), etc.

In formula 1, X₁₁ to X₁S, each independently, represent hydrogen, deuterium, a halogen, a cyano, a carboxyl, a nitro, a hydroxyl, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl; or adjacent ones of X₁ to X₁₅ may be linked to each other to form a ring(s).

According to one embodiment, the formula 1 may be represented by at least one of the following formulas 1-1 to 1-6.

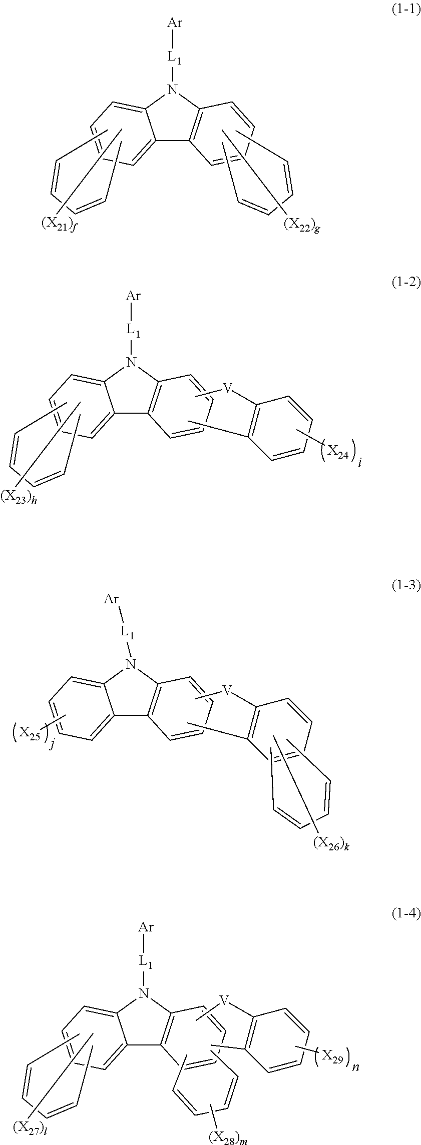

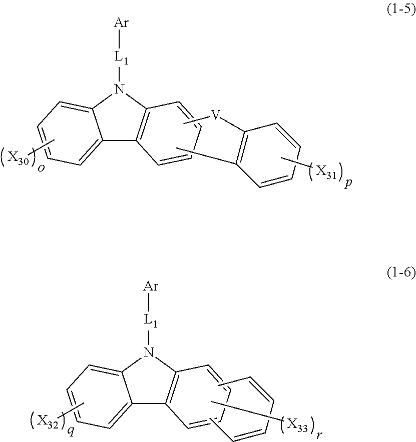

In formulas 1-1 to 1-6, Ar and L, are as defined in formula 1.

In formulas 1-1 to 1-6, V, each independently, represents CX₁₈X₁₉, NX₂₀, O or S. According to one embodiment, V, each independently, may represent NX₂₀.

X₁₈ to X₃₃, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino. According to one embodiment, X₁₈ to X₃₃, each independently, represent hydrogen, deuterium, a substituted or unsubstituted (C1-C20)alkyl, a substituted or unsubstituted (C6-C25)aryl, or a substituted or unsubstituted (5- to 25-membered)heteroaryl. According to another embodiment, X₁₈ to X₂₀, each independently, represent an unsubstituted (C1-C10)alkyl, an unsubstituted (C6-C25)aryl, or an unsubstituted (5- to 20-membered)heteroaryl; and X₂₁ to X₃₃, each independently, represent hydrogen, deuterium, an unsubstituted (C1-C10)alkyl, an unsubstituted (C6-C25)aryl, or an unsubstituted (5- to 25-membered)heteroaryl. For example, X₁₈ to X₂₀, each independently, may represent a phenyl, a naphthyl, a biphenyl, or a terphenyl, etc., and X₂₁ to X₃₃, each independently, may represent hydrogen.

In formulas 1-1 to 1-6, i, j, m, n, o, p, and q, each independently, represent an integer of 1 to 4; f to h, k, l, and r, each independently, represent an integer of 1 to 6; in which if f to r, each independently, are an integer of 2 or more, each of X₂₁, each of X₂₂, each of X₂₃, each of X₂₄, each of X₂₅, each of X₂₆, each of X₂₇, each of X₂₈, each of X₂₉, each of X₃₀, each of X₃₁, each of X₃₂, and each of X₃₃ may be the same or different.

In formula 2, X represents O, S, or CR₁₁R₁₂.

In formula 2, R₁ to R₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, or a substituted or unsubstituted (C6-C30)aryl(3- to 30-membered)heteroarylamino; in which, at least one of R₁ to R₄, each independently, represent a substituted or unsubstituted (C6-C30)aryl, a substituted or unsubstituted (3- to 30-membered)heteroaryl, a substituted or unsubstituted mono- or di-(C6-C30)arylamino, a substituted or unsubstituted mono- or di-(3- to 30-membered)heteroarylamino, or a substituted or unsubstituted (C6-C30)aryl(3- to 30-membered)heteroarylamino; with the proviso that any one of R₁ to R₄ does not represent a triphenylene. According to one embodiment, at least one of R₁ to R₄, each independently, represents a substituted or unsubstituted (C6-C25)aryl, a substituted or unsubstituted (5- to 25-membered)heteroaryl, a substituted or unsubstituted mono- or di-(C6-C25)arylamino, a substituted or unsubstituted mono- or di-(5- to 25-membered)heteroarylamino, or a substituted or unsubstituted (C6-C25)aryl(5- to 25-membered)heteroarylamino; with the proviso that any one of R₁ to R₄ does not represent a triphenylene. According to another embodiment, at least one of R₁ to R₄, each independently, represents a (C6-C18)aryl substituted with at least one of a (5- to 25-membered)heteroaryl(s), a di(C6-C18)arylamino(s), and a (C6-C18)aryl(5- to 25-membered)heteroarylamino(s); a (5- to 20-membered)heteroaryl substituted with a (C6-C18)aryl(s) and/or a (5- to 25-membered)heteroaryl(s); a di(C6-C25)arylamino unsubstituted or substituted with a (C1-C6)alkyl(s) and/or a (C6-C18)aryl(s); an unsubstituted di(5- to 20-membered)heteroarylamino; or an unsubstituted (C6-C18)aryl(5- to 20-membered)heteroarylamino; with the proviso that any one of R₁ to R₄ does not represent a triphenylene. For example, R₁ to R₄, each independently, may represent hydrogen; or at least one of R₁ to R₄, each independently, may represent a substituted phenyl, a substituted naphthyl, a substituted biphenyl, a substituted pyridyl, a substituted pyrimidinyl, a substituted triazinyl, a quinoxalinyl substituted with a naphthyl(s), a quinazolinyl substituted with a biphenyl(s), a benzofuropyrimidinyl substituted with a phenyl(s), a dibiphenylamino, a dimethylfluorenylbiphenylamino, a diphenylfluorenylbiphenylamino, a phenylbiphenylamino substituted with a naphthyl(s), a biphenylphenanthrenylamino, a diphenylfluorenylphenylamino, a spirobifluorenylphenylamino, a biphenyldibenzofuranylamino, a terphenyldibenzofuranylamino, a naphthyldibenzofuranylamino, a phenanthrenyldibenzofuranylamino, a phenylnaphthyldibenzofuranylamino, a biphenyldibenzothiophenylamino, a di-dibenzofluorenylamino, or dibenzofluorenyldibenzofuranylamino; in which the substituent(s) of the substituted phenyl, the substituted naphthyl, the substituted biphenyl, the substituted pyridyl, the substituted pyrimidinyl, and the substituted triazinyl, each independently, may be at least one selected from the group consisting of a phenyl unsubstituted or substituted with a cyano(s); a naphthyl; a biphenyl; a phenylnaphthyl; a naphthylphenyl; a phenanthrenyl; a terphenyl; a dimethylfluorenyl; a dibenzofuranyl; a dibenzothiophenyl; a triazinyl substituted with at least one of a phenyl(s), a naphthyl(s) and a pyridyl(s); a diphenylamino; a dibiphenylamino; a biphenylphenylamino; a dimethylfluorenylphenylamino; and a dibenzofuranylphenylamino.

R₁₁ and R₁₂, each independently, represent a substituted or unsubstituted (C1-C30)alkyl, a substituted or unsubstituted (C6-C30)aryl, or a substituted or unsubstituted (3- to 30-membered)heteroaryl; or R₁₁ and R₁₂ may be linked to each other to form a ring(s). According to one embodiment, R₁₁ and R₁₂, each independently, represent a substituted or unsubstituted (C1-C20)alkyl, or a substituted or unsubstituted (C6-C25)aryl. According to another embodiment, R₁₁ and R₁₂, each independently, represent an unsubstituted (C1-C10)alkyl, or an unsubstituted (C6-C18)aryl. For example, R₁₁ and R₁₂, each independently, may represent a methyl.

In formula 2, a and d, each independently, represent an integer of 1 to 4; b and c, each independently, represent an integer of 1 or 2; in which if a to d, each independently, are an integer of 2 or more, each of R₁, each of R₂, each of R₃, and each of R₄ may be the same or different.

The formula 2 may be represented by at least one of the following formulas 2-1 to 2-8.

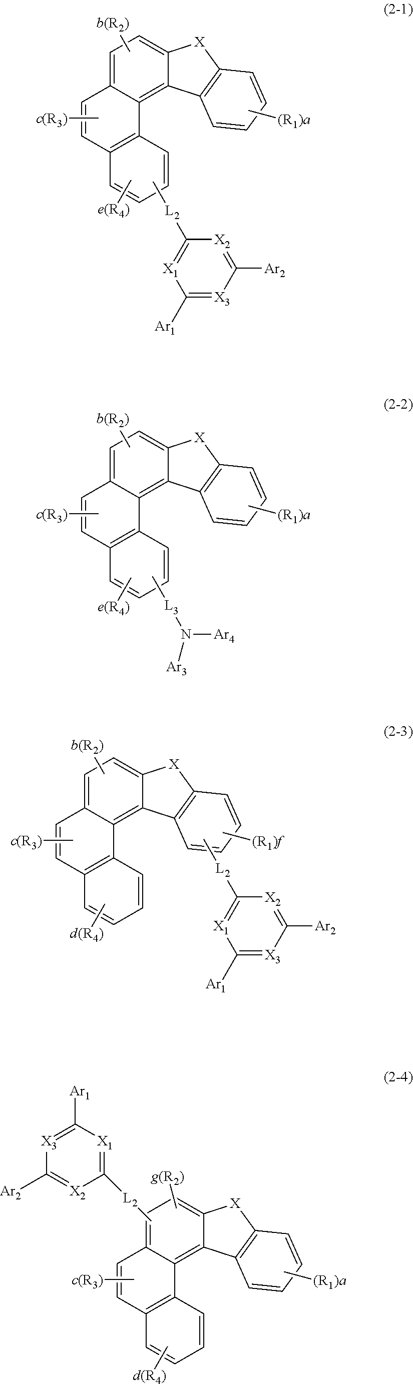

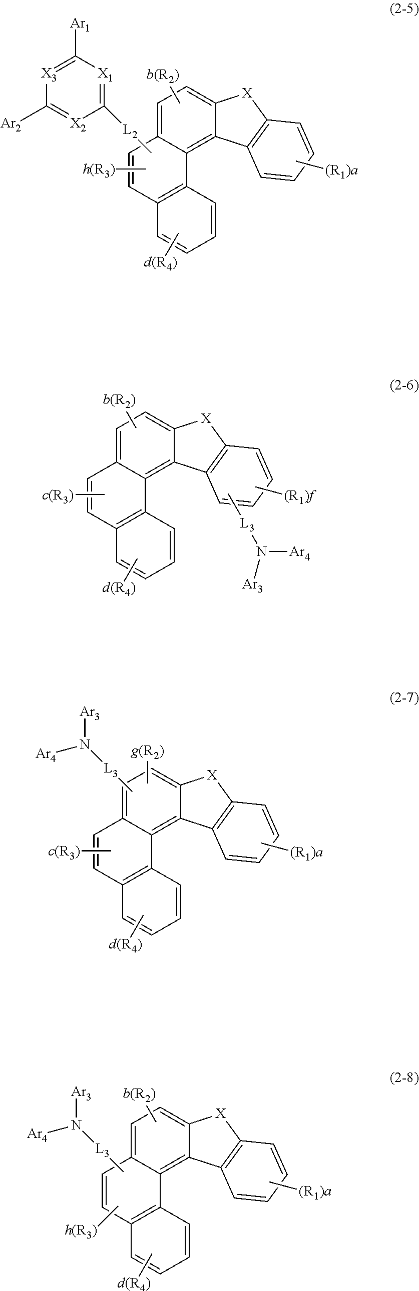

In formulas 2-1 to 2-8, X, and a to d are as defined in formula 2.

In formulas 2-1 to 2-8, R₁ to R₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, or a substituted or unsubstituted (C6-C30)aryl(3- to 30-membered)heteroarylamino. It may be excluded that any one of R₁ to R₄ represents a triphenylene. According to one embodiment of the present disclosure, R₁ to R₄, each independently, represent hydrogen, deuterium, a halogen, a substituted or unsubstituted (C1-C20)alkyl, a substituted or unsubstituted (C6-C25)aryl, a substituted or unsubstituted (5- to 25-membered)heteroaryl, a substituted or unsubstituted mono- or di-(C6-C25)arylamino, a substituted or unsubstituted mono- or di-(5- to 25-membered)heteroarylamino, or a substituted or unsubstituted (C6-C25)aryl(5- to 25-membered)heteroarylamino. For example, R₁ to R₄, each independently, may represent hydrogen.

In formulas 2-1 to 2-8, L₂ and L₃, each independently, represent a single bond, a substituted or unsubstituted (C6-C30)arylene, or a substituted or unsubstituted (3- to 30-membered)heteroarylene. According to one embodiment of the present disclosure, L₂ and L₃, each independently, represent a single bond, a substituted or unsubstituted (C6-C25)arylene, or a substituted or unsubstituted (5- to 25-membered)heteroarylene. According to another embodiment of the present disclosure, L₂ and L₃, each independently, represent a single bond, an unsubstituted (C6-C18)arylene, or an unsubstituted (5- to 20-membered)heteroarylene. For example, L₂ and L₃, each independently, may represent a single bond, a phenylene, a naphthylene, a biphenylene, a pyridylene, etc.

In formulas 2-1, and 2-3 to 2-5, X₁ to X₃, each independently, represent N or CH; with the proviso that at least one of X₁ to X₃ represents N. According to one embodiment of the present disclosure, any one of X₁ to X₃ may represent N; any two of X₁ to X₃ may represent N; or all of X₁ to X₃ may represent N.

In formulas 2-1 to 2-8, Ar₁ to Ar₄, each independently, represent a substituted or unsubstituted (C6-C30)aryl, or a substituted or unsubstituted (3- to 30-membered)heteroaryl. According to one embodiment of the present disclosure, Ar₁ to Ar₄, each independently, represent a substituted or unsubstituted (C6-C25)aryl, or a substituted or unsubstituted (5- to 25-membered)heteroaryl. According to another embodiment of the present disclosure, Ar₁ to Ar₄, each independently, represent a (C6-C25)aryl unsubstituted or a substituted with a cyano(s), a (C1-C6)alkyl(s), and/or a (C6-C18)aryl(s); or an unsubstituted (5- to 20-membered)heteroaryl. For example, Ar₁ to Ar₄, each independently, may represent a phenyl unsubstituted or substituted with a cyano(s), a naphthyl, a biphenyl, a dimethylfluorenyl, a diphenylfluorenyl, a phenanthrenyl, a naphthylphenyl, a phenylnaphthyl, a terphenyl, a spirobifluorenyl, a pyridyl, a dibenzothiophenyl, a dibenzofuranyl, etc.

In formulas 2-1 to 2-8, e and f, each independently, represent an integer of 1 to 3; g and h, each independently, represent an integer of 1; in which if e to h, each independently, are an integer of 2 or more, each of R₁, each of R₂, each of R₃, and each of R₄ may be the same or different.

The compound represented by formula 1 may be specifically exemplified by the following compounds, but is not limited thereto.

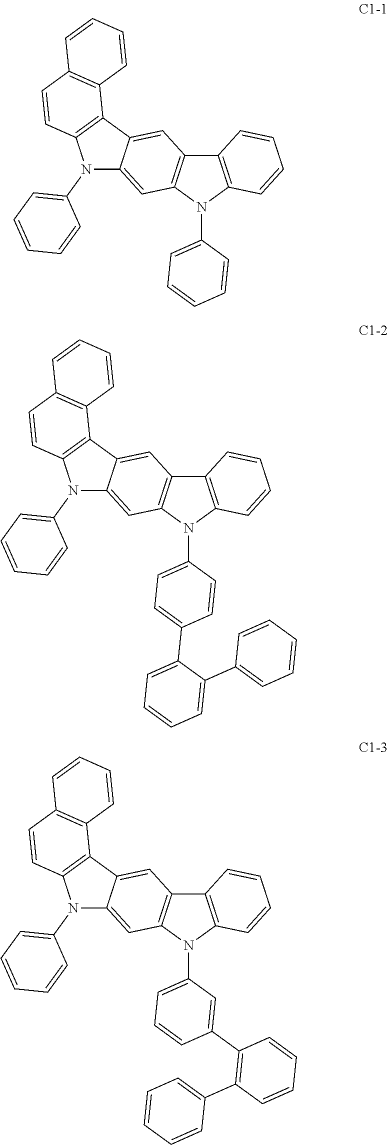 [SMILES: c1ccc(-n2c3ccccc3c3cc4c5c6ccccc6ccc5n(-c5ccccc5)c4cc32)cc1 ; c1ccc(-c2ccccc2-c2ccc(-n3c4ccccc4c4cc5c6c7ccccc7ccc6n(-c6ccccc6)c5cc43)cc2)cc1 ; c1ccc(-c2ccccc2-c2cccc(-n3c4ccccc4c4cc5c6c7ccccc7ccc6n(-c6ccccc6)c5cc43)c2)cc1]

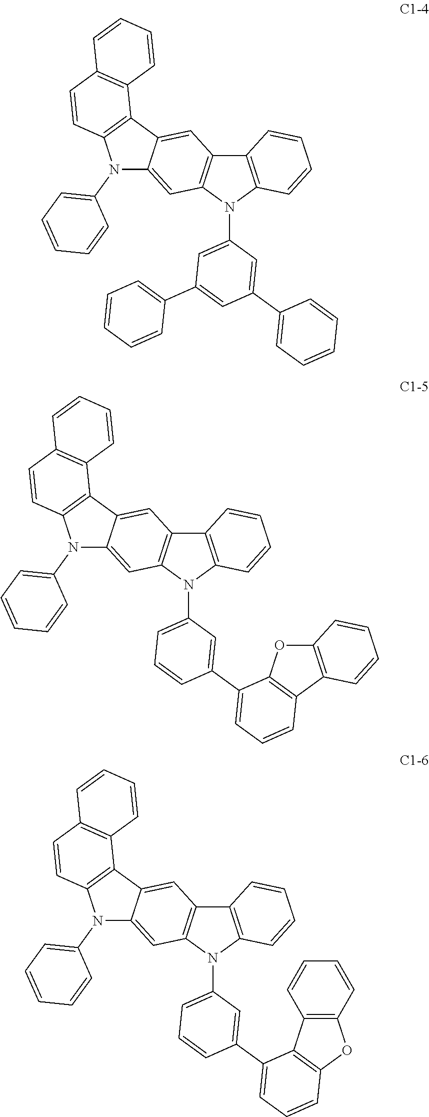 [SMILES: c1ccc(-c2cc(-c3ccccc3)cc(-n3c4ccccc4c4cc5c6c7ccccc7ccc6n(-c6ccccc6)c5cc43)c2)cc1 ; c1ccc(-n2c3cc4c(cc3c3c5ccccc5ccc32)c2ccccc2n4-c2cccc(-c3cccc4c3oc3ccccc34)c2)cc1 ; c1ccc(-n2c3cc4c(cc3c3c5ccccc5ccc32)c2ccccc2n4-c2cccc(-c3cccc4oc5ccccc5c34)c2)cc1]

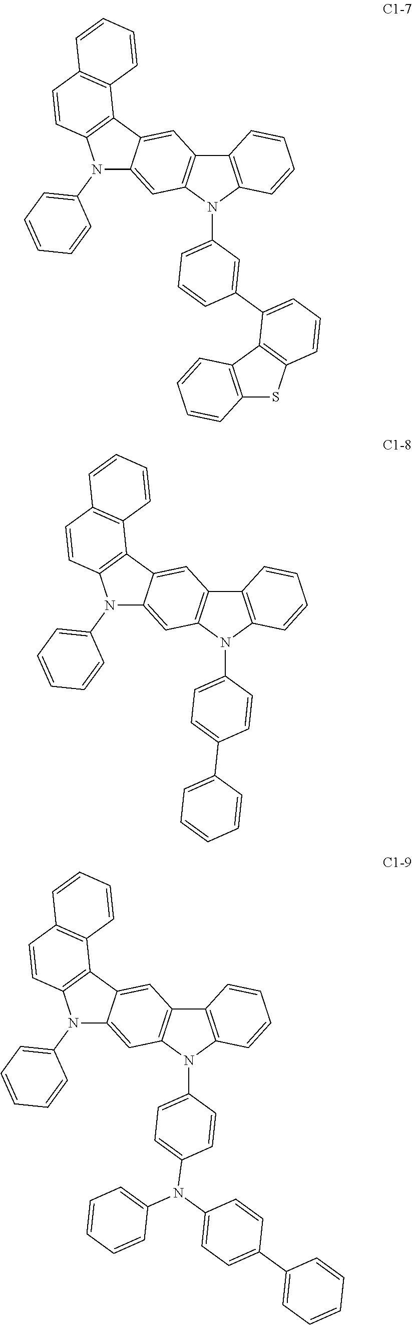 [SMILES: c1ccc(-n2c3cc4c(cc3c3c5ccccc5ccc32)c2ccccc2n4-c2cccc(-c3cccc4sc5ccccc5c34)c2)cc1 ; c1ccc(-c2ccc(-n3c4ccccc4c4cc5c6c7ccccc7ccc6n(-c6ccccc6)c5cc43)cc2)cc1 ; c1ccc(-c2ccc(N(c3ccccc3)c3ccc(-n4c5ccccc5c5cc6c7c8ccccc8ccc7n(-c7ccccc7)c6cc54)cc3)cc2)cc1]

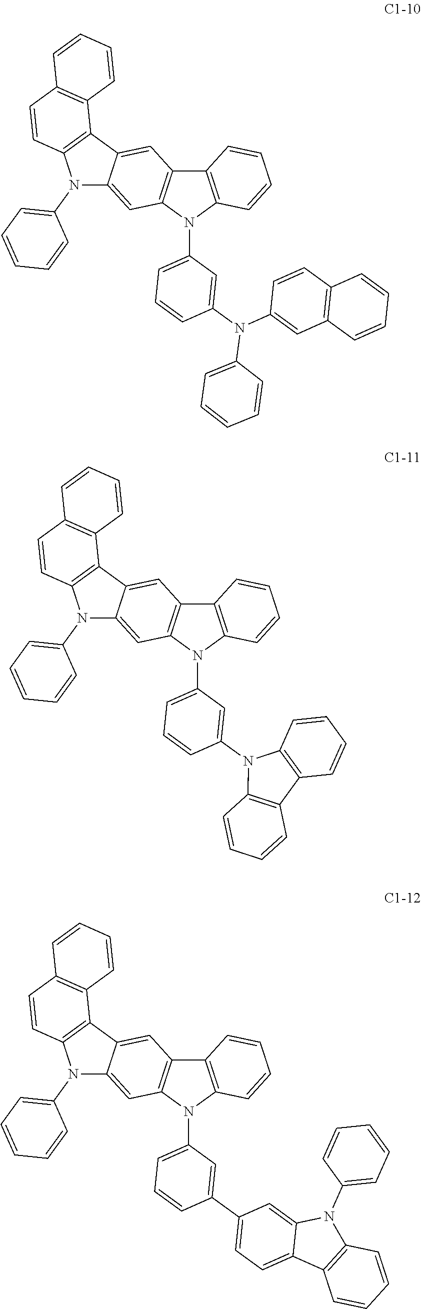 [SMILES: c1ccc(N(c2cccc(-n3c4ccccc4c4cc5c6c7ccccc7ccc6n(-c6ccccc6)c5cc43)c2)c2ccc3ccccc3c2)cc1 ; c1ccc(-n2c3cc4c(cc3c3c5ccccc5ccc32)c2ccccc2n4-c2cccc(-n3c4ccccc4c4ccccc43)c2)cc1 ; c1ccc(-n2c3ccccc3c3ccc(-c4cccc(-n5c6ccccc6c6cc7c8c9ccccc9ccc8n(-c8ccccc8)c7cc65)c4)cc32)cc1]

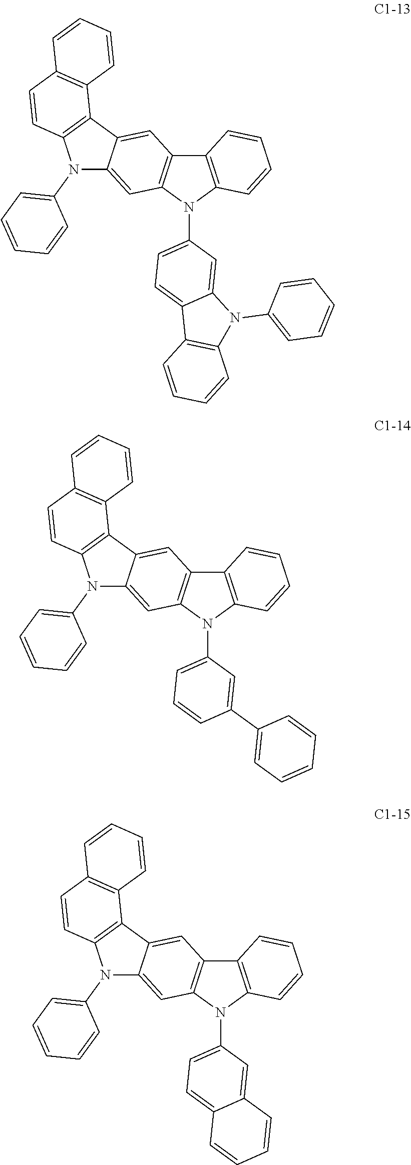 [SMILES: c1ccc(-n2c3ccccc3c3ccc(-n4c5ccccc5c5cc6c7c8ccccc8ccc7n(-c7ccccc7)c6cc54)cc32)cc1 ; c1ccc(-c2cccc(-n3c4ccccc4c4cc5c6c7ccccc7ccc6n(-c6ccccc6)c5cc43)c2)cc1 ; c1ccc(-n2c3cc4c(cc3c3c5ccccc5ccc32)c2ccccc2n4-c2ccc3ccccc3c2)cc1]

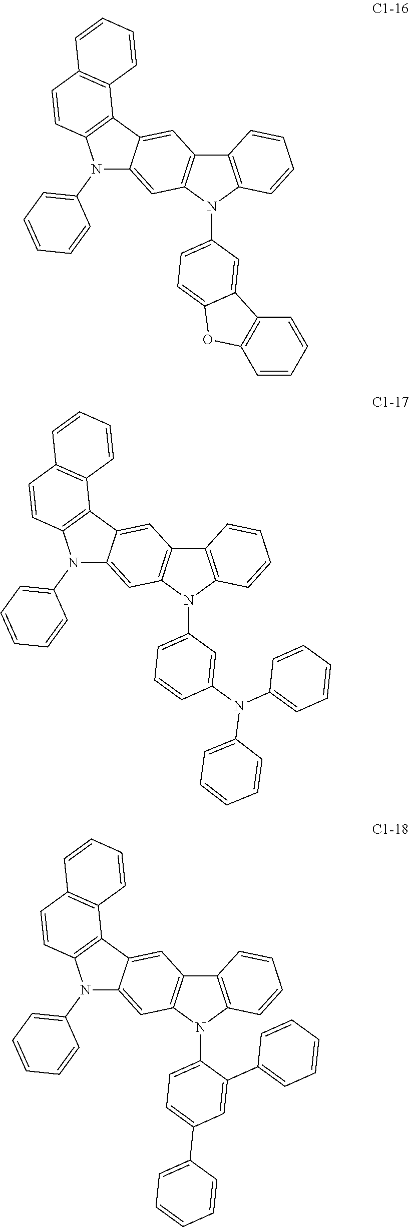 [SMILES: c1ccc(-n2c3cc4c(cc3c3c5ccccc5ccc32)c2ccccc2n4-c2ccc3oc4ccccc4c3c2)cc1 ; c1ccc(N(c2ccccc2)c2cccc(-n3c4ccccc4c4cc5c6c7ccccc7ccc6n(-c6ccccc6)c5cc43)c2)cc1 ; c1ccc(-c2ccc(-n3c4ccccc4c4cc5c6c7ccccc7ccc6n(-c6ccccc6)c5cc43)c(-c3ccccc3)c2)cc1]

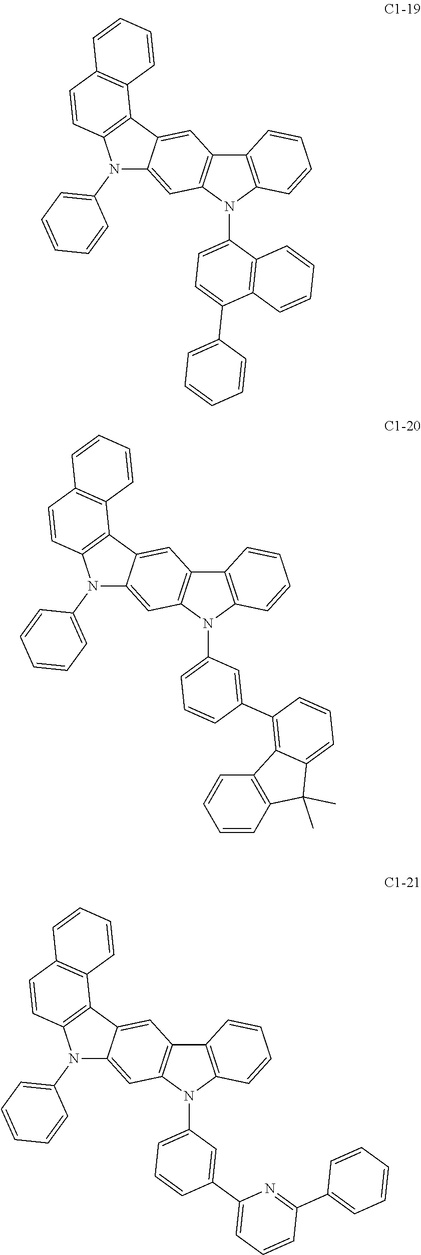 [SMILES: c1ccc(-c2ccc(-n3c4ccccc4c4cc5c6c7ccccc7ccc6n(-c6ccccc6)c5cc43)c3ccccc23)cc1 ; CC1(C)c2ccccc2-c2c(-c3cccc(-n4c5ccccc5c5cc6c7c8ccccc8ccc7n(-c7ccccc7)c6cc54)c3)cccc21 ; c1ccc(-c2cccc(-c3cccc(-n4c5ccccc5c5cc6c7c8ccccc8ccc7n(-c7ccccc7)c6cc54)c3)n2)cc1]

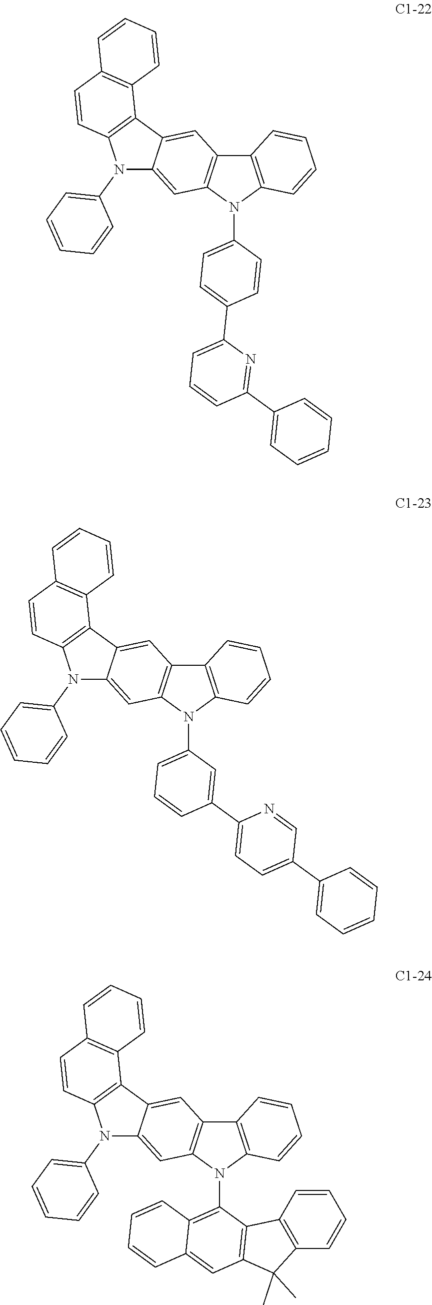 [SMILES: c1ccc(-c2cccc(-c3ccc(-n4c5ccccc5c5cc6c7c8ccccc8ccc7n(-c7ccccc7)c6cc54)cc3)n2)cc1 ; c1ccc(-c2ccc(-c3cccc(-n4c5ccccc5c5cc6c7c8ccccc8ccc7n(-c7ccccc7)c6cc54)c3)nc2)cc1 ; CC1(C)c2ccccc2-c2c1cc1ccccc1c2-n1c2ccccc2c2cc3c4c5ccccc5ccc4n(-c4ccccc4)c3cc21]

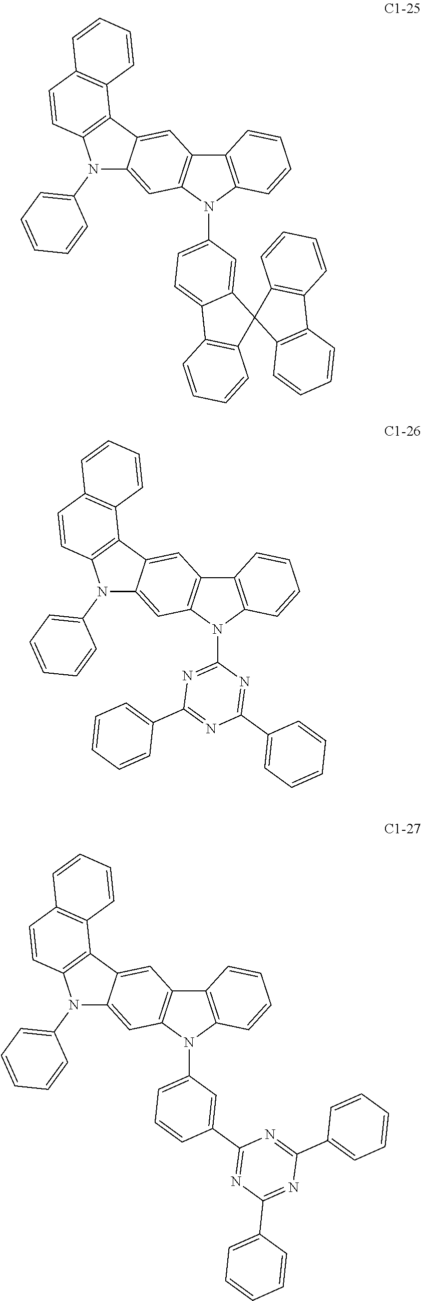 [SMILES: c1ccc(-n2c3cc4c(cc3c3c5ccccc5ccc32)c2ccccc2n4-c2ccc3c(c2)C2(c4ccccc4-c4ccccc42)c2ccccc2-3)cc1 ; c1ccc(-c2nc(-c3ccccc3)nc(-n3c4ccccc4c4cc5c6c7ccccc7ccc6n(-c6ccccc6)c5cc43)n2)cc1 ; c1ccc(-c2nc(-c3ccccc3)nc(-c3cccc(-n4c5ccccc5c5cc6c7c8ccccc8ccc7n(-c7ccccc7)c6cc54)c3)n2)cc1]

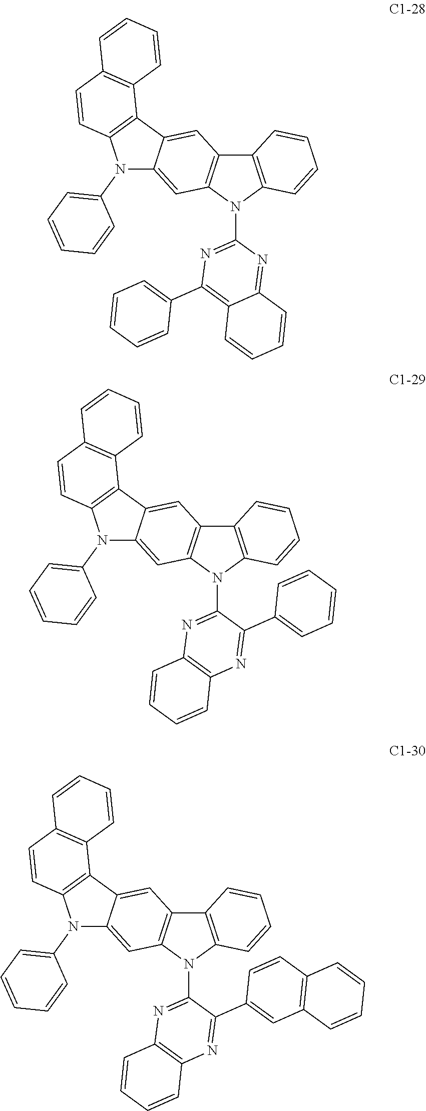 [SMILES: c1ccc(-c2nc(-n3c4ccccc4c4cc5c6c7ccccc7ccc6n(-c6ccccc6)c5cc43)nc3ccccc23)cc1 ; c1ccc(-c2nc3ccccc3nc2-n2c3ccccc3c3cc4c5c6ccccc6ccc5n(-c5ccccc5)c4cc32)cc1 ; c1ccc(-n2c3cc4c(cc3c3c5ccccc5ccc32)c2ccccc2n4-c2nc3ccccc3nc2-c2ccc3ccccc3c2)cc1]

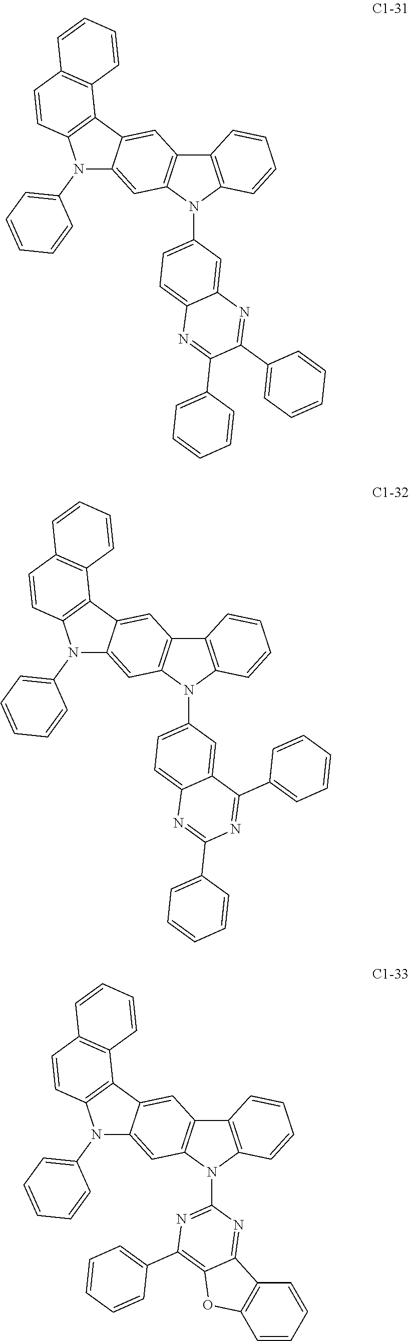 [SMILES: c1ccc(-c2nc3ccc(-n4c5ccccc5c5cc6c7c8ccccc8ccc7n(-c7ccccc7)c6cc54)cc3nc2-c2ccccc2)cc1 ; c1ccc(-c2nc(-c3ccccc3)c3cc(-n4c5ccccc5c5cc6c7c8ccccc8ccc7n(-c7ccccc7)c6cc54)ccc3n2)cc1 ; c1ccc(-c2nc(-n3c4ccccc4c4cc5c6c7ccccc7ccc6n(-c6ccccc6)c5cc43)nc3c2oc2ccccc23)cc1]

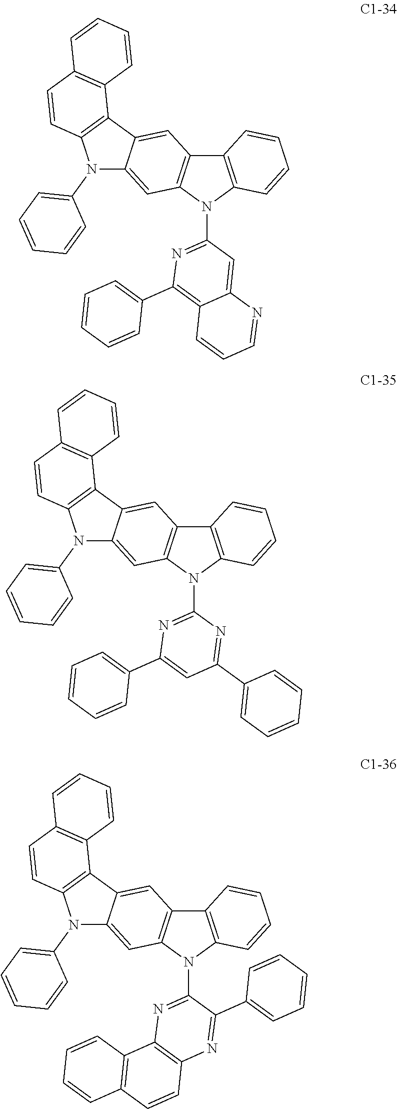 [SMILES: c1ccc(-c2nc(-n3c4ccccc4c4cc5c6c7ccccc7ccc6n(-c6ccccc6)c5cc43)cc3ncccc23)cc1 ; c1ccc(-c2cc(-c3ccccc3)nc(-n3c4ccccc4c4cc5c6c7ccccc7ccc6n(-c6ccccc6)c5cc43)n2)cc1 ; c1ccc(-c2nc3ccc4ccccc4c3nc2-n2c3ccccc3c3cc4c5c6ccccc6ccc5n(-c5ccccc5)c4cc32)cc1]

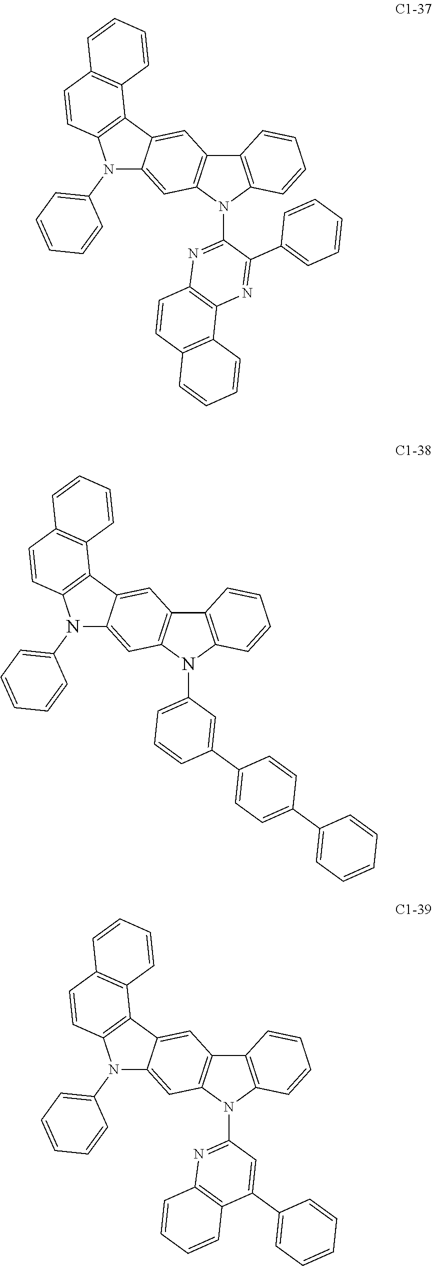 [SMILES: c1ccc(-c2nc3c(ccc4ccccc43)nc2-n2c3ccccc3c3cc4c5c6ccccc6ccc5n(-c5ccccc5)c4cc32)cc1 ; c1ccc(-c2ccc(-c3cccc(-n4c5ccccc5c5cc6c7c8ccccc8ccc7n(-c7ccccc7)c6cc54)c3)cc2)cc1 ; c1ccc(-c2cc(-n3c4ccccc4c4cc5c6c7ccccc7ccc6n(-c6ccccc6)c5cc43)nc3ccccc23)cc1]

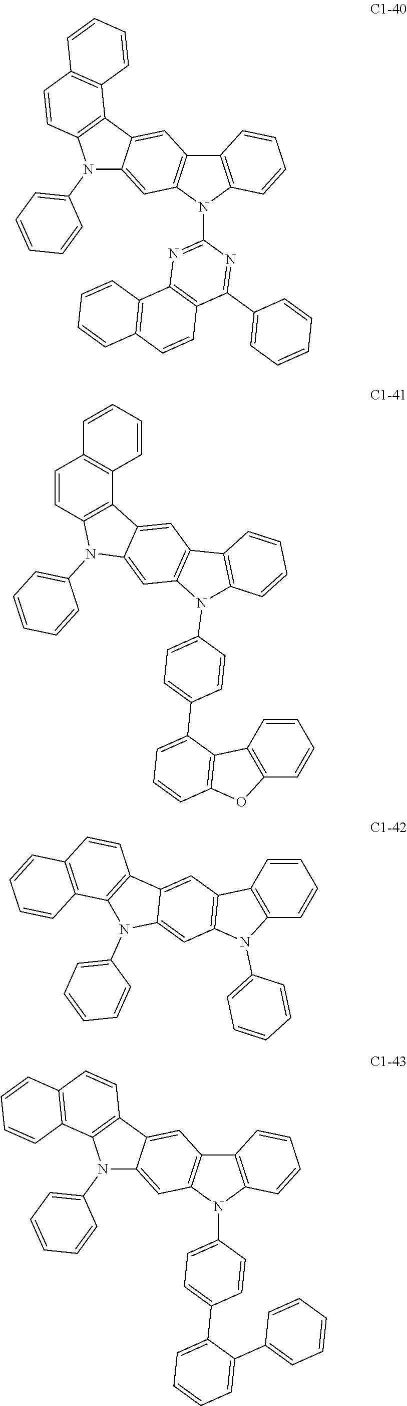 [SMILES: c1ccc(-c2nc(-n3c4ccccc4c4cc5c6c7ccccc7ccc6n(-c6ccccc6)c5cc43)nc3c2ccc2ccccc23)cc1 ; c1ccc(-n2c3cc4c(cc3c3c5ccccc5ccc32)c2ccccc2n4-c2ccc(-c3cccc4oc5ccccc5c34)cc2)cc1 ; c1ccc(-n2c3ccccc3c3cc4c5ccc6ccccc6c5n(-c5ccccc5)c4cc32)cc1 ; c1ccc(-c2ccccc2-c2ccc(-n3c4ccccc4c4cc5c6ccc7ccccc7c6n(-c6ccccc6)c5cc43)cc2)cc1]

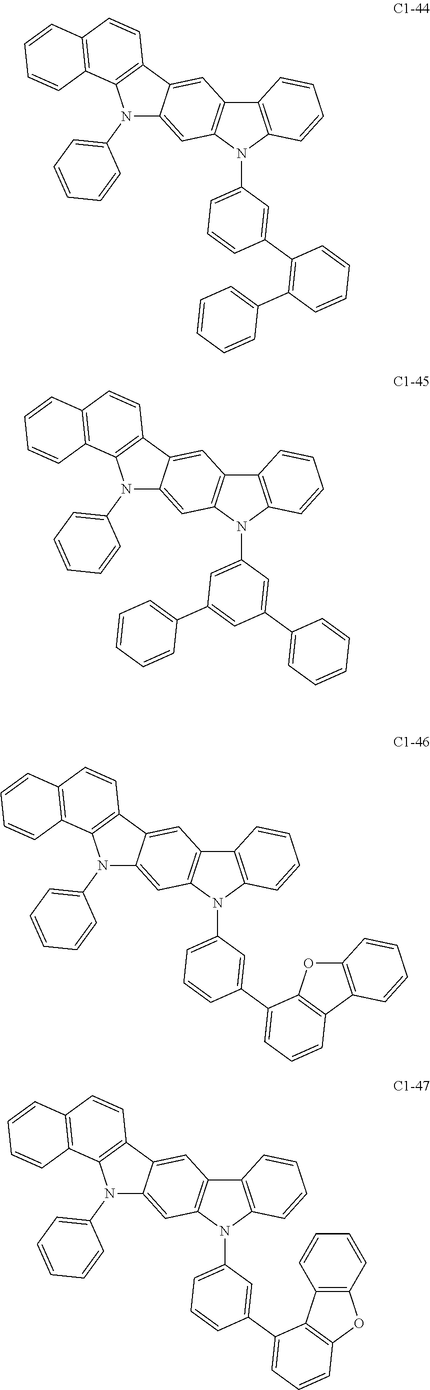 [SMILES: c1ccc(-c2ccccc2-c2cccc(-n3c4ccccc4c4cc5c6ccc7ccccc7c6n(-c6ccccc6)c5cc43)c2)cc1 ; c1ccc(-c2cc(-c3ccccc3)cc(-n3c4ccccc4c4cc5c6ccc7ccccc7c6n(-c6ccccc6)c5cc43)c2)cc1 ; c1ccc(-n2c3cc4c(cc3c3ccc5ccccc5c32)c2ccccc2n4-c2cccc(-c3cccc4c3oc3ccccc34)c2)cc1 ; c1ccc(-n2c3cc4c(cc3c3ccc5ccccc5c32)c2ccccc2n4-c2cccc(-c3cccc4oc5ccccc5c34)c2)cc1]

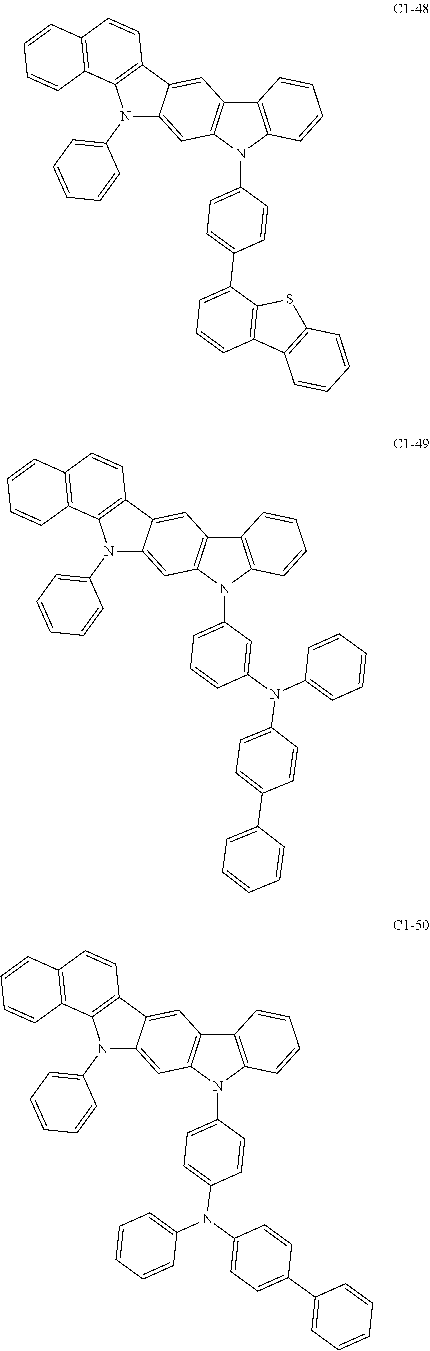 [SMILES: c1ccc(-n2c3cc4c(cc3c3ccc5ccccc5c32)c2ccccc2n4-c2ccc(-c3cccc4c3sc3ccccc34)cc2)cc1 ; c1ccc(-c2ccc(N(c3ccccc3)c3cccc(-n4c5ccccc5c5cc6c7ccc8ccccc8c7n(-c7ccccc7)c6cc54)c3)cc2)cc1 ; c1ccc(-c2ccc(N(c3ccccc3)c3ccc(-n4c5ccccc5c5cc6c7ccc8ccccc8c7n(-c7ccccc7)c6cc54)cc3)cc2)cc1]

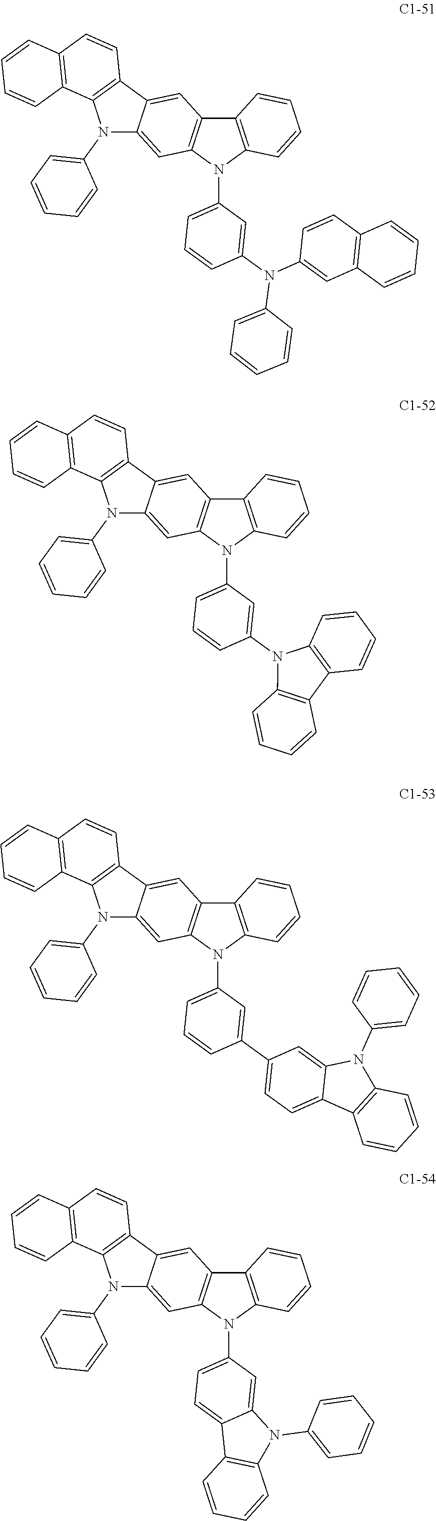 [SMILES: c1ccc(N(c2cccc(-n3c4ccccc4c4cc5c6ccc7ccccc7c6n(-c6ccccc6)c5cc43)c2)c2ccc3ccccc3c2)cc1 ; c1ccc(-n2c3cc4c(cc3c3ccc5ccccc5c32)c2ccccc2n4-c2cccc(-n3c4ccccc4c4ccccc43)c2)cc1 ; c1ccc(-n2c3ccccc3c3ccc(-c4cccc(-n5c6ccccc6c6cc7c8ccc9ccccc9c8n(-c8ccccc8)c7cc65)c4)cc32)cc1 ; c1ccc(-n2c3ccccc3c3ccc(-n4c5ccccc5c5cc6c7ccc8ccccc8c7n(-c7ccccc7)c6cc54)cc32)cc1]

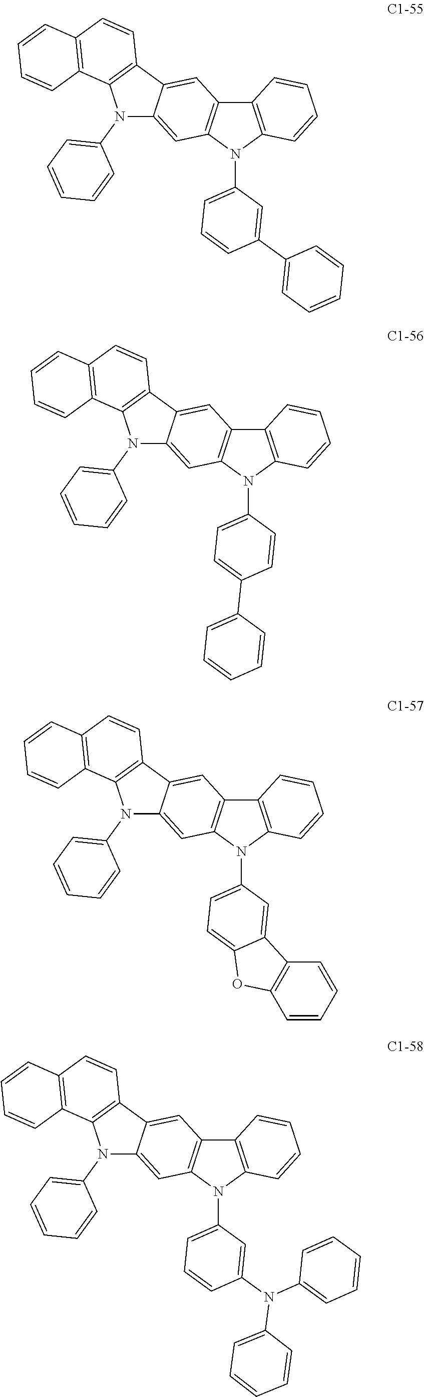 [SMILES: c1ccc(-c2cccc(-n3c4ccccc4c4cc5c6ccc7ccccc7c6n(-c6ccccc6)c5cc43)c2)cc1 ; c1ccc(-c2ccc(-n3c4ccccc4c4cc5c6ccc7ccccc7c6n(-c6ccccc6)c5cc43)cc2)cc1 ; c1ccc(-n2c3cc4c(cc3c3ccc5ccccc5c32)c2ccccc2n4-c2ccc3oc4ccccc4c3c2)cc1 ; c1ccc(N(c2ccccc2)c2cccc(-n3c4ccccc4c4cc5c6ccc7ccccc7c6n(-c6ccccc6)c5cc43)c2)cc1]

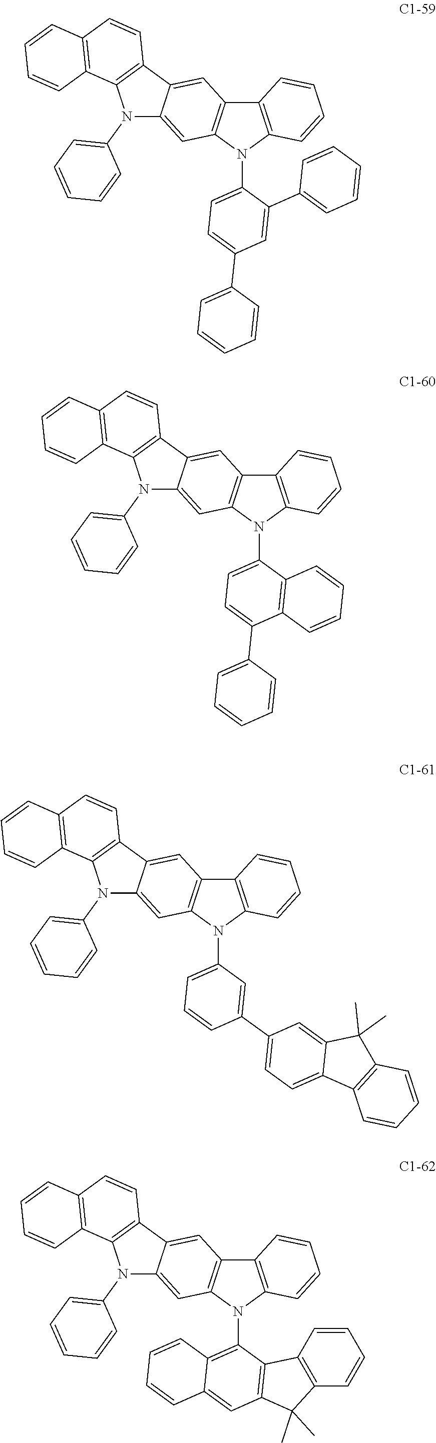 [SMILES: c1ccc(-c2ccc(-n3c4ccccc4c4cc5c6ccc7ccccc7c6n(-c6ccccc6)c5cc43)c(-c3ccccc3)c2)cc1 ; c1ccc(-c2ccc(-n3c4ccccc4c4cc5c6ccc7ccccc7c6n(-c6ccccc6)c5cc43)c3ccccc23)cc1 ; CC1(C)c2ccccc2-c2ccc(-c3cccc(-n4c5ccccc5c5cc6c7ccc8ccccc8c7n(-c7ccccc7)c6cc54)c3)cc21 ; CC1(C)c2ccccc2-c2c1cc1ccccc1c2-n1c2ccccc2c2cc3c4ccc5ccccc5c4n(-c4ccccc4)c3cc21]

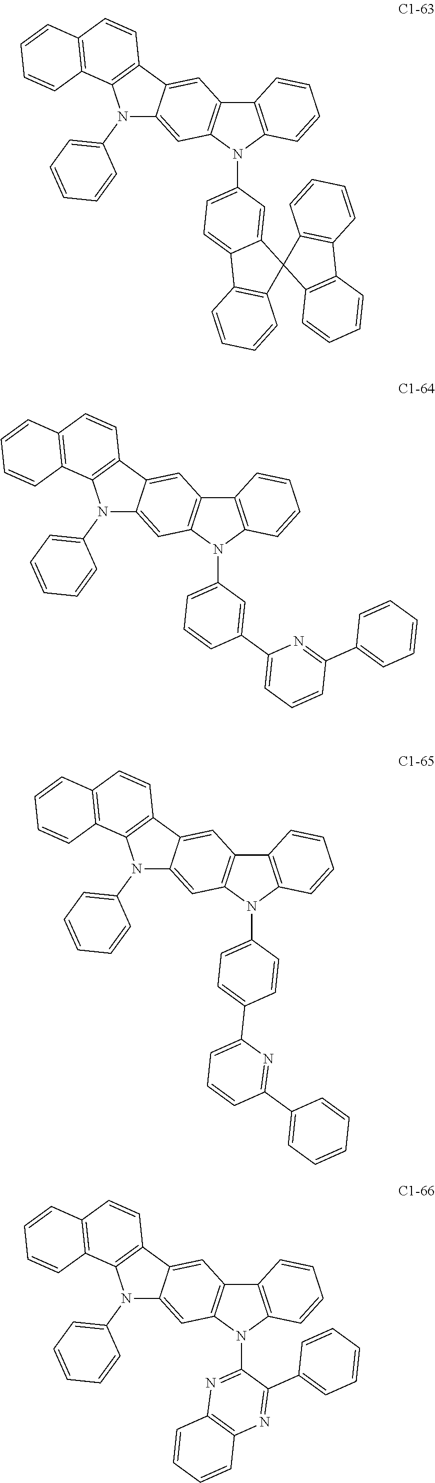 [SMILES: c1ccc(-n2c3cc4c(cc3c3ccc5ccccc5c32)c2ccccc2n4-c2ccc3c(c2)C2(c4ccccc4-c4ccccc42)c2ccccc2-3)cc1 ; c1ccc(-c2cccc(-c3cccc(-n4c5ccccc5c5cc6c7ccc8ccccc8c7n(-c7ccccc7)c6cc54)c3)n2)cc1 ; c1ccc(-c2cccc(-c3ccc(-n4c5ccccc5c5cc6c7ccc8ccccc8c7n(-c7ccccc7)c6cc54)cc3)n2)cc1 ; c1ccc(-c2nc3ccccc3nc2-n2c3ccccc3c3cc4c5ccc6ccccc6c5n(-c5ccccc5)c4cc32)cc1]

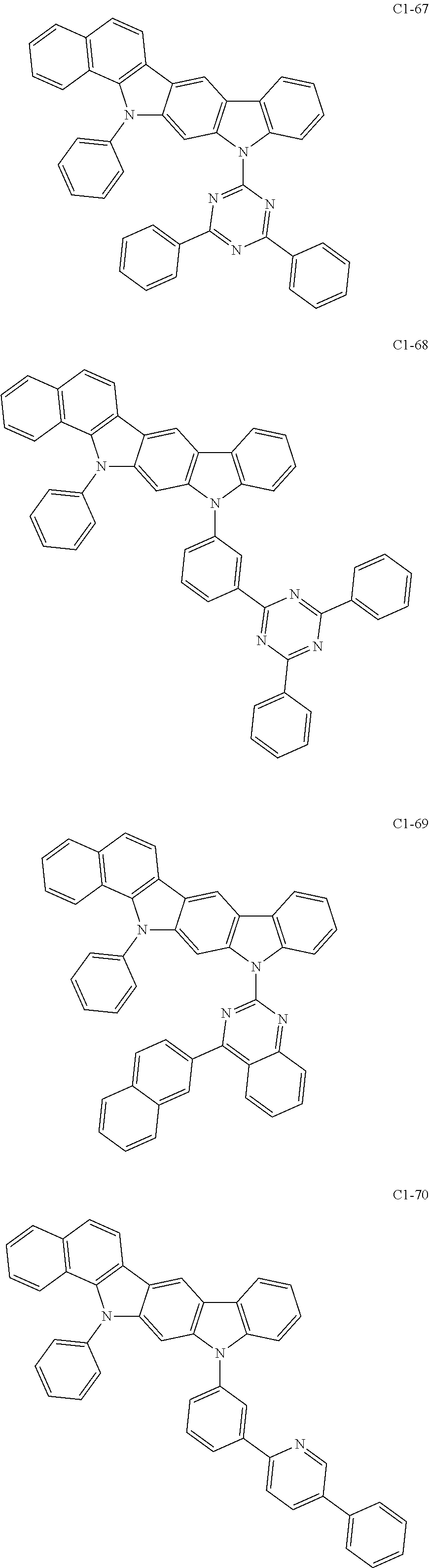 [SMILES: c1ccc(-c2nc(-c3ccccc3)nc(-n3c4ccccc4c4cc5c6ccc7ccccc7c6n(-c6ccccc6)c5cc43)n2)cc1 ; c1ccc(-c2nc(-c3ccccc3)nc(-c3cccc(-n4c5ccccc5c5cc6c7ccc8ccccc8c7n(-c7ccccc7)c6cc54)c3)n2)cc1 ; c1ccc(-n2c3cc4c(cc3c3ccc5ccccc5c32)c2ccccc2n4-c2nc(-c3ccc4ccccc4c3)c3ccccc3n2)cc1 ; c1ccc(-c2ccc(-c3cccc(-n4c5ccccc5c5cc6c7ccc8ccccc8c7n(-c7ccccc7)c6cc54)c3)nc2)cc1]

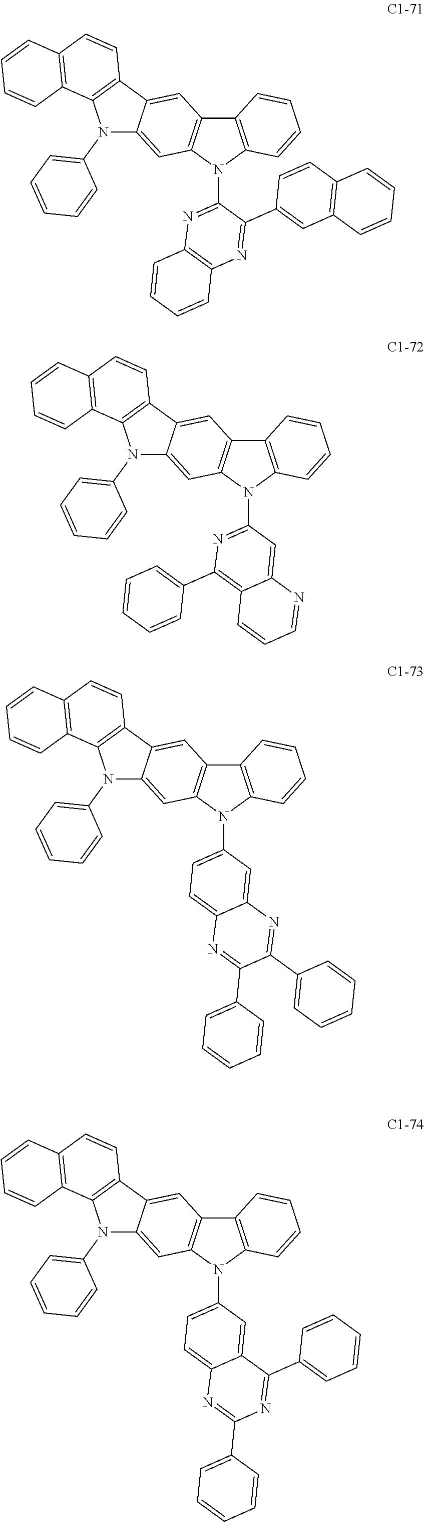 [SMILES: c1ccc(-n2c3cc4c(cc3c3ccc5ccccc5c32)c2ccccc2n4-c2nc3ccccc3nc2-c2ccc3ccccc3c2)cc1 ; c1ccc(-c2nc(-n3c4ccccc4c4cc5c6ccc7ccccc7c6n(-c6ccccc6)c5cc43)cc3ncccc23)cc1 ; c1ccc(-c2nc3ccc(-n4c5ccccc5c5cc6c7ccc8ccccc8c7n(-c7ccccc7)c6cc54)cc3nc2-c2ccccc2)cc1 ; c1ccc(-c2nc(-c3ccccc3)c3cc(-n4c5ccccc5c5cc6c7ccc8ccccc8c7n(-c7ccccc7)c6cc54)ccc3n2)cc1]

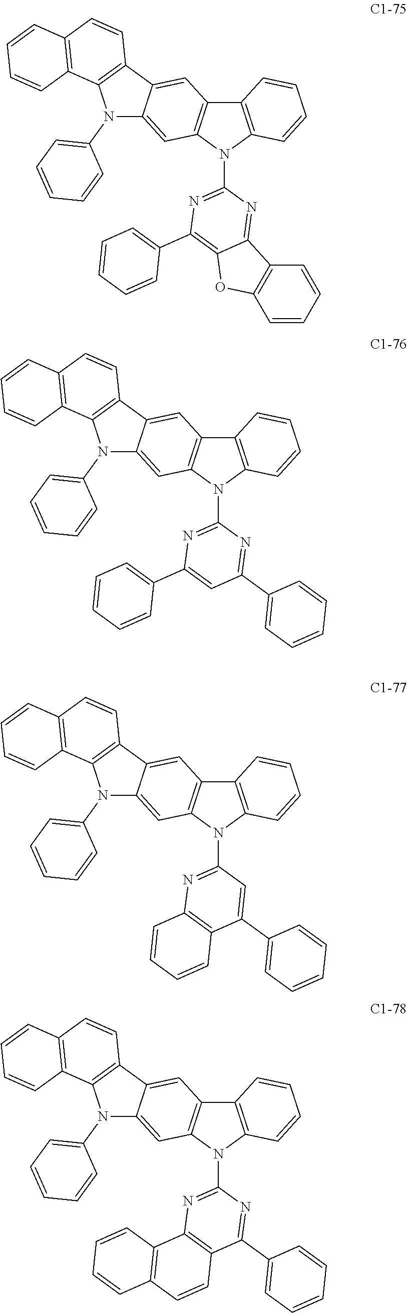 [SMILES: c1ccc(-c2nc(-n3c4ccccc4c4cc5c6ccc7ccccc7c6n(-c6ccccc6)c5cc43)nc3c2oc2ccccc23)cc1 ; c1ccc(-c2cc(-c3ccccc3)nc(-n3c4ccccc4c4cc5c6ccc7ccccc7c6n(-c6ccccc6)c5cc43)n2)cc1 ; c1ccc(-c2cc(-n3c4ccccc4c4cc5c6ccc7ccccc7c6n(-c6ccccc6)c5cc43)nc3ccccc23)cc1 ; c1ccc(-c2nc(-n3c4ccccc4c4cc5c6ccc7ccccc7c6n(-c6ccccc6)c5cc43)nc3c2ccc2ccccc23)cc1]

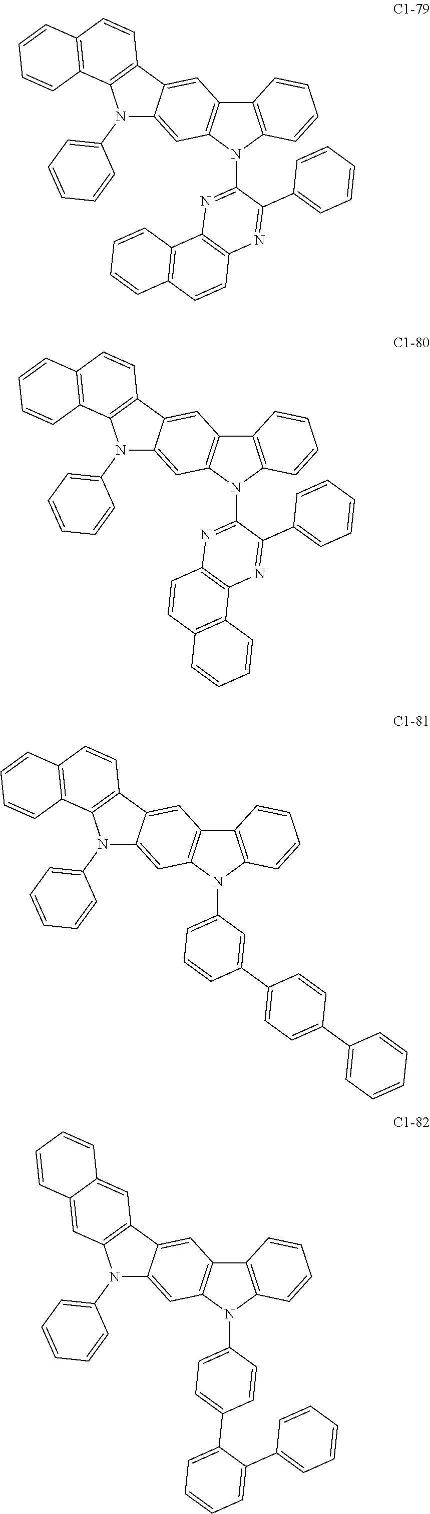 [SMILES: c1ccc(-c2nc3ccc4ccccc4c3nc2-n2c3ccccc3c3cc4c5ccc6ccccc6c5n(-c5ccccc5)c4cc32)cc1 ; c1ccc(-c2nc3c(ccc4ccccc43)nc2-n2c3ccccc3c3cc4c5ccc6ccccc6c5n(-c5ccccc5)c4cc32)cc1 ; c1ccc(-c2ccc(-c3cccc(-n4c5ccccc5c5cc6c7ccc8ccccc8c7n(-c7ccccc7)c6cc54)c3)cc2)cc1 ; c1ccc(-c2ccccc2-c2ccc(-n3c4ccccc4c4cc5c6cc7ccccc7cc6n(-c6ccccc6)c5cc43)cc2)cc1]

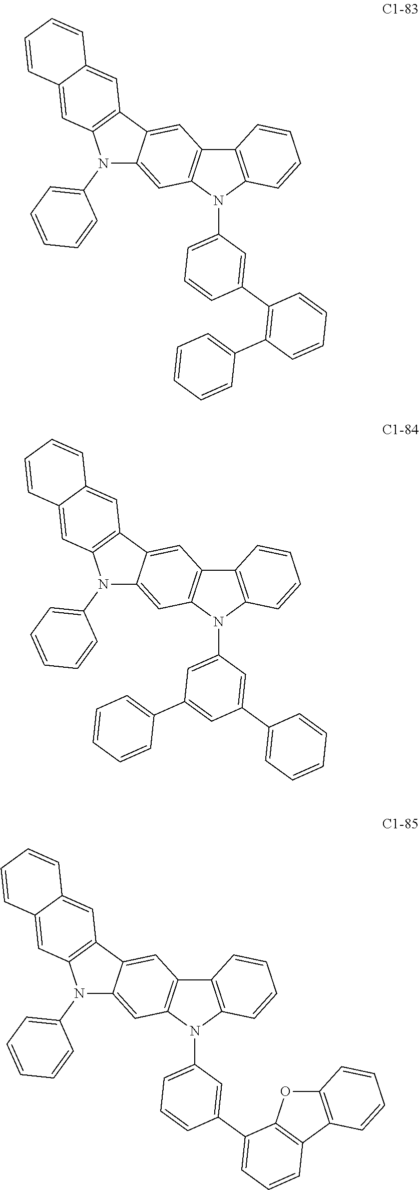 [SMILES: c1ccc(-c2ccccc2-c2cccc(-n3c4ccccc4c4cc5c6cc7ccccc7cc6n(-c6ccccc6)c5cc43)c2)cc1 ; c1ccc(-c2cc(-c3ccccc3)cc(-n3c4ccccc4c4cc5c6cc7ccccc7cc6n(-c6ccccc6)c5cc43)c2)cc1 ; c1ccc(-n2c3cc4ccccc4cc3c3cc4c5ccccc5n(-c5cccc(-c6cccc7c6oc6ccccc67)c5)c4cc32)cc1]

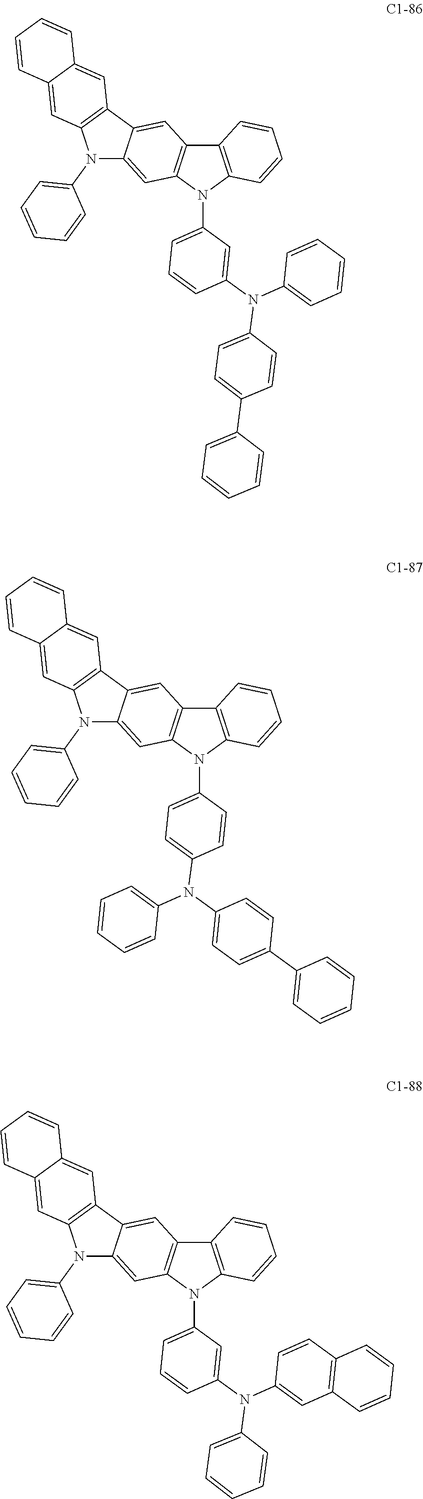 [SMILES: c1ccc(-c2ccc(N(c3ccccc3)c3cccc(-n4c5ccccc5c5cc6c7cc8ccccc8cc7n(-c7ccccc7)c6cc54)c3)cc2)cc1 ; c1ccc(-c2ccc(N(c3ccccc3)c3ccc(-n4c5ccccc5c5cc6c7cc8ccccc8cc7n(-c7ccccc7)c6cc54)cc3)cc2)cc1 ; c1ccc(N(c2cccc(-n3c4ccccc4c4cc5c6cc7ccccc7cc6n(-c6ccccc6)c5cc43)c2)c2ccc3ccccc3c2)cc1]

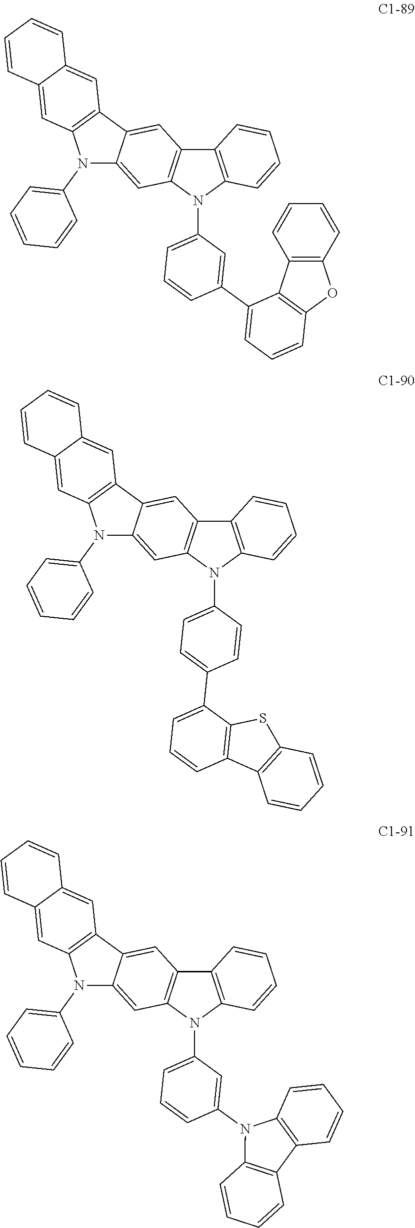 [SMILES: c1ccc(-n2c3cc4ccccc4cc3c3cc4c5ccccc5n(-c5cccc(-c6cccc7oc8ccccc8c67)c5)c4cc32)cc1 ; c1ccc(-n2c3cc4ccccc4cc3c3cc4c5ccccc5n(-c5ccc(-c6cccc7c6sc6ccccc67)cc5)c4cc32)cc1 ; c1ccc(-n2c3cc4ccccc4cc3c3cc4c5ccccc5n(-c5cccc(-n6c7ccccc7c7ccccc76)c5)c4cc32)cc1]

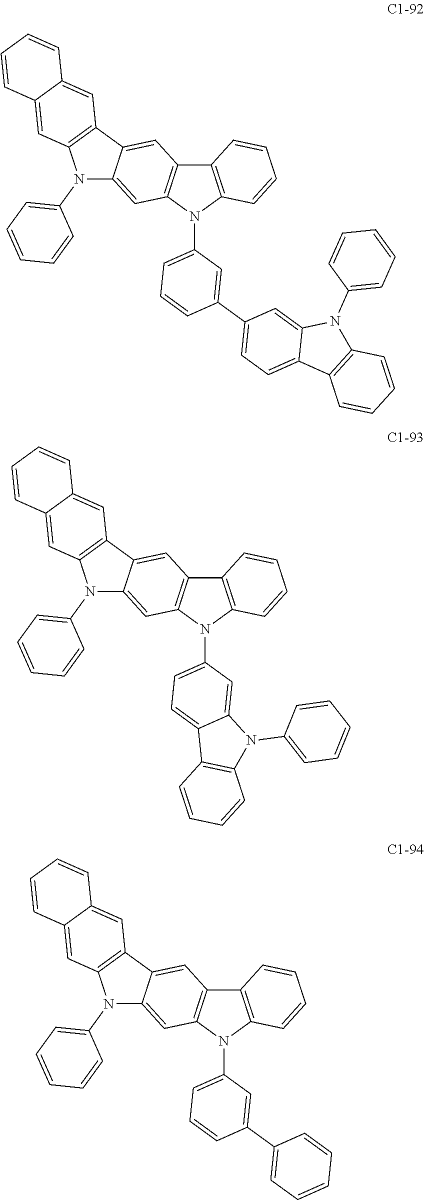 [SMILES: c1ccc(-n2c3ccccc3c3ccc(-c4cccc(-n5c6ccccc6c6cc7c8cc9ccccc9cc8n(-c8ccccc8)c7cc65)c4)cc32)cc1 ; c1ccc(-n2c3ccccc3c3ccc(-n4c5ccccc5c5cc6c7cc8ccccc8cc7n(-c7ccccc7)c6cc54)cc32)cc1 ; c1ccc(-c2cccc(-n3c4ccccc4c4cc5c6cc7ccccc7cc6n(-c6ccccc6)c5cc43)c2)cc1]

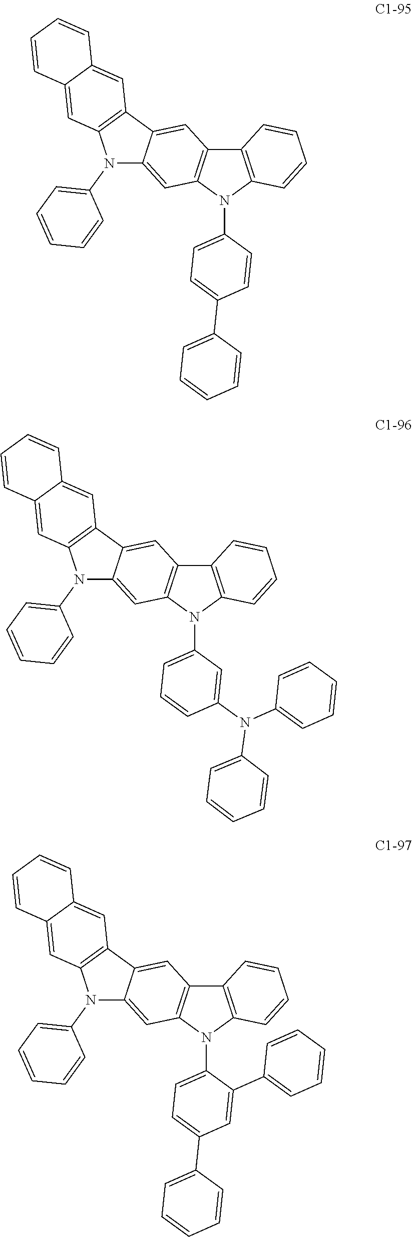 [SMILES: c1ccc(-c2ccc(-n3c4ccccc4c4cc5c6cc7ccccc7cc6n(-c6ccccc6)c5cc43)cc2)cc1 ; c1ccc(N(c2ccccc2)c2cccc(-n3c4ccccc4c4cc5c6cc7ccccc7cc6n(-c6ccccc6)c5cc43)c2)cc1 ; c1ccc(-c2ccc(-n3c4ccccc4c4cc5c6cc7ccccc7cc6n(-c6ccccc6)c5cc43)c(-c3ccccc3)c2)cc1]

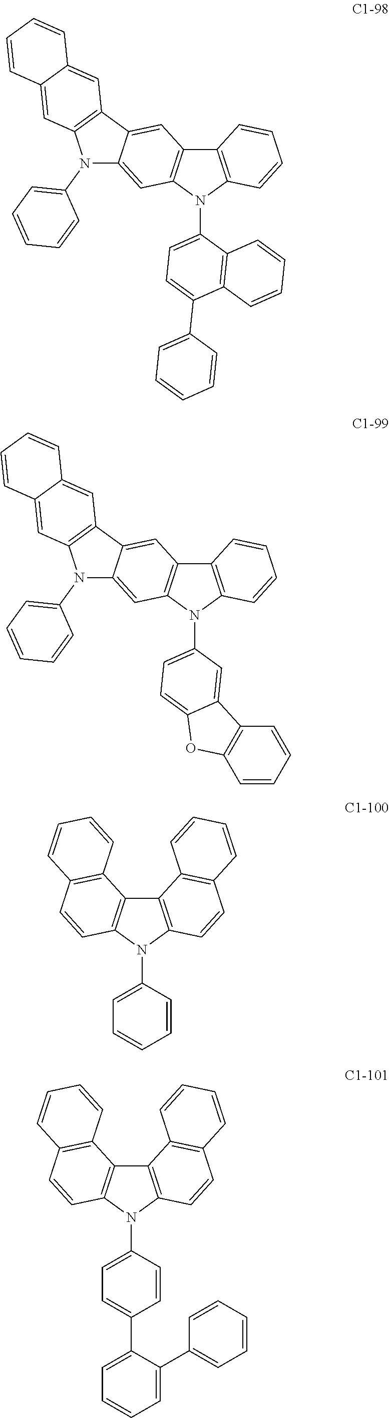

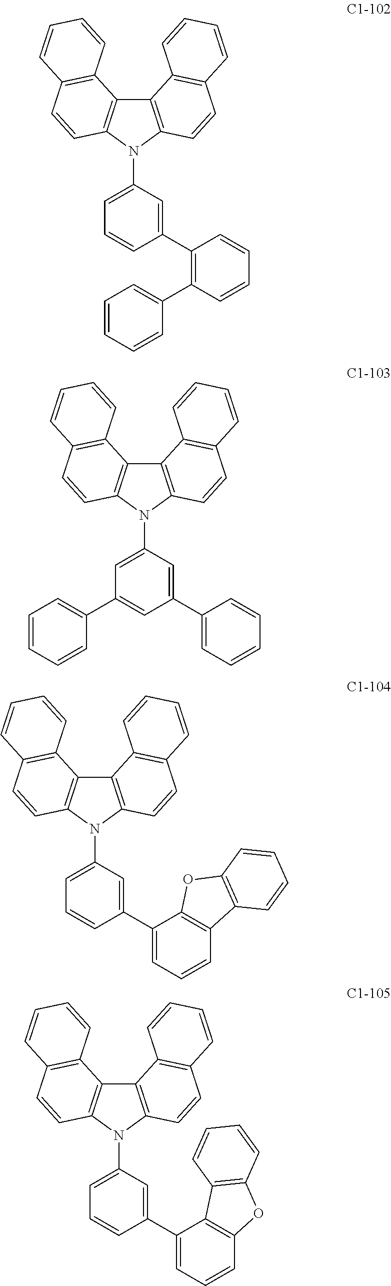

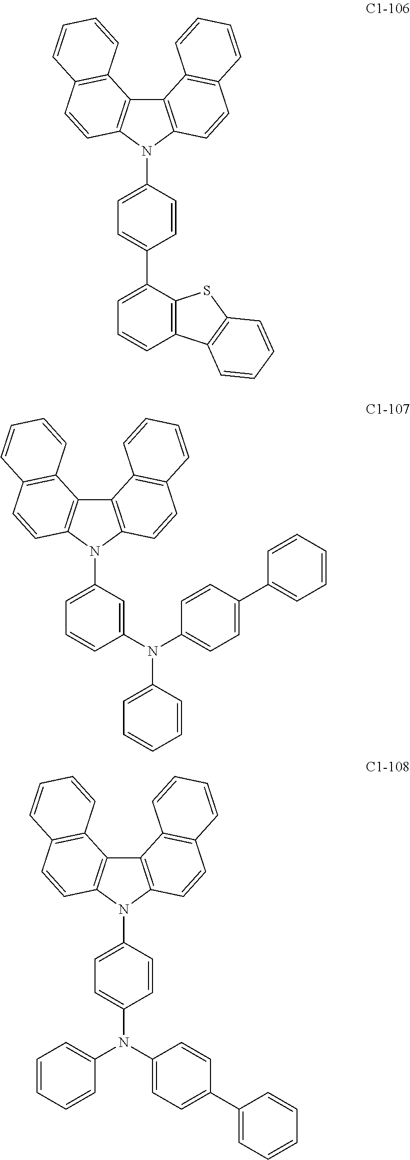

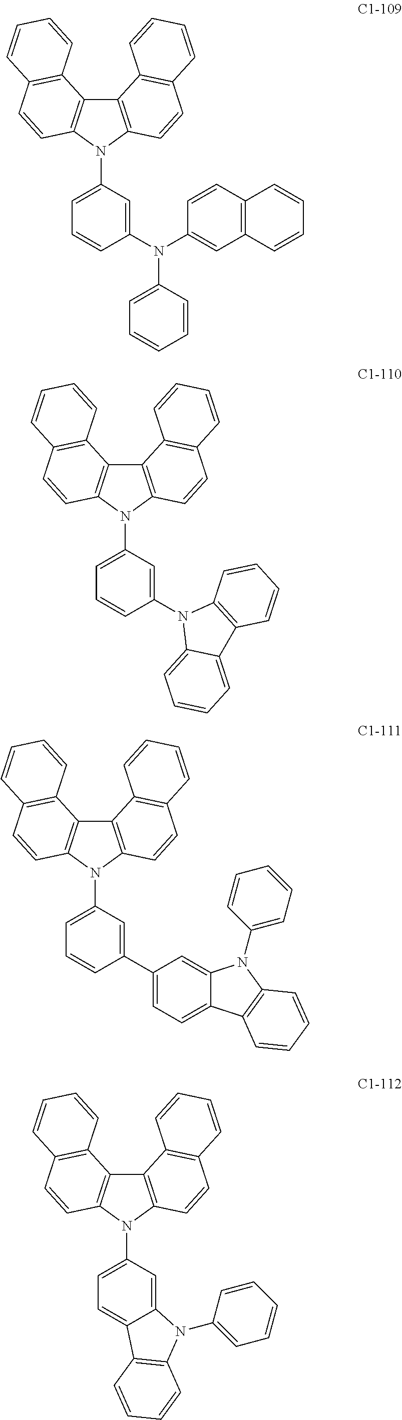

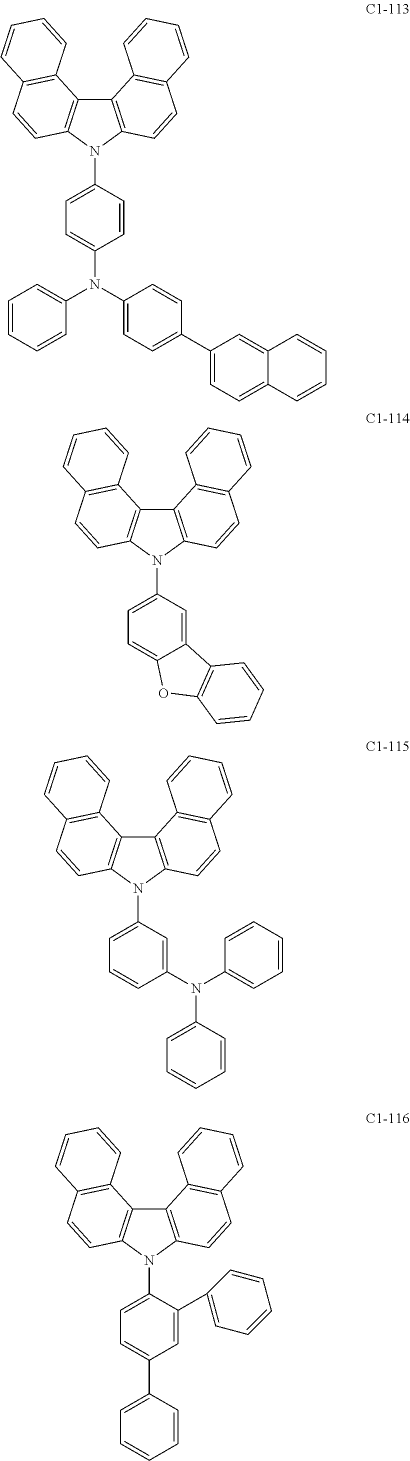

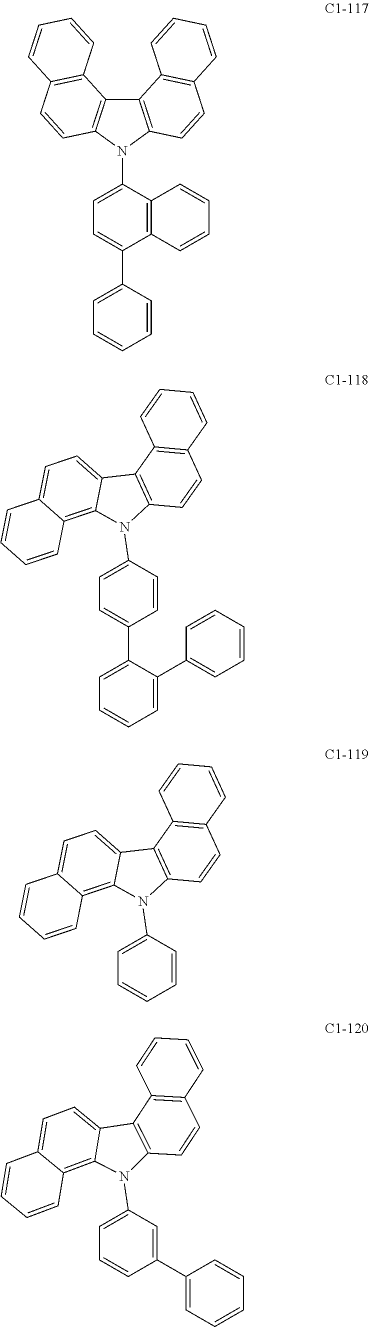

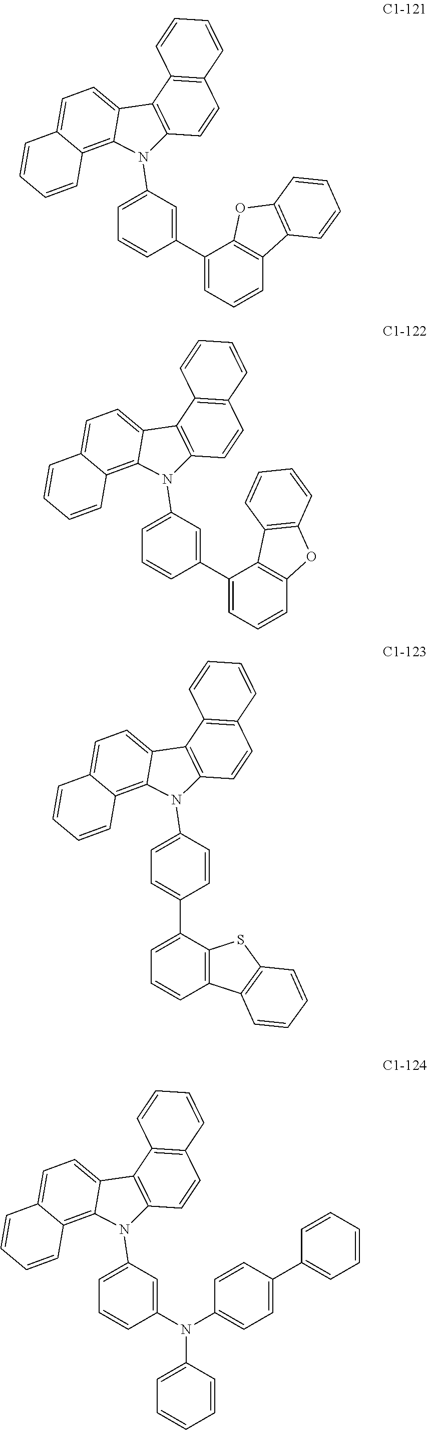

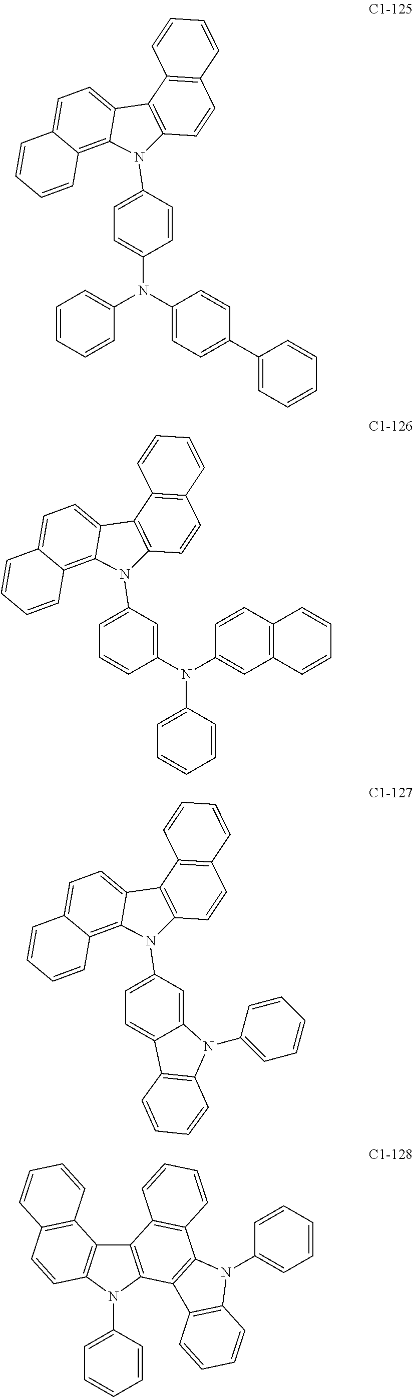

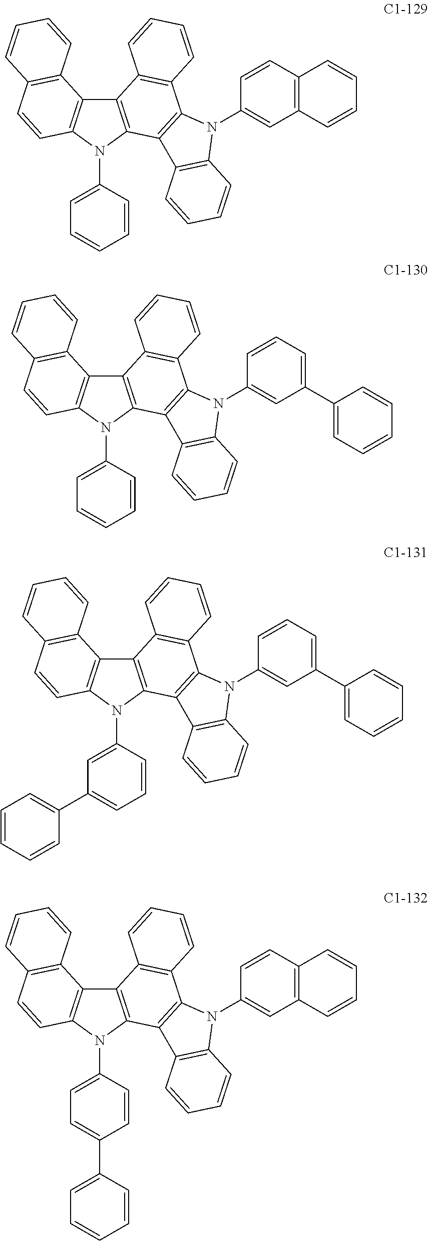 [SMILES: c1ccc(-n2c3ccc4ccccc4c3c3c4ccccc4c4c(c5ccccc5n4-c4ccc5ccccc5c4)c32)cc1 ; c1ccc(-c2cccc(-n3c4ccccc4c4c3c3ccccc3c3c5c6ccccc6ccc5n(-c5ccccc5)c34)c2)cc1 ; c1ccc(-c2cccc(-n3c4ccccc4c4c3c3ccccc3c3c5c6ccccc6ccc5n(-c5cccc(-c6ccccc6)c5)c34)c2)cc1 ; c1ccc(-c2ccc(-n3c4ccc5ccccc5c4c4c5ccccc5c5c(c6ccccc6n5-c5ccc6ccccc6c5)c43)cc2)cc1]

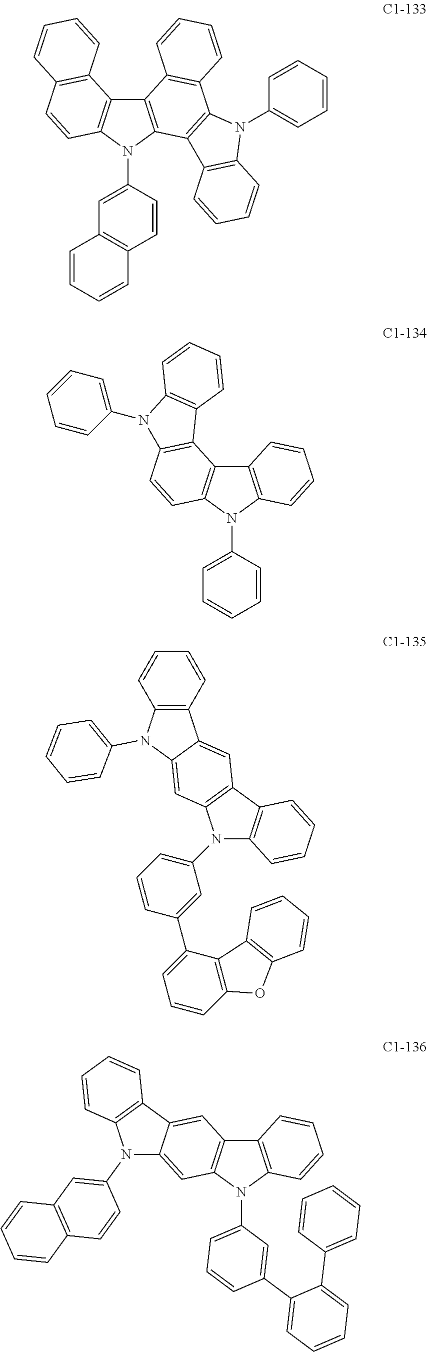 [SMILES: c1ccc(-n2c3ccccc3c3c2c2ccccc2c2c4c5ccccc5ccc4n(-c4ccc5ccccc5c4)c23)cc1 ; c1ccc(-n2c3ccccc3c3c4c5ccccc5n(-c5ccccc5)c4ccc32)cc1 ; c1ccc(-n2c3ccccc3c3cc4c5ccccc5n(-c5cccc(-c6cccc7oc8ccccc8c67)c5)c4cc32)cc1 ; c1ccc(-c2ccccc2-c2cccc(-n3c4ccccc4c4cc5c6ccccc6n(-c6ccc7ccccc7c6)c5cc43)c2)cc1]

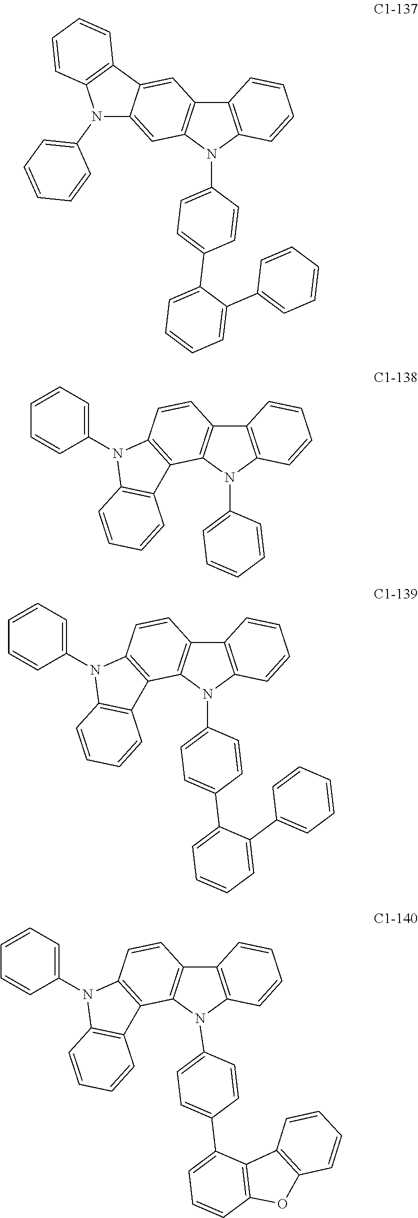

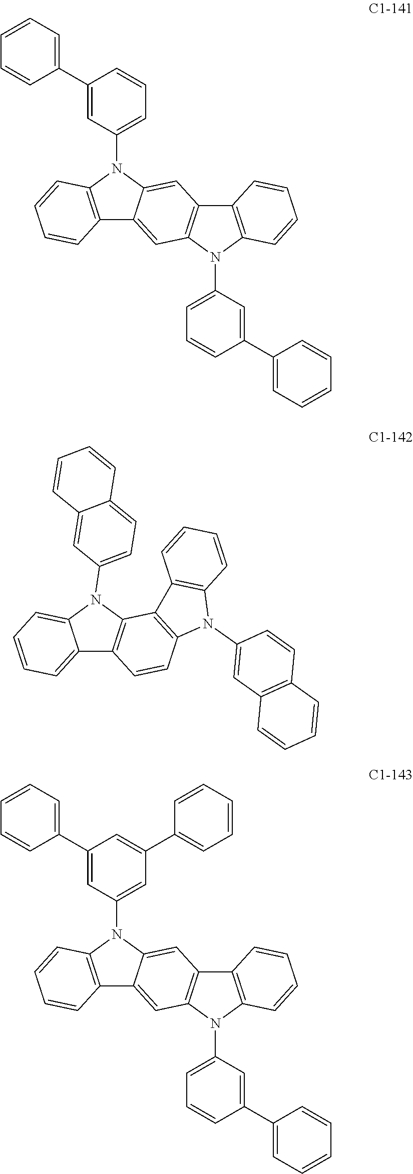 [SMILES: c1ccc(-c2cccc(-n3c4ccccc4c4cc5c(cc43)c3ccccc3n5-c3cccc(-c4ccccc4)c3)c2)cc1 ; c1ccc2cc(-n3c4ccccc4c4c3ccc3c5ccccc5n(-c5ccc6ccccc6c5)c34)ccc2c1 ; c1ccc(-c2cccc(-n3c4ccccc4c4cc5c(cc43)c3ccccc3n5-c3cc(-c4ccccc4)cc(-c4ccccc4)c3)c2)cc1]

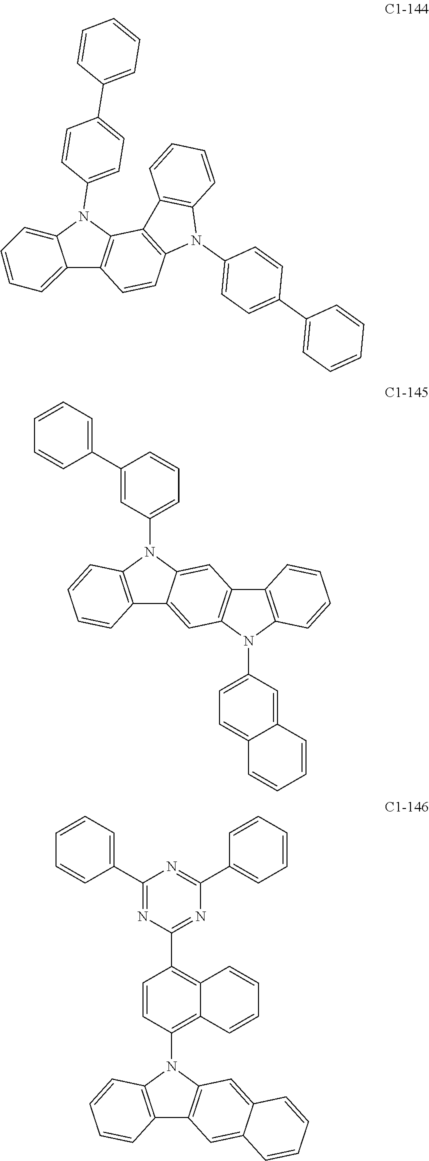

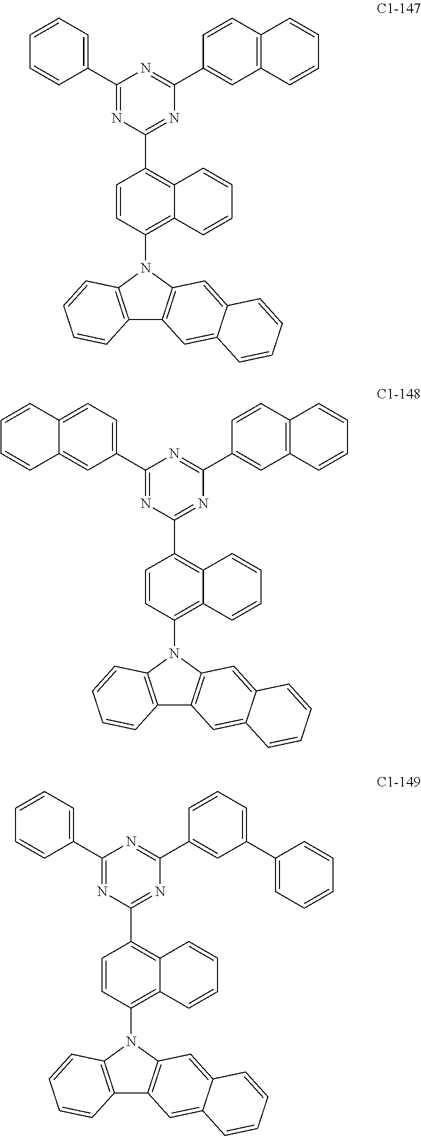 [SMILES: c1ccc(-c2nc(-c3ccc4ccccc4c3)nc(-c3ccc(-n4c5ccccc5c5cc6ccccc6cc54)c4ccccc34)n2)cc1 ; c1ccc2cc(-c3nc(-c4ccc5ccccc5c4)nc(-c4ccc(-n5c6ccccc6c6cc7ccccc7cc65)c5ccccc45)n3)ccc2c1 ; c1ccc(-c2cccc(-c3nc(-c4ccccc4)nc(-c4ccc(-n5c6ccccc6c6cc7ccccc7cc65)c5ccccc45)n3)c2)cc1]

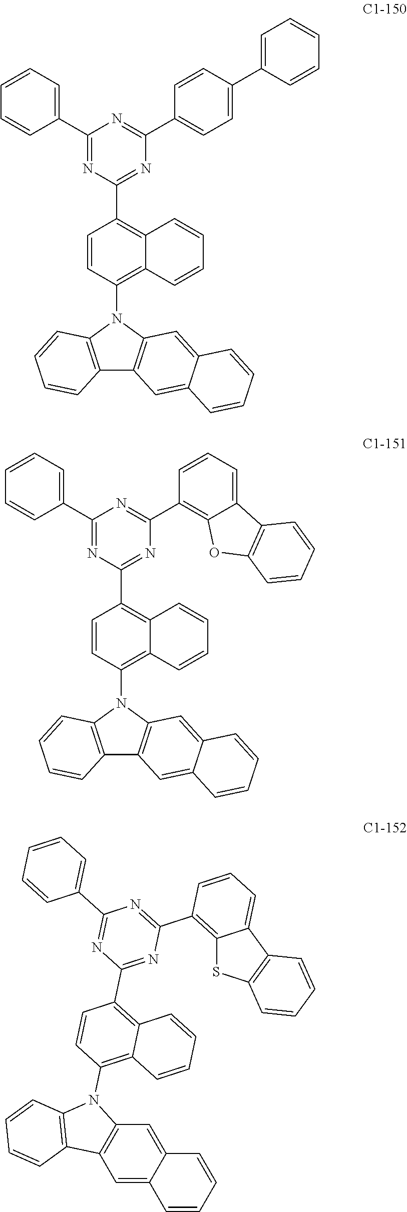

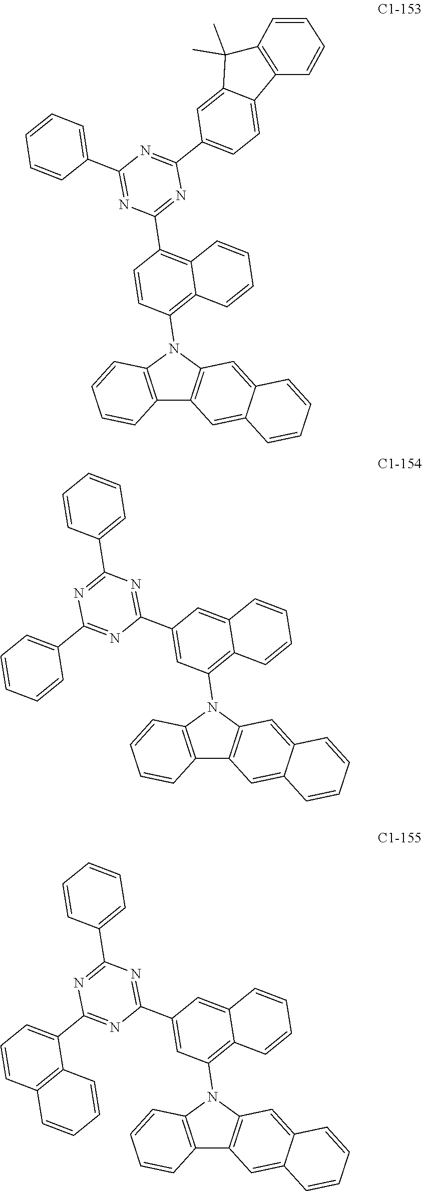 [SMILES: CC1(C)c2ccccc2-c2ccc(-c3nc(-c4ccccc4)nc(-c4ccc(-n5c6ccccc6c6cc7ccccc7cc65)c5ccccc45)n3)cc21 ; c1ccc(-c2nc(-c3ccccc3)nc(-c3cc(-n4c5ccccc5c5cc6ccccc6cc54)c4ccccc4c3)n2)cc1 ; c1ccc(-c2nc(-c3cc(-n4c5ccccc5c5cc6ccccc6cc54)c4ccccc4c3)nc(-c3cccc4ccccc34)n2)cc1]

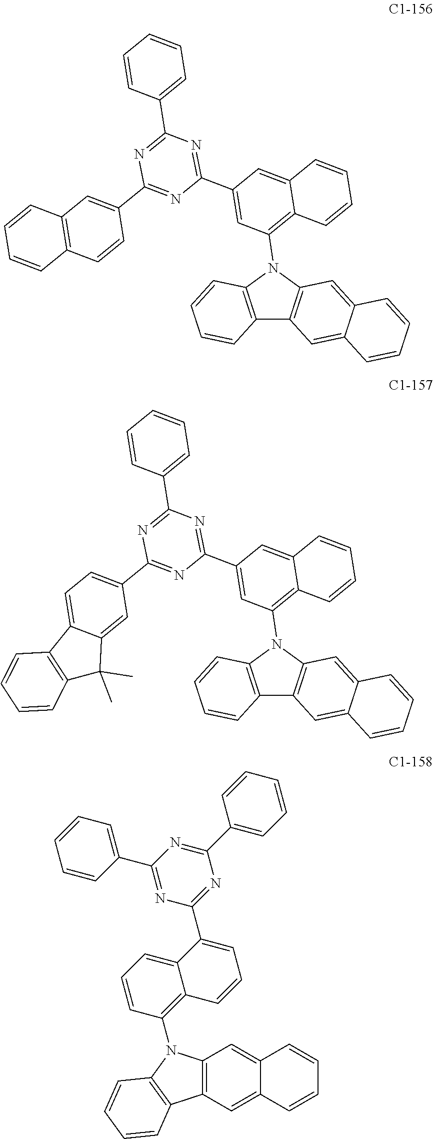 [SMILES: c1ccc(-c2nc(-c3ccc4ccccc4c3)nc(-c3cc(-n4c5ccccc5c5cc6ccccc6cc54)c4ccccc4c3)n2)cc1 ; CC1(C)c2ccccc2-c2ccc(-c3nc(-c4ccccc4)nc(-c4cc(-n5c6ccccc6c6cc7ccccc7cc65)c5ccccc5c4)n3)cc21 ; c1ccc(-c2nc(-c3ccccc3)nc(-c3cccc4c(-n5c6ccccc6c6cc7ccccc7cc65)cccc34)n2)cc1]

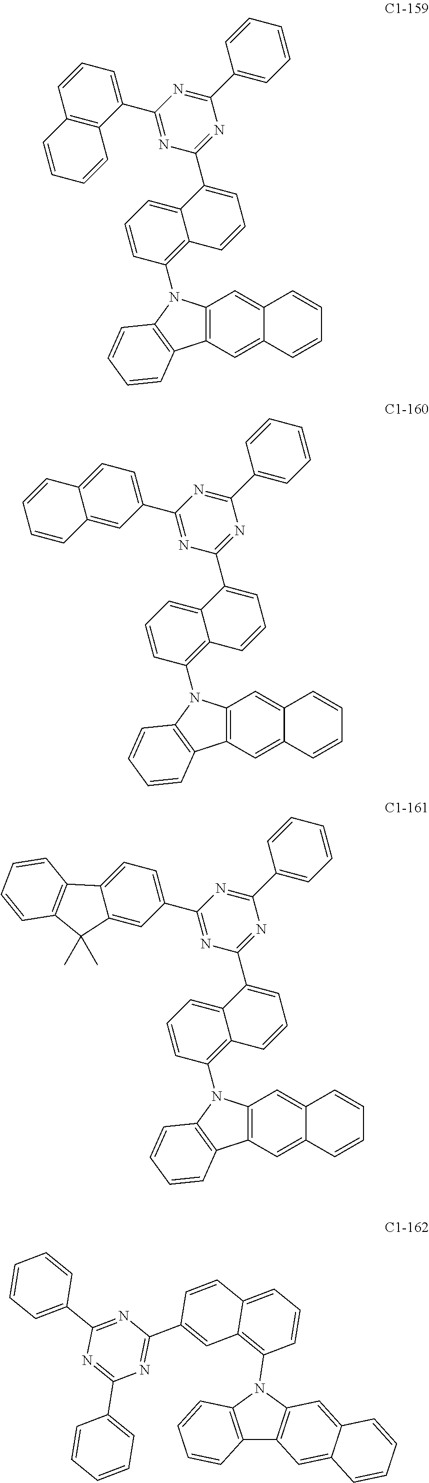 [SMILES: c1ccc(-c2nc(-c3cccc4ccccc34)nc(-c3cccc4c(-n5c6ccccc6c6cc7ccccc7cc65)cccc34)n2)cc1 ; c1ccc(-c2nc(-c3ccc4ccccc4c3)nc(-c3cccc4c(-n5c6ccccc6c6cc7ccccc7cc65)cccc34)n2)cc1 ; CC1(C)c2ccccc2-c2ccc(-c3nc(-c4ccccc4)nc(-c4cccc5c(-n6c7ccccc7c7cc8ccccc8cc76)cccc45)n3)cc21 ; c1ccc(-c2nc(-c3ccccc3)nc(-c3ccc4cccc(-n5c6ccccc6c6cc7ccccc7cc65)c4c3)n2)cc1]

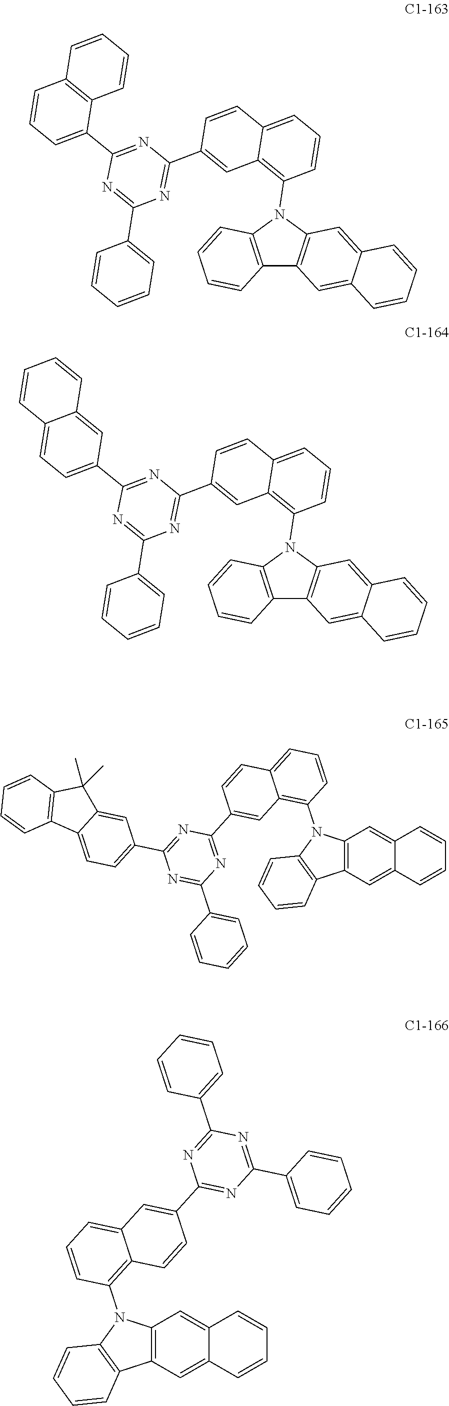 [SMILES: c1ccc(-c2nc(-c3ccc4cccc(-n5c6ccccc6c6cc7ccccc7cc65)c4c3)nc(-c3cccc4ccccc34)n2)cc1 ; c1ccc(-c2nc(-c3ccc4ccccc4c3)nc(-c3ccc4cccc(-n5c6ccccc6c6cc7ccccc7cc65)c4c3)n2)cc1 ; CC1(C)c2ccccc2-c2ccc(-c3nc(-c4ccccc4)nc(-c4ccc5cccc(-n6c7ccccc7c7cc8ccccc8cc76)c5c4)n3)cc21 ; c1ccc(-c2nc(-c3ccccc3)nc(-c3ccc4c(-n5c6ccccc6c6cc7ccccc7cc65)cccc4c3)n2)cc1]

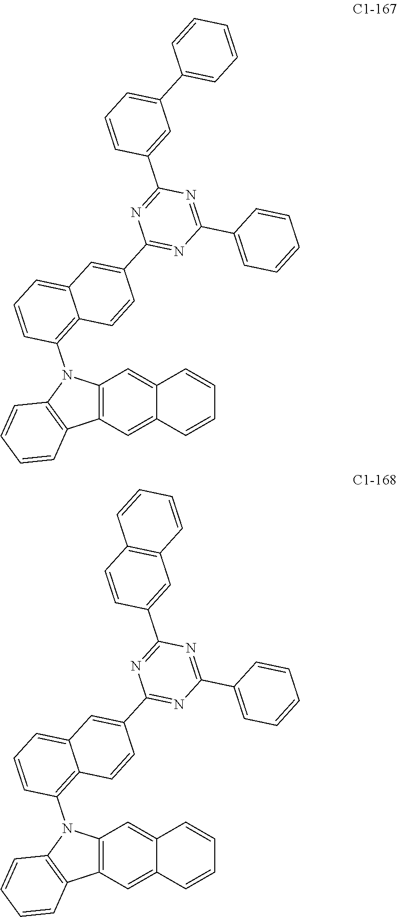 [SMILES: c1ccc(-c2cccc(-c3nc(-c4ccccc4)nc(-c4ccc5c(-n6c7ccccc7c7cc8ccccc8cc76)cccc5c4)n3)c2)cc1 ; c1ccc(-c2nc(-c3ccc4ccccc4c3)nc(-c3ccc4c(-n5c6ccccc6c6cc7ccccc7cc65)cccc4c3)n2)cc1]

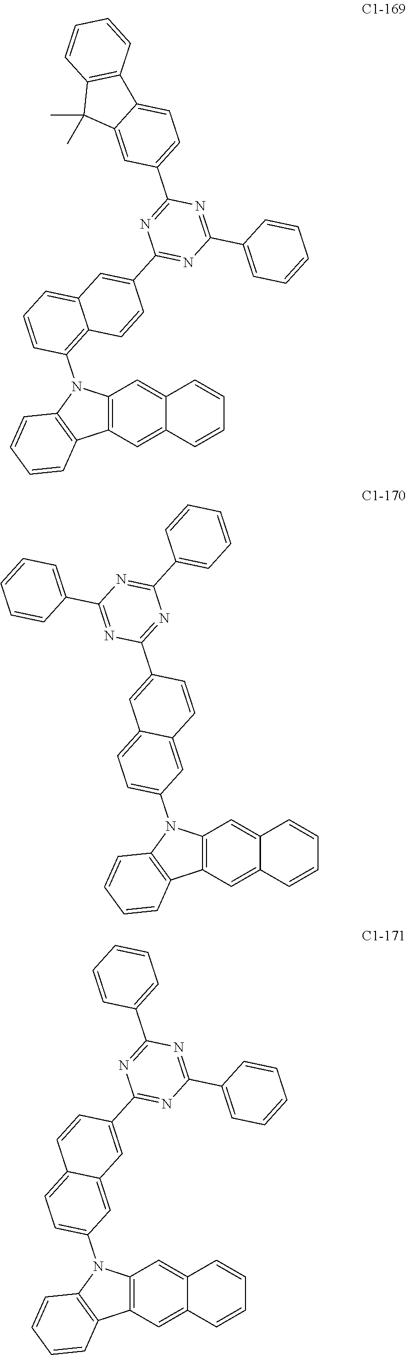

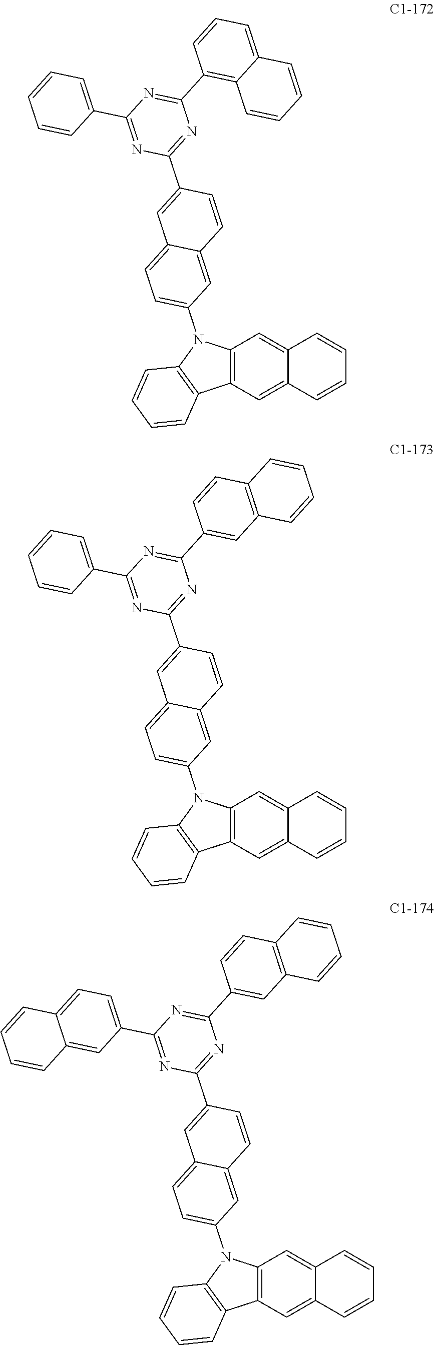 [SMILES: c1ccc(-c2nc(-c3ccc4cc(-n5c6ccccc6c6cc7ccccc7cc65)ccc4c3)nc(-c3cccc4ccccc34)n2)cc1 ; c1ccc(-c2nc(-c3ccc4ccccc4c3)nc(-c3ccc4cc(-n5c6ccccc6c6cc7ccccc7cc65)ccc4c3)n2)cc1 ; c1ccc2cc(-c3nc(-c4ccc5ccccc5c4)nc(-c4ccc5cc(-n6c7ccccc7c7cc8ccccc8cc76)ccc5c4)n3)ccc2c1]

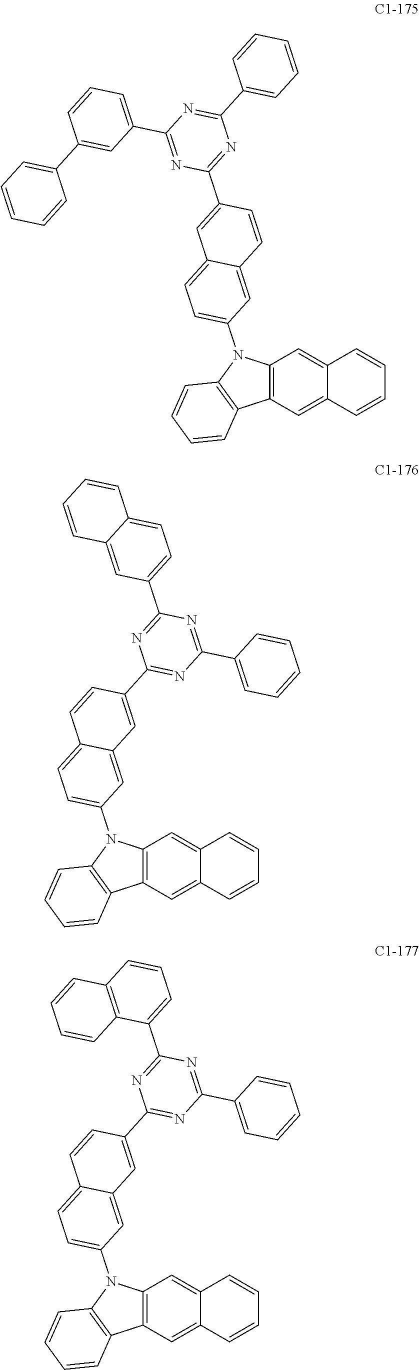 [SMILES: c1ccc(-c2cccc(-c3nc(-c4ccccc4)nc(-c4ccc5cc(-n6c7ccccc7c7cc8ccccc8cc76)ccc5c4)n3)c2)cc1 ; c1ccc(-c2nc(-c3ccc4ccccc4c3)nc(-c3ccc4ccc(-n5c6ccccc6c6cc7ccccc7cc65)cc4c3)n2)cc1 ; c1ccc(-c2nc(-c3ccc4ccc(-n5c6ccccc6c6cc7ccccc7cc65)cc4c3)nc(-c3cccc4ccccc34)n2)cc1]

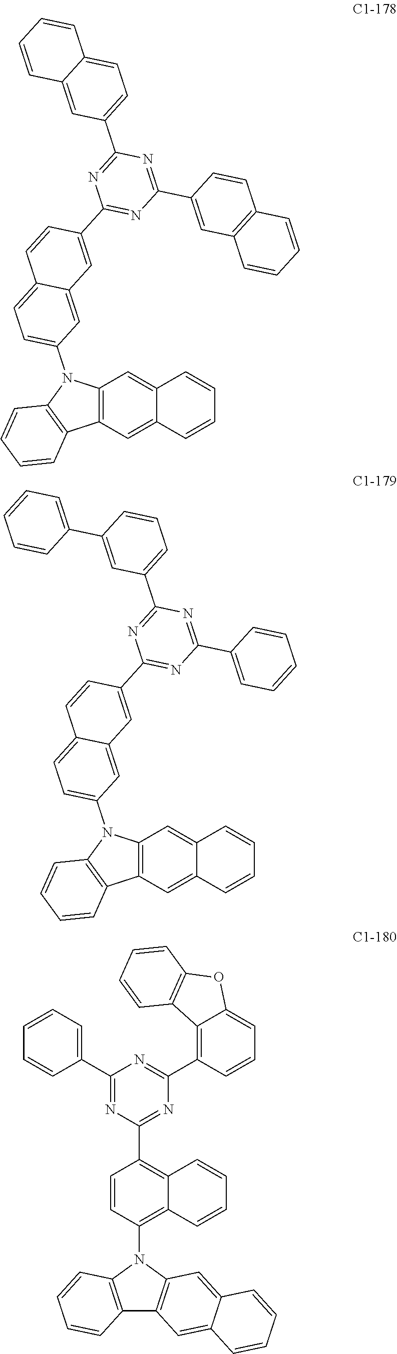

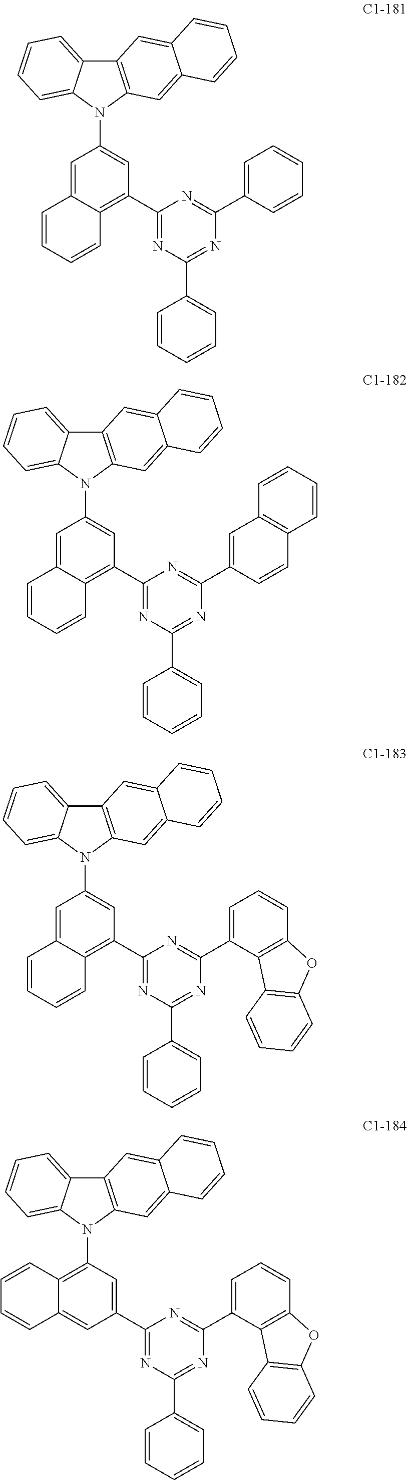

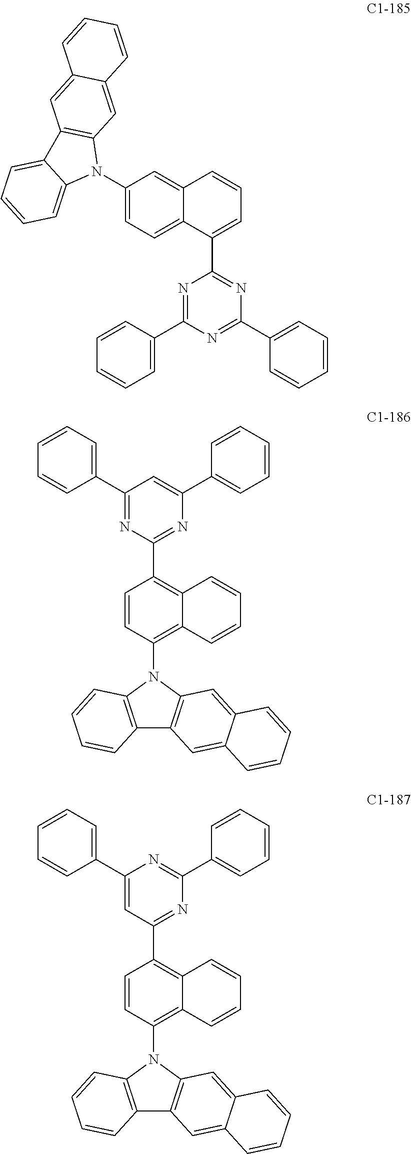

The compound represented by formula 2 may be specifically exemplified by the following compounds, but is not limited thereto.

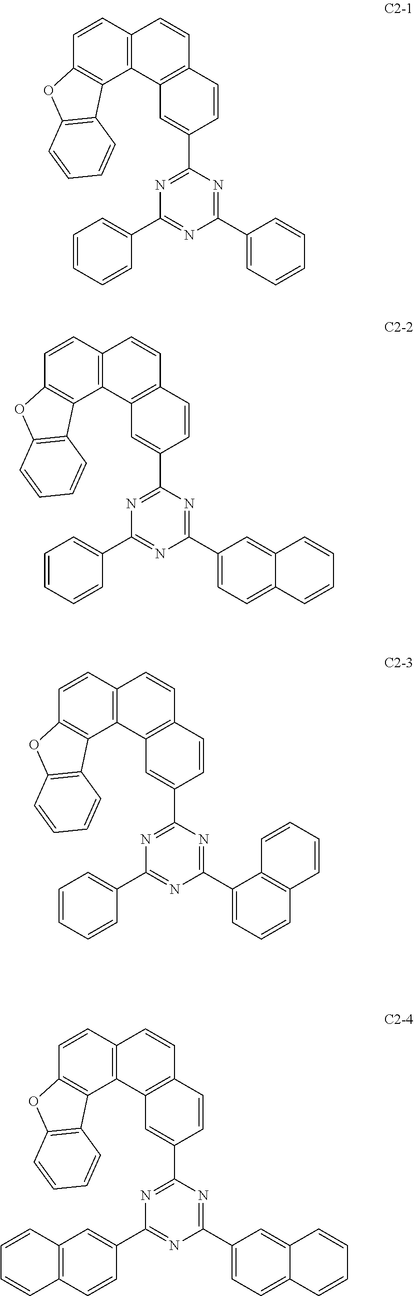 [SMILES: c1ccc(-c2nc(-c3ccccc3)nc(-c3ccc4ccc5ccc6oc7ccccc7c6c5c4c3)n2)cc1 ; c1ccc(-c2nc(-c3ccc4ccccc4c3)nc(-c3ccc4ccc5ccc6oc7ccccc7c6c5c4c3)n2)cc1 ; c1ccc(-c2nc(-c3ccc4ccc5ccc6oc7ccccc7c6c5c4c3)nc(-c3cccc4ccccc34)n2)cc1 ; c1ccc2cc(-c3nc(-c4ccc5ccccc5c4)nc(-c4ccc5ccc6ccc7oc8ccccc8c7c6c5c4)n3)ccc2c1]

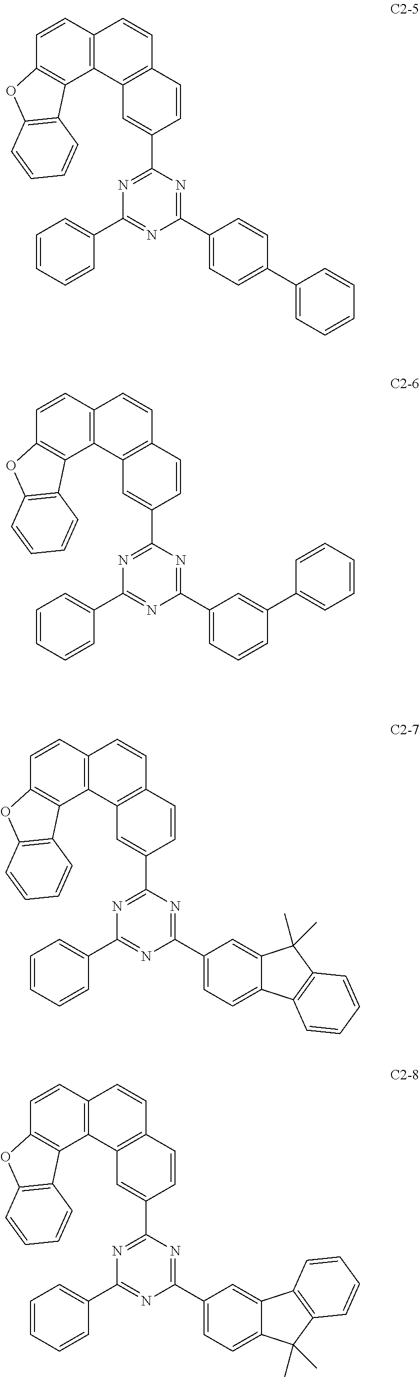 [SMILES: c1ccc(-c2ccc(-c3nc(-c4ccccc4)nc(-c4ccc5ccc6ccc7oc8ccccc8c7c6c5c4)n3)cc2)cc1 ; c1ccc(-c2cccc(-c3nc(-c4ccccc4)nc(-c4ccc5ccc6ccc7oc8ccccc8c7c6c5c4)n3)c2)cc1 ; CC1(C)c2ccccc2-c2ccc(-c3nc(-c4ccccc4)nc(-c4ccc5ccc6ccc7oc8ccccc8c7c6c5c4)n3)cc21 ; CC1(C)c2ccccc2-c2cc(-c3nc(-c4ccccc4)nc(-c4ccc5ccc6ccc7oc8ccccc8c7c6c5c4)n3)ccc21]

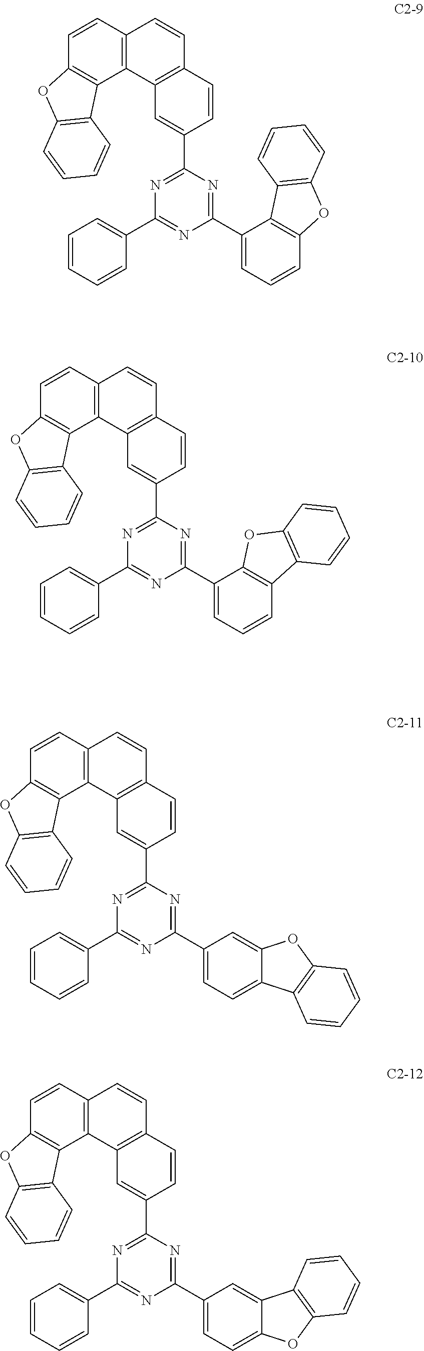 [SMILES: c1ccc(-c2nc(-c3ccc4ccc5ccc6oc7ccccc7c6c5c4c3)nc(-c3cccc4oc5ccccc5c34)n2)cc1 ; c1ccc(-c2nc(-c3ccc4ccc5ccc6oc7ccccc7c6c5c4c3)nc(-c3cccc4c3oc3ccccc34)n2)cc1 ; c1ccc(-c2nc(-c3ccc4c(c3)oc3ccccc34)nc(-c3ccc4ccc5ccc6oc7ccccc7c6c5c4c3)n2)cc1 ; c1ccc(-c2nc(-c3ccc4oc5ccccc5c4c3)nc(-c3ccc4ccc5ccc6oc7ccccc7c6c5c4c3)n2)cc1]

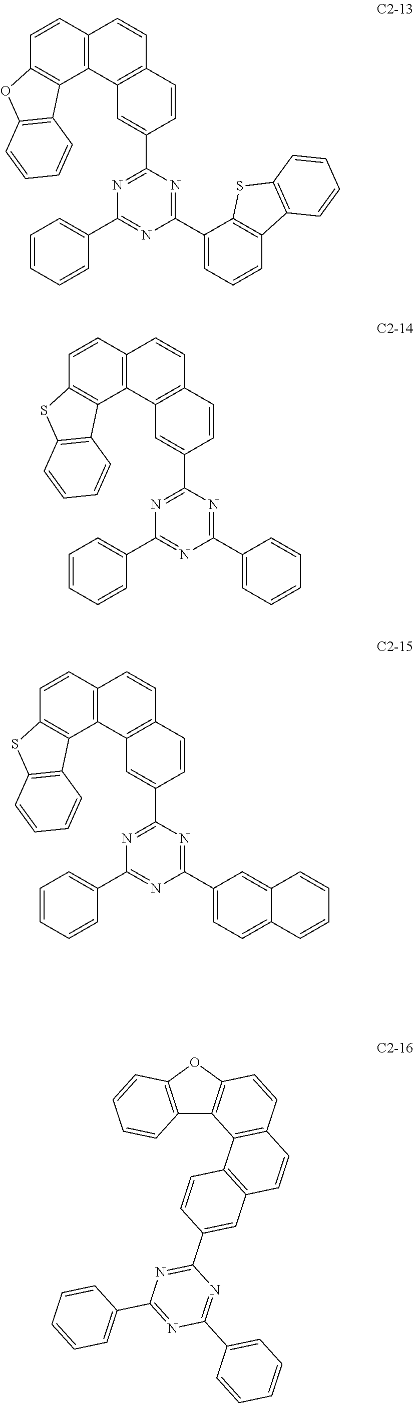 [SMILES: c1ccc(-c2nc(-c3ccc4ccc5ccc6oc7ccccc7c6c5c4c3)nc(-c3cccc4c3sc3ccccc34)n2)cc1 ; c1ccc(-c2nc(-c3ccccc3)nc(-c3ccc4ccc5ccc6sc7ccccc7c6c5c4c3)n2)cc1 ; c1ccc(-c2nc(-c3ccc4ccccc4c3)nc(-c3ccc4ccc5ccc6sc7ccccc7c6c5c4c3)n2)cc1 ; c1ccc(-c2nc(-c3ccccc3)nc(-c3ccc4c(ccc5ccc6oc7ccccc7c6c54)c3)n2)cc1]

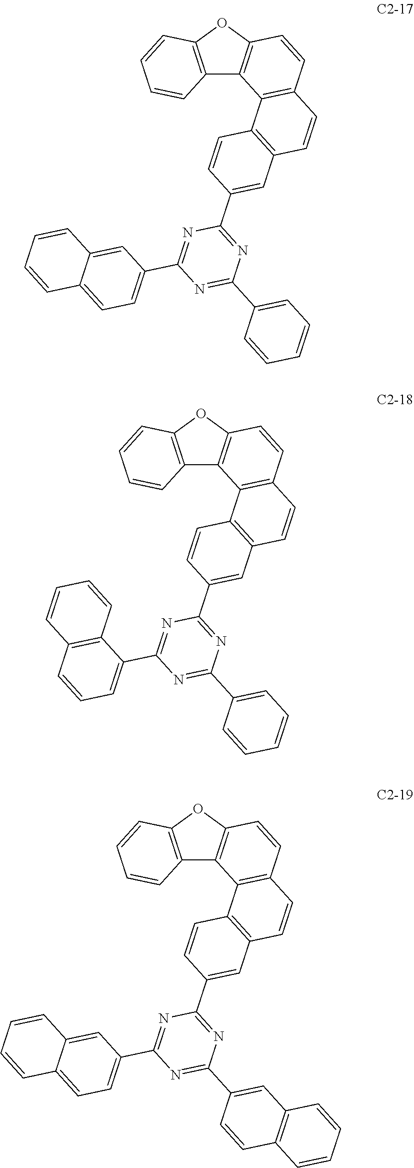 [SMILES: c1ccc(-c2nc(-c3ccc4ccccc4c3)nc(-c3ccc4c(ccc5ccc6oc7ccccc7c6c54)c3)n2)cc1 ; c1ccc(-c2nc(-c3ccc4c(ccc5ccc6oc7ccccc7c6c54)c3)nc(-c3cccc4ccccc34)n2)cc1 ; c1ccc2cc(-c3nc(-c4ccc5ccccc5c4)nc(-c4ccc5c(ccc6ccc7oc8ccccc8c7c65)c4)n3)ccc2c1]

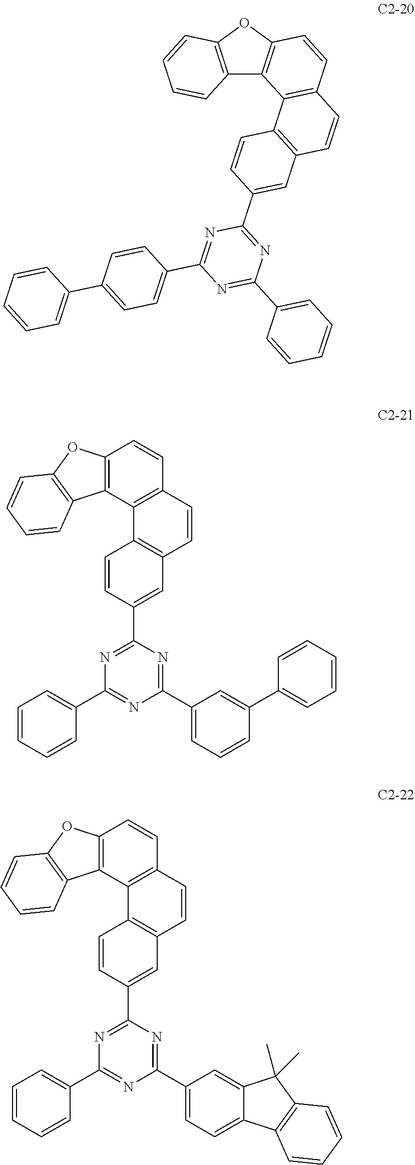 [SMILES: c1ccc(-c2ccc(-c3nc(-c4ccccc4)nc(-c4ccc5c(ccc6ccc7oc8ccccc8c7c65)c4)n3)cc2)cc1 ; c1ccc(-c2cccc(-c3nc(-c4ccccc4)nc(-c4ccc5c(ccc6ccc7oc8ccccc8c7c65)c4)n3)c2)cc1 ; CC1(C)c2ccccc2-c2ccc(-c3nc(-c4ccccc4)nc(-c4ccc5c(ccc6ccc7oc8ccccc8c7c65)c4)n3)cc21]

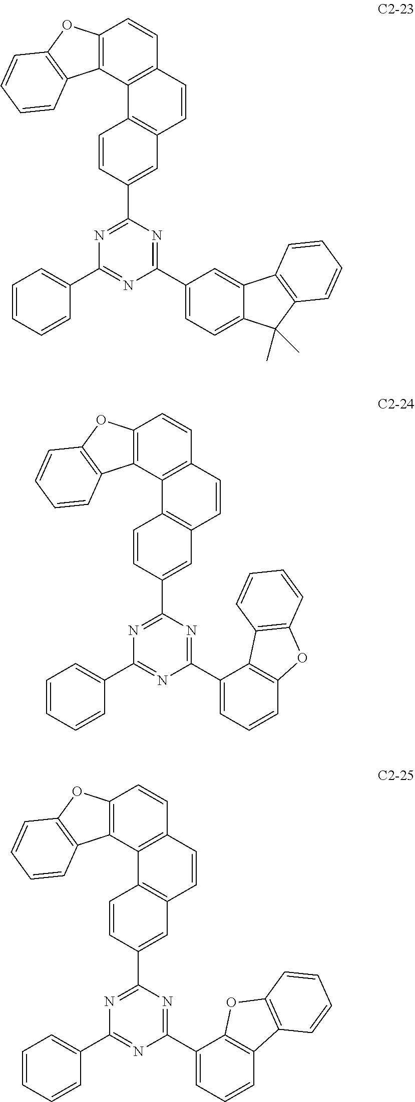 [SMILES: CC1(C)c2ccccc2-c2cc(-c3nc(-c4ccccc4)nc(-c4ccc5c(ccc6ccc7oc8ccccc8c7c65)c4)n3)ccc21 ; c1ccc(-c2nc(-c3ccc4c(ccc5ccc6oc7ccccc7c6c54)c3)nc(-c3cccc4oc5ccccc5c34)n2)cc1 ; c1ccc(-c2nc(-c3ccc4c(ccc5ccc6oc7ccccc7c6c54)c3)nc(-c3cccc4c3oc3ccccc34)n2)cc1]

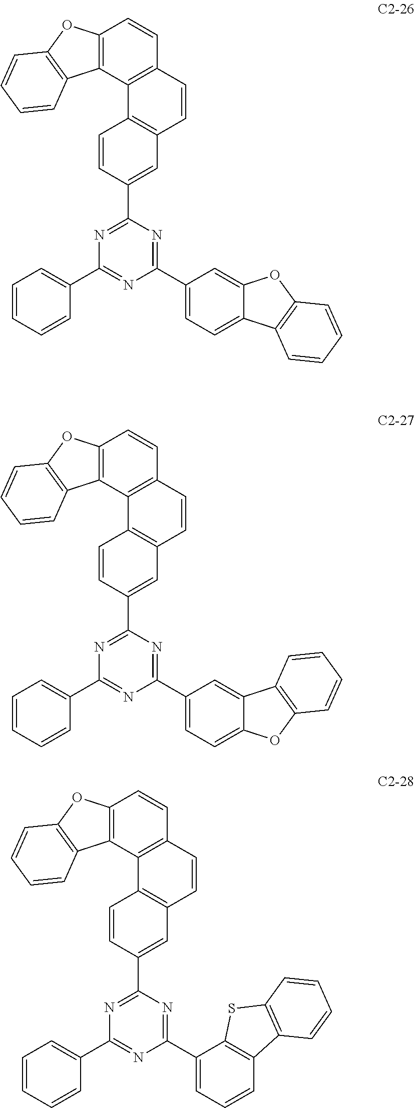 [SMILES: c1ccc(-c2nc(-c3ccc4c(ccc5ccc6oc7ccccc7c6c54)c3)nc(-c3ccc4c(c3)oc3ccccc34)n2)cc1 ; c1ccc(-c2nc(-c3ccc4c(ccc5ccc6oc7ccccc7c6c54)c3)nc(-c3ccc4oc5ccccc5c4c3)n2)cc1 ; c1ccc(-c2nc(-c3ccc4c(ccc5ccc6oc7ccccc7c6c54)c3)nc(-c3cccc4c3sc3ccccc34)n2)cc1]

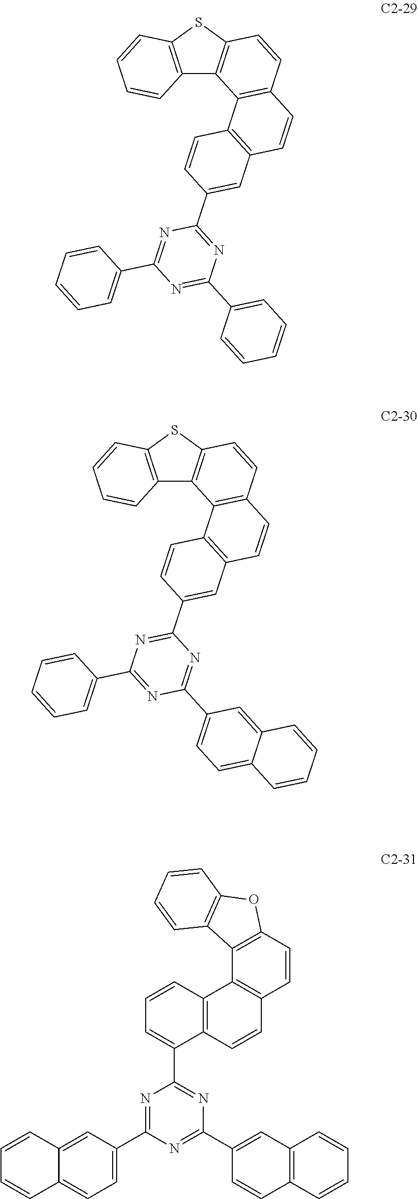 [SMILES: c1ccc(-c2nc(-c3ccccc3)nc(-c3ccc4c(ccc5ccc6sc7ccccc7c6c54)c3)n2)cc1 ; c1ccc(-c2nc(-c3ccc4ccccc4c3)nc(-c3ccc4c(ccc5ccc6sc7ccccc7c6c54)c3)n2)cc1 ; c1ccc2cc(-c3nc(-c4ccc5ccccc5c4)nc(-c4cccc5c4ccc4ccc6oc7ccccc7c6c45)n3)ccc2c1]

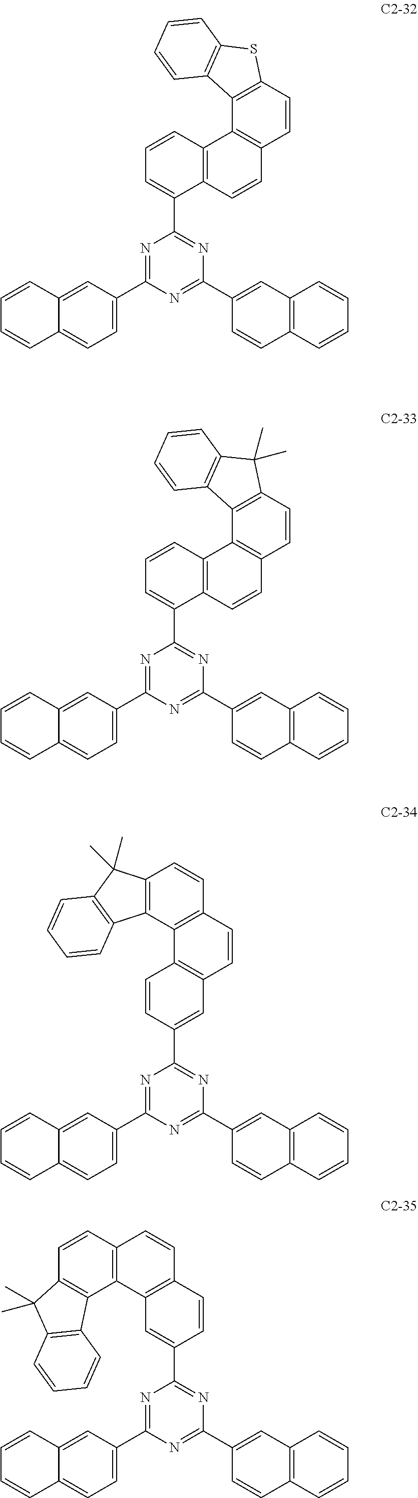 [SMILES: c1ccc2cc(-c3nc(-c4ccc5ccccc5c4)nc(-c4cccc5c4ccc4ccc6sc7ccccc7c6c45)n3)ccc2c1 ; CC1(C)c2ccccc2-c2c1ccc1ccc3c(-c4nc(-c5ccc6ccccc6c5)nc(-c5ccc6ccccc6c5)n4)cccc3c21 ; CC1(C)c2ccccc2-c2c1ccc1ccc3cc(-c4nc(-c5ccc6ccccc6c5)nc(-c5ccc6ccccc6c5)n4)ccc3c21 ; CC1(C)c2ccccc2-c2c1ccc1ccc3ccc(-c4nc(-c5ccc6ccccc6c5)nc(-c5ccc6ccccc6c5)n4)cc3c21]

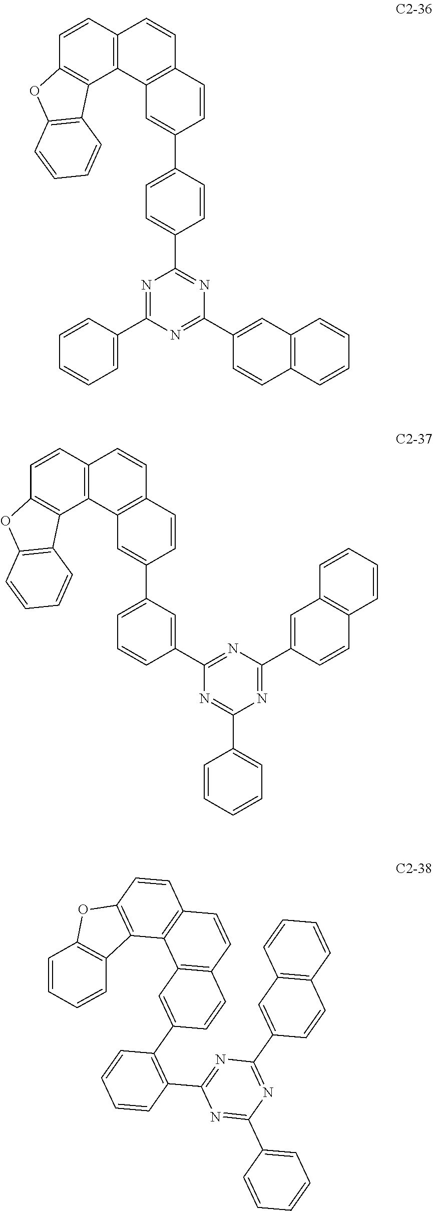 [SMILES: c1ccc(-c2nc(-c3ccc(-c4ccc5ccc6ccc7oc8ccccc8c7c6c5c4)cc3)nc(-c3ccc4ccccc4c3)n2)cc1 ; c1ccc(-c2nc(-c3cccc(-c4ccc5ccc6ccc7oc8ccccc8c7c6c5c4)c3)nc(-c3ccc4ccccc4c3)n2)cc1 ; c1ccc(-c2nc(-c3ccc4ccccc4c3)nc(-c3ccccc3-c3ccc4ccc5ccc6oc7ccccc7c6c5c4c3)n2)cc1]

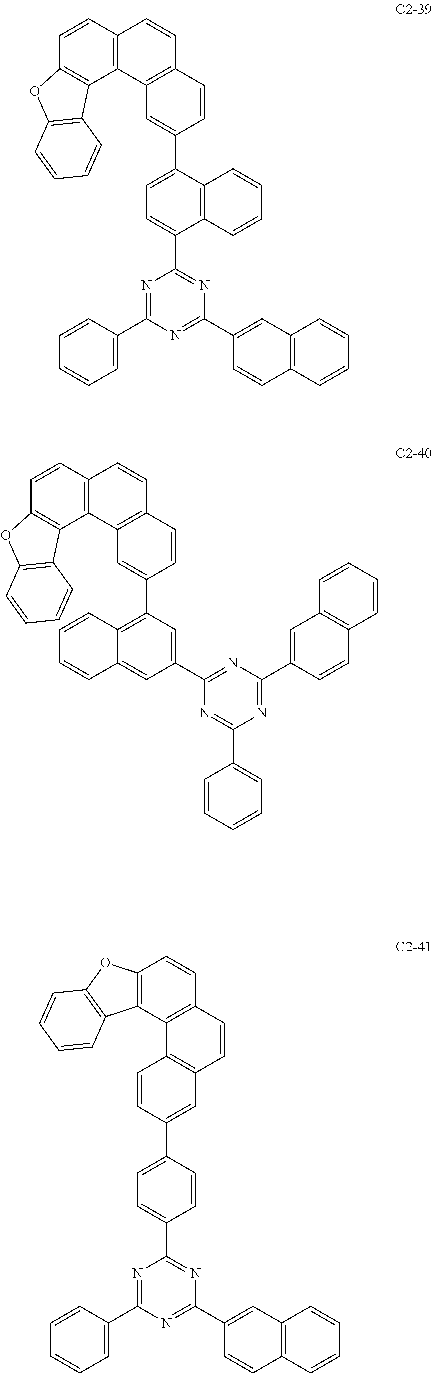 [SMILES: c1ccc(-c2nc(-c3ccc4ccccc4c3)nc(-c3ccc(-c4ccc5ccc6ccc7oc8ccccc8c7c6c5c4)c4ccccc34)n2)cc1 ; c1ccc(-c2nc(-c3ccc4ccccc4c3)nc(-c3cc(-c4ccc5ccc6ccc7oc8ccccc8c7c6c5c4)c4ccccc4c3)n2)cc1 ; c1ccc(-c2nc(-c3ccc(-c4ccc5c(ccc6ccc7oc8ccccc8c7c65)c4)cc3)nc(-c3ccc4ccccc4c3)n2)cc1]

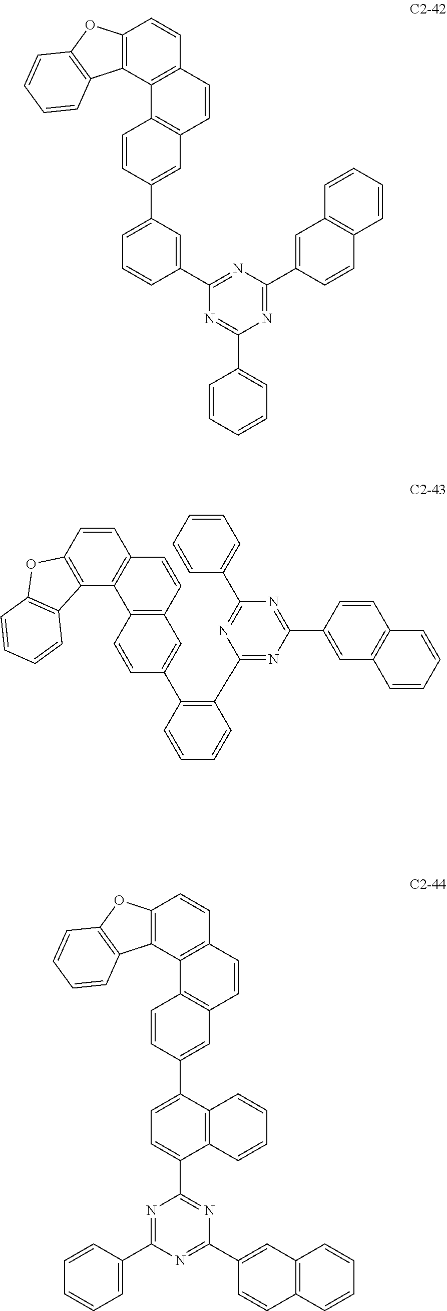 [SMILES: c1ccc(-c2nc(-c3cccc(-c4ccc5c(ccc6ccc7oc8ccccc8c7c65)c4)c3)nc(-c3ccc4ccccc4c3)n2)cc1 ; c1ccc(-c2nc(-c3ccc4ccccc4c3)nc(-c3ccccc3-c3ccc4c(ccc5ccc6oc7ccccc7c6c54)c3)n2)cc1 ; c1ccc(-c2nc(-c3ccc4ccccc4c3)nc(-c3ccc(-c4ccc5c(ccc6ccc7oc8ccccc8c7c65)c4)c4ccccc34)n2)cc1]

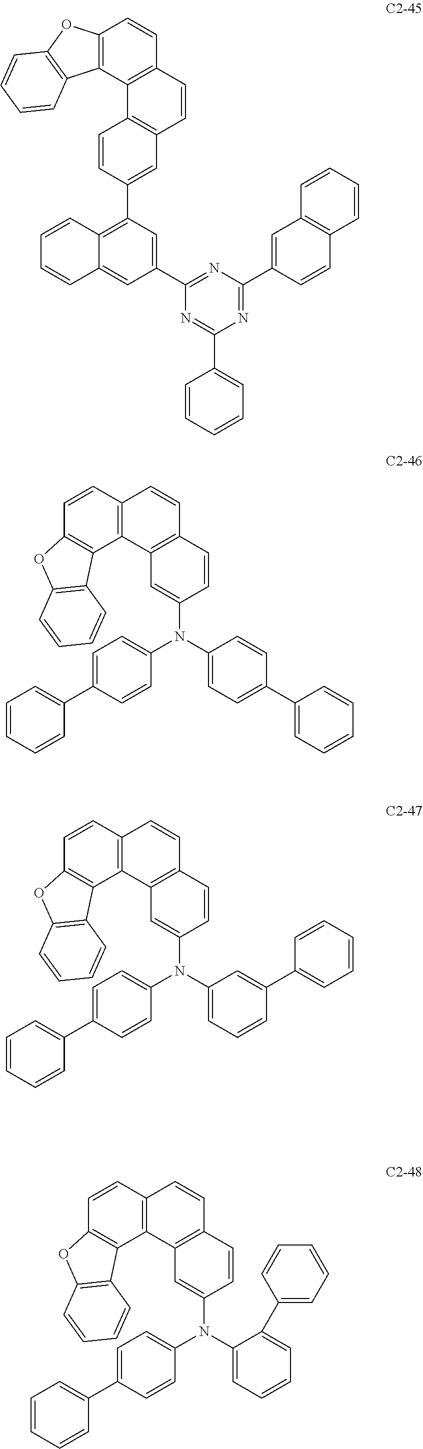 [SMILES: c1ccc(-c2nc(-c3ccc4ccccc4c3)nc(-c3cc(-c4ccc5c(ccc6ccc7oc8ccccc8c7c65)c4)c4ccccc4c3)n2)cc1 ; c1ccc(-c2ccc(N(c3ccc(-c4ccccc4)cc3)c3ccc4ccc5ccc6oc7ccccc7c6c5c4c3)cc2)cc1 ; c1ccc(-c2ccc(N(c3cccc(-c4ccccc4)c3)c3ccc4ccc5ccc6oc7ccccc7c6c5c4c3)cc2)cc1 ; c1ccc(-c2ccc(N(c3ccc4ccc5ccc6oc7ccccc7c6c5c4c3)c3ccccc3-c3ccccc3)cc2)cc1]

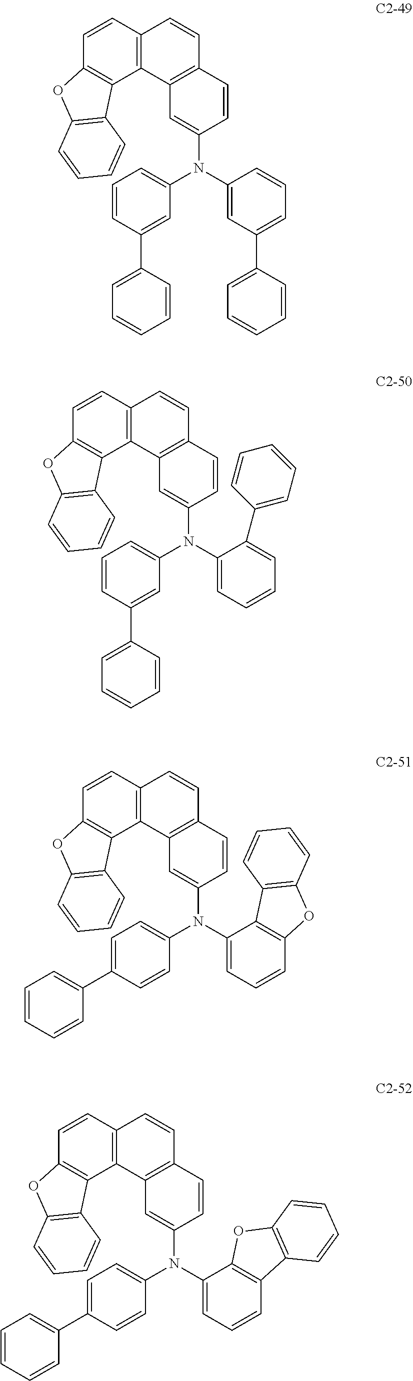 [SMILES: c1ccc(-c2cccc(N(c3cccc(-c4ccccc4)c3)c3ccc4ccc5ccc6oc7ccccc7c6c5c4c3)c2)cc1 ; c1ccc(-c2cccc(N(c3ccc4ccc5ccc6oc7ccccc7c6c5c4c3)c3ccccc3-c3ccccc3)c2)cc1 ; c1ccc(-c2ccc(N(c3ccc4ccc5ccc6oc7ccccc7c6c5c4c3)c3cccc4oc5ccccc5c34)cc2)cc1 ; c1ccc(-c2ccc(N(c3ccc4ccc5ccc6oc7ccccc7c6c5c4c3)c3cccc4c3oc3ccccc34)cc2)cc1]

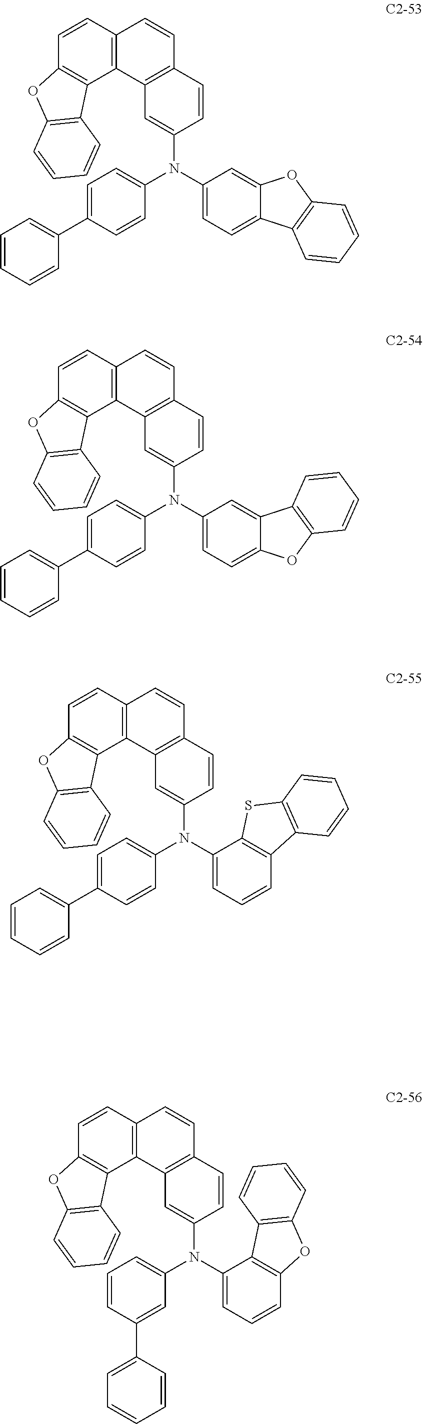 [SMILES: c1ccc(-c2ccc(N(c3ccc4c(c3)oc3ccccc34)c3ccc4ccc5ccc6oc7ccccc7c6c5c4c3)cc2)cc1 ; c1ccc(-c2ccc(N(c3ccc4oc5ccccc5c4c3)c3ccc4ccc5ccc6oc7ccccc7c6c5c4c3)cc2)cc1 ; c1ccc(-c2ccc(N(c3ccc4ccc5ccc6oc7ccccc7c6c5c4c3)c3cccc4c3sc3ccccc34)cc2)cc1 ; c1ccc(-c2cccc(N(c3ccc4ccc5ccc6oc7ccccc7c6c5c4c3)c3cccc4oc5ccccc5c34)c2)cc1]

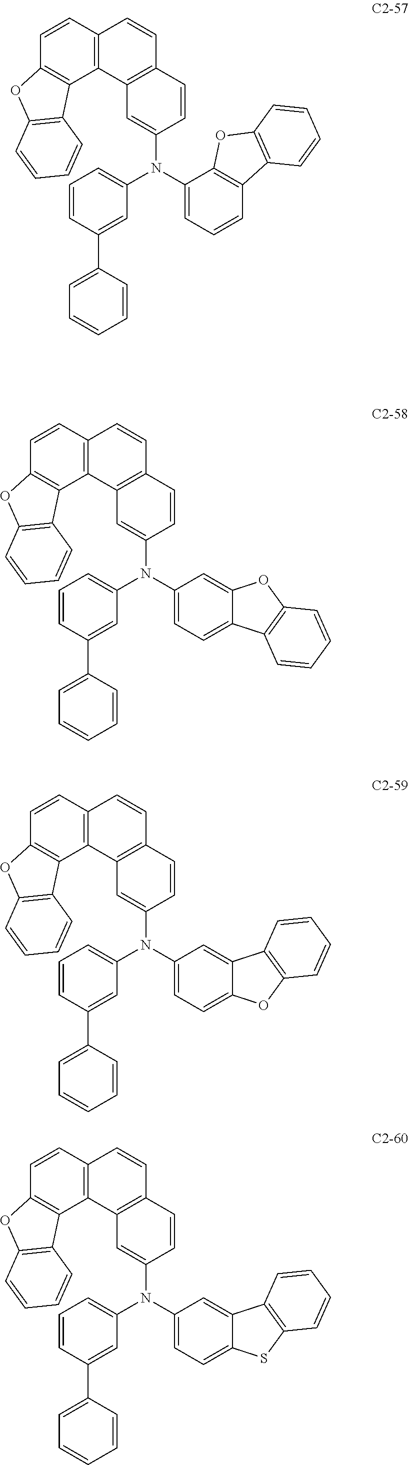

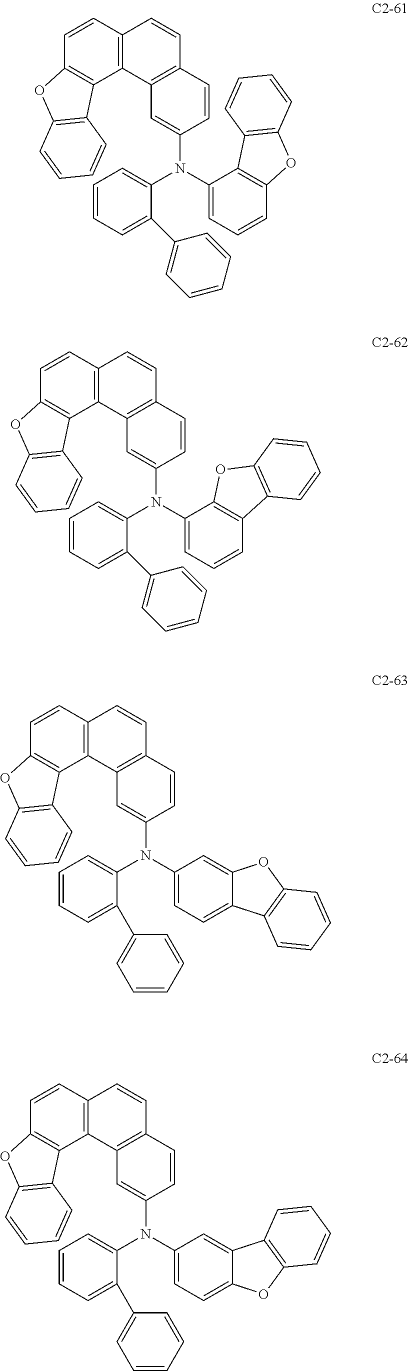 [SMILES: c1ccc(-c2ccccc2N(c2ccc3ccc4ccc5oc6ccccc6c5c4c3c2)c2cccc3oc4ccccc4c23)cc1 ; c1ccc(-c2ccccc2N(c2ccc3ccc4ccc5oc6ccccc6c5c4c3c2)c2cccc3c2oc2ccccc23)cc1 ; c1ccc(-c2ccccc2N(c2ccc3c(c2)oc2ccccc23)c2ccc3ccc4ccc5oc6ccccc6c5c4c3c2)cc1 ; c1ccc(-c2ccccc2N(c2ccc3oc4ccccc4c3c2)c2ccc3ccc4ccc5oc6ccccc6c5c4c3c2)cc1]

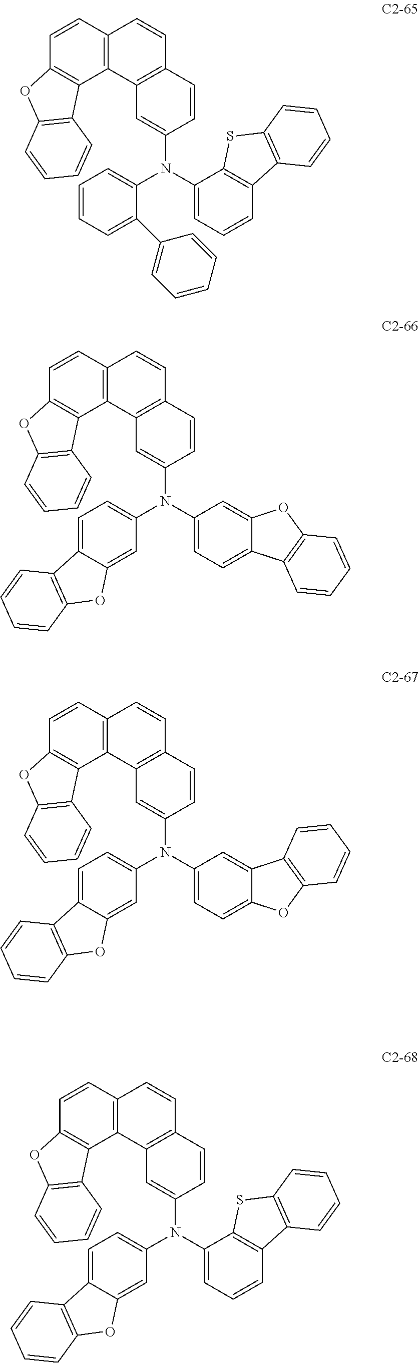 [SMILES: c1ccc(-c2ccccc2N(c2ccc3ccc4ccc5oc6ccccc6c5c4c3c2)c2cccc3c2sc2ccccc23)cc1 ; c1ccc2c(c1)oc1cc(N(c3ccc4c(c3)oc3ccccc34)c3ccc4ccc5ccc6oc7ccccc7c6c5c4c3)ccc12 ; c1ccc2c(c1)oc1cc(N(c3ccc4oc5ccccc5c4c3)c3ccc4ccc5ccc6oc7ccccc7c6c5c4c3)ccc12 ; c1ccc2c(c1)oc1cc(N(c3ccc4ccc5ccc6oc7ccccc7c6c5c4c3)c3cccc4c3sc3ccccc34)ccc12]

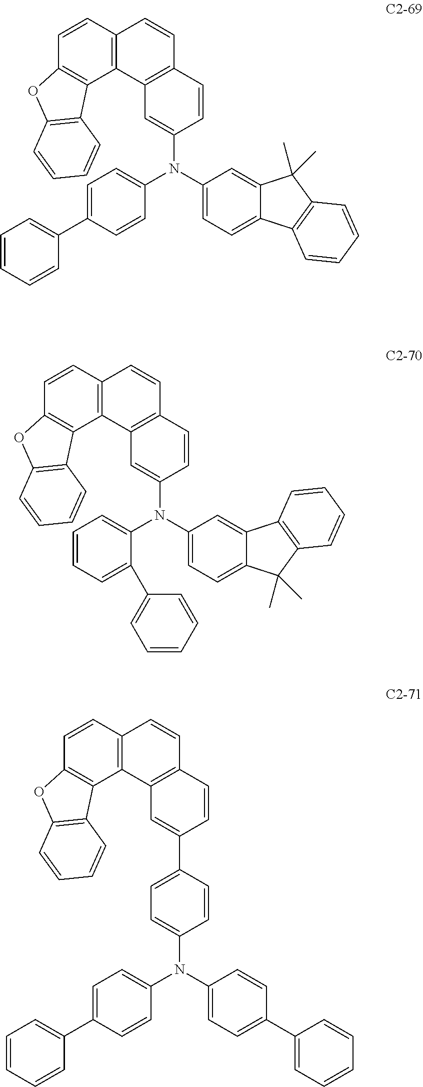 [SMILES: CC1(C)c2ccccc2-c2ccc(N(c3ccc(-c4ccccc4)cc3)c3ccc4ccc5ccc6oc7ccccc7c6c5c4c3)cc21 ; CC1(C)c2ccccc2-c2cc(N(c3ccc4ccc5ccc6oc7ccccc7c6c5c4c3)c3ccccc3-c3ccccc3)ccc21 ; c1ccc(-c2ccc(N(c3ccc(-c4ccccc4)cc3)c3ccc(-c4ccc5ccc6ccc7oc8ccccc8c7c6c5c4)cc3)cc2)cc1]

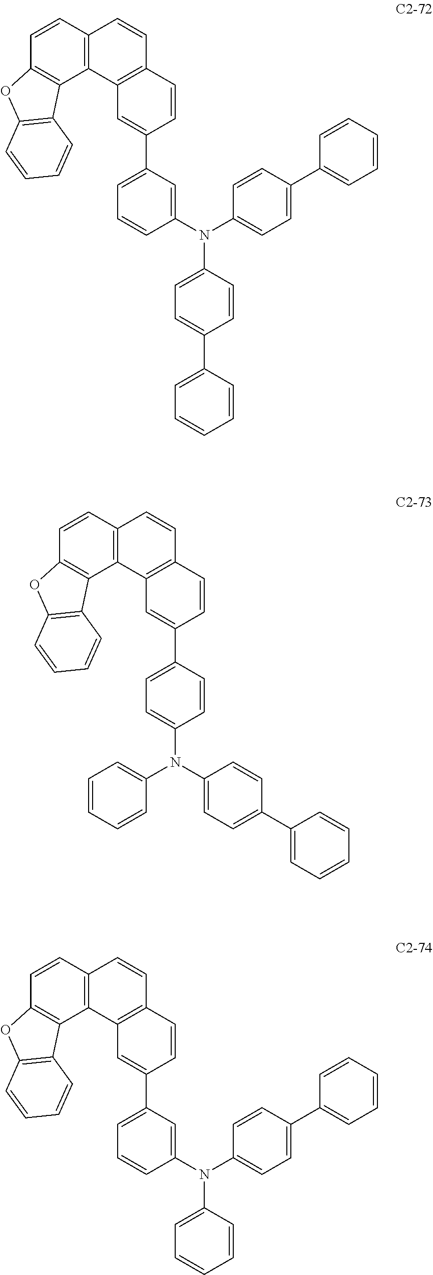 [SMILES: c1ccc(-c2ccc(N(c3ccc(-c4ccccc4)cc3)c3cccc(-c4ccc5ccc6ccc7oc8ccccc8c7c6c5c4)c3)cc2)cc1 ; c1ccc(-c2ccc(N(c3ccccc3)c3ccc(-c4ccc5ccc6ccc7oc8ccccc8c7c6c5c4)cc3)cc2)cc1 ; c1ccc(-c2ccc(N(c3ccccc3)c3cccc(-c4ccc5ccc6ccc7oc8ccccc8c7c6c5c4)c3)cc2)cc1]

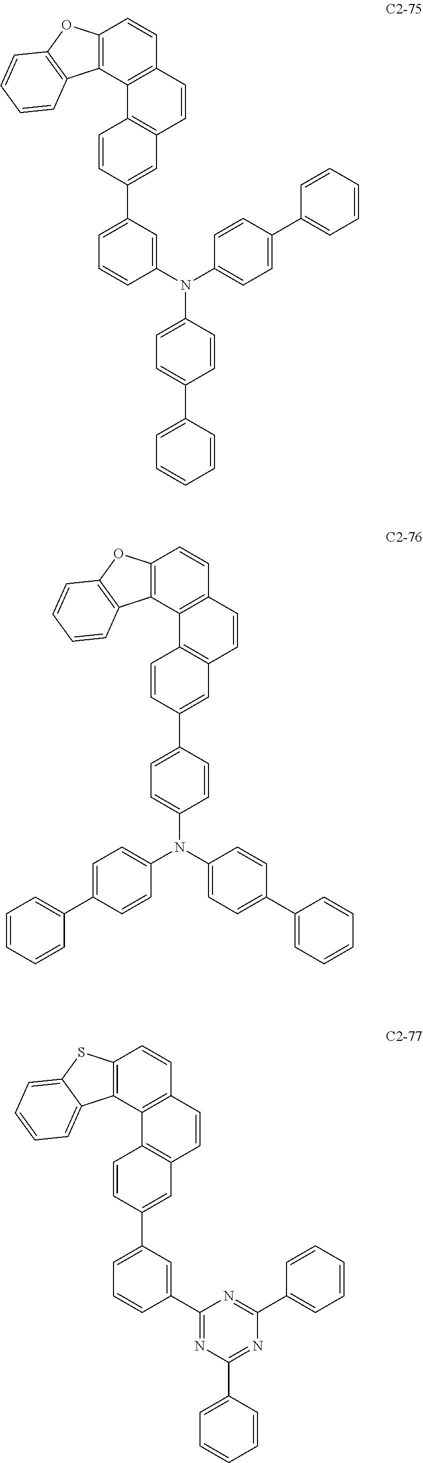 [SMILES: c1ccc(-c2ccc(N(c3ccc(-c4ccccc4)cc3)c3cccc(-c4ccc5c(ccc6ccc7oc8ccccc8c7c65)c4)c3)cc2)cc1 ; c1ccc(-c2ccc(N(c3ccc(-c4ccccc4)cc3)c3ccc(-c4ccc5c(ccc6ccc7oc8ccccc8c7c65)c4)cc3)cc2)cc1 ; c1ccc(-c2nc(-c3ccccc3)nc(-c3cccc(-c4ccc5c(ccc6ccc7sc8ccccc8c7c65)c4)c3)n2)cc1]

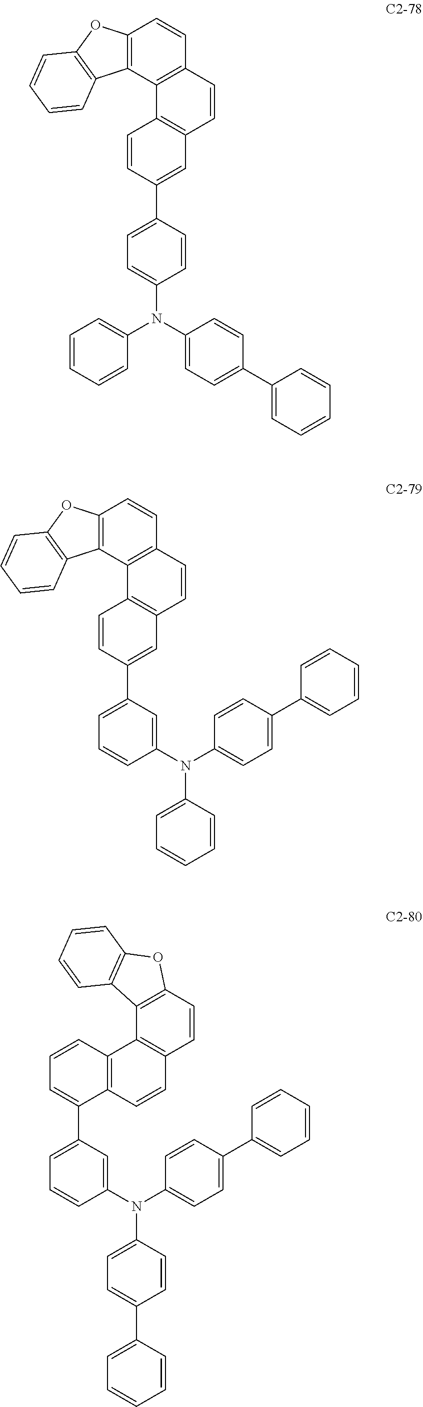 [SMILES: c1ccc(-c2ccc(N(c3ccccc3)c3ccc(-c4ccc5c(ccc6ccc7oc8ccccc8c7c65)c4)cc3)cc2)cc1 ; c1ccc(-c2ccc(N(c3ccccc3)c3cccc(-c4ccc5c(ccc6ccc7oc8ccccc8c7c65)c4)c3)cc2)cc1 ; c1ccc(-c2ccc(N(c3ccc(-c4ccccc4)cc3)c3cccc(-c4cccc5c4ccc4ccc6oc7ccccc7c6c45)c3)cc2)cc1]

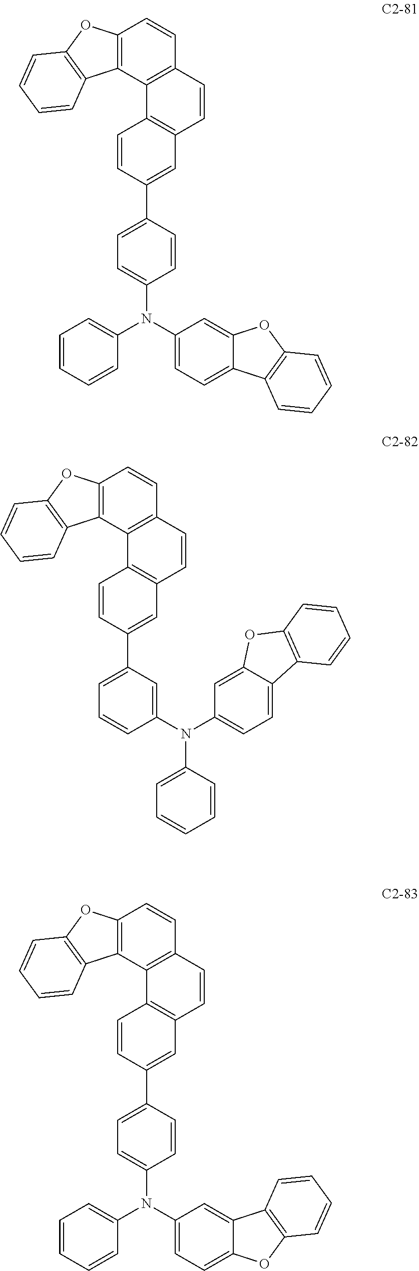 [SMILES: c1ccc(N(c2ccc(-c3ccc4c(ccc5ccc6oc7ccccc7c6c54)c3)cc2)c2ccc3c(c2)oc2ccccc23)cc1 ; c1ccc(N(c2cccc(-c3ccc4c(ccc5ccc6oc7ccccc7c6c54)c3)c2)c2ccc3c(c2)oc2ccccc23)cc1 ; c1ccc(N(c2ccc(-c3ccc4c(ccc5ccc6oc7ccccc7c6c54)c3)cc2)c2ccc3oc4ccccc4c3c2)cc1]

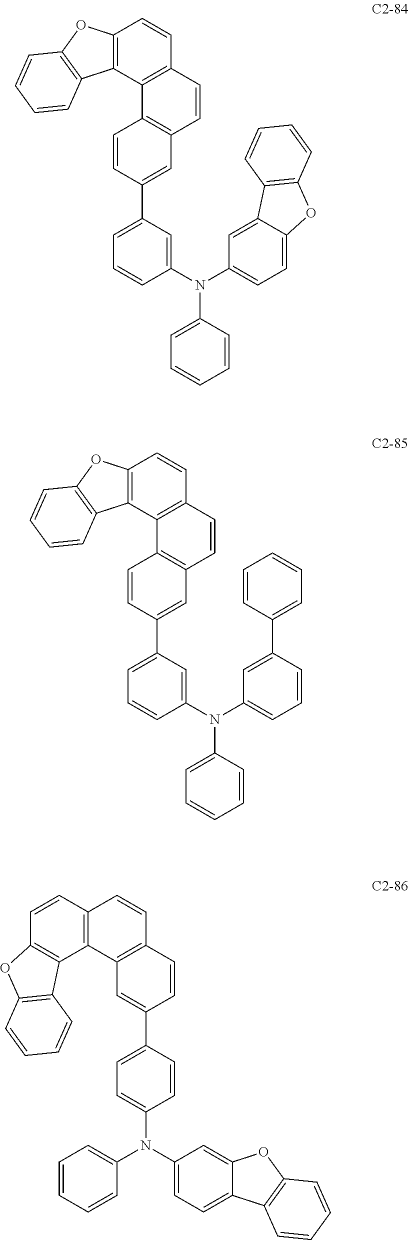 [SMILES: c1ccc(N(c2cccc(-c3ccc4c(ccc5ccc6oc7ccccc7c6c54)c3)c2)c2ccc3oc4ccccc4c3c2)cc1 ; c1ccc(-c2cccc(N(c3ccccc3)c3cccc(-c4ccc5c(ccc6ccc7oc8ccccc8c7c65)c4)c3)c2)cc1 ; c1ccc(N(c2ccc(-c3ccc4ccc5ccc6oc7ccccc7c6c5c4c3)cc2)c2ccc3c(c2)oc2ccccc23)cc1]

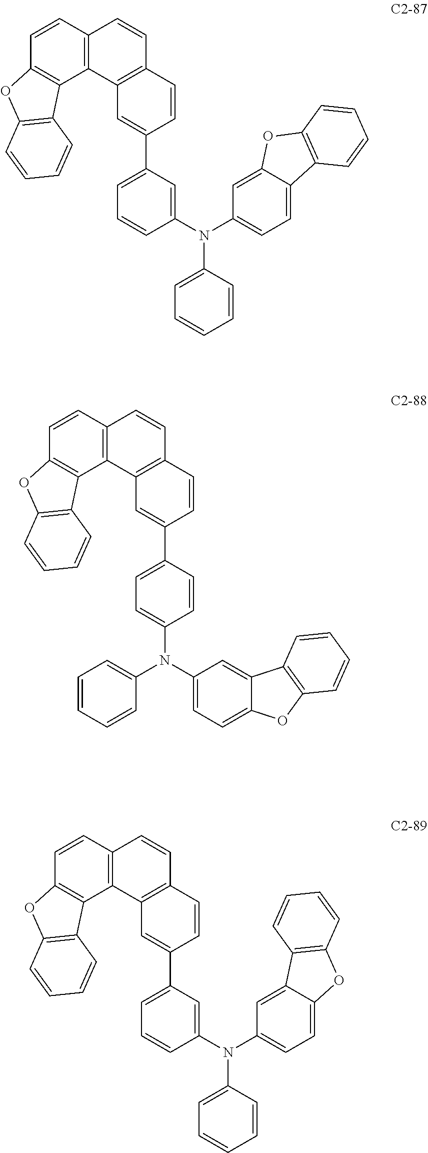 [SMILES: c1ccc(N(c2cccc(-c3ccc4ccc5ccc6oc7ccccc7c6c5c4c3)c2)c2ccc3c(c2)oc2ccccc23)cc1 ; c1ccc(N(c2ccc(-c3ccc4ccc5ccc6oc7ccccc7c6c5c4c3)cc2)c2ccc3oc4ccccc4c3c2)cc1 ; c1ccc(N(c2cccc(-c3ccc4ccc5ccc6oc7ccccc7c6c5c4c3)c2)c2ccc3oc4ccccc4c3c2)cc1]

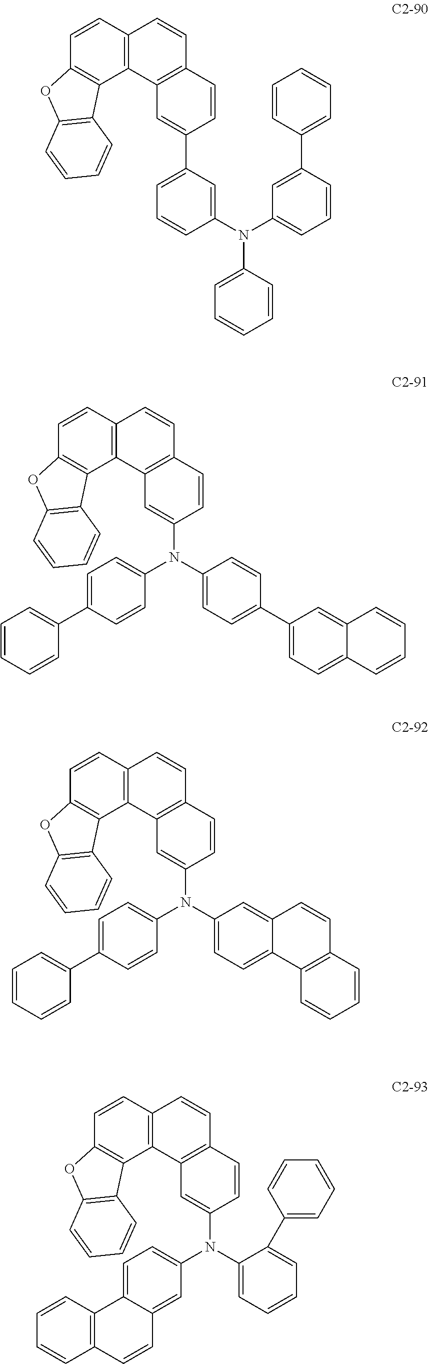 [SMILES: c1ccc(-c2cccc(N(c3ccccc3)c3cccc(-c4ccc5ccc6ccc7oc8ccccc8c7c6c5c4)c3)c2)cc1 ; c1ccc(-c2ccc(N(c3ccc(-c4ccc5ccccc5c4)cc3)c3ccc4ccc5ccc6oc7ccccc7c6c5c4c3)cc2)cc1 ; c1ccc(-c2ccc(N(c3ccc4c(ccc5ccccc54)c3)c3ccc4ccc5ccc6oc7ccccc7c6c5c4c3)cc2)cc1 ; c1ccc(-c2ccccc2N(c2ccc3c(ccc4ccccc43)c2)c2ccc3ccc4ccc5oc6ccccc6c5c4c3c2)cc1]

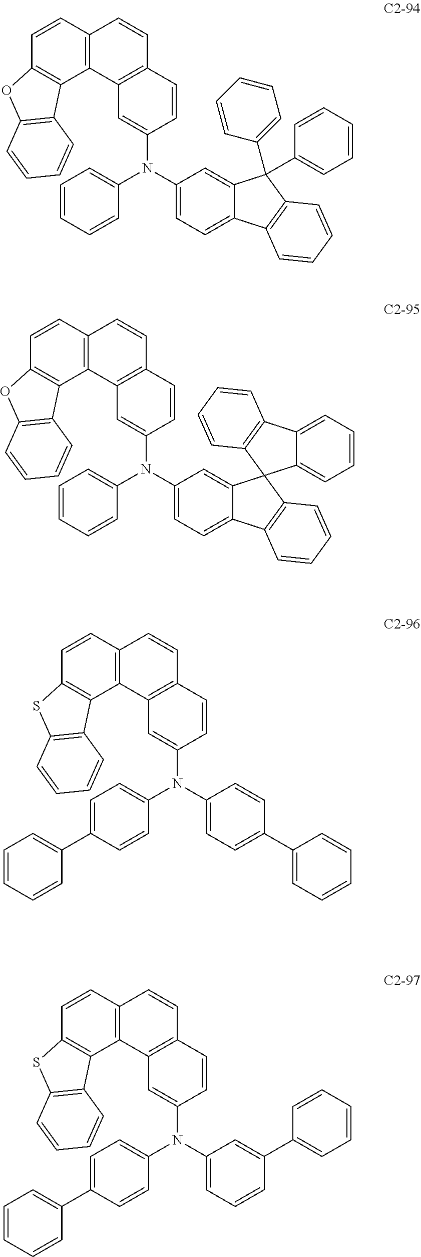 [SMILES: c1ccc(N(c2ccc3c(c2)C(c2ccccc2)(c2ccccc2)c2ccccc2-3)c2ccc3ccc4ccc5oc6ccccc6c5c4c3c2)cc1 ; c1ccc(N(c2ccc3c(c2)C2(c4ccccc4-c4ccccc42)c2ccccc2-3)c2ccc3ccc4ccc5oc6ccccc6c5c4c3c2)cc1 ; c1ccc(-c2ccc(N(c3ccc(-c4ccccc4)cc3)c3ccc4ccc5ccc6sc7ccccc7c6c5c4c3)cc2)cc1 ; c1ccc(-c2ccc(N(c3cccc(-c4ccccc4)c3)c3ccc4ccc5ccc6sc7ccccc7c6c5c4c3)cc2)cc1]

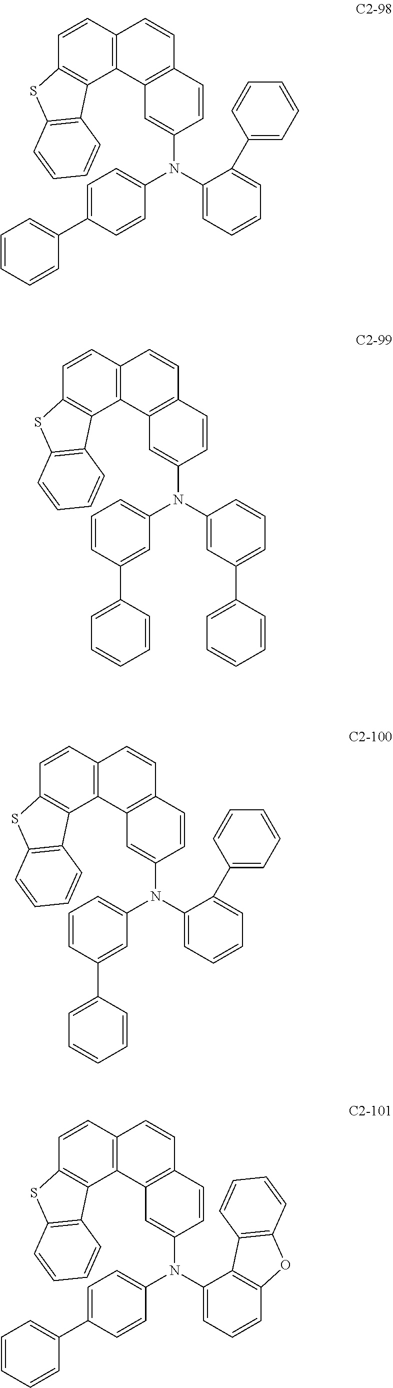 [SMILES: c1ccc(-c2ccc(N(c3ccc4ccc5ccc6sc7ccccc7c6c5c4c3)c3ccccc3-c3ccccc3)cc2)cc1 ; c1ccc(-c2cccc(N(c3cccc(-c4ccccc4)c3)c3ccc4ccc5ccc6sc7ccccc7c6c5c4c3)c2)cc1 ; c1ccc(-c2cccc(N(c3ccc4ccc5ccc6sc7ccccc7c6c5c4c3)c3ccccc3-c3ccccc3)c2)cc1 ; c1ccc(-c2ccc(N(c3ccc4ccc5ccc6sc7ccccc7c6c5c4c3)c3cccc4oc5ccccc5c34)cc2)cc1]

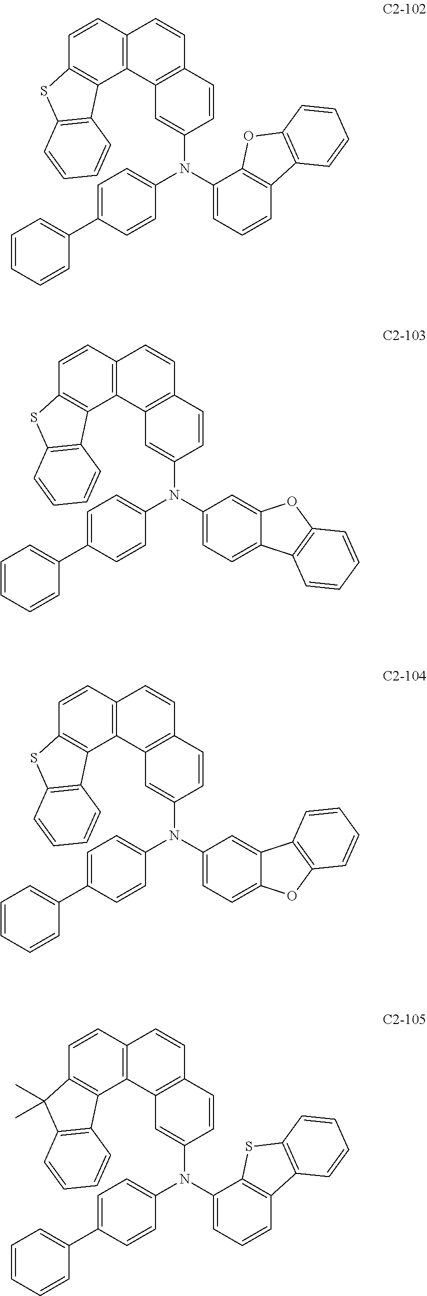 [SMILES: c1ccc(-c2ccc(N(c3ccc4ccc5ccc6sc7ccccc7c6c5c4c3)c3cccc4c3oc3ccccc34)cc2)cc1 ; c1ccc(-c2ccc(N(c3ccc4c(c3)oc3ccccc34)c3ccc4ccc5ccc6sc7ccccc7c6c5c4c3)cc2)cc1 ; c1ccc(-c2ccc(N(c3ccc4oc5ccccc5c4c3)c3ccc4ccc5ccc6sc7ccccc7c6c5c4c3)cc2)cc1 ; CC1(C)c2ccccc2-c2c1ccc1ccc3ccc(N(c4ccc(-c5ccccc5)cc4)c4cccc5c4sc4ccccc45)cc3c21]

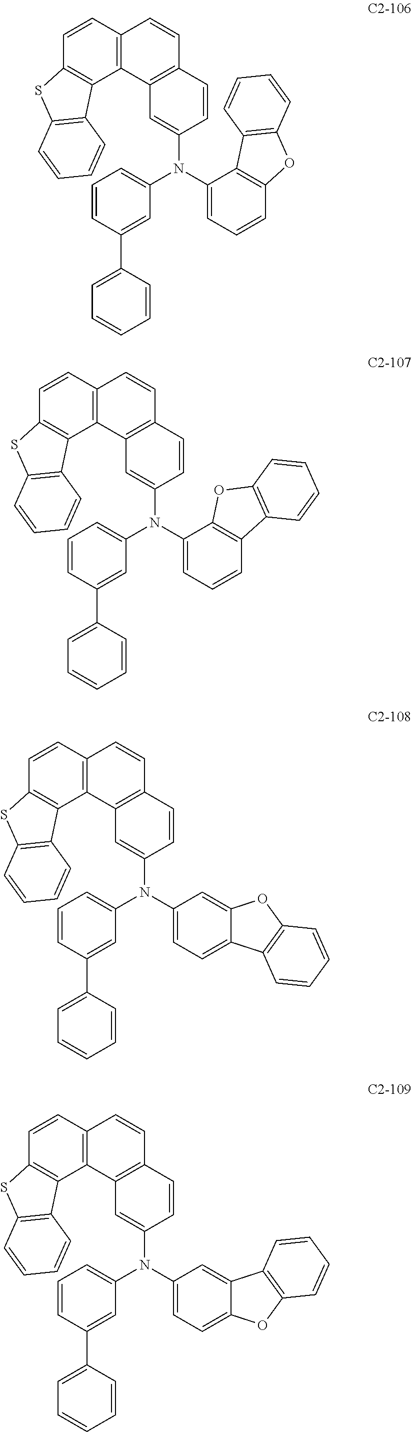

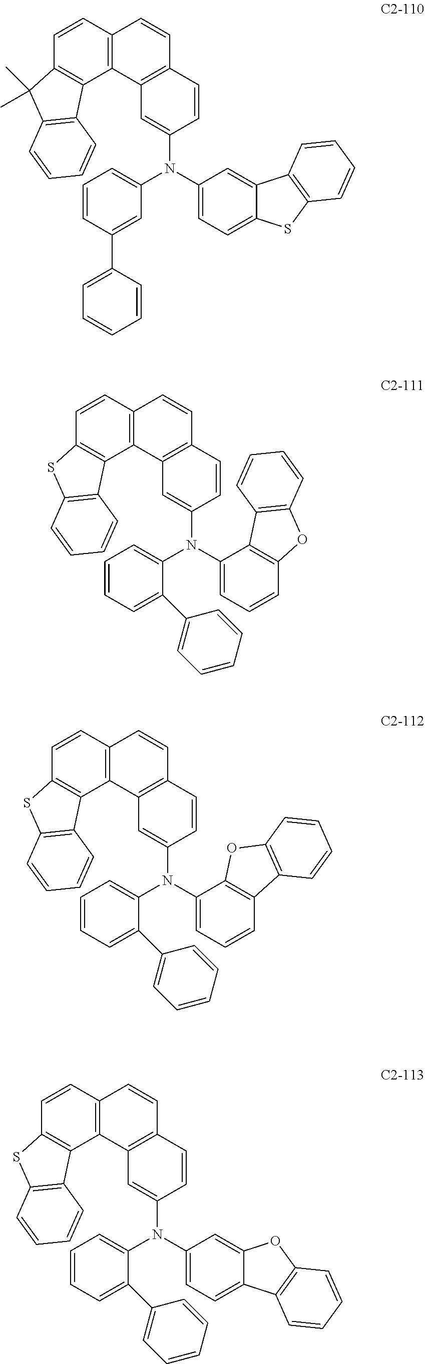 [SMILES: CC1(C)c2ccccc2-c2c1ccc1ccc3ccc(N(c4cccc(-c5ccccc5)c4)c4ccc5sc6ccccc6c5c4)cc3c21 ; c1ccc(-c2ccccc2N(c2ccc3ccc4ccc5sc6ccccc6c5c4c3c2)c2cccc3oc4ccccc4c23)cc1 ; c1ccc(-c2ccccc2N(c2ccc3ccc4ccc5sc6ccccc6c5c4c3c2)c2cccc3c2oc2ccccc23)cc1 ; c1ccc(-c2ccccc2N(c2ccc3c(c2)oc2ccccc23)c2ccc3ccc4ccc5sc6ccccc6c5c4c3c2)cc1]

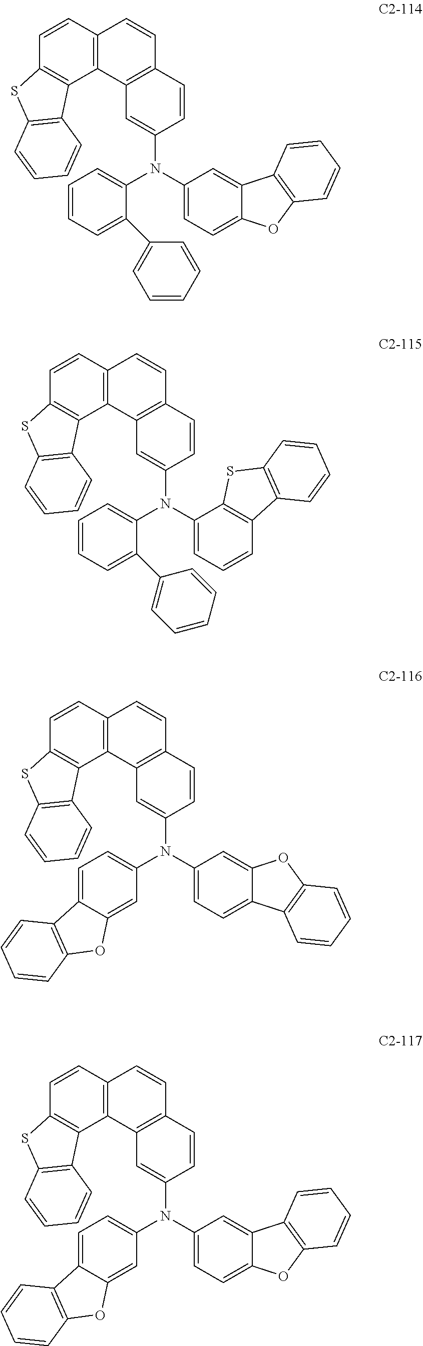 [SMILES: c1ccc(-c2ccccc2N(c2ccc3oc4ccccc4c3c2)c2ccc3ccc4ccc5sc6ccccc6c5c4c3c2)cc1 ; c1ccc(-c2ccccc2N(c2ccc3ccc4ccc5sc6ccccc6c5c4c3c2)c2cccc3c2sc2ccccc23)cc1 ; c1ccc2c(c1)oc1cc(N(c3ccc4c(c3)oc3ccccc34)c3ccc4ccc5ccc6sc7ccccc7c6c5c4c3)ccc12 ; c1ccc2c(c1)oc1cc(N(c3ccc4oc5ccccc5c4c3)c3ccc4ccc5ccc6sc7ccccc7c6c5c4c3)ccc12]

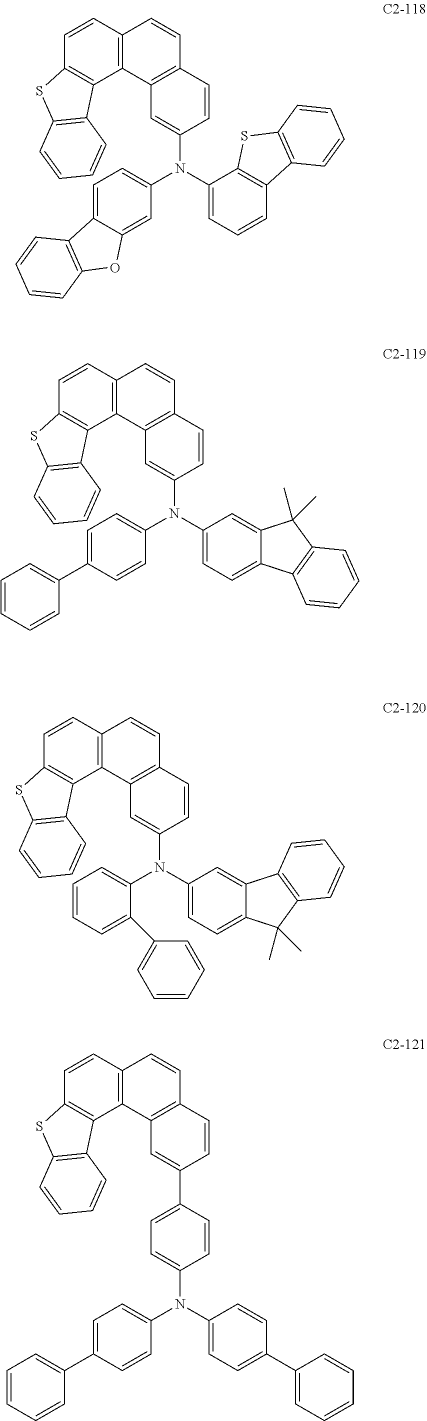 [SMILES: c1ccc2c(c1)oc1cc(N(c3ccc4ccc5ccc6sc7ccccc7c6c5c4c3)c3cccc4c3sc3ccccc34)ccc12 ; CC1(C)c2ccccc2-c2ccc(N(c3ccc(-c4ccccc4)cc3)c3ccc4ccc5ccc6sc7ccccc7c6c5c4c3)cc21 ; CC1(C)c2ccccc2-c2cc(N(c3ccc4ccc5ccc6sc7ccccc7c6c5c4c3)c3ccccc3-c3ccccc3)ccc21 ; c1ccc(-c2ccc(N(c3ccc(-c4ccccc4)cc3)c3ccc(-c4ccc5ccc6ccc7sc8ccccc8c7c6c5c4)cc3)cc2)cc1]

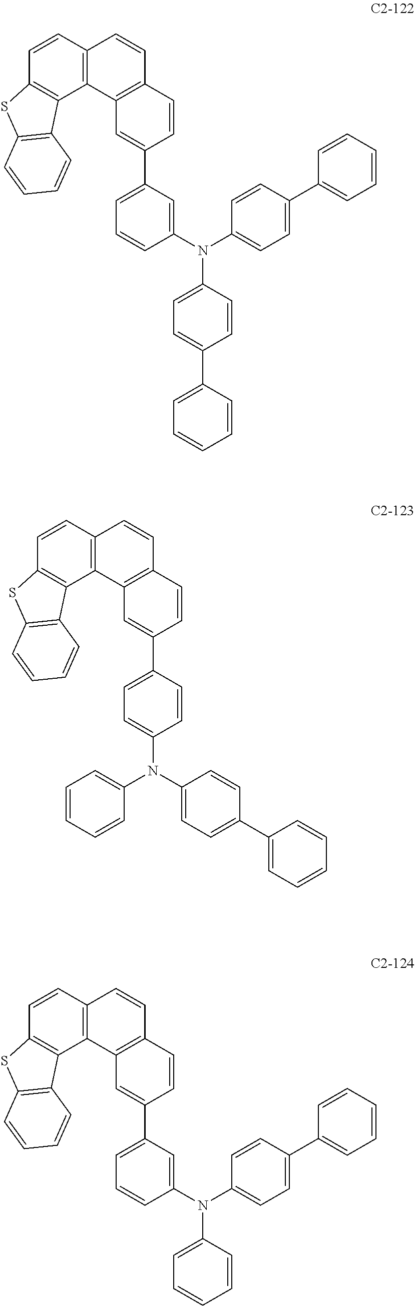 [SMILES: c1ccc(-c2ccc(N(c3ccc(-c4ccccc4)cc3)c3cccc(-c4ccc5ccc6ccc7sc8ccccc8c7c6c5c4)c3)cc2)cc1 ; c1ccc(-c2ccc(N(c3ccccc3)c3ccc(-c4ccc5ccc6ccc7sc8ccccc8c7c6c5c4)cc3)cc2)cc1 ; c1ccc(-c2ccc(N(c3ccccc3)c3cccc(-c4ccc5ccc6ccc7sc8ccccc8c7c6c5c4)c3)cc2)cc1]

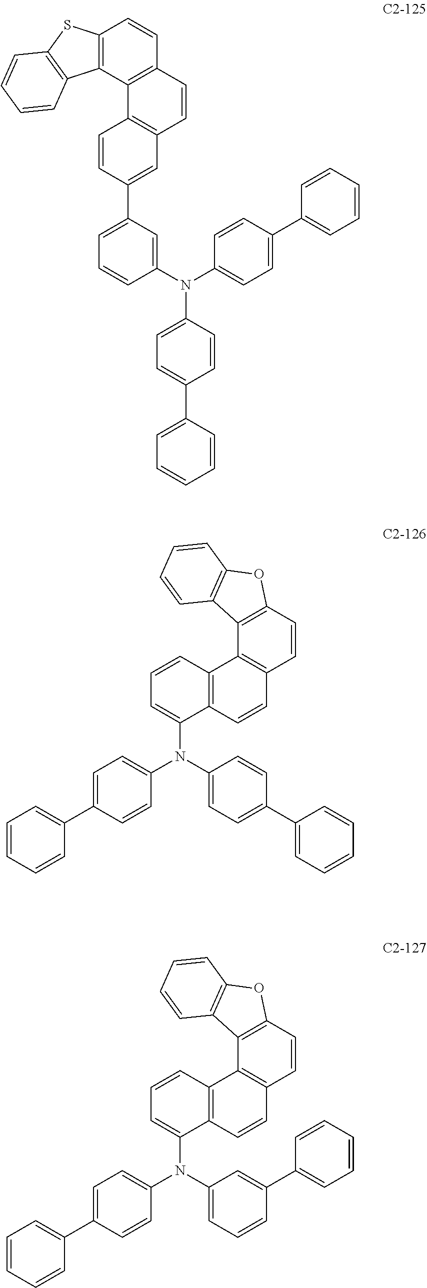 [SMILES: c1ccc(-c2ccc(N(c3ccc(-c4ccccc4)cc3)c3cccc(-c4ccc5c(ccc6ccc7sc8ccccc8c7c65)c4)c3)cc2)cc1 ; c1ccc(-c2ccc(N(c3ccc(-c4ccccc4)cc3)c3cccc4c3ccc3ccc5oc6ccccc6c5c34)cc2)cc1 ; c1ccc(-c2ccc(N(c3cccc(-c4ccccc4)c3)c3cccc4c3ccc3ccc5oc6ccccc6c5c34)cc2)cc1]

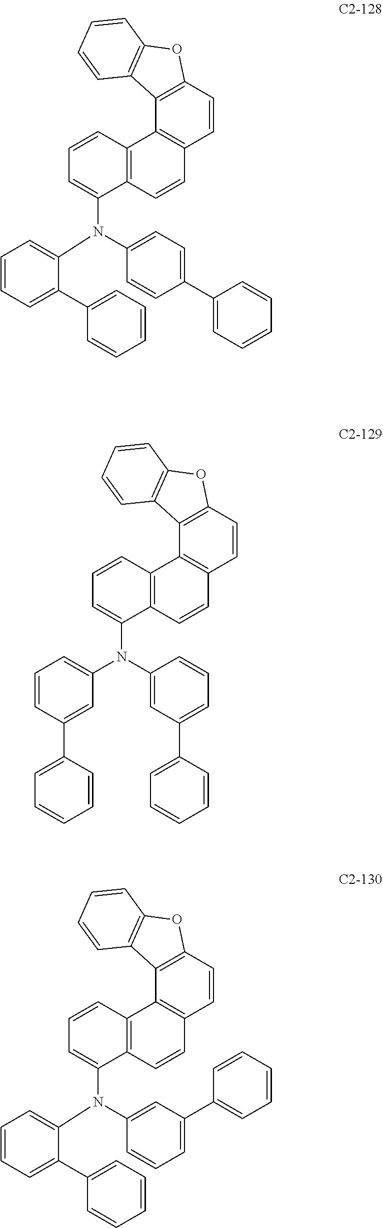 [SMILES: c1ccc(-c2ccc(N(c3ccccc3-c3ccccc3)c3cccc4c3ccc3ccc5oc6ccccc6c5c34)cc2)cc1 ; c1ccc(-c2cccc(N(c3cccc(-c4ccccc4)c3)c3cccc4c3ccc3ccc5oc6ccccc6c5c34)c2)cc1 ; c1ccc(-c2cccc(N(c3ccccc3-c3ccccc3)c3cccc4c3ccc3ccc5oc6ccccc6c5c34)c2)cc1]

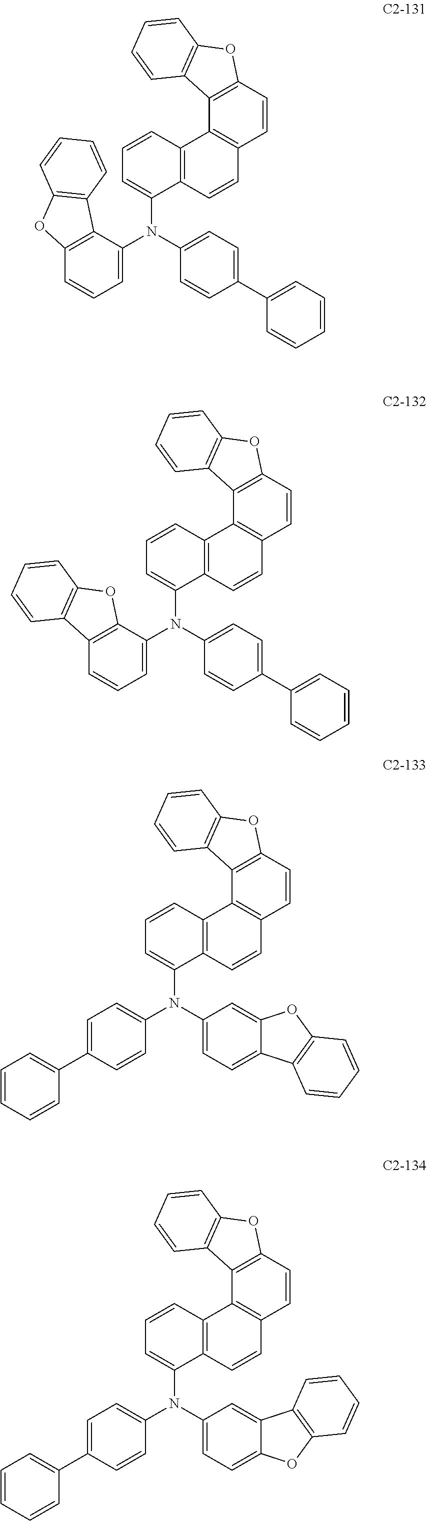 [SMILES: c1ccc(-c2ccc(N(c3cccc4c3ccc3ccc5oc6ccccc6c5c34)c3cccc4oc5ccccc5c34)cc2)cc1 ; c1ccc(-c2ccc(N(c3cccc4c3ccc3ccc5oc6ccccc6c5c34)c3cccc4c3oc3ccccc34)cc2)cc1 ; c1ccc(-c2ccc(N(c3ccc4c(c3)oc3ccccc34)c3cccc4c3ccc3ccc5oc6ccccc6c5c34)cc2)cc1 ; c1ccc(-c2ccc(N(c3ccc4oc5ccccc5c4c3)c3cccc4c3ccc3ccc5oc6ccccc6c5c34)cc2)cc1]

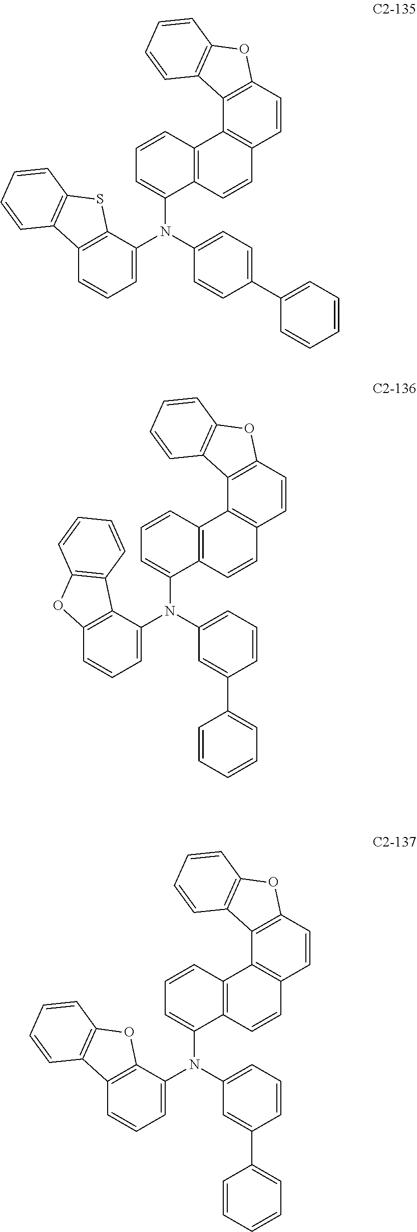 [SMILES: c1ccc(-c2ccc(N(c3cccc4c3ccc3ccc5oc6ccccc6c5c34)c3cccc4c3sc3ccccc34)cc2)cc1 ; c1ccc(-c2cccc(N(c3cccc4c3ccc3ccc5oc6ccccc6c5c34)c3cccc4oc5ccccc5c34)c2)cc1 ; c1ccc(-c2cccc(N(c3cccc4c3ccc3ccc5oc6ccccc6c5c34)c3cccc4c3oc3ccccc34)c2)cc1]

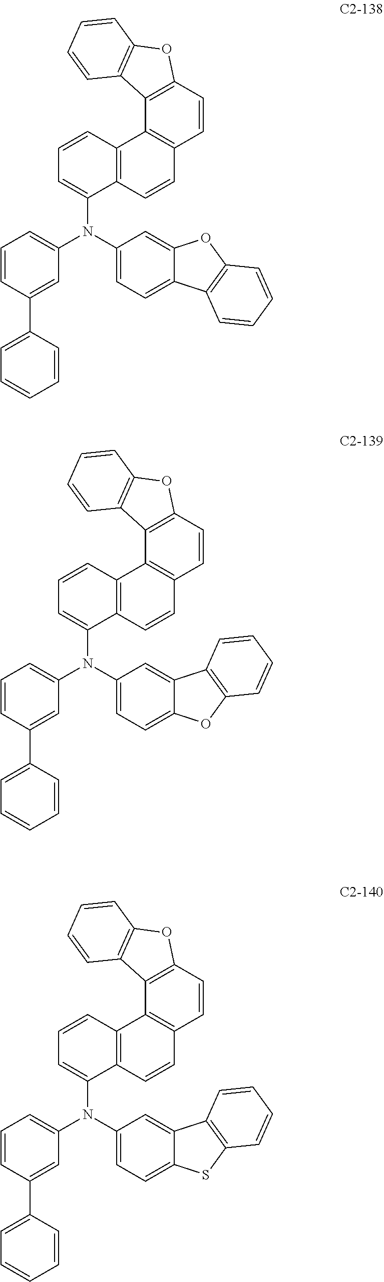 [SMILES: c1ccc(-c2cccc(N(c3ccc4c(c3)oc3ccccc34)c3cccc4c3ccc3ccc5oc6ccccc6c5c34)c2)cc1 ; c1ccc(-c2cccc(N(c3ccc4oc5ccccc5c4c3)c3cccc4c3ccc3ccc5oc6ccccc6c5c34)c2)cc1 ; c1ccc(-c2cccc(N(c3ccc4sc5ccccc5c4c3)c3cccc4c3ccc3ccc5oc6ccccc6c5c34)c2)cc1]

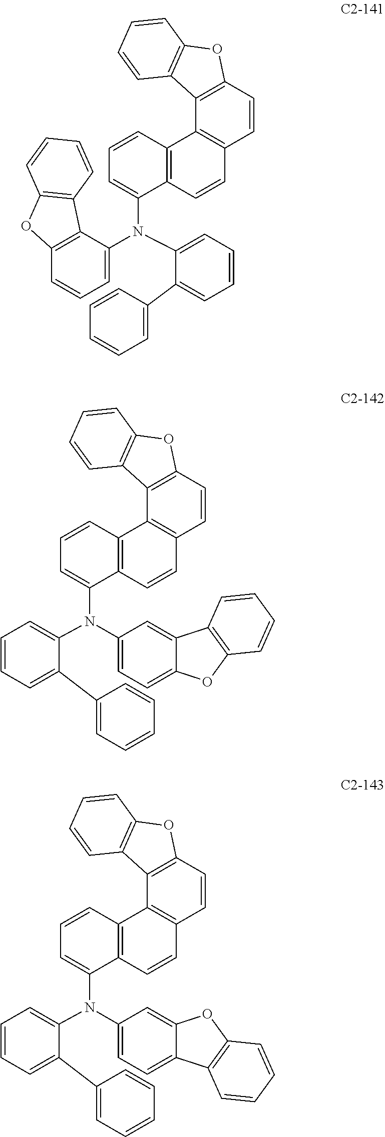

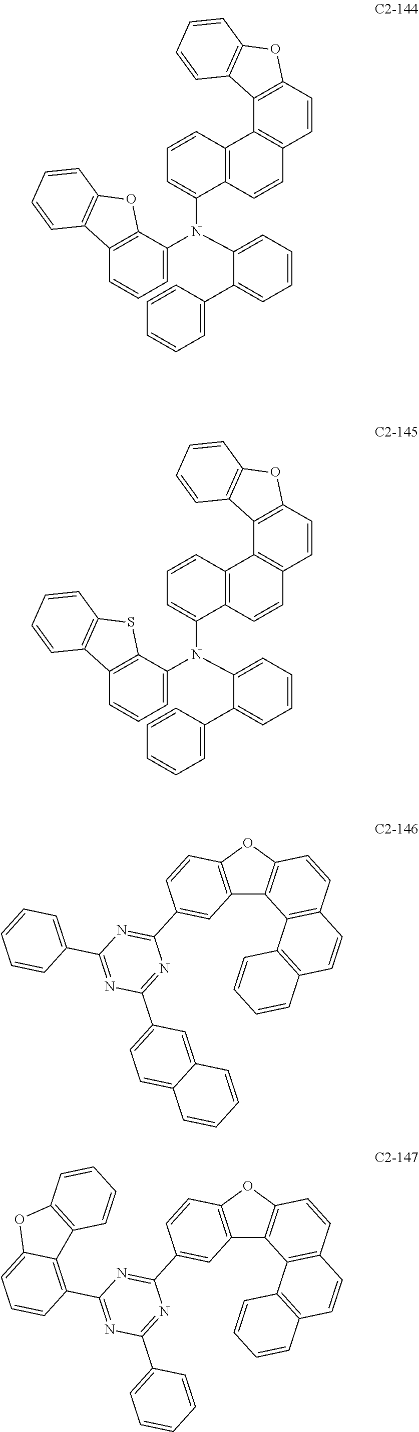 [SMILES: c1ccc(-c2ccccc2N(c2cccc3c2ccc2ccc4oc5ccccc5c4c23)c2cccc3c2oc2ccccc23)cc1 ; c1ccc(-c2ccccc2N(c2cccc3c2ccc2ccc4oc5ccccc5c4c23)c2cccc3c2sc2ccccc23)cc1 ; c1ccc(-c2nc(-c3ccc4ccccc4c3)nc(-c3ccc4oc5ccc6ccc7ccccc7c6c5c4c3)n2)cc1 ; c1ccc(-c2nc(-c3ccc4oc5ccc6ccc7ccccc7c6c5c4c3)nc(-c3cccc4oc5ccccc5c34)n2)cc1]

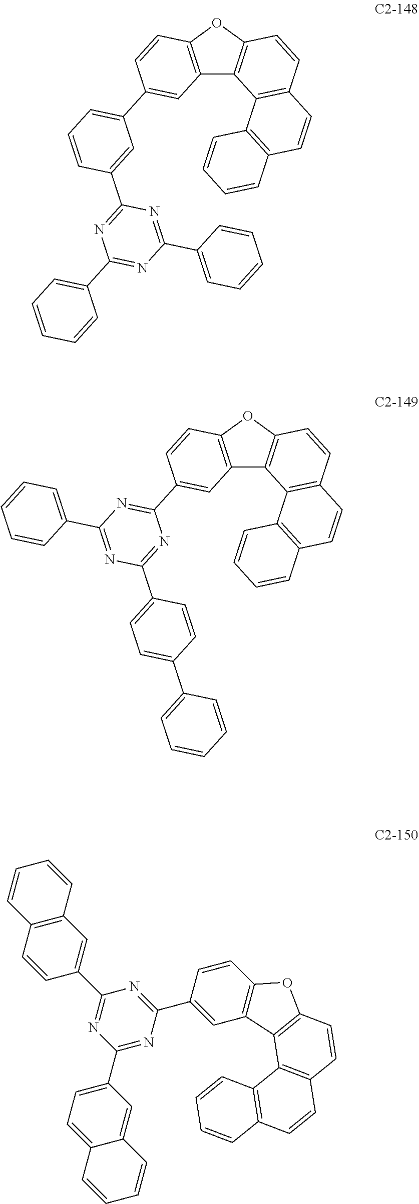 [SMILES: c1ccc(-c2nc(-c3ccccc3)nc(-c3cccc(-c4ccc5oc6ccc7ccc8ccccc8c7c6c5c4)c3)n2)cc1 ; c1ccc(-c2ccc(-c3nc(-c4ccccc4)nc(-c4ccc5oc6ccc7ccc8ccccc8c7c6c5c4)n3)cc2)cc1 ; c1ccc2cc(-c3nc(-c4ccc5ccccc5c4)nc(-c4ccc5oc6ccc7ccc8ccccc8c7c6c5c4)n3)ccc2c1]

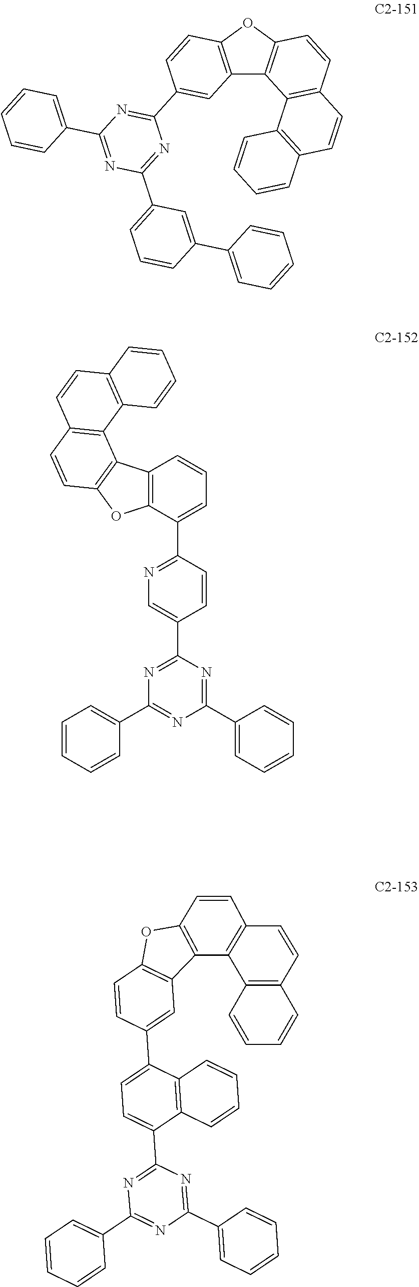 [SMILES: c1ccc(-c2cccc(-c3nc(-c4ccccc4)nc(-c4ccc5oc6ccc7ccc8ccccc8c7c6c5c4)n3)c2)cc1 ; c1ccc(-c2nc(-c3ccccc3)nc(-c3ccc(-c4cccc5c4oc4ccc6ccc7ccccc7c6c45)nc3)n2)cc1 ; c1ccc(-c2nc(-c3ccccc3)nc(-c3ccc(-c4ccc5oc6ccc7ccc8ccccc8c7c6c5c4)c4ccccc34)n2)cc1]

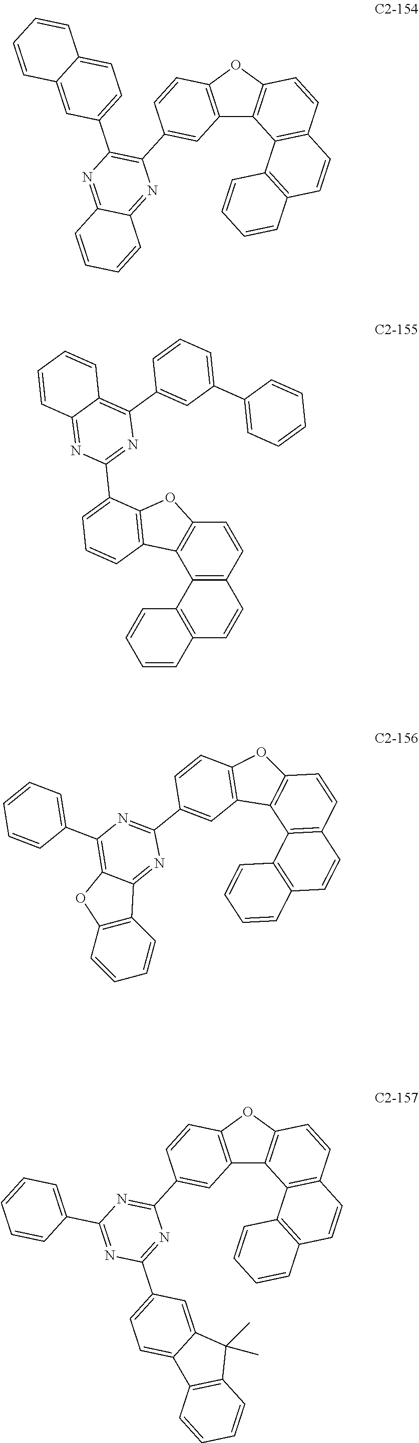 [SMILES: c1ccc2cc(-c3nc4ccccc4nc3-c3ccc4oc5ccc6ccc7ccccc7c6c5c4c3)ccc2c1 ; c1ccc(-c2cccc(-c3nc(-c4cccc5c4oc4ccc6ccc7ccccc7c6c45)nc4ccccc34)c2)cc1 ; c1ccc(-c2nc(-c3ccc4oc5ccc6ccc7ccccc7c6c5c4c3)nc3c2oc2ccccc23)cc1 ; CC1(C)c2ccccc2-c2ccc(-c3nc(-c4ccccc4)nc(-c4ccc5oc6ccc7ccc8ccccc8c7c6c5c4)n3)cc21]

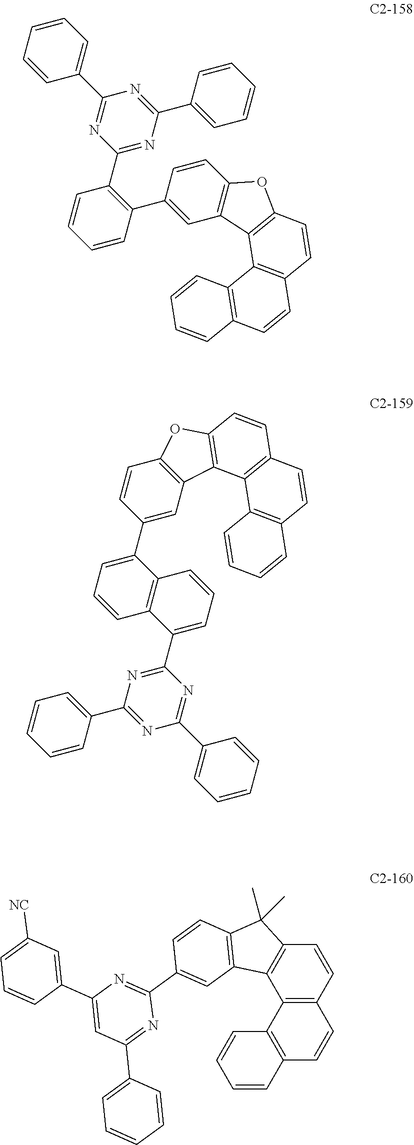 [SMILES: c1ccc(-c2nc(-c3ccccc3)nc(-c3ccccc3-c3ccc4oc5ccc6ccc7ccccc7c6c5c4c3)n2)cc1 ; c1ccc(-c2nc(-c3ccccc3)nc(-c3cccc4c(-c5ccc6oc7ccc8ccc9ccccc9c8c7c6c5)cccc34)n2)cc1 ; CC1(C)c2ccc(-c3nc(-c4ccccc4)cc(-c4cccc(C#N)c4)n3)cc2-c2c1ccc1ccc3ccccc3c21]

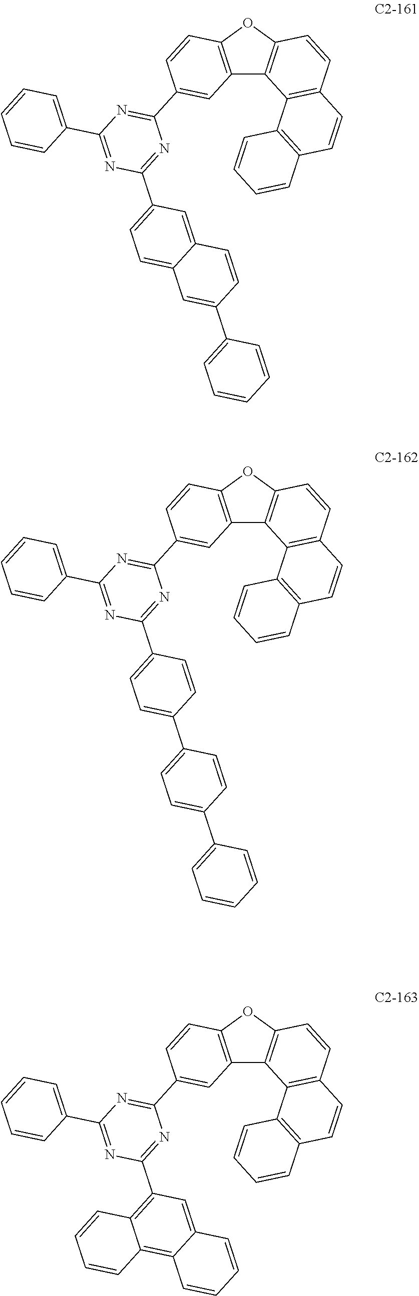 [SMILES: c1ccc(-c2ccc3cc(-c4nc(-c5ccccc5)nc(-c5ccc6oc7ccc8ccc9ccccc9c8c7c6c5)n4)ccc3c2)cc1 ; c1ccc(-c2ccc(-c3ccc(-c4nc(-c5ccccc5)nc(-c5ccc6oc7ccc8ccc9ccccc9c8c7c6c5)n4)cc3)cc2)cc1 ; c1ccc(-c2nc(-c3ccc4oc5ccc6ccc7ccccc7c6c5c4c3)nc(-c3cc4ccccc4c4ccccc34)n2)cc1]

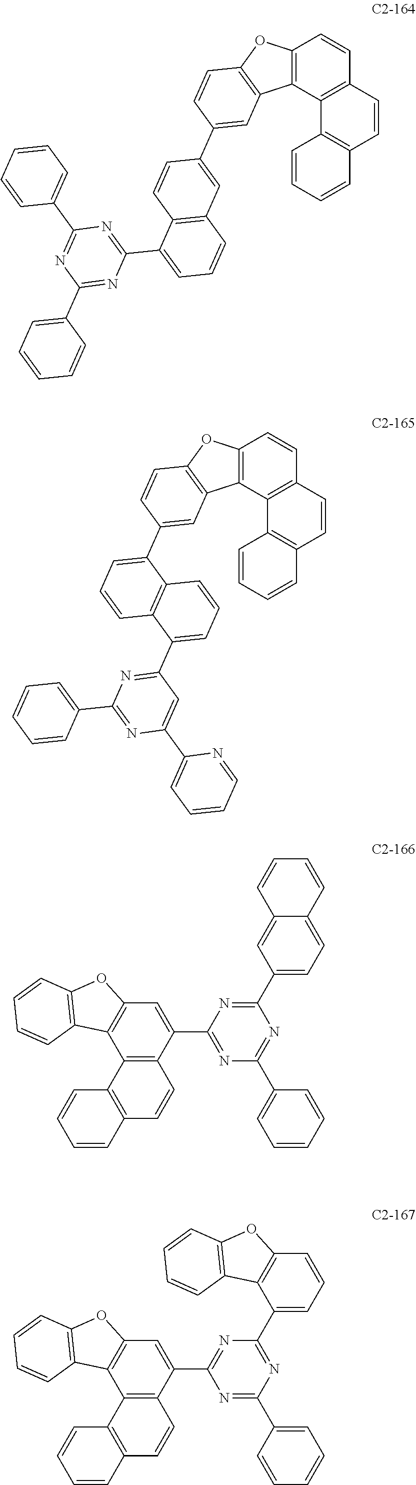 [SMILES: c1ccc(-c2nc(-c3ccccc3)nc(-c3cccc4cc(-c5ccc6oc7ccc8ccc9ccccc9c8c7c6c5)ccc34)n2)cc1 ; c1ccc(-c2nc(-c3ccccn3)cc(-c3cccc4c(-c5ccc6oc7ccc8ccc9ccccc9c8c7c6c5)cccc34)n2)cc1 ; c1ccc(-c2nc(-c3ccc4ccccc4c3)nc(-c3cc4oc5ccccc5c4c4c3ccc3ccccc34)n2)cc1 ; c1ccc(-c2nc(-c3cc4oc5ccccc5c4c4c3ccc3ccccc34)nc(-c3cccc4oc5ccccc5c34)n2)cc1]

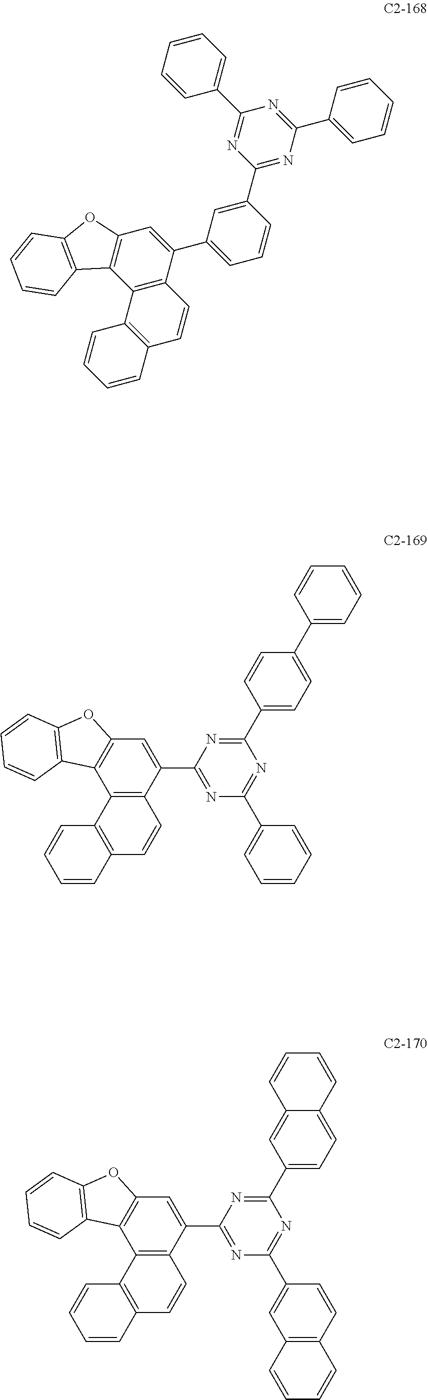 [SMILES: c1ccc(-c2nc(-c3ccccc3)nc(-c3cccc(-c4cc5oc6ccccc6c5c5c4ccc4ccccc45)c3)n2)cc1 ; c1ccc(-c2ccc(-c3nc(-c4ccccc4)nc(-c4cc5oc6ccccc6c5c5c4ccc4ccccc45)n3)cc2)cc1 ; c1ccc2cc(-c3nc(-c4ccc5ccccc5c4)nc(-c4cc5oc6ccccc6c5c5c4ccc4ccccc45)n3)ccc2c1]

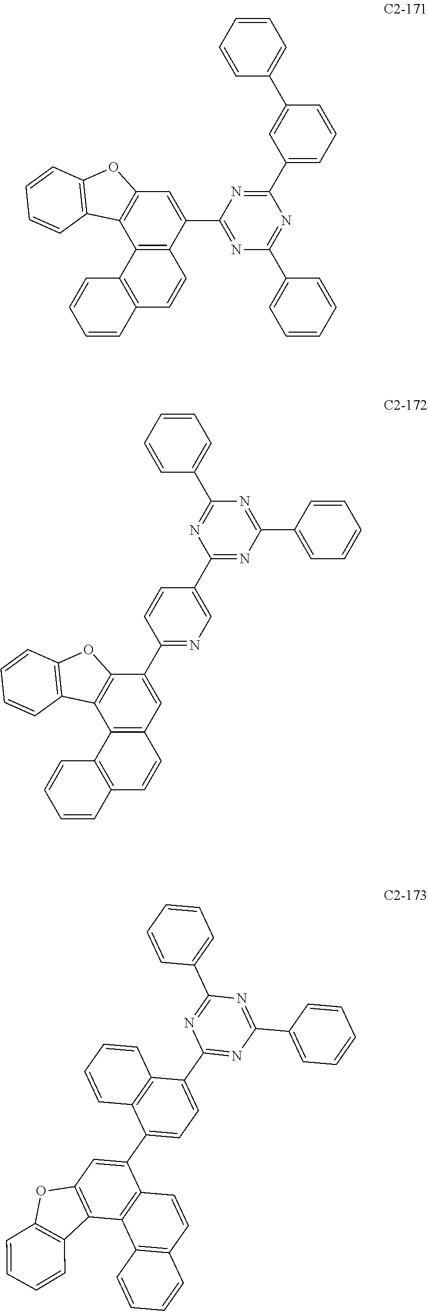 [SMILES: c1ccc(-c2cccc(-c3nc(-c4ccccc4)nc(-c4cc5oc6ccccc6c5c5c4ccc4ccccc45)n3)c2)cc1 ; c1ccc(-c2nc(-c3ccccc3)nc(-c3ccc(-c4cc5ccc6ccccc6c5c5c4oc4ccccc45)nc3)n2)cc1 ; c1ccc(-c2nc(-c3ccccc3)nc(-c3ccc(-c4cc5oc6ccccc6c5c5c4ccc4ccccc45)c4ccccc34)n2)cc1]

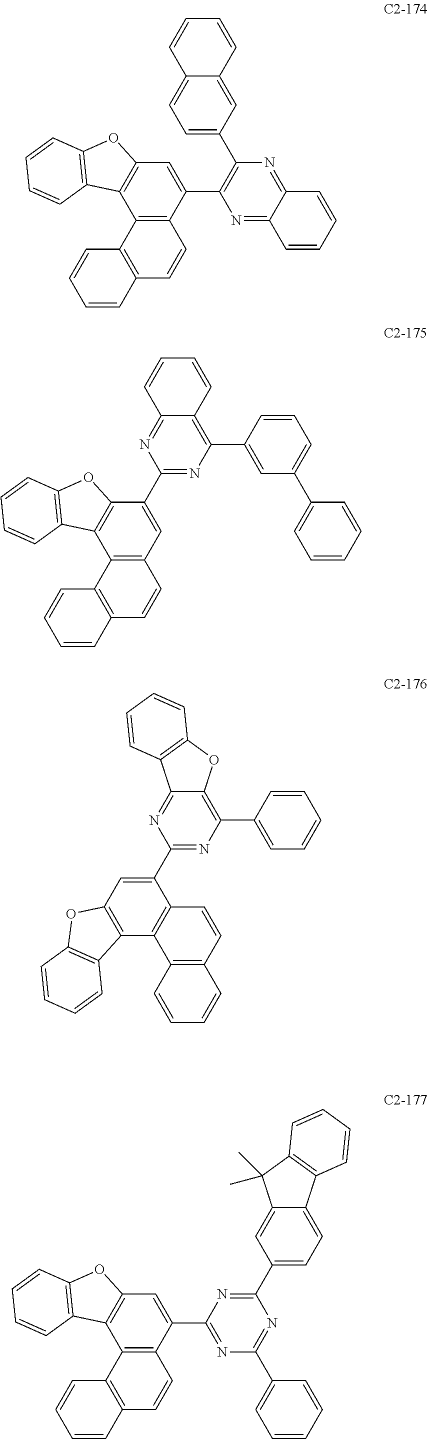 [SMILES: c1ccc2cc(-c3nc4ccccc4nc3-c3cc4oc5ccccc5c4c4c3ccc3ccccc34)ccc2c1 ; c1ccc(-c2cccc(-c3nc(-c4cc5ccc6ccccc6c5c5c4oc4ccccc45)nc4ccccc34)c2)cc1 ; c1ccc(-c2nc(-c3cc4oc5ccccc5c4c4c3ccc3ccccc34)nc3c2oc2ccccc23)cc1 ; CC1(C)c2ccccc2-c2ccc(-c3nc(-c4ccccc4)nc(-c4cc5oc6ccccc6c5c5c4ccc4ccccc45)n3)cc21]

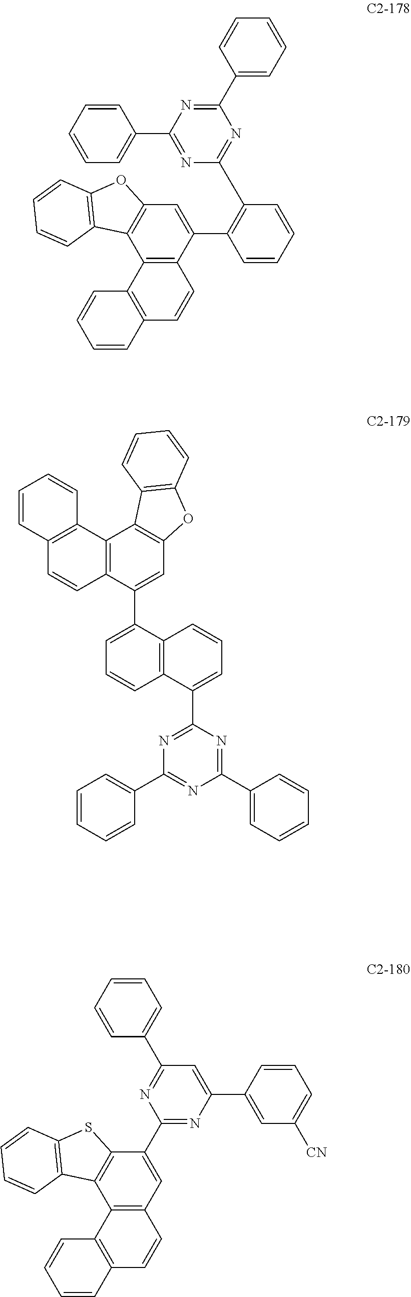 [SMILES: c1ccc(-c2nc(-c3ccccc3)nc(-c3ccccc3-c3cc4oc5ccccc5c4c4c3ccc3ccccc34)n2)cc1 ; c1ccc(-c2nc(-c3ccccc3)nc(-c3cccc4c(-c5cc6oc7ccccc7c6c6c5ccc5ccccc56)cccc34)n2)cc1 ; N#Cc1cccc(-c2cc(-c3ccccc3)nc(-c3cc4ccc5ccccc5c4c4c3sc3ccccc34)n2)c1]

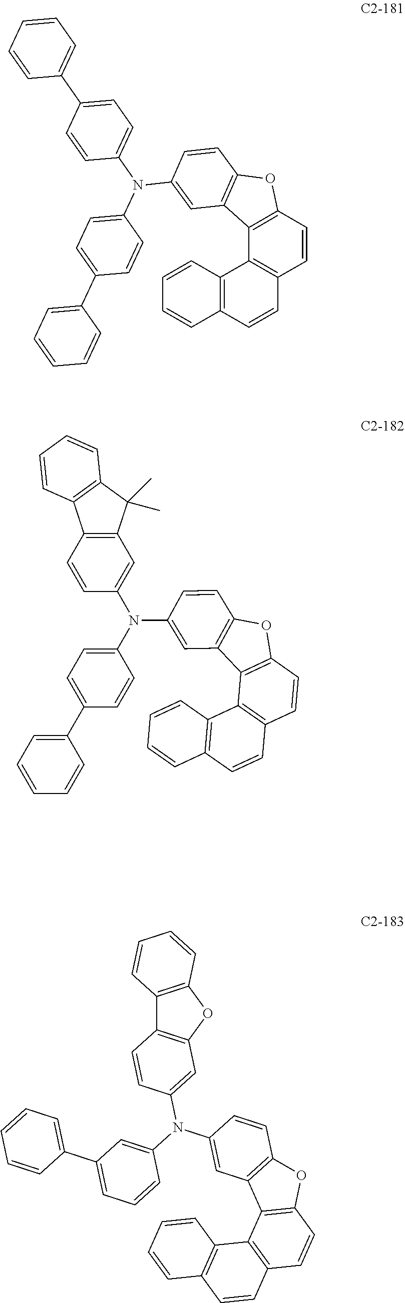 [SMILES: c1ccc(-c2ccc(N(c3ccc(-c4ccccc4)cc3)c3ccc4oc5ccc6ccc7ccccc7c6c5c4c3)cc2)cc1 ; CC1(C)c2ccccc2-c2ccc(N(c3ccc(-c4ccccc4)cc3)c3ccc4oc5ccc6ccc7ccccc7c6c5c4c3)cc21 ; c1ccc(-c2cccc(N(c3ccc4c(c3)oc3ccccc34)c3ccc4oc5ccc6ccc7ccccc7c6c5c4c3)c2)cc1]

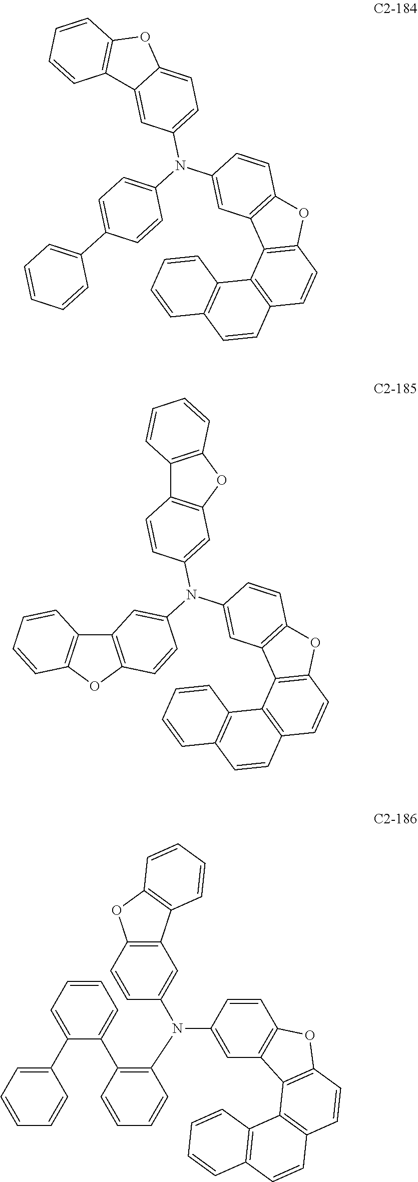 [SMILES: c1ccc(-c2ccc(N(c3ccc4oc5ccccc5c4c3)c3ccc4oc5ccc6ccc7ccccc7c6c5c4c3)cc2)cc1 ; c1ccc2c(c1)ccc1ccc3oc4ccc(N(c5ccc6c(c5)oc5ccccc56)c5ccc6oc7ccccc7c6c5)cc4c3c12 ; c1ccc(-c2ccccc2-c2ccccc2N(c2ccc3oc4ccccc4c3c2)c2ccc3oc4ccc5ccc6ccccc6c5c4c3c2)cc1]

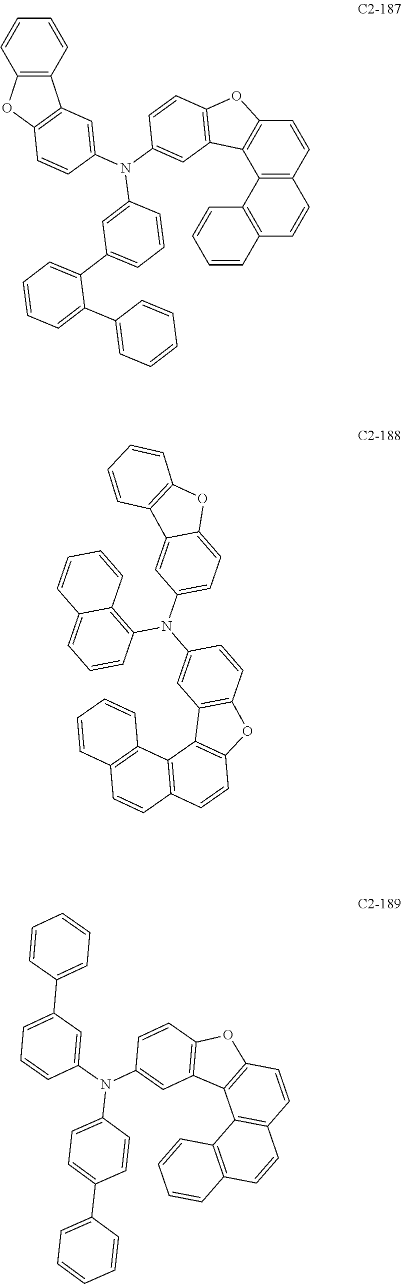 [SMILES: c1ccc(-c2ccccc2-c2cccc(N(c3ccc4oc5ccccc5c4c3)c3ccc4oc5ccc6ccc7ccccc7c6c5c4c3)c2)cc1 ; c1ccc2c(N(c3ccc4oc5ccccc5c4c3)c3ccc4oc5ccc6ccc7ccccc7c6c5c4c3)cccc2c1 ; c1ccc(-c2ccc(N(c3cccc(-c4ccccc4)c3)c3ccc4oc5ccc6ccc7ccccc7c6c5c4c3)cc2)cc1]

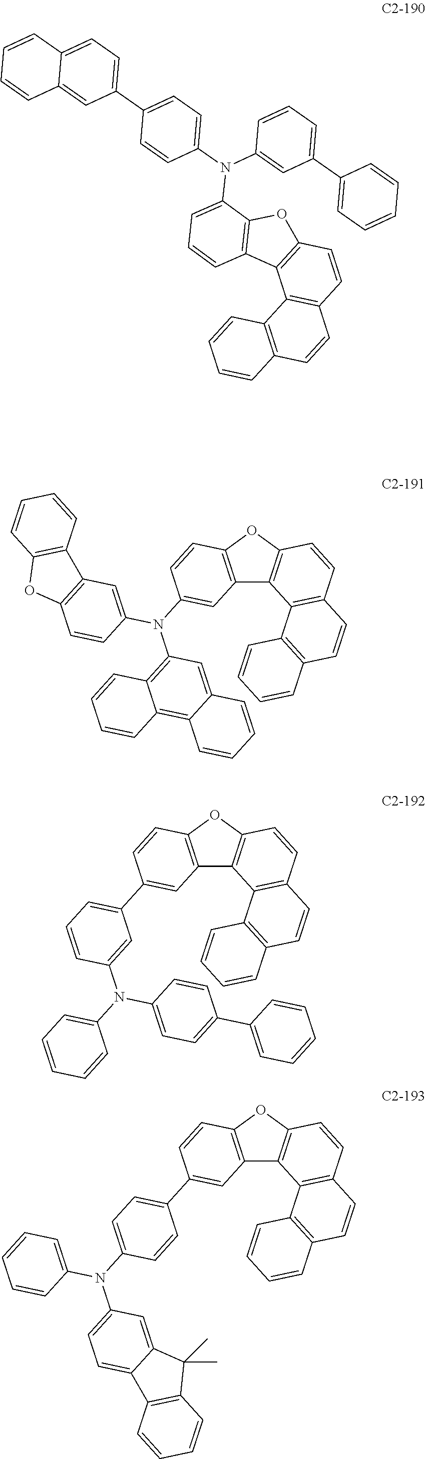 [SMILES: c1ccc(-c2cccc(N(c3ccc(-c4ccc5ccccc5c4)cc3)c3cccc4c3oc3ccc5ccc6ccccc6c5c34)c2)cc1 ; c1ccc2c(c1)cc(N(c1ccc3oc4ccccc4c3c1)c1ccc3oc4ccc5ccc6ccccc6c5c4c3c1)c1ccccc12 ; c1ccc(-c2ccc(N(c3ccccc3)c3cccc(-c4ccc5oc6ccc7ccc8ccccc8c7c6c5c4)c3)cc2)cc1 ; CC1(C)c2ccccc2-c2ccc(N(c3ccccc3)c3ccc(-c4ccc5oc6ccc7ccc8ccccc8c7c6c5c4)cc3)cc21]

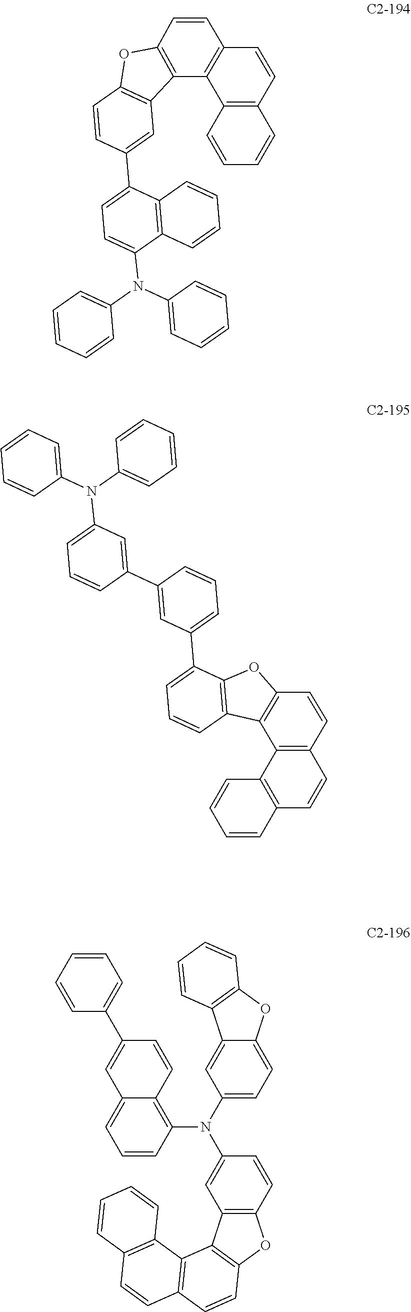 [SMILES: c1ccc(N(c2ccccc2)c2ccc(-c3ccc4oc5ccc6ccc7ccccc7c6c5c4c3)c3ccccc23)cc1 ; c1ccc(N(c2ccccc2)c2cccc(-c3cccc(-c4cccc5c4oc4ccc6ccc7ccccc7c6c45)c3)c2)cc1 ; c1ccc(-c2ccc3c(N(c4ccc5oc6ccccc6c5c4)c4ccc5oc6ccc7ccc8ccccc8c7c6c5c4)cccc3c2)cc1]

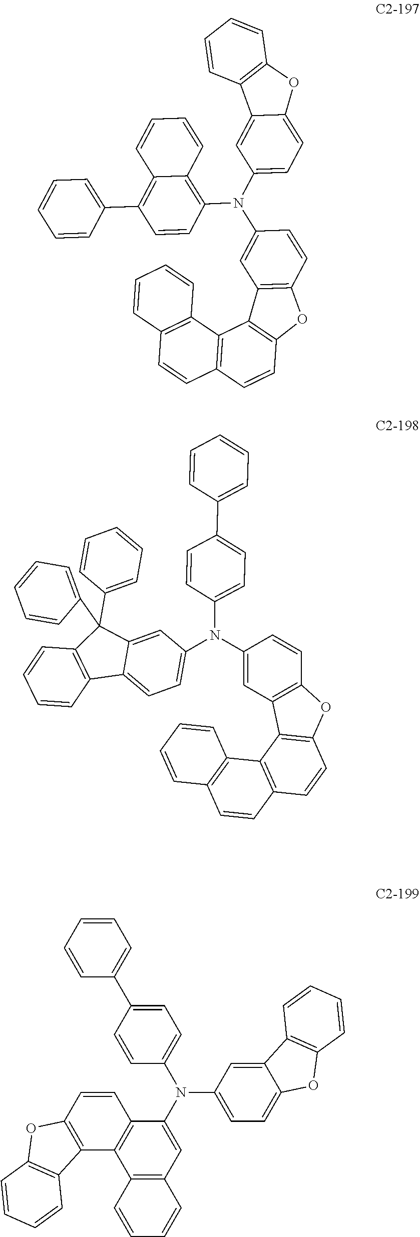 [SMILES: c1ccc(-c2ccc(N(c3ccc4oc5ccccc5c4c3)c3ccc4oc5ccc6ccc7ccccc7c6c5c4c3)c3ccccc23)cc1 ; c1ccc(-c2ccc(N(c3ccc4c(c3)C(c3ccccc3)(c3ccccc3)c3ccccc3-4)c3ccc4oc5ccc6ccc7ccccc7c6c5c4c3)cc2)cc1 ; c1ccc(-c2ccc(N(c3ccc4oc5ccccc5c4c3)c3cc4ccccc4c4c3ccc3oc5ccccc5c34)cc2)cc1]

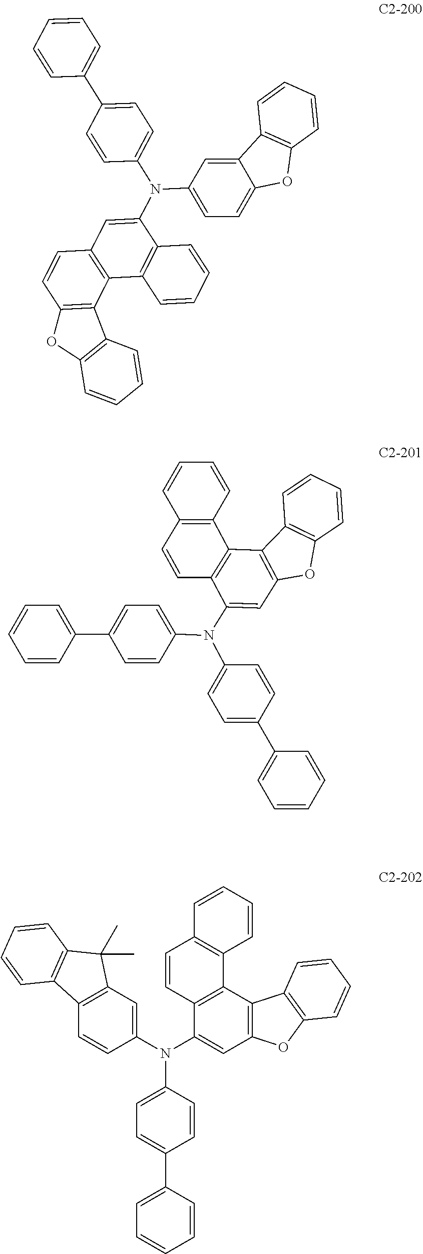 [SMILES: c1ccc(-c2ccc(N(c3ccc4oc5ccccc5c4c3)c3cc4ccc5oc6ccccc6c5c4c4ccccc34)cc2)cc1 ; c1ccc(-c2ccc(N(c3ccc(-c4ccccc4)cc3)c3cc4oc5ccccc5c4c4c3ccc3ccccc34)cc2)cc1 ; CC1(C)c2ccccc2-c2ccc(N(c3ccc(-c4ccccc4)cc3)c3cc4oc5ccccc5c4c4c3ccc3ccccc34)cc21]

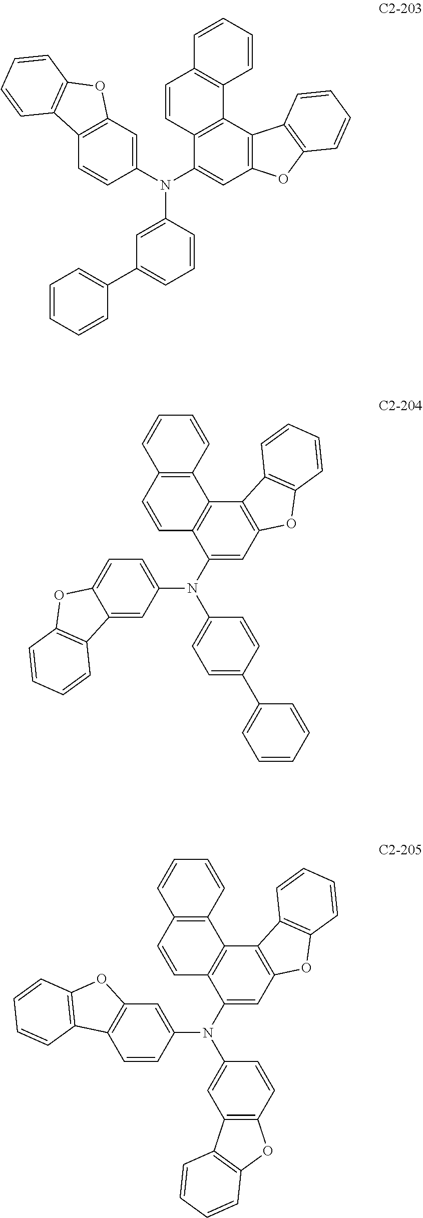 [SMILES: c1ccc(-c2cccc(N(c3ccc4c(c3)oc3ccccc34)c3cc4oc5ccccc5c4c4c3ccc3ccccc34)c2)cc1 ; c1ccc(-c2ccc(N(c3ccc4oc5ccccc5c4c3)c3cc4oc5ccccc5c4c4c3ccc3ccccc34)cc2)cc1 ; c1ccc2c(c1)ccc1c(N(c3ccc4c(c3)oc3ccccc34)c3ccc4oc5ccccc5c4c3)cc3oc4ccccc4c3c12]

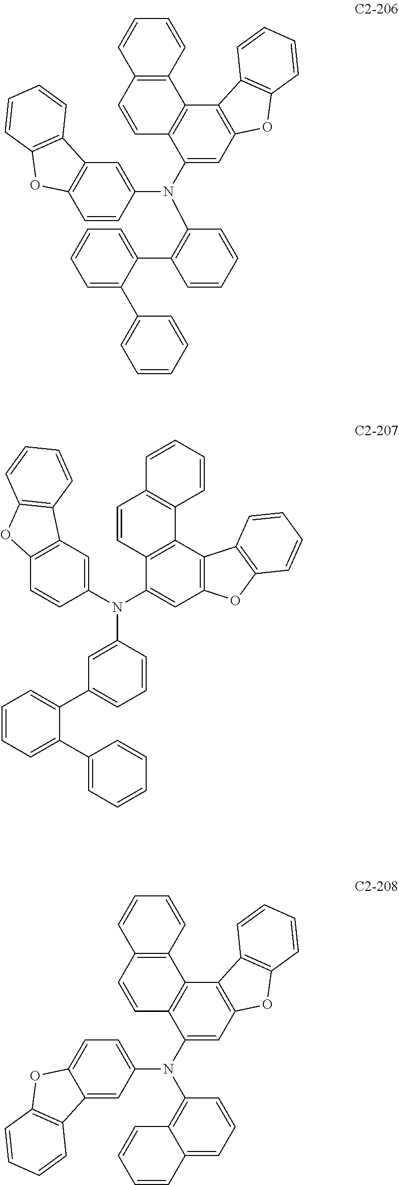 [SMILES: c1ccc(-c2ccccc2-c2ccccc2N(c2ccc3oc4ccccc4c3c2)c2cc3oc4ccccc4c3c3c2ccc2ccccc23)cc1 ; c1ccc(-c2ccccc2-c2cccc(N(c3ccc4oc5ccccc5c4c3)c3cc4oc5ccccc5c4c4c3ccc3ccccc34)c2)cc1 ; c1ccc2c(N(c3ccc4oc5ccccc5c4c3)c3cc4oc5ccccc5c4c4c3ccc3ccccc34)cccc2c1]

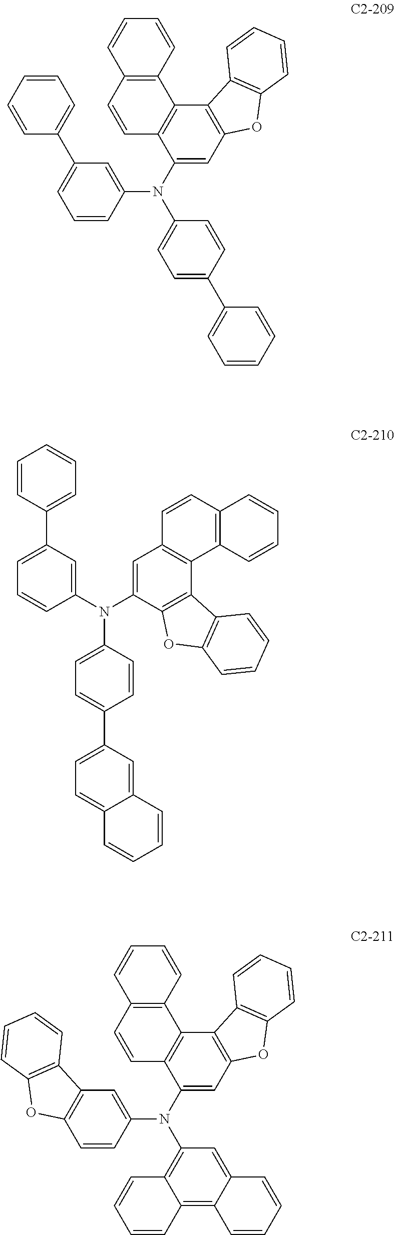 [SMILES: c1ccc(-c2ccc(N(c3cccc(-c4ccccc4)c3)c3cc4oc5ccccc5c4c4c3ccc3ccccc34)cc2)cc1 ; c1ccc(-c2cccc(N(c3ccc(-c4ccc5ccccc5c4)cc3)c3cc4ccc5ccccc5c4c4c3oc3ccccc34)c2)cc1 ; c1ccc2c(c1)cc(N(c1ccc3oc4ccccc4c3c1)c1cc3oc4ccccc4c3c3c1ccc1ccccc13)c1ccccc12]

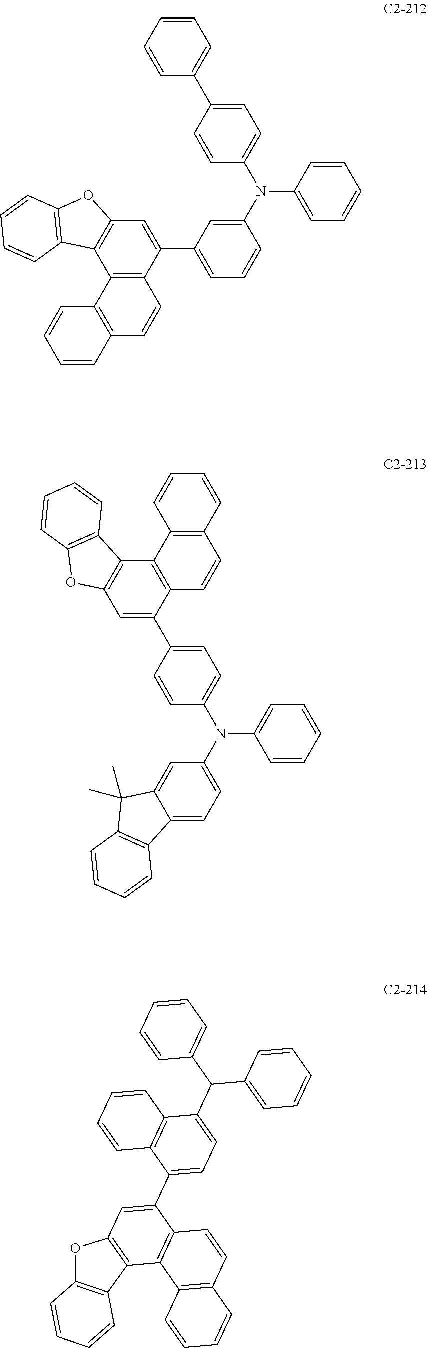 [SMILES: c1ccc(-c2ccc(N(c3ccccc3)c3cccc(-c4cc5oc6ccccc6c5c5c4ccc4ccccc45)c3)cc2)cc1 ; CC1(C)c2ccccc2-c2ccc(N(c3ccccc3)c3ccc(-c4cc5oc6ccccc6c5c5c4ccc4ccccc45)cc3)cc21 ; c1ccc(C(c2ccccc2)c2ccc(-c3cc4oc5ccccc5c4c4c3ccc3ccccc34)c3ccccc23)cc1]

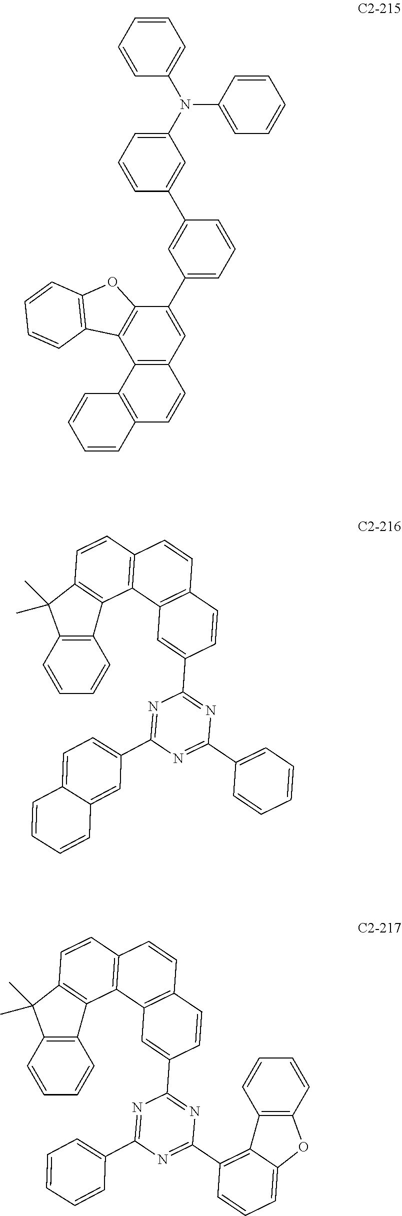 [SMILES: c1ccc(N(c2ccccc2)c2cccc(-c3cccc(-c4cc5ccc6ccccc6c5c5c4oc4ccccc45)c3)c2)cc1 ; CC1(C)c2ccccc2-c2c1ccc1ccc3ccc(-c4nc(-c5ccccc5)nc(-c5ccc6ccccc6c5)n4)cc3c21 ; CC1(C)c2ccccc2-c2c1ccc1ccc3ccc(-c4nc(-c5ccccc5)nc(-c5cccc6oc7ccccc7c56)n4)cc3c21]

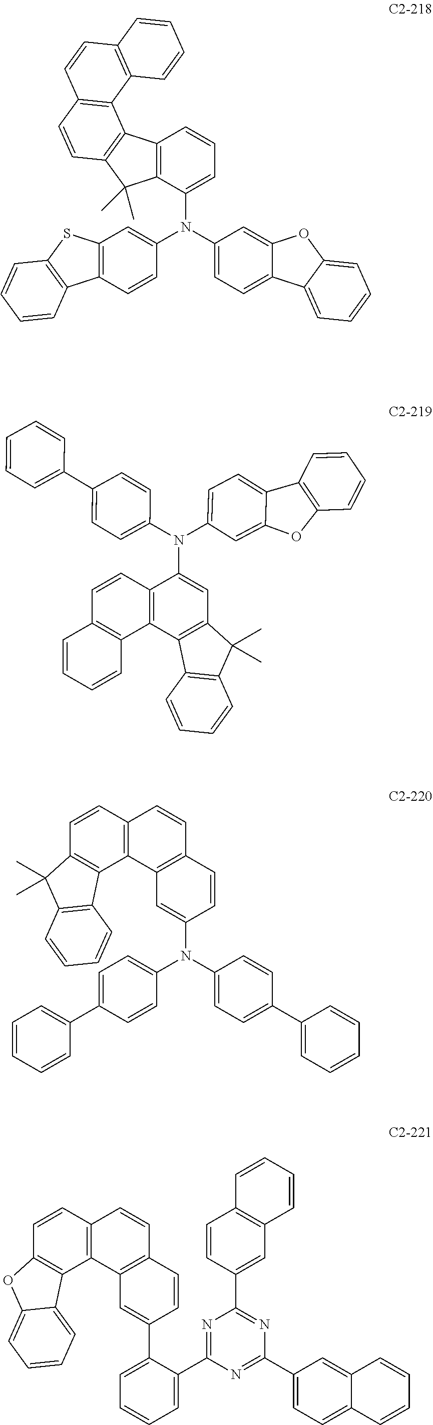 [SMILES: CC1(C)c2ccc3ccc4ccccc4c3c2-c2cccc(N(c3ccc4c(c3)oc3ccccc34)c3ccc4c(c3)sc3ccccc34)c21 ; CC1(C)c2ccccc2-c2c1cc(N(c1ccc(-c3ccccc3)cc1)c1ccc3c(c1)oc1ccccc13)c1ccc3ccccc3c21 ; CC1(C)c2ccccc2-c2c1ccc1ccc3ccc(N(c4ccc(-c5ccccc5)cc4)c4ccc(-c5ccccc5)cc4)cc3c21 ; c1ccc(-c2nc(-c3ccc4ccccc4c3)nc(-c3ccc4ccccc4c3)n2)c(-c2ccc3ccc4ccc5oc6ccccc6c5c4c3c2)c1]

The combination of at least one of compounds C1-1 to C1-187 and at least one of compounds C2-1 to C2-221 may be used in an organic electroluminescent device.

According to one embodiment of the present disclosure, the present disclosure may provide a compound represented by formula 1 or a compound represented by formula 2. Specifically, the present disclosure may provide at least one compound selected from the group consisting of compounds C1-1 to C1-187 and compounds C2-1 to C2-221.

The compound represented by formula 1 according to the present disclosure may be prepared by a synthetic method known to one skilled in the art. For example, the compound represented by formula 1 can be prepared by referring to Korean Patent Application Laid-Open Nos. 2015-0135109 (published on Dec. 2, 2015), 2016-0099471 (published on Aug. 22, 2016), 2015-0077513 (published on Jul. 8, 2015), 2017-0129599 (published on Nov. 27, 2017), and 2018-0066818 (published on Jun. 19, 2018), and Korean Patent No. 1786749 (published on Oct. 17, 2017), but is not limited thereto.

The compound represented by formula 2 according to the present disclosure may be prepared as shown in the Examples described herein and by a synthetic method known to one skilled in the art. For example, the compound represented by formula 2 can be prepared by referring to Korean Patent Application Laid-Open No. 2017-0043439 (published on Apr. 21, 2017), but is not limited thereto.

The organic electroluminescent device according to the present disclosure comprises an anode, a cathode, and at least one organic layer between the anode and the cathode. The organic layer may comprise a plurality of organic electroluminescent materials in which the compound represented by formula 1 is comprised as a first organic electroluminescent material, and the compound represented by formula 2 is comprised as a second organic electroluminescent material. According to one embodiment of the present disclosure, the organic electroluminescent device comprises an anode, a cathode, and at least one light-emitting layer between the anode and the cathode, and the light-emitting layer comprises a compound(s) represented by formula 1, and a compound(s) represented by formula 2.

The electrode may be a transflective electrode or a reflective electrode, and may be a top emission type, a bottom emission type, or a both-sides emission type, depending on the materials. The hole injection layer may be further doped with a p-dopant, and the electron injection layer may be further doped with an n-dopant.

The light-emitting layer comprises a host and a dopant. The host comprises a plurality of host materials. The compound represented by formula 1 may be comprised as a first host compound of the plurality of host materials, and the compound represented by formula 2 may be comprised as a second host compound of the plurality of host materials. The weight ratio of the first host compound to the second host compound is in the range of about 1:99 to about 99:1, preferably about 10:90 to about 90:10, more preferably about 30:70 to about 70:30, even more preferably about 40:60 to about 60:40, and still more preferably about 50:50. When two or more materials are included in one layer, they may be mixture-evaporated to form a layer, or may be separately co-evaporated at the same time to form a layer.

The light-emitting layer is a layer from which light is emitted, and can be a single layer or a multi-layer in which two or more layers are stacked. In the plurality of host materials according to the present disclosure, the first and second host materials may both be comprised in one layer, or may be respectively comprised in different light-emitting layers. According to one embodiment of the present disclosure, the doping concentration of the dopant compound with respect to the host compound in the light-emitting layer is less than about 20 wt %.

The organic electroluminescent device of the present disclosure may further comprise at least one layer selected from a hole injection layer, a hole transport layer, a hole auxiliary layer, a light-emitting auxiliary layer, an electron transport layer, an electron injection layer, an interlayer, an electron buffer layer, a hole blocking layer, and an electron blocking layer. According to one embodiment of the present disclosure, the organic electroluminescent device may further comprise amine-based compounds in addition to the plurality of host materials of the present disclosure as at least one of a hole injection material, a hole transport material, a hole auxiliary material, a light-emitting material, a light-emitting auxiliary material, and an electron blocking material. Also, according to one embodiment of the present disclosure, the organic electroluminescent device of the present disclosure may further comprise azine-based compounds in addition to the plurality of host materials of the present disclosure as at least one of an electron transport material, an electron injection material, an electron buffer material, and a hole blocking material.

The dopant comprised in the organic electroluminescent device according to the present disclosure may be at least one phosphorescent or fluorescent dopant, preferably at least one phosphorescent dopant. The phosphorescent dopant materials applied to the organic electroluminescent device according to the present disclosure are not particularly limited, but may be selected from metallated complex compounds of iridium (Ir), osmium (Os), copper (Cu), and platinum (Pt), may be preferably selected from ortho-metallated complex compounds of iridium (Ir), osmium (Os), copper (Cu), and platinum (Pt), and may be more preferably an ortho-metallated iridium complex compound.

The dopant comprised in the organic electroluminescent device of the present disclosure may include the compound represented by the following formula 101, but is not limited thereto.

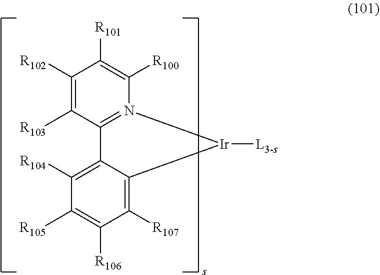

In formula 101, L is selected from the following structures 1 and 2:

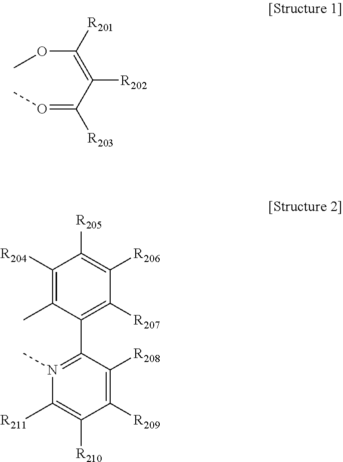

R₁₀₀ to R₁₀₃, each independently, represent hydrogen, deuterium, a halogen, a (C1-C30)alkyl unsubstituted or substituted with deuterium(s) and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent one(s) of R₁₀₀ to R₁₀₃ to form a ring(s), e.g., a substituted or unsubstituted, quinoline, isoquinoline, benzofuropyridine, benzothienopyridine, indenopyridine, benzofuroquinoline, benzothienoquinoline, or indenoquinoline ring, together with pyridine;
R₁₀₄ to R₁₀₇, each independently, represent hydrogen, deuterium, a halogen, a (C1-C30)alkyl unsubstituted or substituted with deuterium(s) and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent one(s) of R₁₀₄ to R₁₀₇ to form a ring(s), e.g., a substituted or unsubstituted, naphthalene, fluorene, dibenzothiophene, dibenzofuran, indenopyridine, benzofuropyridine or benzothienopyridine ring, together with benzene;
R₂₀₁ to R₂₁₁, each independently, represent hydrogen, deuterium, a halogen, a (C1-C30)alkyl unsubstituted or substituted with deuterium(s) and/or a halogen(s), a substituted or unsubstituted (C3-C30)cycloalkyl, or a substituted or unsubstituted (C6-C30)aryl; or may be linked to an adjacent one(s) of R₂₀₁ to R₂₁₁ to form a ring(s); and
s represents an integer of 1 to 3.

The specific examples of the dopant compound are as follows, but are not limited thereto.

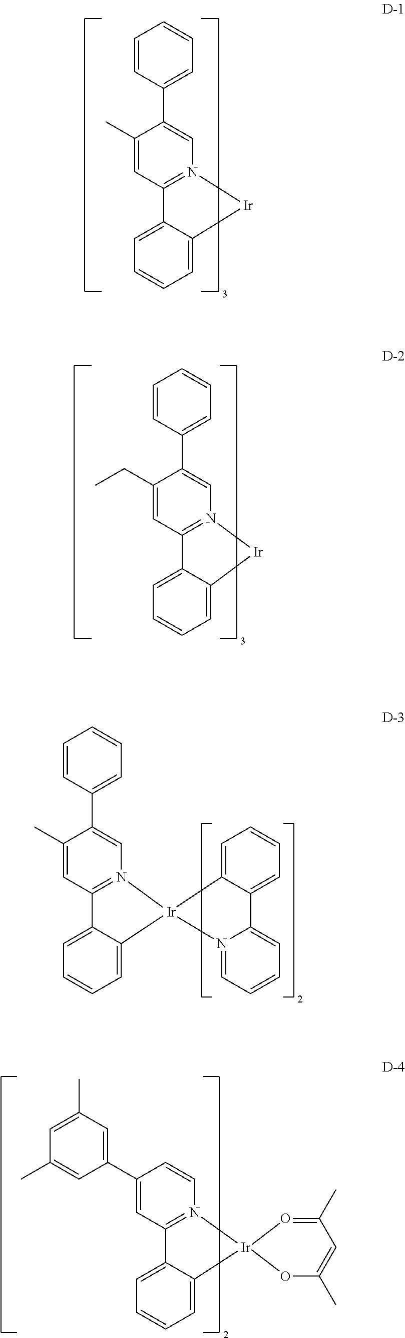

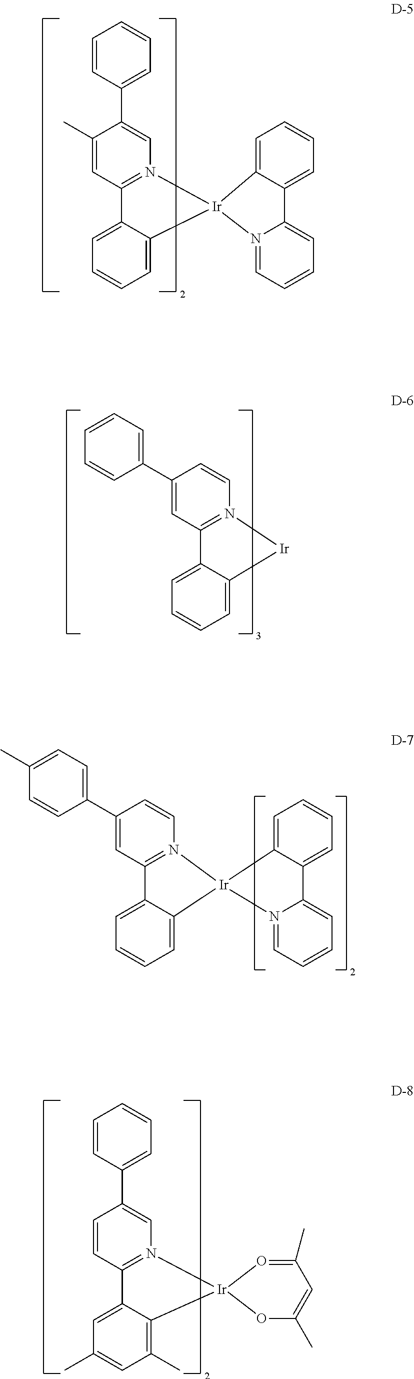

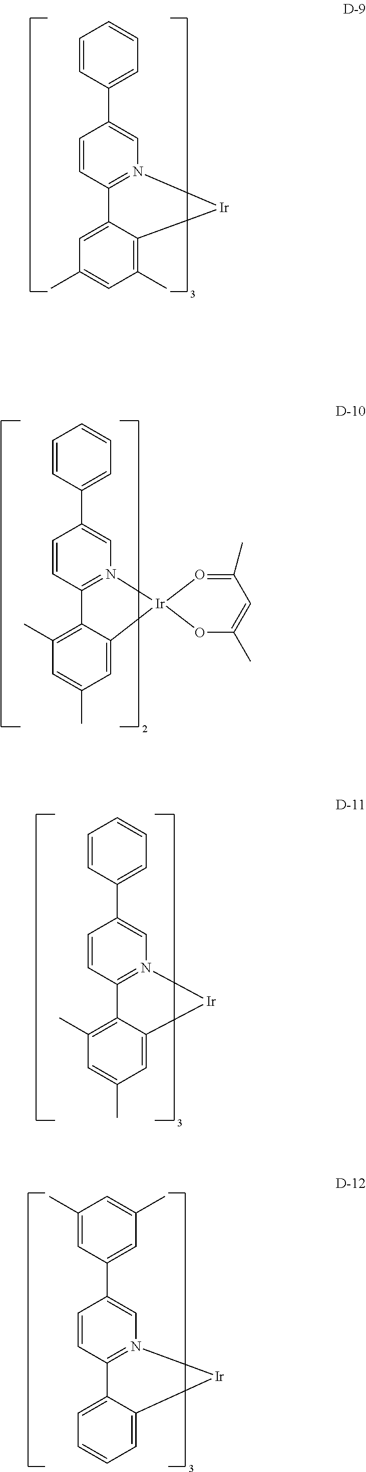

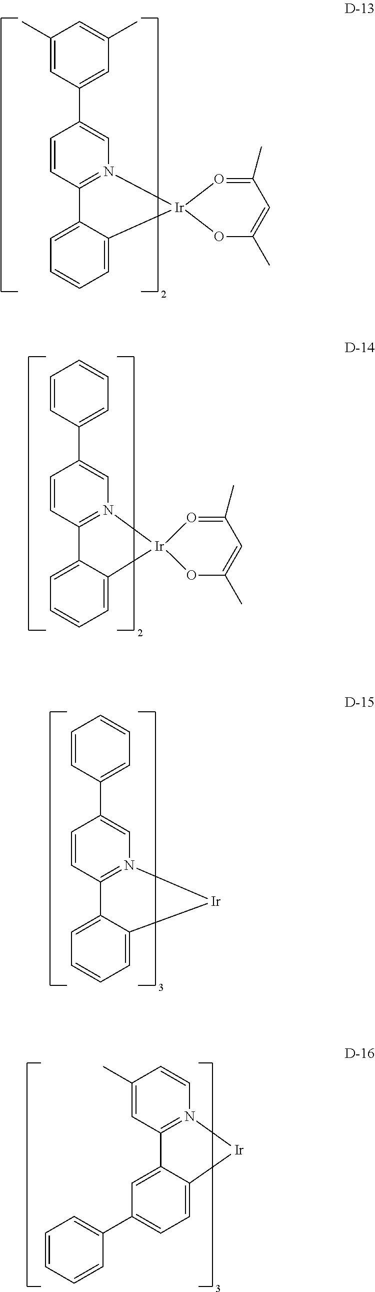

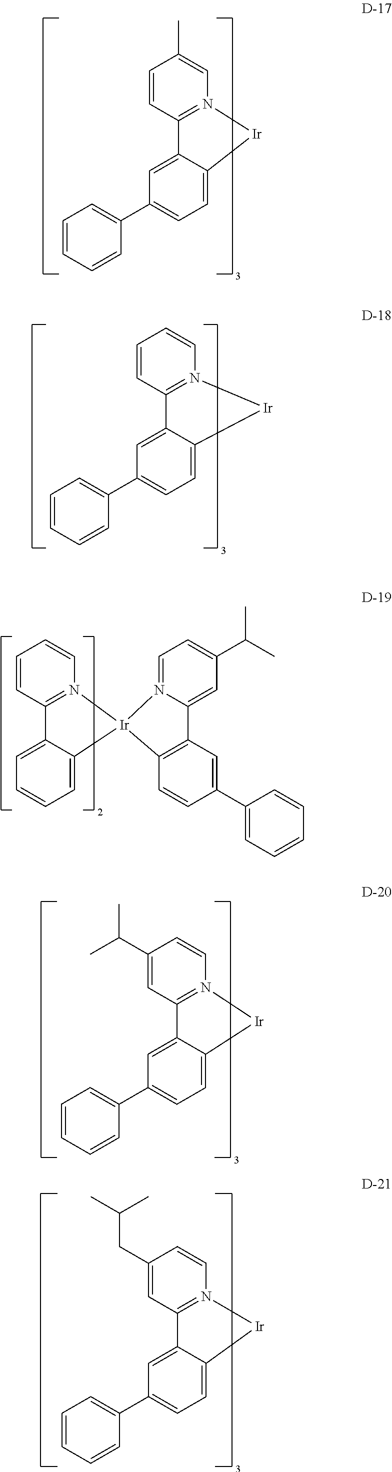

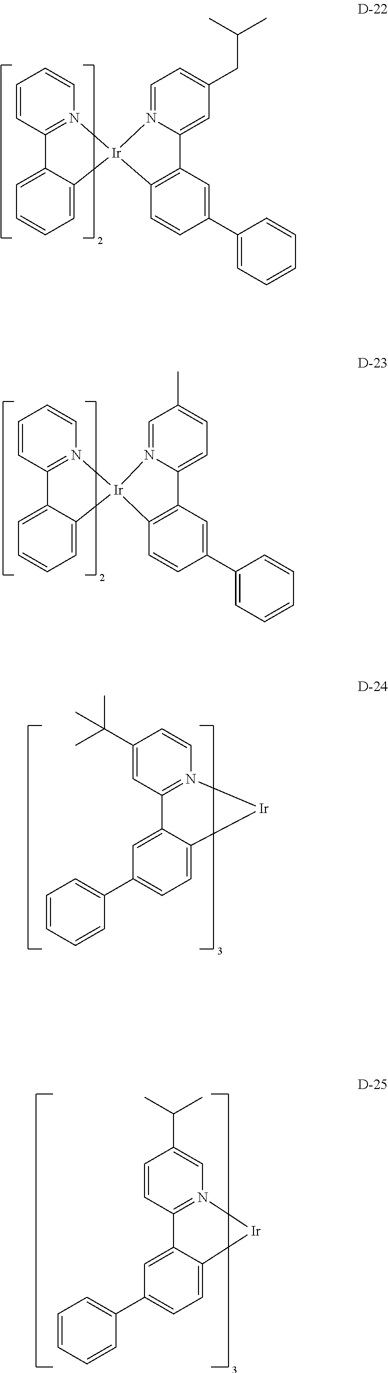

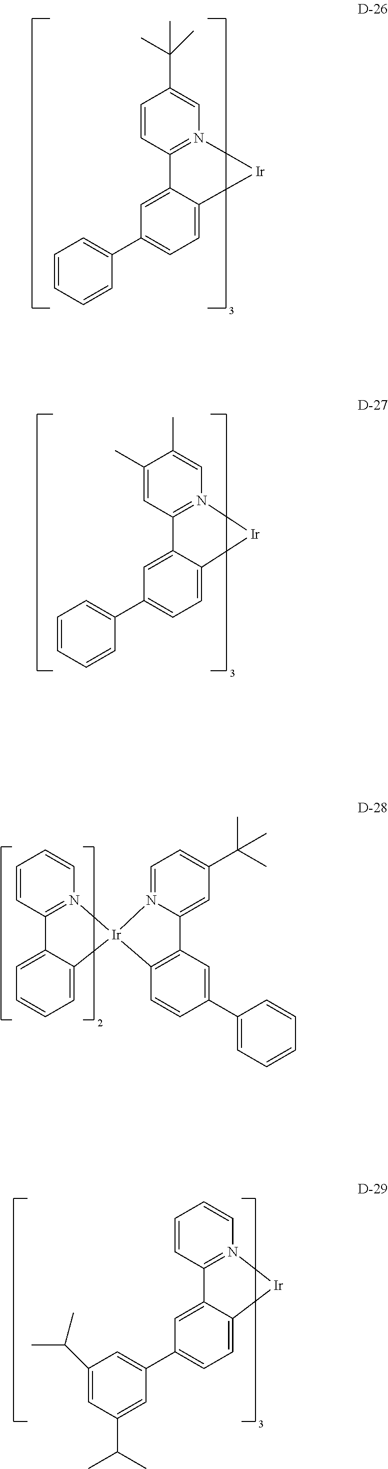

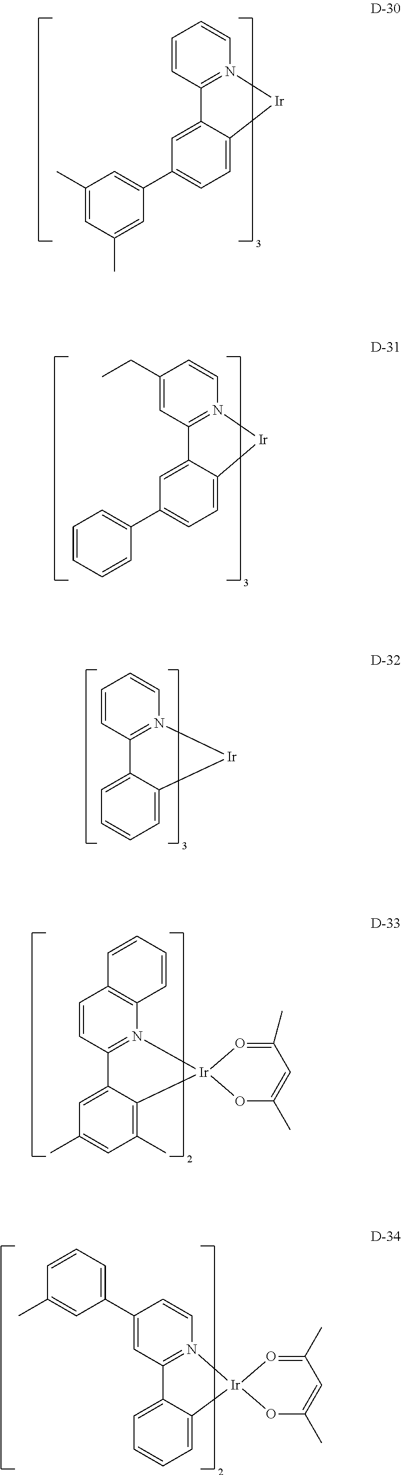

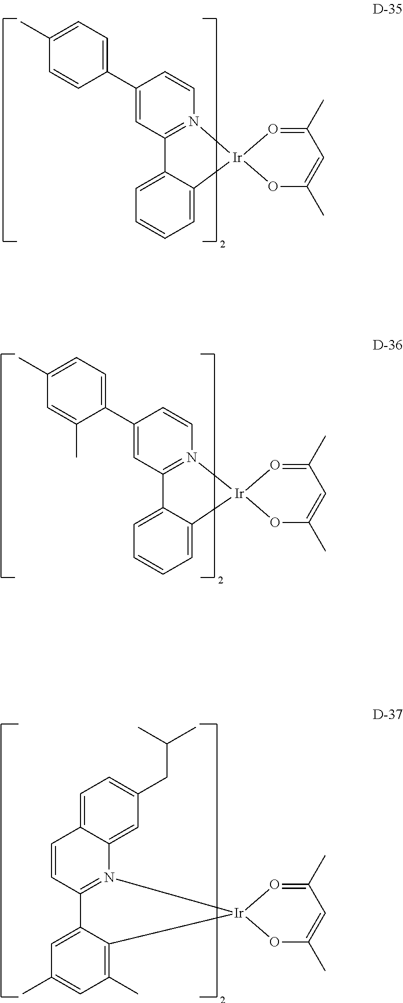

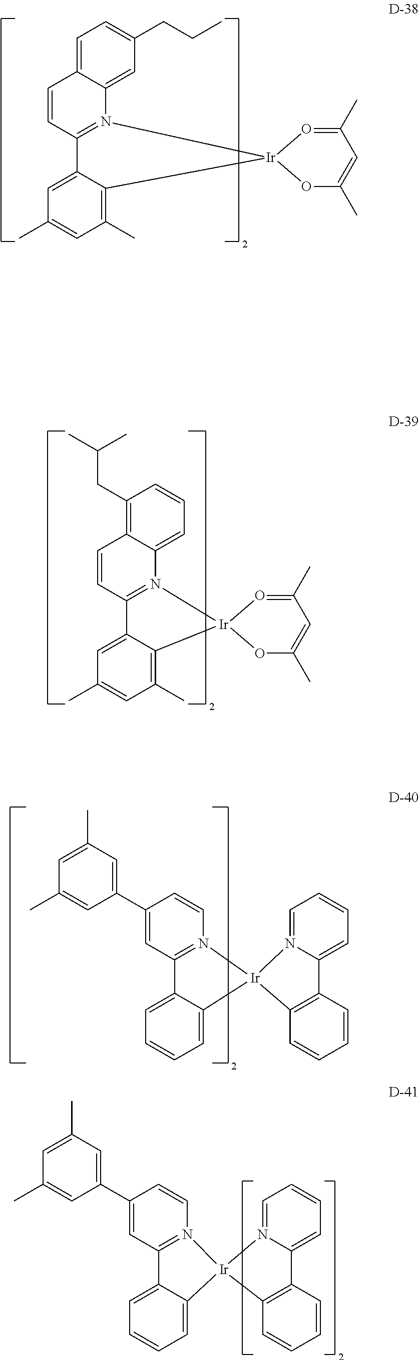

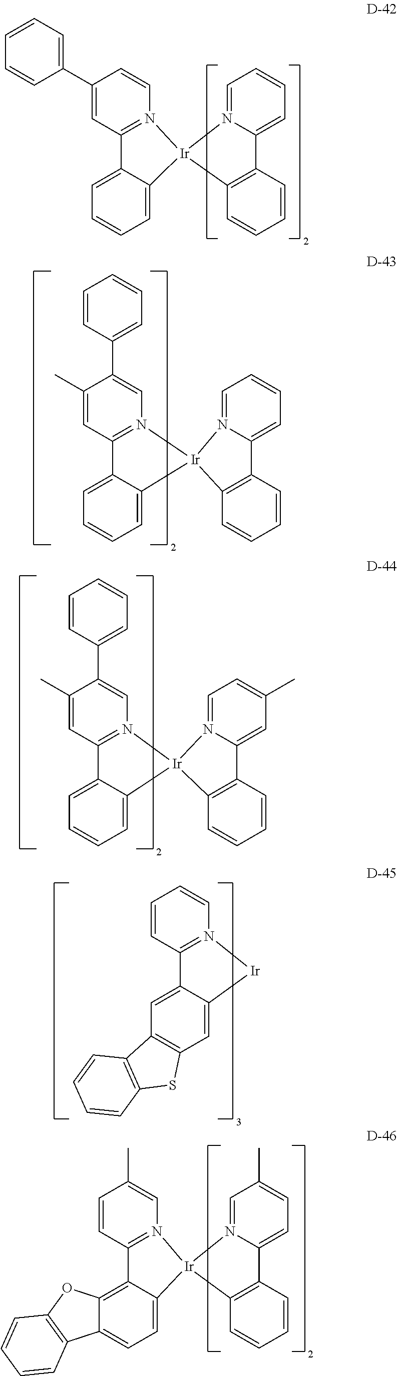

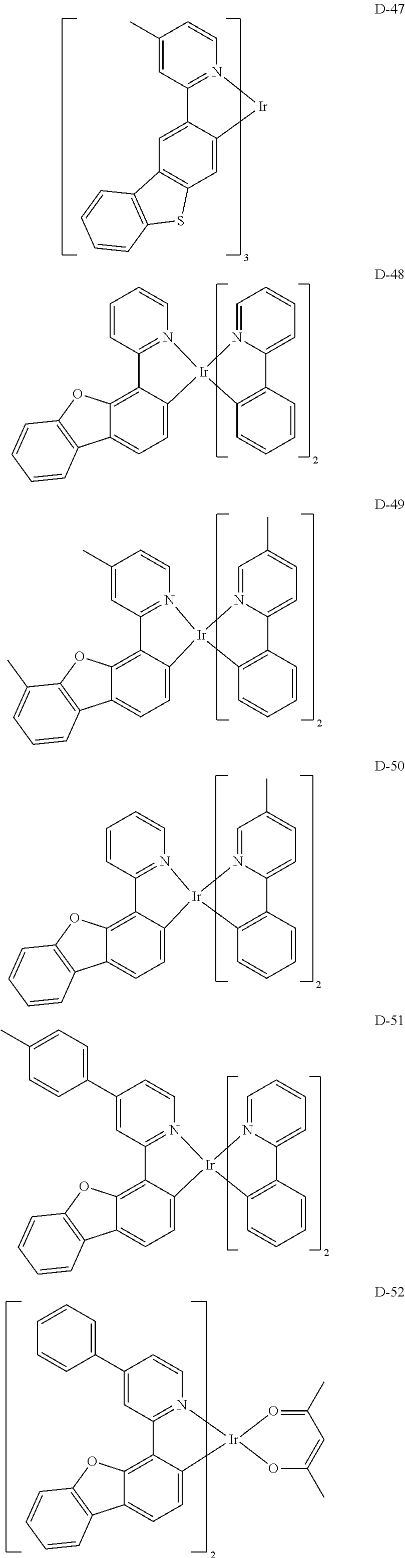

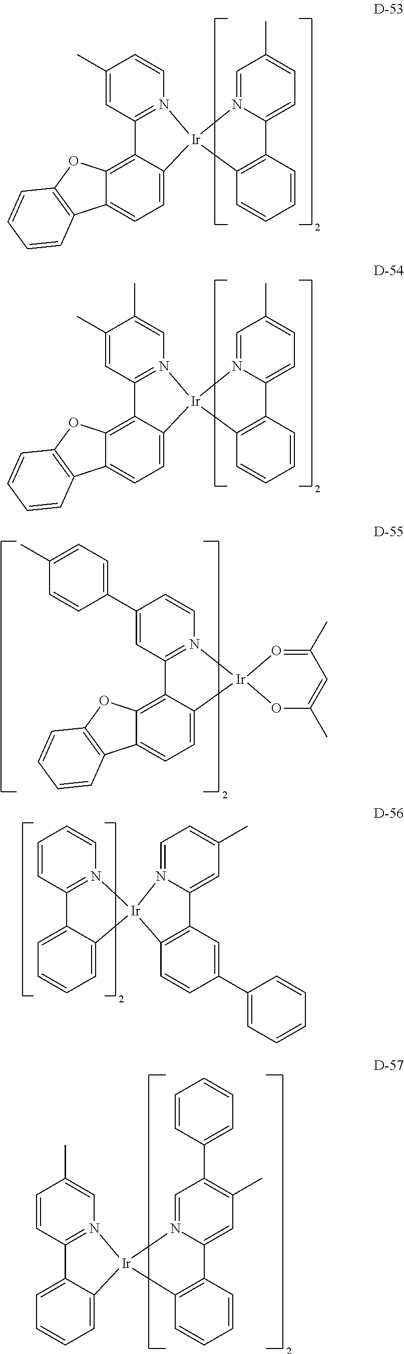

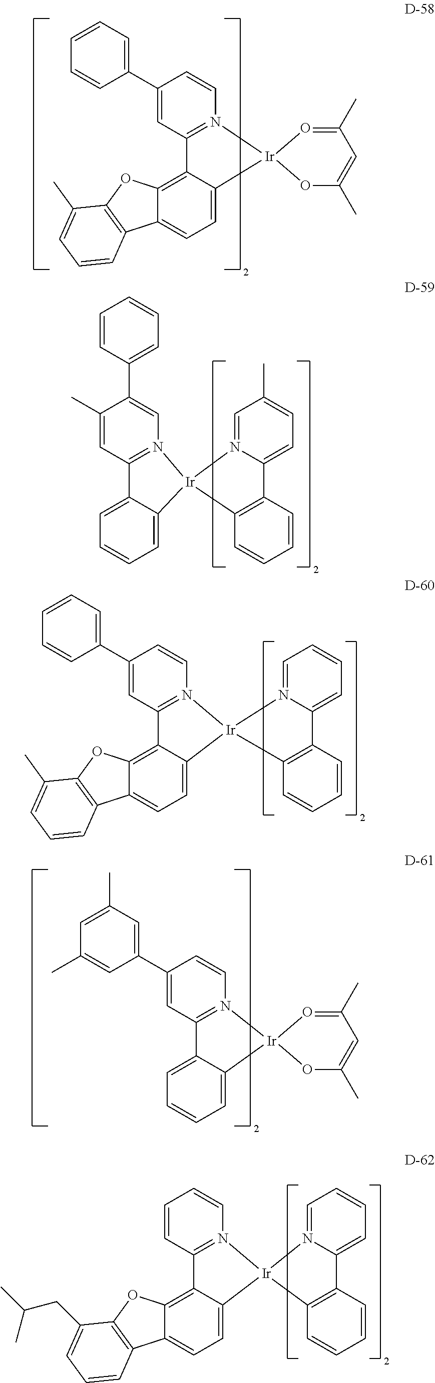

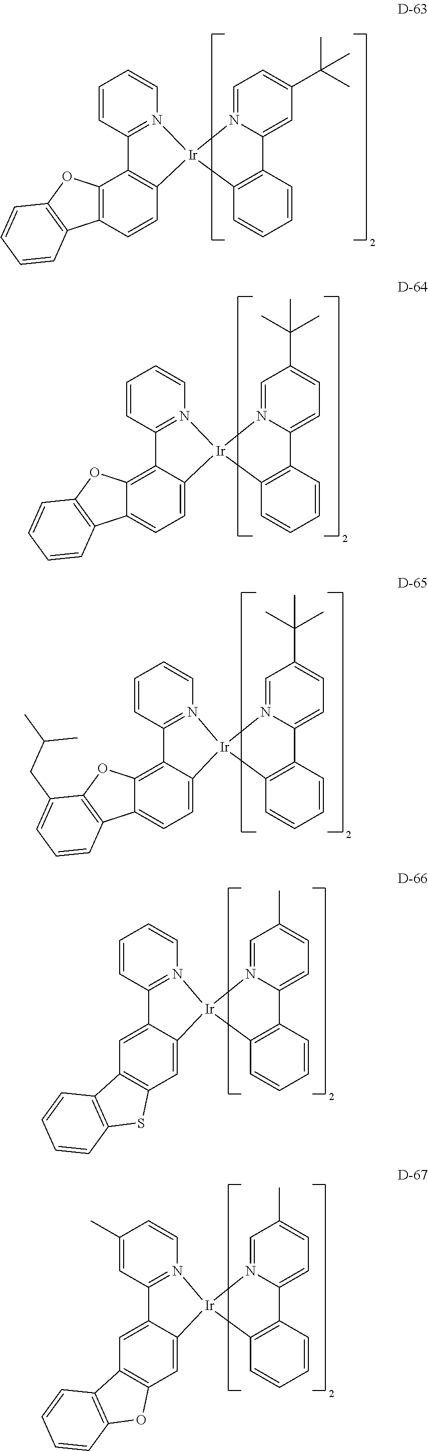

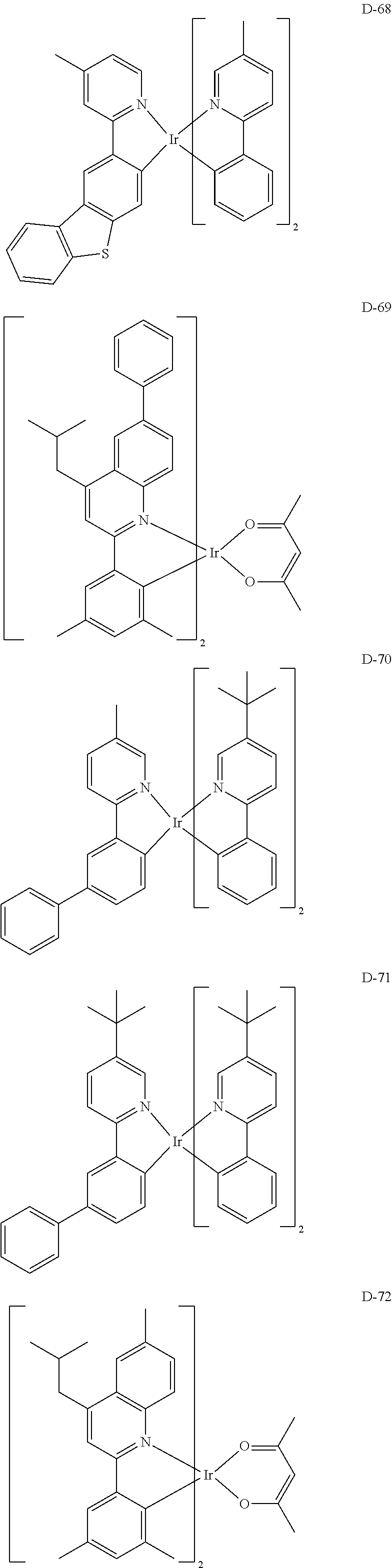

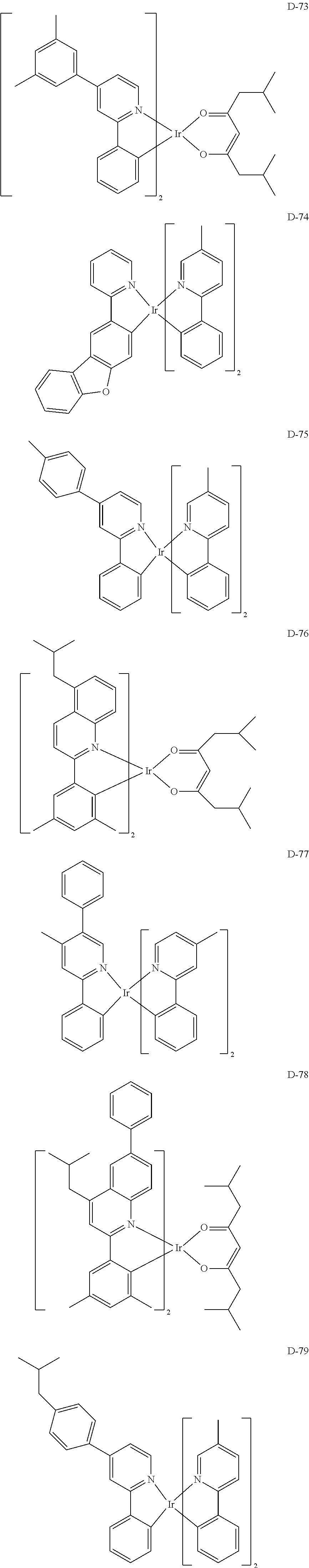

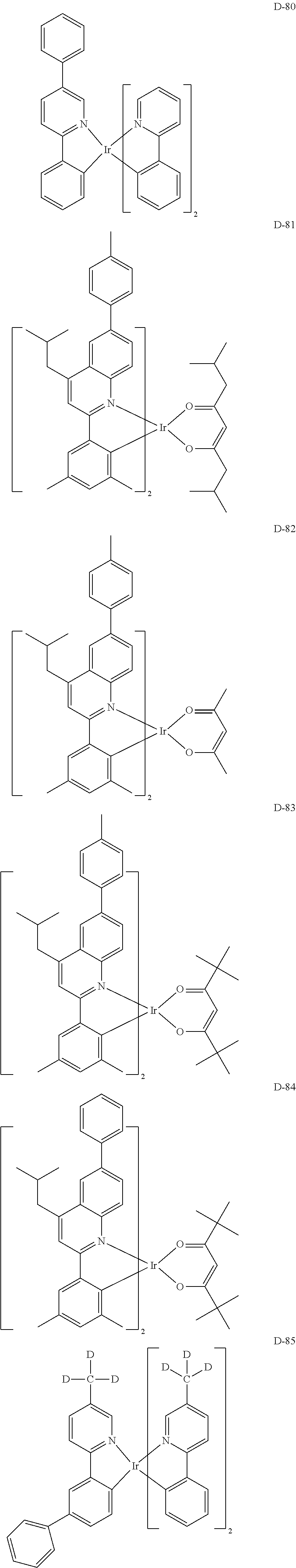

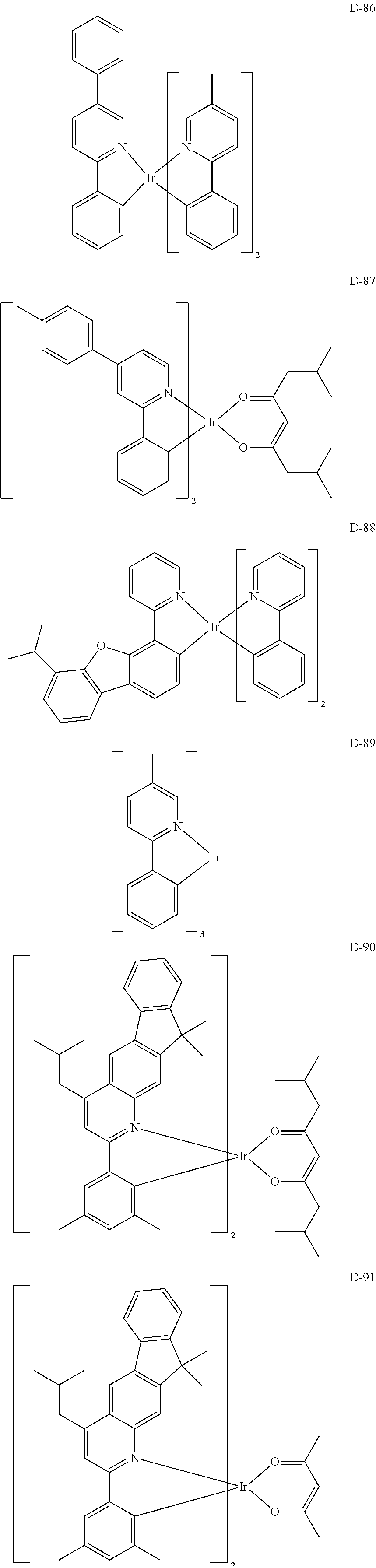

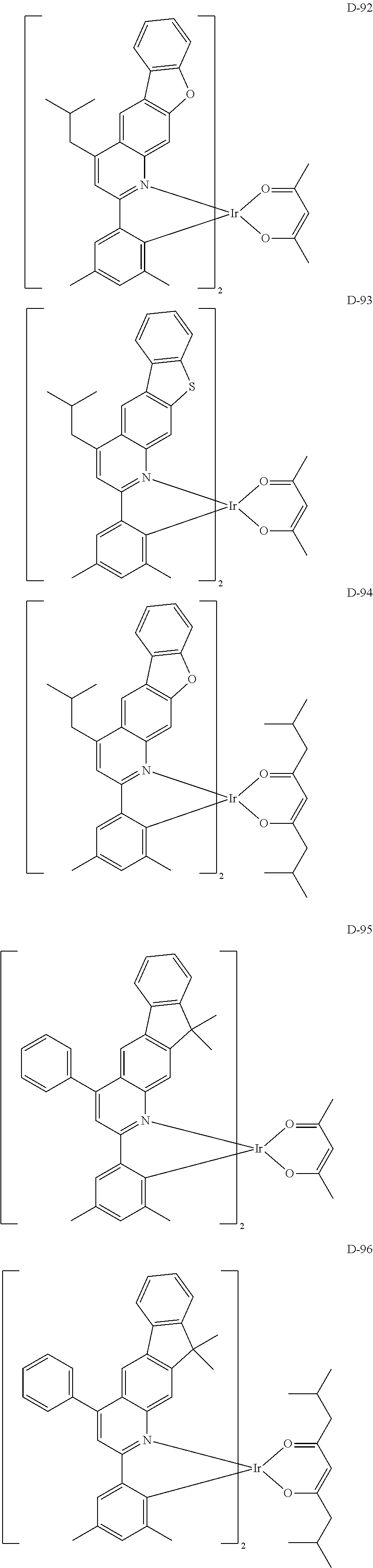

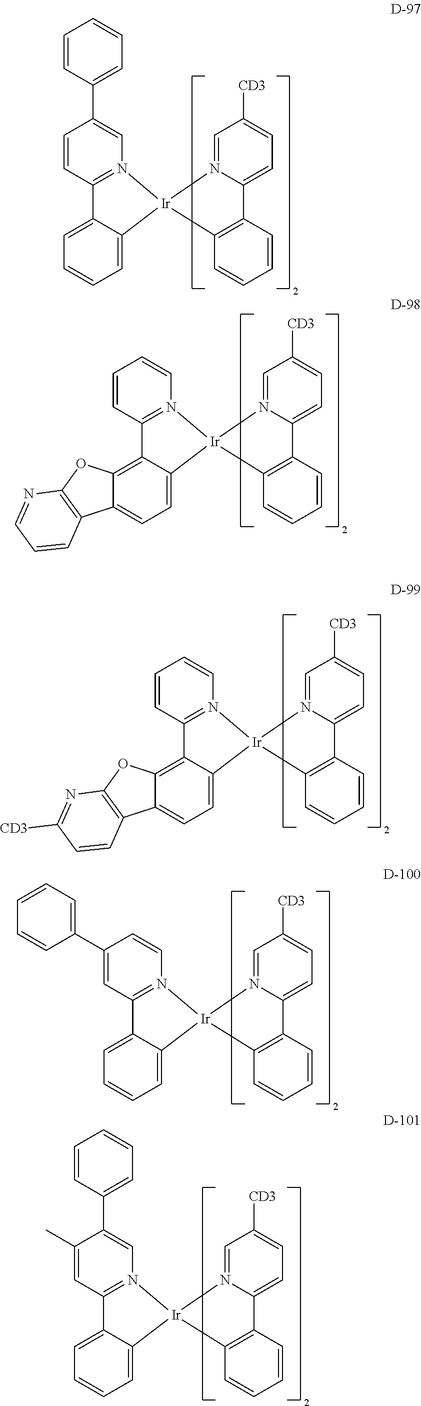

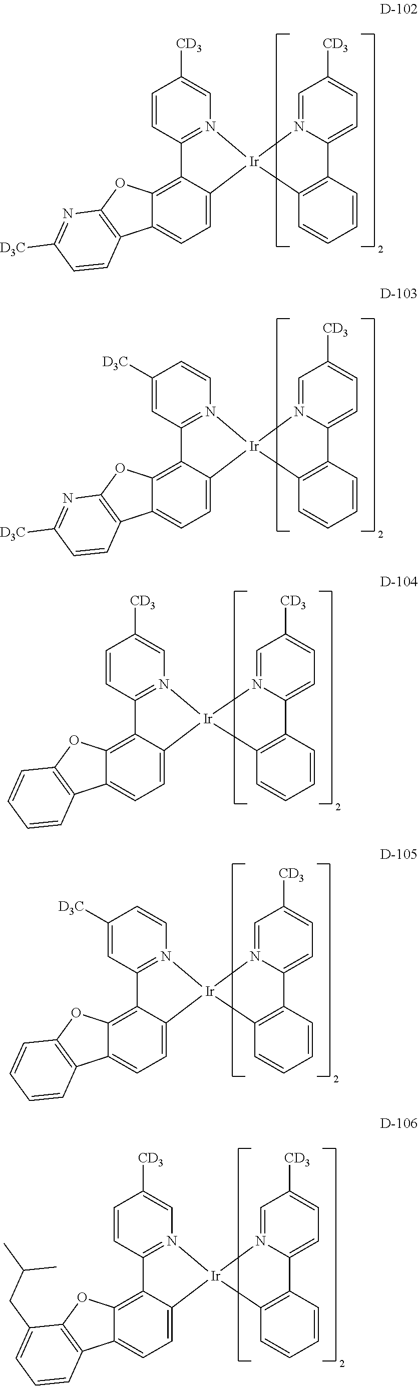

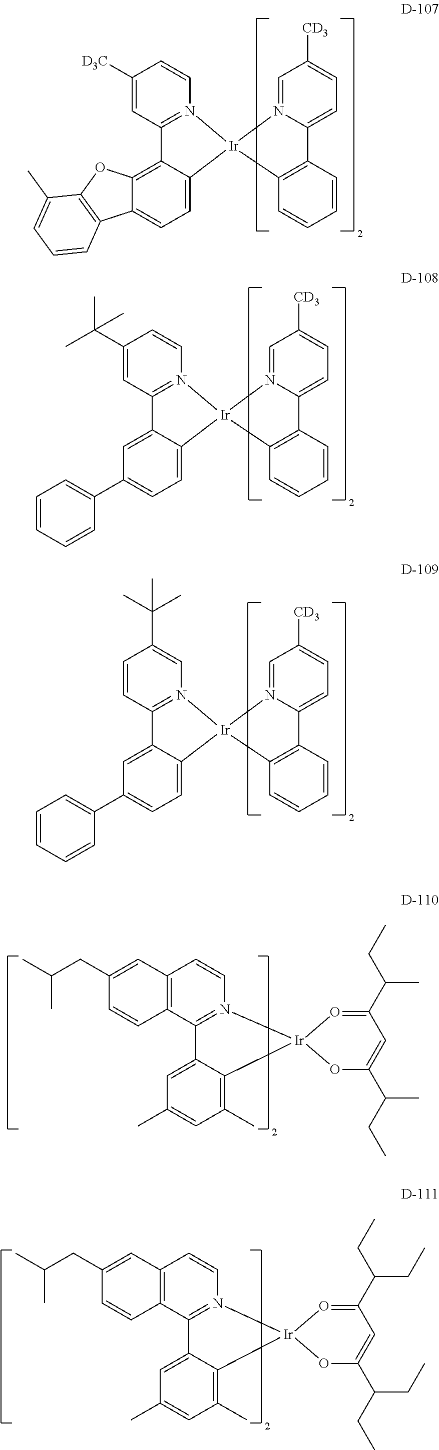

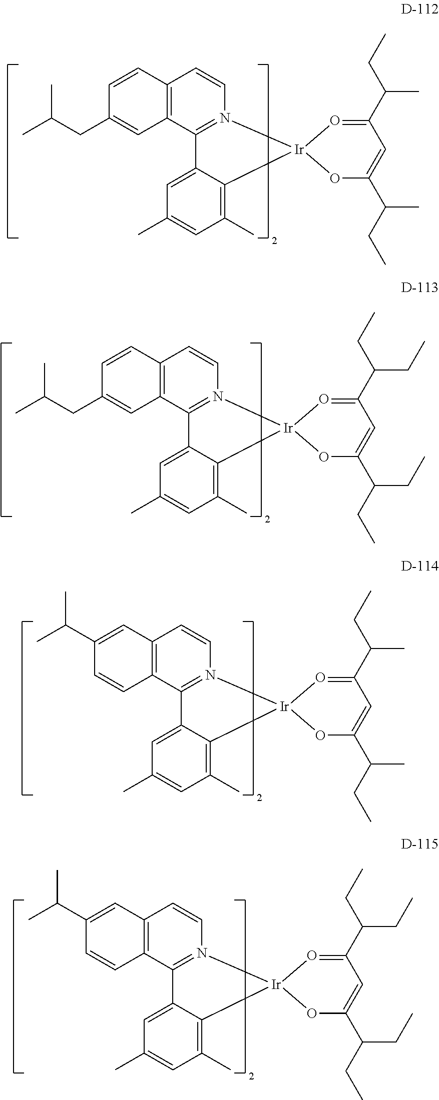

In order to form each layer of the organic electroluminescent device of the present disclosure, dry film-forming methods such as vacuum evaporation, sputtering, plasma, and ion plating methods, or wet film-forming methods such as ink jet printing, nozzle printing, slot coating, spin coating, dip coating, and flow coating methods can be used.

In a wet film-forming method, a thin film can be formed by dissolving or diffusing materials forming each layer into any suitable solvent such as ethanol, chloroform, tetrahydrofuran, dioxane, etc. The solvent can be any solvent where the materials forming each layer can be dissolved or diffused, and where there are no problems in film-formation capability.

In addition, the compound represented by formula 1 and the compound represented by formula 2 may be film-formed in the above-listed methods, commonly by a co-evaporation process or a mixture-evaporation process. The co-evaporation is a mixed deposition method in which two or more materials are placed in a respective individual crucible source and an electric current is applied to both cells at the same time to evaporate the materials. The mixture-evaporation is a mixed deposition method in which two or more materials are mixed in one crucible source before evaporating them, and an electric current is applied to the cell to evaporate the materials.

The organic electroluminescent materials according to the present disclosure may be used as light-emitting materials for a white organic light-emitting device. The white organic light-emitting device has been suggested to have various structures such as a side-by-side structure or a stacking structure depending on the arrangement of R (red), G (green) or YG (yellow green), and B (blue) light-emitting parts, or a color conversion material (CCM) method, etc., and the present disclosure may also be applied to such white organic light-emitting device. In addition, the organic electroluminescent materials according to the present disclosure may also be used in an organic electroluminescent device comprising a quantum dot (QD).

The present disclosure may provide a display system comprising the plurality of host materials of the present disclosure. In addition, it is possible to produce a display system or a lighting system by using the organic electroluminescent device of the present disclosure. Specifically, it is possible to produce a display system, e.g., a display system for smartphones, tablets, notebooks, PCs, TVs, or cars, or a lighting system, e.g., an outdoor or indoor lighting system, by using the organic electroluminescent device of the present disclosure.

Hereinafter, the preparation method of the compound of the present disclosure and the properties thereof will be explained in detail with reference to the representative compounds of the present disclosure. However, the present disclosure is not limited by the following examples.

### Example 1: Preparation of Compound C2-53

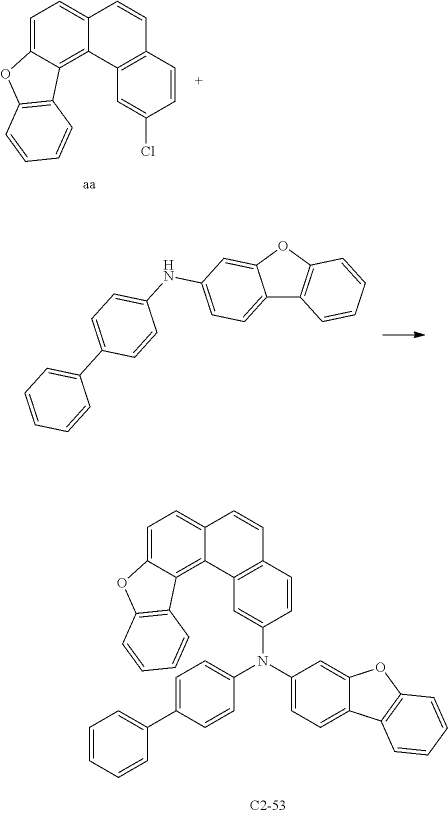 [SMILES: Clc1ccc2ccc3ccc4oc5ccccc5c4c3c2c1 ; c1ccc(-c2ccc(Nc3ccc4c(c3)oc3ccccc34)cc2)cc1 ; c1ccc(-c2ccc(N(c3ccc4c(c3)oc3ccccc34)c3ccc4ccc5ccc6oc7ccccc7c6c5c4c3)cc2)cc1]

8.0 g of compound aa (26.4 mmol), 8.85 g of N-([1,1′-biphenyl]-4-yl)dibenzo[b,d]furan-3-amine (26.4 mmol), 1.21 g of Pd₂(dba)₃ (1.32 mmol), 1.08 g of SPhos (2.64 mmol), and 3.81 g of NaOtBu (39.6 mmol) were added to 140 mL of o-xylene, and the mixture was stirred under reflux for 5 hours. After completion of the reaction, the reaction mixture was cooled to room temperature, and then filtered through silica gel. The organic layer was distilled under reduced pressure, and then recrystallized with toluene to obtain 5.0 g of compound C2-53 (yield: 31%).

| | MW | M.P. |
|---|---|---|
| C2-53 | 601.7 | 250° C. |

### Example 2: Preparation of Compound C2-54

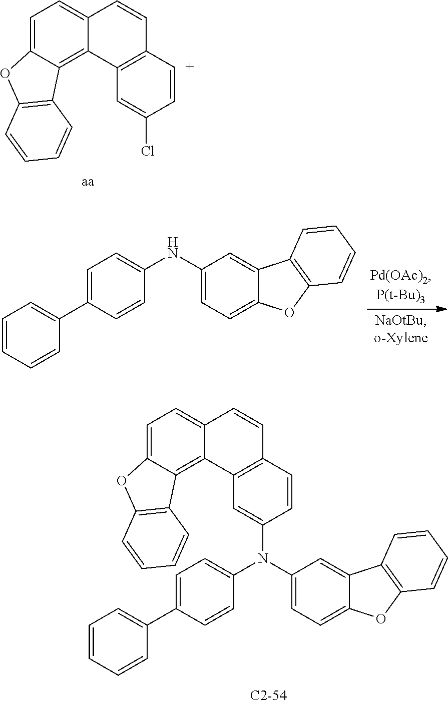 [SMILES: ClC1=Cc2c(ccc3ccc4oc5ccccc5c4c23)[C+]=C1 ; c1ccc(-c2ccc(Nc3ccc4oc5ccccc5c4c3)cc2)cc1 ; c1ccc(-c2ccc(N(c3ccc4oc5ccccc5c4c3)c3ccc4ccc5ccc6oc7ccccc7c6c5c4c3)cc2)cc1]

7 g of compound aa (23.15 mmol), 7.8 g of N-([1,1′-biphenyl]-4-yl)dibenzo[b,d]furan-2-amine (23.15 mmol), 0.26 g of Pd(OAc)₂ (1.158 mmol), 0.47 g of P(t-Bu)₃ (2.3 mmol), and 4.4 g of sodium tert-butoxide (46.3 mmol) were dissolved in 115 mL of o-xylene, and the mixture was stirred under reflux for 2 hours. After completion of the reaction, an organic layer was extracted with ethyl acetate, and then separated by column chromatography to obtain 3 g of compound C2-54 (yield: 21%).

| | MW | M.P. |
|---|---|---|
| C2-54 | 601.69 | 215° C. |

### Example 3: Preparation of Compound C2-46

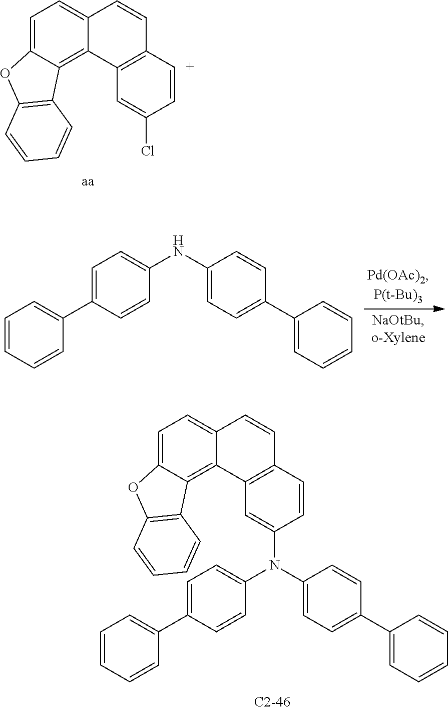 [SMILES: ClC1=Cc2c(ccc3ccc4oc5ccccc5c4c23)[C+]=C1 ; c1ccc(-c2ccc(Nc3ccc(-c4ccccc4)cc3)cc2)cc1 ; c1ccc(-c2ccc(N(c3ccc(-c4ccccc4)cc3)c3ccc4ccc5ccc6oc7ccccc7c6c5c4c3)cc2)cc1]

In a flask, 5.0 g of compound aa (16.5 mmol), 5.3 g of di([1,1′-biphenyl]-4-yl)amine (16.5 mmol), 0.19 g of Pd(OAc)₂ (0.83 mmol), 0.82 mL of P(t-Bu)₃ (1.65 mmol), and 3.2 g of NaOtBu (33.0 mmol) were dissolved in 83 mL of o-xylene, and the mixture was stirred under reflux for 3 hours. After completion of the reaction, an organic layer was extracted with ethyl acetate, and then separated by column chromatography to obtain 3 g of compound C2-46 (yield: 30%).

| | MW | M.P. |
|---|---|---|
| C2-46 | 587.71 | 237° C. |

### Example 4: Preparation of Compound C2-9

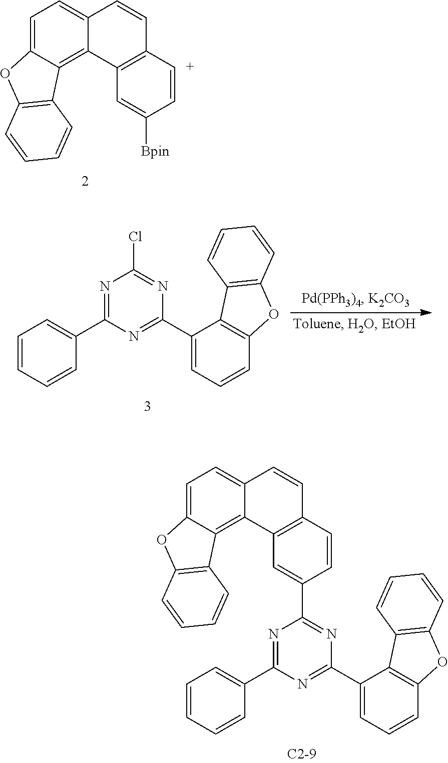

5.0 g of compound 2 (12.7 mmol), 5.5 g of compound 3 (15.3 mmol), 3.5 g of K₂CO₃ (25.4 mmol), and 0.73 g of Pd(PPh₃)₄ (0.63 mmol) were added in a flask, and dissolved in 39 mL of toluene, 10 mL of ethanol, and 13 mL of water, and then the mixture was stirred under reflux at 130° C. for 6 hours. After completion of the reaction, an organic layer was extracted with ethyl acetate, and the residual moisture was removed with magnesium sulfate. The residue was dried and separated by column chromatography to obtain 4.4 g of compound C2-9 (yield: 20%).

| | MW | M.P. |
|---|---|---|
| C2-9 | 589.65 | 318° C. |

### Example 5: Preparation of Compound C2-2

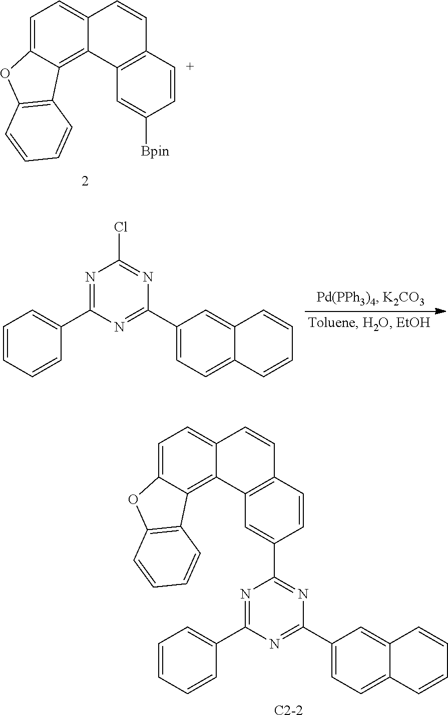

5.0 g of compound 2 (12.7 mmol), 4.8 g of 2-chloro-4-(naphthalene-2-yl)-6-phenyl-1,3,5-triazine (15.2 mmol), 3.5 g of K₂CO₃ (25.4 mmol), and 0.73 g of Pd(PPh₃)₄ (0.63 mmol) were added in a flask, and dissolved in 39 mL of toluene, 10 mL of ethanol, and 13 mL of water, and then the mixture was stirred under reflux at 130° C. for 6 hours. After completion of the reaction, an organic layer was extracted with ethyl acetate, and the residual moisture was removed with magnesium sulfate. The residue was dried and separated by column chromatography to obtain 4.4 g of compound C2-2 (yield: 20%).

| | MW | M.P. |
|---|---|---|
| C2-2 | 549.62 | 229° C. |

### Example 6: Preparation of Compound C2-166

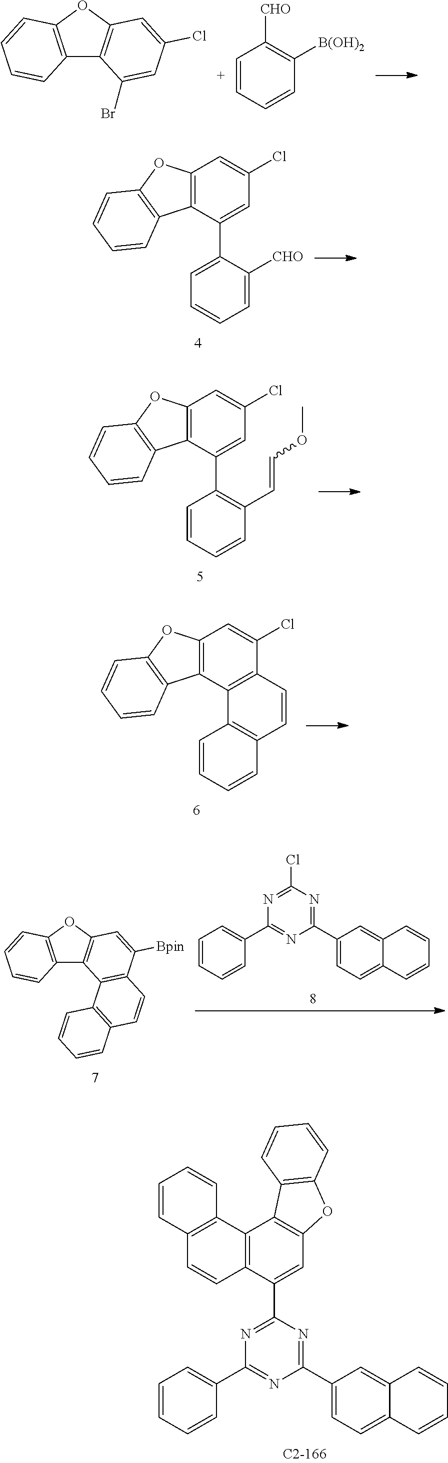

### 1) Synthesis of Compound 4

In a reaction vessel, 39.2 g of 1-bromo-3-chlorodibenzo[b,d]furan (139.3 mmol), 52.2 g of (2-formylphenyl)boronic acid (348.1 mmol), 16.1 g of tetrakis(triphenylphosphine)palladium(0) (13.9 mmol), 136.1 g of Cs₂CO₃ (418 mmol), 840 mL of toluene, 160 mL of ethanol, and 210 mL of distilled water were added, and the mixture was stirred at 140° C. for 5 hours. After completion of the reaction, the reaction mixture was cooled to room temperature and an organic layer was extracted with ethyl acetate. The extracted organic layer was dried with magnesium sulfate and the solvent was removed by a rotary evaporator. The residue was purified by column chromatography to obtain 32.1 g of compound 4 (yield: 75%).

### 2) Synthesis of Compound 5

In a reaction vessel, 31.6 g of compound 4 (103 mmol), 45.9 g of (methoxymethyl)triphenylphosphonium chloride (133.9 mmol) and 515 mL of tetrahydrofuran were added, and the mixture was stirred for 10 minutes. 150 mL of potassium tert-butoxide (1M in TH F) was slowly added dropwise to the mixture under 0° C. conditions. The temperature was slowly raised to room temperature and the reaction solution was stirred for 3 hours. Distilled water was added to the reaction solution to complete the reaction, and an organic layer was extracted with ethyl acetate. The extracted organic layer was dried with magnesium sulfate and the solvent was removed by a rotary evaporator. The residue was purified by column chromatography to obtain 31.2 g of compound 5 (yield: 90%).

### 3) Synthesis of Compound 6

In a reaction vessel, 29.8 g of compound 5 (89.0 mmol), 22.4 mL of boron trifluoride etherate, and 890 mL of methylene chloride (MC) were added, and the mixture was stirred for 3 hours. After completion of the reaction, an organic layer was extracted with water and methylene chloride (MC). The extracted organic layer was dried with magnesium sulfate and the solvent was removed by a rotary evaporator. The residue was purified by column chromatography to obtain 24.2 g of compound 6 (yield: 90%).

### 4) Synthesis of Compound 7

In a reaction vessel, 18.0 g of compound 6 (59.5 mmol), 19.7 g of bis(pinacolato)diboron (77.3 mmol), 2.8 g of tris(dibenzylideneacetone)dipalladium (0) (2.9 mmol), 2.4 g of 2-dicyclohexylphosphino-2′,6′-dimethoxybiphenyl (SPhos) (5.9 mmol), 17.5 g of potassium acetate (178.5 mmol) and 300 mL of 1,4-dioxane were added, and the mixture was stirred at 150° C. for 6 hours. After completion of the reaction, the reaction mixture was cooled to room temperature, and an organic layer was extracted with ethyl acetate. The extracted organic layer was dried with magnesium sulfate and the solvent was removed by a rotary evaporator. The residue was purified by column chromatography to obtain 18.4 g of compound 7 (yield: 78%).

### 5) Synthesis of Compound C2-166

In a reaction vessel, 4.0 g of compound 7 (10.1 mmol), 3.9 g of compound 8 (12.2 mmol), 0.6 g of tetrakis(triphenylphosphine)palladium(0) (0.51 mmol), 2.8 g of potassium carbonate (20.2 mmol), 30 mL of toluene, 7 mL of ethanol, and 10 mL of distilled water were added, and the mixture was stirred at 130° C. for 6 hours. After completion of the reaction, methanol was added dropwise to the mixture, and the resulting solid was filtered. The resulting solid was purified by column chromatography to obtain 4.5 g of compound C2-166 (yield: 81%).

| | MW | M.P. |
|---|---|---|
| C2-166 | 547.6 | 228° C. |

### Example 7: Preparation of Compound C2-167

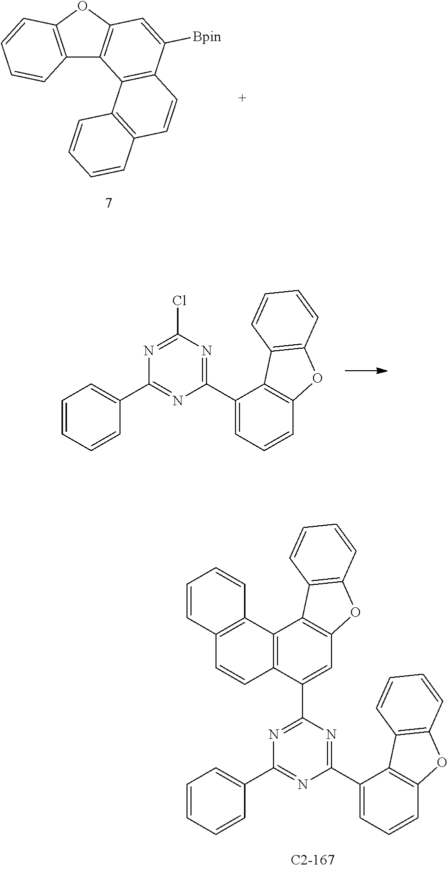

In a reaction vessel, 4.0 g of compound 7 (10.1 mmol), 4.4 g of 2-chloro-4-(dibenzo[b,d]furan-1-yl)-6-phenyl-1,3,5-triazine (12.2 mmol), 0.6 g of tetrakis(triphenylphosphine)palladium(0) (0.5 mmol), 2.8 g of potassium carbonate (20.2 mmol), 30 mL of toluene, 7 mL of ethanol, and 10 mL of distilled water were added, and the mixture was stirred at 130° C. for 6 hours. After completion of the reaction, methanol was added dropwise to the mixture, and the resulting solid was filtered. The resulting solid was purified by column chromatography to obtain 3.13 g of compound C2-167 (yield: 53%).

| | MW | M.P. |
|---|---|---|
| C2-167 | 589.6 | 250° C. |

### Example 8: Preparation of Compound C2-204

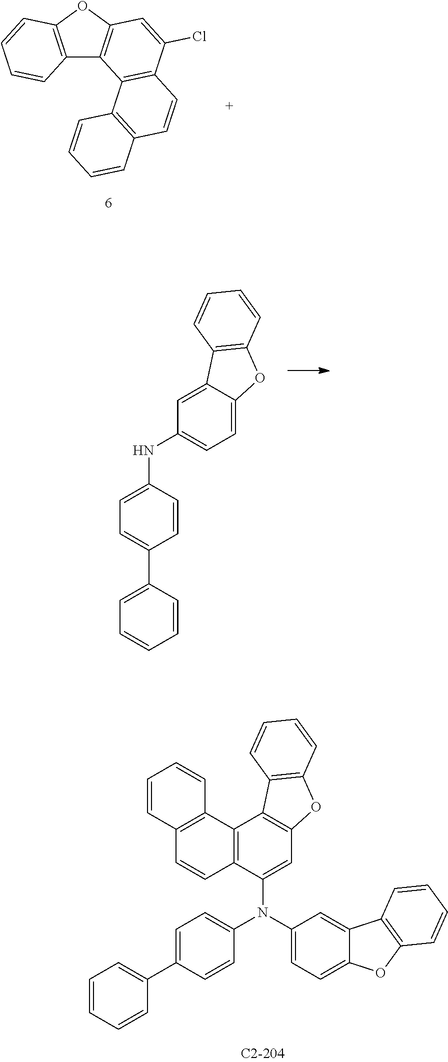 [SMILES: Clc1cc2oc3ccccc3c2c2c1ccc1ccccc12 ; c1ccc(-c2ccc(Nc3ccc4oc5ccccc5c4c3)cc2)cc1 ; c1ccc(-c2ccc(N(c3ccc4oc5ccccc5c4c3)c3cc4oc5ccccc5c4c4c3ccc3ccccc34)cc2)cc1]

In a reaction vessel, 4.0 g of compound 6 (13.2 mmol), 4.4 g of N-([1,1′-biphenyl]-4-yl)dibenzo[b,d]furan-2-amine (13.2 mmol), 0.15 g of Pd(OAc)₂ (0.66 mmol), 0.65 mL of P(tert-Bu)₃ (1.32 mmol), 2.5 g of sodium tert-butoxide (26.4 mmol), and 66 mL of xylene were added, and the mixture was stirred at 165° C. for 5 hours. After completion of the reaction, the reaction mixture was cooled to room temperature, and an organic layer was extracted with ethyl acetate. The extracted organic layer was dried with magnesium sulfate and the solvent was removed by a rotary evaporator. The residue was purified by column chromatography to obtain 4.9 g of compound C2-204 (yield: 61%).

| | MW | M.P. |
|---|---|---|
| C2-204 | 601.7 | 200° C. |

### Example 9: Preparation of Compound C2-146

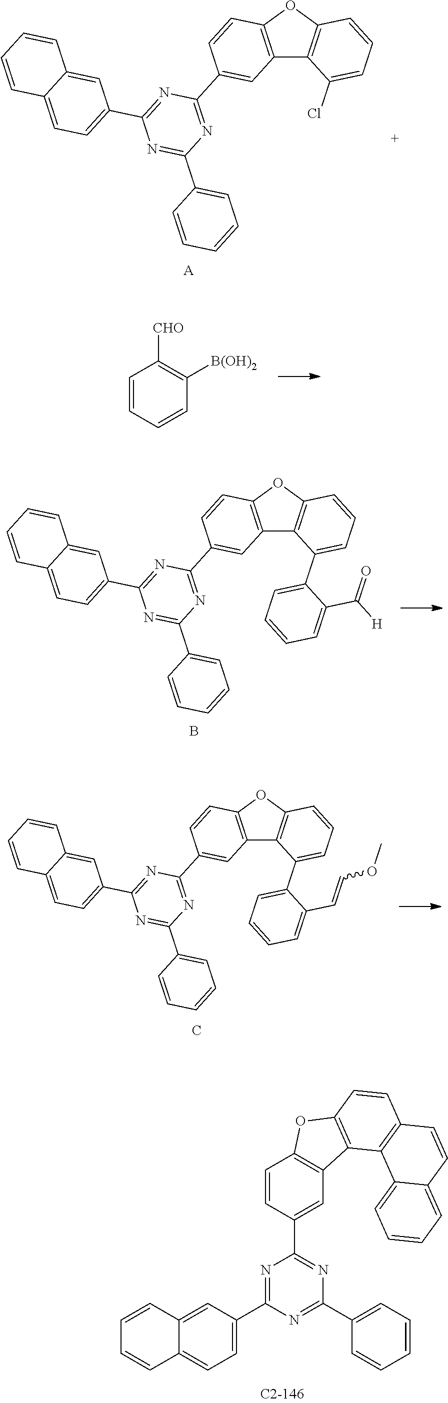

### 1) Synthesis of Compound B

In a reaction vessel, 5.0 g of compound A (10.3 mmol), 2.3 g of (2-formylphenyl)boronic acid (15.5 mmol), 0.47 g of Pd₂(dba)₃ (0.52 mmol), 0.43 g of 2-dicyclohexylphosphino-2′,6′-dimethoxybiphenyl (SPhos) (1.03 mmol), 5.5 g of K₃PO₄ (25.8 mmol), and 52 mL of xylene were added, and the mixture was stirred at 165° C. for 6 hours. After completion of the reaction, the reaction mixture was cooled to room temperature, and an organic layer was extracted with ethyl acetate. The extracted organic layer was dried with magnesium sulfate and the solvent was removed by a rotary evaporator. The residue was purified by column chromatography to obtain 4.55 g of compound B (yield: 80%).

### 2) Synthesis of Compound C

In a reaction vessel, 4.55 g of compound B (8.22 mmol), 3.66 g of (methoxymethyl)triphenylphosphonium chloride (10.7 mmol), and 41 mL of tetrahydrofuran were added, and the mixture was stirred for 10 minutes. 11 mL of potassium tert-butoxide (1M in THF) was slowly added dropwise to the mixture under 0° C. conditions. The temperature was slowly raised to room temperature and the reaction solution was stirred for 3 hours. Distilled water was added to the reaction solution to complete the reaction, and an organic layer was extracted with ethyl acetate. The extracted organic layer was dried with magnesium sulfate and the solvent was removed by a rotary evaporator. The residue was purified by column chromatography to obtain 3.06 g of compound C (yield: 64%).

### 3) Synthesis of Compound C2-146

In a reaction vessel, 2.3 g of compound C (3.95 mmol), 0.23 mL of Eaton's reagent, and 23 mL of chlorobenzene were added, and mixture was refluxed for 2 hours. After completion of the reaction, the reaction mixture was cooled to room temperature, and an organic layer was extracted with methylene chloride (MC). The extracted organic layer was dried with magnesium sulfate and the solvent was removed by a rotary evaporator. The residue was purified by column chromatography to obtain 1.93 g of compound C2-146 (yield: 89%).

| | MW | M.P. |
|---|---|---|
| C2-146 | 549.62 | 204° C. |

### Example 10: Preparation of Compound C2-217

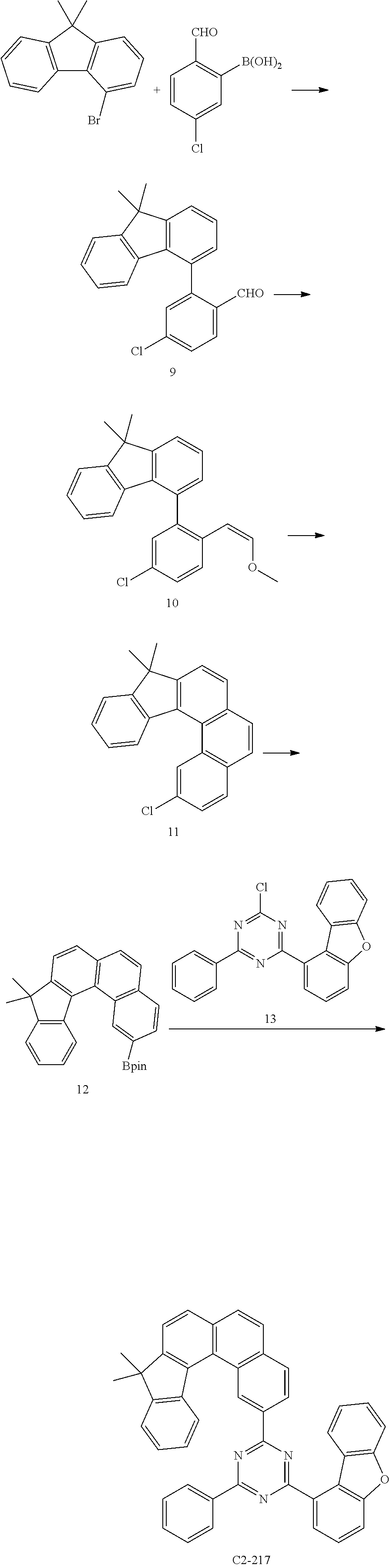

### 1) Synthesis of Compound 9

In a reaction vessel, 50 g of 4-bromo-9,9-dimethyl-9H-fluorene (183 mmol), 40.5 g of (5-chloro-2-formylphenyl)boronic acid (219 mmol), 10.6 g of tetrakis(triphenylphosphine)palladium(0) (9.15 mmol), 63 g of potassium carbonate (457 mmol), 690 mL of toluene, 180 mL of ethanol, and 230 mL of distilled water were added, and the mixture was stirred at 140° C. for 5 hours. After completion of the reaction, the reaction mixture was cooled to room temperature, and an organic layer was extracted with ethyl acetate. The extracted organic layer was dried with magnesium sulfate and the solvent was removed by a rotary evaporator. The residue was purified by column chromatography to obtain 40.3 g of compound 9 (yield: 66%).

### 2) Synthesis of Compound 10

In a reaction vessel, 40.3 g of compound 9 (121 mmol), 53.9 g of (methoxymethyl)triphenylphosphonium chloride (157.4 mmol), and 600 mL of tetrahydrofuran were added, and the mixture was stirred for 10 minutes. 162 mL of potassium tert-butoxide (1M in TH F) was slowly added dropwise to the mixture under 0° C. conditions. The temperature was slowly raised to room temperature and the reaction solution was stirred for 3 hours. Distilled water was added to the reaction solution to complete the reaction, and an organic layer was extracted with ethyl acetate. The extracted organic layer was dried with magnesium sulfate and the solvent was removed by a rotary evaporator. The residue was purified by column chromatography to obtain 39 g of compound 10 (yield: 89%).

### 3) Synthesis of Compound 11

In a reaction vessel, 38 g of compound 10 (105.3 mmol), 26.5 mL of boron trifluoride etherate, and 1000 mL of methylene chloride (MC) were added, and the mixture was stirred for 3 hours. After completion of the reaction, an organic layer was extracted with water and methylene chloride (MC). The extracted organic layer was dried with magnesium sulfate and the solvent was removed by a rotary evaporator. The residue was purified by column chromatography to obtain 23.2 g of compound 11 (yield: 67%).

### 4) Synthesis of Compound 12

In a reaction vessel, 19.1 g of compound 11 (58.1 mmol), 19.1 g of bis(pinacolato)diboron (75.5 mmol), 2.7 g of tris(dibenzylideneacetone)dipalladium (0) (2.9 mmol), 2.4 g of 2-dicyclohexylphosphino-2′,6′-dimethoxybiphenyl (SPhos) (5.81 mmol), 17.1 g of potassium acetate (174.3 mmol) and 290 mL of 1,4-dioxane were added, and the mixture was stirred at 150° C. for 6 hours. After completion of the reaction, the reaction mixture was cooled to room temperature, and an organic layer was extracted with ethyl acetate. The extracted organic layer was dried with magnesium sulfate and the solvent was removed by a rotary evaporator. The residue was purified by column chromatography to obtain 12.7 g of compound 12 (yield: 52%).

### 5) Synthesis of Compound C2-217

In a reaction vessel, 4 g of compound 12 (9.5 mmol), 4.1 g of compound 13 (11.4 mmol), 0.55 g of tetrakis(triphenylphosphine)palladium(0) (0.48 mmol), 2.6 g of potassium carbonate (19.0 mmol), 30 mL of toluene, 7 mL of ethanol, and 10 mL of distilled water were added, and the mixture was stirred at 130° C. for 6 hours. After completion of the reaction, methanol was added dropwise to the mixture, and the resulting solid was filtered. The resulting solid was purified by column chromatography to obtain 4.73 g of compound C2-217 (yield: 80%).

| | MW | M.P. |
|---|---|---|
| C2-217 | 615.7 | 237° C. |

### Example 11: Preparation of Compound C2-216

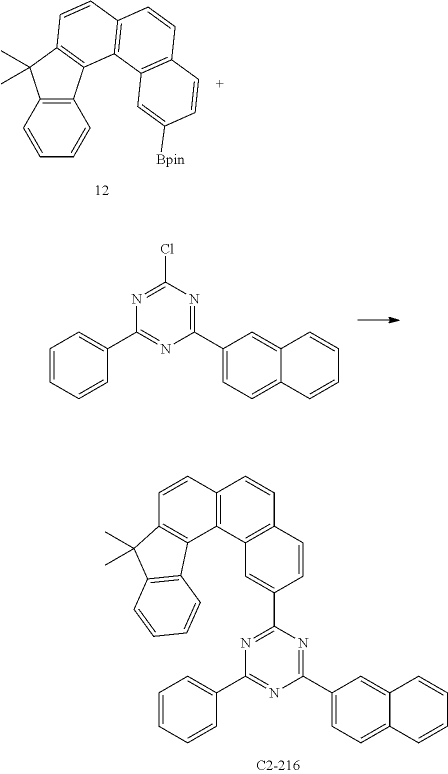

In a reaction vessel, 5.0 g of compound 12 (11.9 mmol), 4.5 g of 2-chloro-4-(naphthalene-2-yl)-6-phenyl-1,3,5-triazine (14.3 mmol), 0.7 g of tetrakis(triphenylphosphine)palladium(0) (0.6 mmol), 3.3 g of potassium carbonate (23.8 mmol), 36 mL of toluene, 10 mL of ethanol, and 12 mL of distilled water were added, and the mixture was stirred at 130° C. for 6 hours. After completion of the reaction, methanol was added dropwise to the mixture, and the resulting solid was filtered. The resulting solid was purified by column chromatography to obtain 3.64 g of compound C2-216 (yield: 53%).

| | MW | M.P. |
|---|---|---|
| C2-216 | 575.7 | 209° C. |

### Example 12: Preparation of Compound C2-67

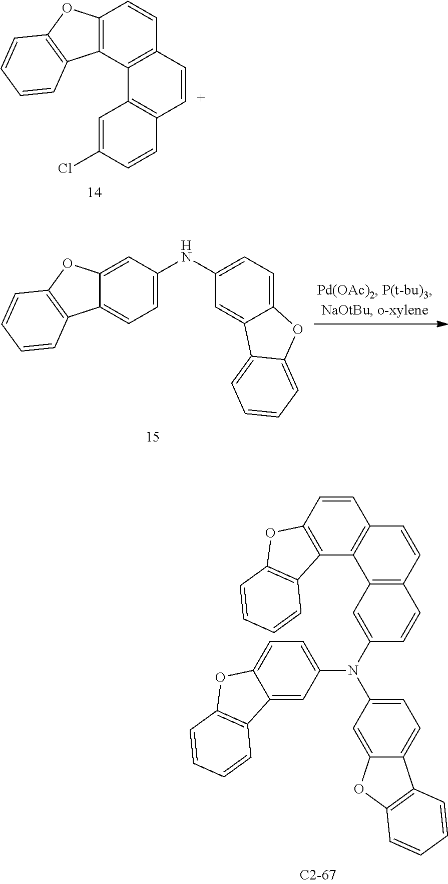 [SMILES: Clc1ccc2c(c1)-c1c(ccc3oc4ccccc4c13)C=[C+]2 ; c1ccc2c(c1)oc1cc(Nc3ccc4oc5ccccc5c4c3)ccc12 ; c1ccc2c(c1)oc1cc(N(c3ccc4oc5ccccc5c4c3)c3ccc4ccc5ccc6oc7ccccc7c6c5c4c3)ccc12]

5.0 g of compound 14 (16.5 mmol), 5.7 g of compound 15 (16.5 mmol), 0.19 g of Pd(OAc)₂ (0.82 mmol), 0.82 mL of P(t-Bu)₃ (1.65 mmol), and 3.2 g of NaOtBu (33.0 mmol) were added in a flask, and dissolved in 83 mL of o-xylene, and then the mixture was stirred under reflux for 2 hours. After completion of the reaction, an organic layer was extracted with EA/H₂O, and separated by column chromatography to obtain 4.46 g of compound C2-67 (yield: 43%).

| | MW | M.P. |
|---|---|---|
| C2-67 | 615.69 | 239° C. |

### Example 13: Preparation of Compound C2-221

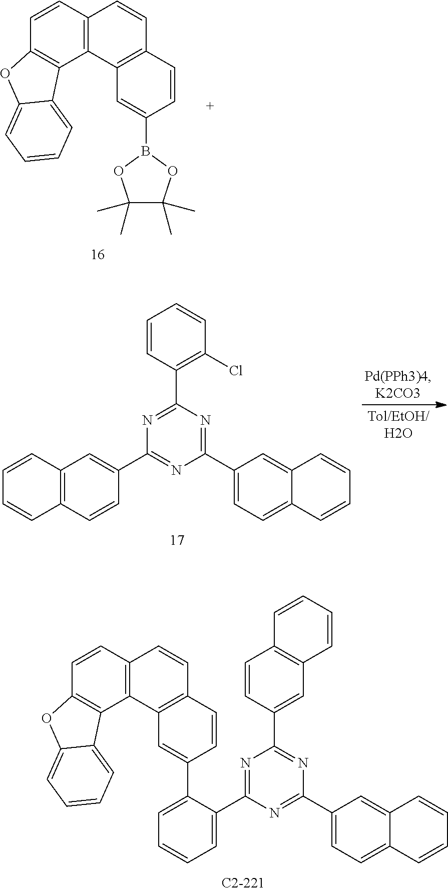 [SMILES: CC1(C)OB(c2ccc3ccc4ccc5oc6ccccc6c5c4c3c2)OC1(C)C ; Clc1ccccc1-c1nc(-c2ccc3ccccc3c2)nc(-c2ccc3ccccc3c2)n1 ; c1ccc(-c2nc(-c3ccc4ccccc4c3)nc(-c3ccc4ccccc4c3)n2)c(-c2ccc3ccc4ccc5oc6ccccc6c5c4c3c2)c1]

In a flask, 4 g of compound 16 (10.14 mmol), 4.3 g of compound 17 (10.14 mmol), 586 mg of Pd(PPh₃)₄ (0.507 mmol), and 2.8 g of K₂CO₃ (20.29 mmol) were dissolved in 50 mL toluene, 12 mL of EtOH, and 13 mL of H₂O, and the mixture was stirred under reflux at 140° C. for 6 hours. After completion of the reaction, the reaction mixture was cooled to room temperature, and the resulting solid was filtered under reduced pressure. The solid was dissolved in CHCl₃, and separated by SiO₂ filter, and then recrystallized with o-xylene and o-dichlorobenzene (o-DCB) to obtain 5.8 g of compound C2-221 (yield: 65%).

| | MW | M.P. |
|---|---|---|
| C2-221 | 675.7 | 270.8° C. |

### Example 14: Preparation of Compound C2-220

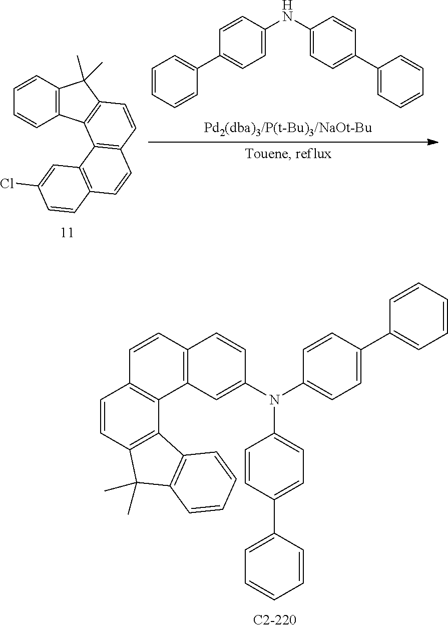

In a reaction vessel, 3.3 g of compound 11 (10.04 mmol), 3.2 g of di([1,1′-biphenyl]-4-yl)amine (10.04 mmol), 0.5 g of tris(dibenzylideneacetone)dipalladium(0) (0.50 mmol), 0.5 mL of tri-tert-butyl phosphine (1.04 mmol), 1.5 g of sodium tert-butoxide (15.06 mmol), and 50 mL of toluene were added, and the mixture was stirred under reflux for 4 hours. The reaction mixture was cooled to room temperature, and then the solid was filtered and washed with ethyl acetate. The filtrate was distilled under reduced pressure, and purified by column chromatography to obtain 3.2 g of a compound C2-220 (yield: 52%).

| | MW | M.P. |
|---|---|---|
| C2-220 | 613.79 | 213° C. |

### Example 15: Preparation of Compound C2-205

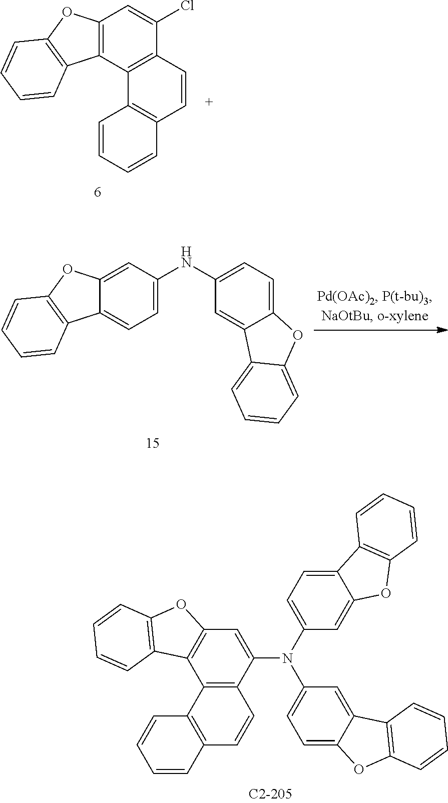 [SMILES: Clc1cc2oc3ccccc3c2c2c1ccc1ccccc12 ; c1ccc2c(c1)oc1cc(Nc3ccc4oc5ccccc5c4c3)ccc12 ; c1ccc2c(c1)ccc1c(N(c3ccc4c(c3)oc3ccccc34)c3ccc4oc5ccccc5c4c3)cc3oc4ccccc4c3c12]

In a reaction vessel, 2.14 g of compound 6 (7.1 mmol), 2.5 g of compound 15 (7.1 mmol), 0.08 g of Pd(OAc)₂ (0.36 mmol), 0.35 mL of tri-tert-butyl phosphine (0.71 mmol), 1.4 g of sodium tert-butoxide (14.2 mmol), and 36 mL of o-xylene were added, and the mixture was stirred at 165° C. for 6 hours. After completion of the reaction, the reaction mixture was cooled to room temperature, and an organic layer was extracted with ethyl acetate. The extracted organic layer was dried with magnesium sulfate and the solvent was removed by a rotary evaporator. The residue was purified by column chromatography to obtain 2.0 g of compound C2-205 (yield: 12%).

| | MW |
|---|---|
| C2-205 | 615.69 |

Hereinafter, the luminous efficiency and lifetime properties of an OLED according to the present disclosure will be explained in detail. However, the following examples merely illustrate the properties of an OLED according to the present disclosure, but the present disclosure is not limited to the following examples.

### Device Examples 1 to 7: Producing an OLED According to the Present Disclosure

An OLED according to the present disclosure was produced as follows: A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropanol. The ITO substrate was mounted on a substrate holder of a vacuum vapor deposition apparatus. Compound HI-1 shown in Table 2 below was introduced into a cell of the vacuum vapor deposition apparatus, and compound HT-1 shown in Table 2 below was introduced into another cell of the vacuum vapor deposition apparatus. The two materials were evaporated at different rates to be deposited in a doping amount of compound HI-1 of 3 wt % based on the total amount of compound HI-1 and compound HT-1 to form a hole injection layer having a thickness of 10 nm on the ITO substrate. Next, compound HT-1 was deposited as a first hole transport layer having a thickness of 80 nm on the hole injection layer. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: The first host compound and the second host compound shown in Table 1 below were introduced into two cells of the vacuum vapor depositing apparatus, respectively, as hosts, and compound D-39 was introduced into another cell as a dopant. The two host materials were evaporated at a rate of 1:1, and at the same time the dopant material was evaporated at a different rate to be deposited in a doping amount of 3 wt % based on the total amount of the hosts and dopant to form a light-emitting layer having a thickness of 40 nm on the second hole transport layer. Next, compound ETL-1 and compound EIL-1 were deposited at a weight ratio of 50:50 on the light-emitting layer to form an electron transport layer having a thickness of 35 nm. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, an OLED was produced. All the materials used for producing the OLED were purified by vacuum sublimation at 10⁻⁶ torr.

### Comparative Examples 1 to 5: Producing an OILED Comprising a Comparative Compound as a Host

An OLED was produced in the same manner as in Device Example 1, except that the first host compound and the second host compound shown in Table 1 below were respectively used as a host(s) of the light-emitting layer.

The time taken to reduce the initial luminance of 100% to a luminance of 95% in a luminance of 5,500 nit (T95) of the OLEDs produced in Device Examples and Comparative Examples are shown in Table 1 below.

TABLE 1

| | | | Light- | |
|---|---|---|---|---|
| | First | Second | Emitting | Lifetime |
| | Host | Host | Color | (T95) [hr] |
| Device Example 1 | C1-6 | C2-2 | Red | 126.6 |
| Device Example 2 | C1-6 | C2-9 | Red | 212.2 |
| Device Example 3 | C1-146 | C2-46 | Red | 786.3 |
| Device Example 4 | C1-146 | C2-53 | Red | 737.4 |
| Device Example 5 | C1-146 | C2-54 | Red | 883.0 |
| Device Example 6 | C1-146 | C2-67 | Red | 863.0 |
| Device Example 7 | C1-138 | C2-9 | Red | 230.0 |
| Comparative | T-1 | C2-77 | Red | 0.14 |
| Example 1 | | | | |
| Comparative | — | C2-9 | Red | 64.8 |
| Example 2 | | | | |
| Comparative | — | C2-46 | Red | 5.0 |
| Example 3 | | | | |
| Comparative | — | C2-54 | Red | 4.4 |
| Example 4 | | | | |
| Comparative | — | C2-53 | Red | 4.4 |
| Example 5 | | | | |

From Table 1 above, it can be confirmed that the organic electroluminescent devices comprising the compounds of the present disclosure as host materials have improved lifetime properties as compared with conventional organic electroluminescent devices. By using the compound represented by formula 1 of the present disclosure in combination with the compound represented by formula 2 of the present disclosure, the highest occupied molecular orbital (HOMO) energy level can be increased compared to the case of using a conventional compound having a carbazole-carbazole backbone, and thus the hole mobility can be improved. As the hole injection from the hole transport layer becomes easier, it is possible to improve the balance of holes and electrons and the formation of excitons. It is thought that this can improve the lifetime properties of the OLED.

The compounds used in the Device Examples and the Comparative Examples are shown in Table 2 below.

TABLE 2
Hole Injection Layer/Hole Transport Layer

| | |
|---|---|
| 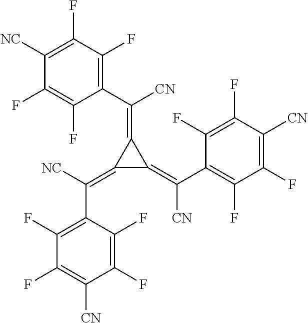 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] | HI-1 |
| 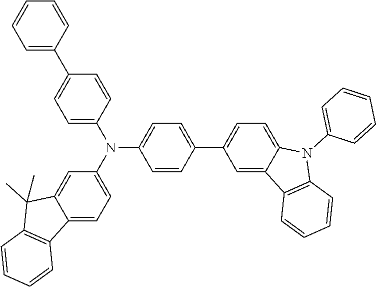 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21] | HT-1 |
| 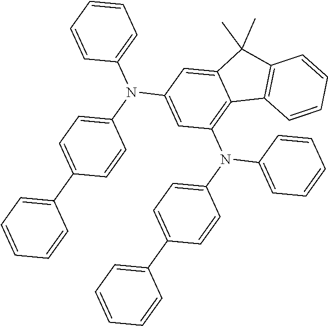 [SMILES: CC1(C)c2ccccc2-c2c(N(c3ccccc3)c3ccc(-c4ccccc4)cc3)cc(N(c3ccccc3)c3ccc(-c4ccccc4)cc3)cc21] | HT-2 |

Light-Emitting Layer

| | |
|---|---|
| 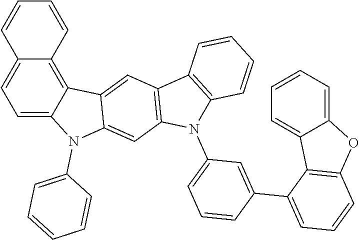 [SMILES: c1ccc(-n2c3cc4c(cc3c3c5ccccc5ccc32)c2ccccc2n4-c2cccc(-c3cccc4oc5ccccc5c34)c2)cc1] | C1-6 |
| 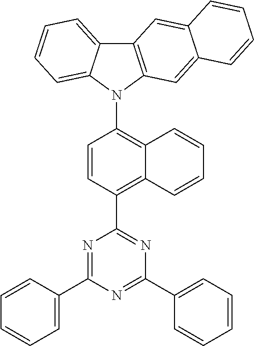 [SMILES: c1ccc(-c2nc(-c3ccccc3)nc(-c3ccc(-n4c5ccccc5c5cc6ccccc6cc54)c4ccccc34)n2)cc1] | C1-146 |
| 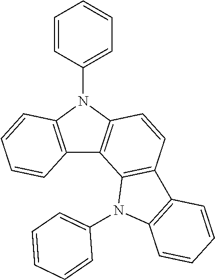 [SMILES: c1ccc(-n2c3ccccc3c3c2ccc2c4ccccc4n(-c4ccccc4)c23)cc1] | C1-138 |
| 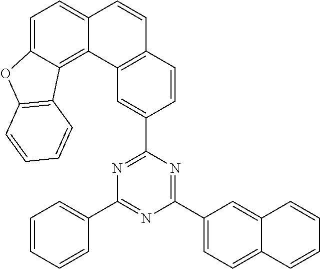 [SMILES: c1ccc(-c2nc(-c3ccc4ccccc4c3)nc(-c3ccc4ccc5ccc6oc7ccccc7c6c5c4c3)n2)cc1] | C2-2 |
| 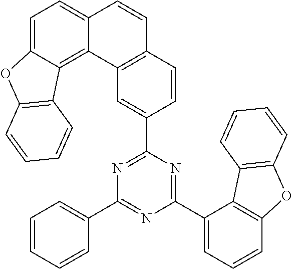 [SMILES: c1ccc(-c2nc(-c3ccc4ccc5ccc6oc7ccccc7c6c5c4c3)nc(-c3cccc4oc5ccccc5c34)n2)cc1] | C2-9 |
| 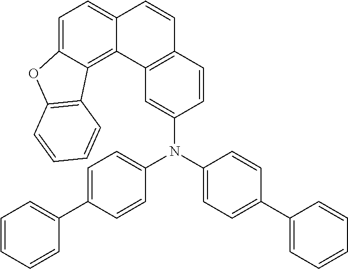 [SMILES: c1ccc(-c2ccc(N(c3ccc(-c4ccccc4)cc3)c3ccc4ccc5ccc6oc7ccccc7c6c5c4c3)cc2)cc1] | C2-46 |
| 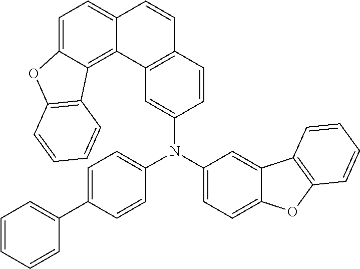 [SMILES: c1ccc(-c2ccc(N(c3ccc4oc5ccccc5c4c3)c3ccc4ccc5ccc6oc7ccccc7c6c5c4c3)cc2)cc1] | C2-54 |
| 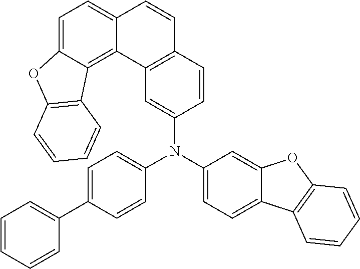 [SMILES: c1ccc(-c2ccc(N(c3ccc4c(c3)oc3ccccc34)c3ccc4ccc5ccc6oc7ccccc7c6c5c4c3)cc2)cc1] | C2-53 |
| 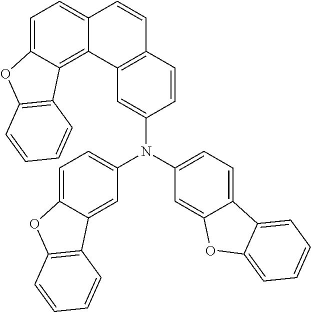 [SMILES: c1ccc2c(c1)oc1cc(N(c3ccc4oc5ccccc5c4c3)c3ccc4ccc5ccc6oc7ccccc7c6c5c4c3)ccc12] | C2-67 |
| 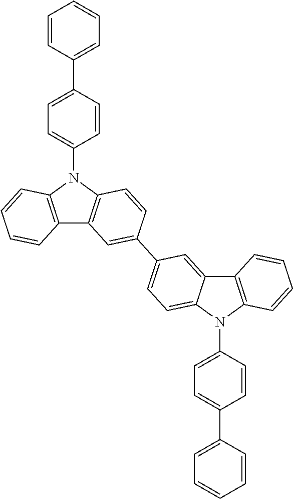 [SMILES: c1ccc(-c2ccc(-n3c4ccccc4c4cc(-c5ccc6c(c5)c5ccccc5n6-c5ccc(-c6ccccc6)cc5)ccc43)cc2)cc1] | T-1 |
| 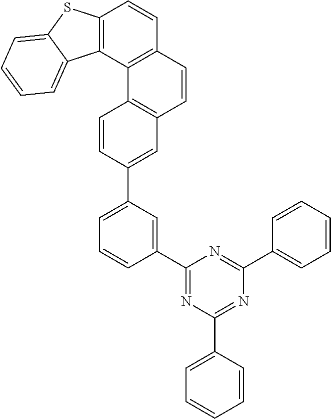 [SMILES: c1ccc(-c2nc(-c3ccccc3)nc(-c3cccc(-c4ccc5c(ccc6ccc7sc8ccccc8c7c65)c4)c3)n2)cc1] | C2-77 |
| 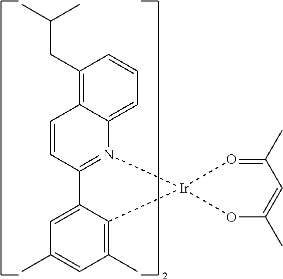 | D-39 |

Electron transport Layer/Electron Injection Layer

| | |
|---|---|
| 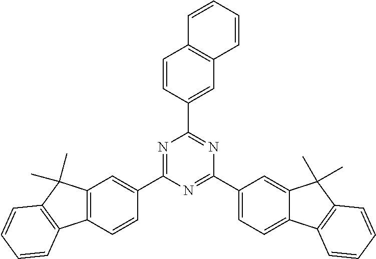 [SMILES: CC1(C)c2ccccc2-c2ccc(-c3nc(-c4ccc5c(c4)C(C)(C)c4ccccc4-5)nc(-c4ccc5ccccc5c4)n3)cc21] | ETL-1 |
| 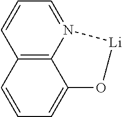 | EIL-1 |

1. A plurality of host materials comprising a first host material comprising a compound represented by the following formula 1, and a second host material comprising a compound represented by the following formula 2: 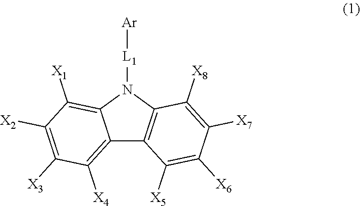
wherein,
Ar represents a substituted or unsubstituted (C6-C30)aryl, a substituted or unsubstituted pyridyl, a substituted or unsubstituted carbazolyl, a substituted or unsubstituted dibenzothiophenyl, a substituted or unsubstituted benzothiophenyl, a substituted or unsubstituted dibenzofuranyl, a substituted or unsubstituted benzofuranyl, a substituted or unsubstituted naphthyridinyl, a substituted or unsubstituted benzonaphthofuranyl, a substituted or unsubstituted benzonaphthothiophenyl, or —NX₉X₁₀;
L₁ represents a substituted or unsubstituted (C6-C30)arylene;
X₁ to X₈, each independently, represent hydrogen, deuterium, a halogen, a cyano, a carboxyl, a nitro, a hydroxyl, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NX₁₁X₁₂, or —SiX₁₃X₁₄X₁₅; or adjacent X₂ and X₃ or adjacent X₆ and X₇ may be linked to each other to form a substituted or unsubstituted, monocyclic ring(s) or polycyclic ring(s) having 2 to 5 rings, in which at least one ring must be formed; with the proviso that any one of X₁ to X₈ does not contain a carbazole ring; wherein the substituents of the substituted monocyclic ring(s) or polycyclic ring(s) are at least one selected from the group consisting of deuterium; a halogen; a cyano; a carboxyl; a nitro; a hydroxyl; a phosphineoxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (C6-C30)aryl unsubstituted or substituted with at least one of a cyano(s), a (C1-C30)alkyl(s) and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C6-C30)arylamino unsubstituted or substituted with a (C1-C30)alkyl(s); a (C1-C30)alkyl(C6-C30)arylamino; a mono- or di-(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl;
X₉ and X₁₀, each independently, represent a substituted or unsubstituted (C1-C30)alkyl, a substituted or unsubstituted (C3-C30)cycloalkyl, a substituted or unsubstituted (C6-C30)aryl, or a substituted or unsubstituted (3- to 30-membered)heteroaryl; and
X₁₁ to X₁₅, each independently, represent hydrogen, deuterium, a halogen, a cyano, a carboxyl, a nitro, a hydroxyl, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl; or adjacent ones of X₁₁ to X₁₅ may be linked to each other to form a rings; 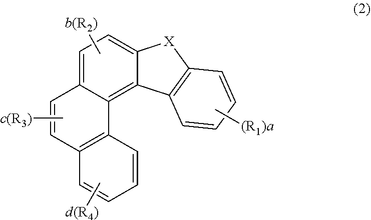
wherein,
X represents O, S, or CR₁₁R₁₂;
R₁ to R₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, or a substituted or unsubstituted (C6-C30)aryl(3- to 30-membered)heteroarylamino, provided that at least one of R₁ to R₄, each independently, represents a substituted or unsubstituted (C6-C30)aryl, or a substituted or unsubstituted (3- to 30-membered)heteroaryl, wherein the substituents of the substituted (C6-C30)aryl and the substituted (3- to 30-membered)heteroaryl are at least one selected from the group consisting of deuterium, a cyano, (C6-C30)aryl and (3- to 30-membered)heteroaryl;
R₁₁ and R₁₂, each independently, represent a substituted or unsubstituted (C1-C30)alkyl, a substituted or unsubstituted (C6-C30)aryl, or a substituted or unsubstituted (3- to 30-membered)heteroaryl; or R₁₁ and R₁₂ may be linked to each other to form a ring(s);
a and d, each independently, represent an integer of 1 to 4; b and c, each independently, represent an integer of 1 or 2; in which if a to d, each independently, are an integer of 2 or more, each of R₁, each of R₂, each of R₃, and each of R₄ may be the same or different; and the heteroaryl contains at least one heteroatom selected from B, N, O, S, Si, and P.

2. The plurality of host materials according to claim 1, wherein the substituents of the substituted alkyl, the substituted aryl, the substituted arylene, the substituted heteroaryl, the substituted heteroarylene, the substituted cycloalkyl, the substituted cycloalkenyl, the substituted heterocycloalkyl, the substituted alkoxy, the substituted trialkylsilyl, the substituted dialkylarylsilyl, the substituted alkyldiarylsilyl, the substituted triarylsilyl, the substituted mono- or di-alkylamino, the substituted mono- or di-arylamino, the substituted alkylarylamino, the substituted mono- or di-heteroarylamino, the substituted arylheteroarylamino, and the substituted monocyclic ring, each independently, are at least one selected from the group consisting of deuterium; a halogen; a cyano; a carboxyl; a nitro; a hydroxyl; a phosphineoxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of a (C1-C30)alkyl(s), a (C6-C30)aryl(s) and a (3- to 30-membered)heteroaryl(s); a (C6-C30)aryl unsubstituted or substituted with at least one of a cyano(s), a (C1-C30)alkyl(s) and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C6-C30)arylamino unsubstituted or substituted with a (C1-C30)alkyl(s); a (C1-C30)alkyl(C6-C30)arylamino; a mono- or di-(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

3. The plurality of host materials according to claim 1, wherein Ar in formula 1 represents a substituted or unsubstituted phenyl, a substituted or unsubstituted naphthyl, a substituted or unsubstituted biphenyl, a substituted or unsubstituted terphenyl, a substituted or unsubstituted fluorenyl, a substituted or unsubstituted benzofluorenyl, a substituted or unsubstituted triphenylenyl, a substituted or unsubstituted spirobifluorenyl, a substituted or unsubstituted pyridyl, a substituted or unsubstituted carbazolyl, a substituted or unsubstituted dibenzothiophenyl, a substituted or unsubstituted benzothiophenyl, a substituted or unsubstituted dibenzofuranyl, a substituted or unsubstituted benzofuranyl, a substituted or unsubstituted naphthyridinyl, a substituted or unsubstituted benzonaphthofuranyl, a substituted or unsubstituted benzonaphthothiophenyl, or —NX₉X₁₀.

4. The plurality of host materials according to claim 1, wherein the formula 2 is represented by at least one of the following formulas 2-1 and 2-3 to 2-5: 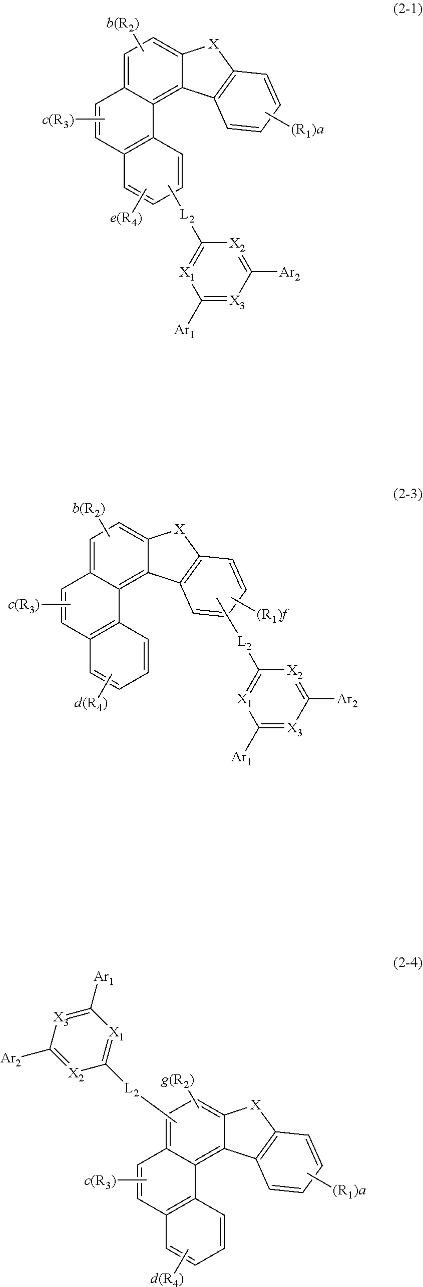 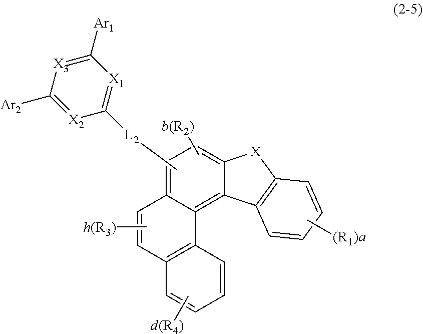
wherein,
X, and a to d are as defined in claim 1;
R₁ to R₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, or a substituted or unsubstituted (C6-C30)aryl(3- to 30-membered)heteroarylamino;
L₂ represents a single bond, a substituted or unsubstituted (C6-C30)arylene, or a substituted or unsubstituted (3- to 30-membered)heteroarylene;
X₁ to X₃, each independently, represent N or CH; with the proviso that at least one of X₁ to X₃ represents N;
Ar₁ and Ar₂, each independently, represent a substituted or unsubstituted (C6-C30)aryl, or a substituted or unsubstituted (3- to 30-membered)heteroaryl; and
e and f, each independently, represent an integer of 1 to 3; g and h, each independently, represent an integer of 1; in which if e to h, each independently, are an integer of 2 or more, each of R₁, each of R₂, each of R₃, and each of R₄ may be the same or different.

5. The plurality of host materials according to claim 1, wherein the compound represented by formula 1 is at least one selected from the following compounds:

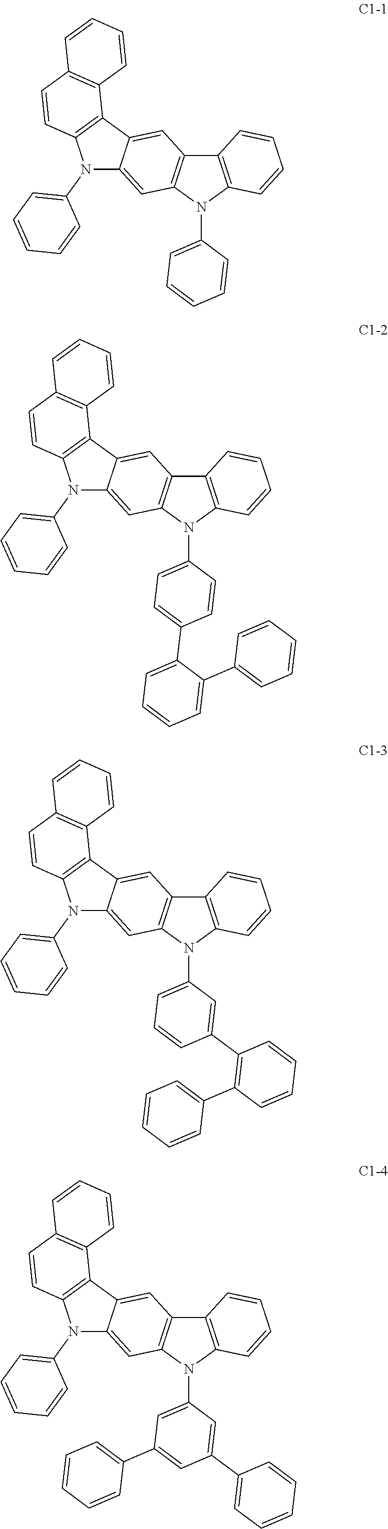 [SMILES: c1ccc(-n2c3ccccc3c3cc4c5c6ccccc6ccc5n(-c5ccccc5)c4cc32)cc1 ; c1ccc(-c2ccccc2-c2ccc(-n3c4ccccc4c4cc5c6c7ccccc7ccc6n(-c6ccccc6)c5cc43)cc2)cc1 ; c1ccc(-c2ccccc2-c2cccc(-n3c4ccccc4c4cc5c6c7ccccc7ccc6n(-c6ccccc6)c5cc43)c2)cc1 ; c1ccc(-c2cc(-c3ccccc3)cc(-n3c4ccccc4c4cc5c6c7ccccc7ccc6n(-c6ccccc6)c5cc43)c2)cc1]

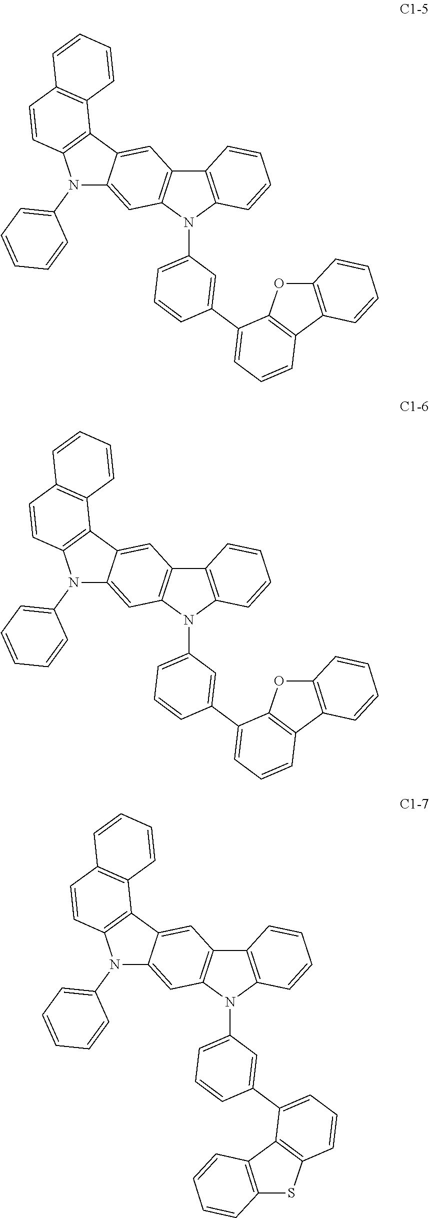 [SMILES: c1ccc(-n2c3cc4c(cc3c3c5ccccc5ccc32)c2ccccc2n4-c2cccc(-c3cccc4c3oc3ccccc34)c2)cc1 ; c1ccc(-n2c3cc4c(cc3c3c5ccccc5ccc32)c2ccccc2n4-c2cccc(-c3cccc4c3oc3ccccc34)c2)cc1 ; c1ccc(-n2c3cc4c(cc3c3c5ccccc5ccc32)c2ccccc2n4-c2cccc(-c3cccc4sc5ccccc5c34)c2)cc1]

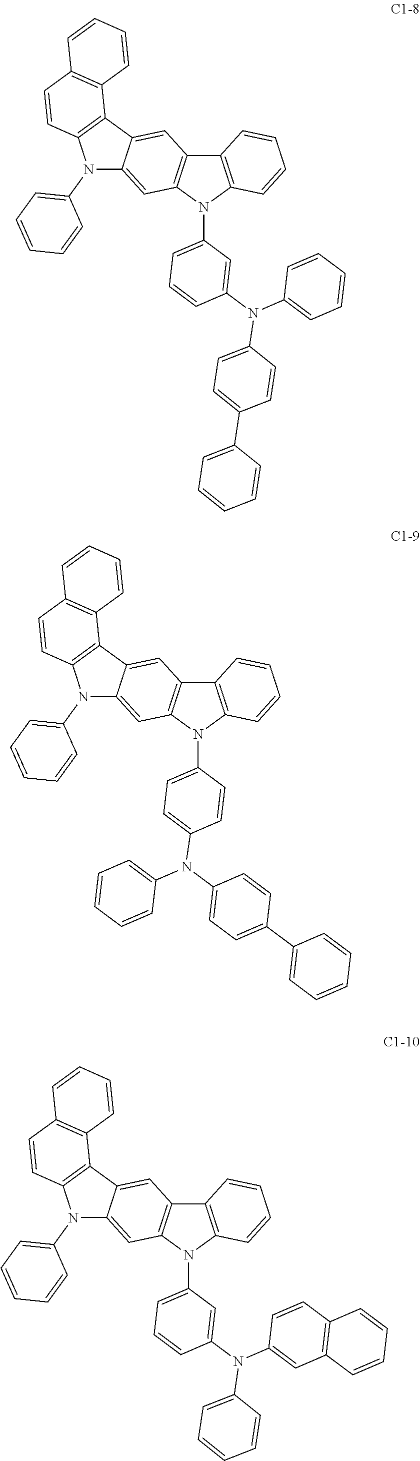 [SMILES: c1ccc(-c2ccc(N(c3ccccc3)c3cccc(-n4c5ccccc5c5cc6c7c8ccccc8ccc7n(-c7ccccc7)c6cc54)c3)cc2)cc1 ; c1ccc(-c2ccc(N(c3ccccc3)c3ccc(-n4c5ccccc5c5cc6c7c8ccccc8ccc7n(-c7ccccc7)c6cc54)cc3)cc2)cc1 ; c1ccc(N(c2cccc(-n3c4ccccc4c4cc5c6c7ccccc7ccc6n(-c6ccccc6)c5cc43)c2)c2ccc3ccccc3c2)cc1]

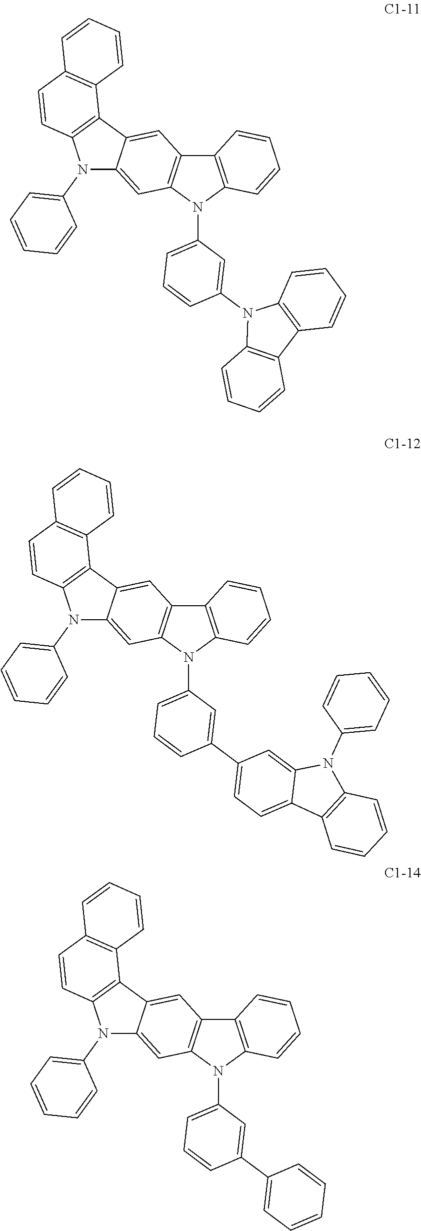 [SMILES: c1ccc(-n2c3cc4c(cc3c3c5ccccc5ccc32)c2ccccc2n4-c2cccc(-n3c4ccccc4c4ccccc43)c2)cc1 ; c1ccc(-n2c3ccccc3c3ccc(-c4cccc(-n5c6ccccc6c6cc7c8c9ccccc9ccc8n(-c8ccccc8)c7cc65)c4)cc32)cc1 ; c1ccc(-c2cccc(-n3c4ccccc4c4cc5c6c7ccccc7ccc6n(-c6ccccc6)c5cc43)c2)cc1]

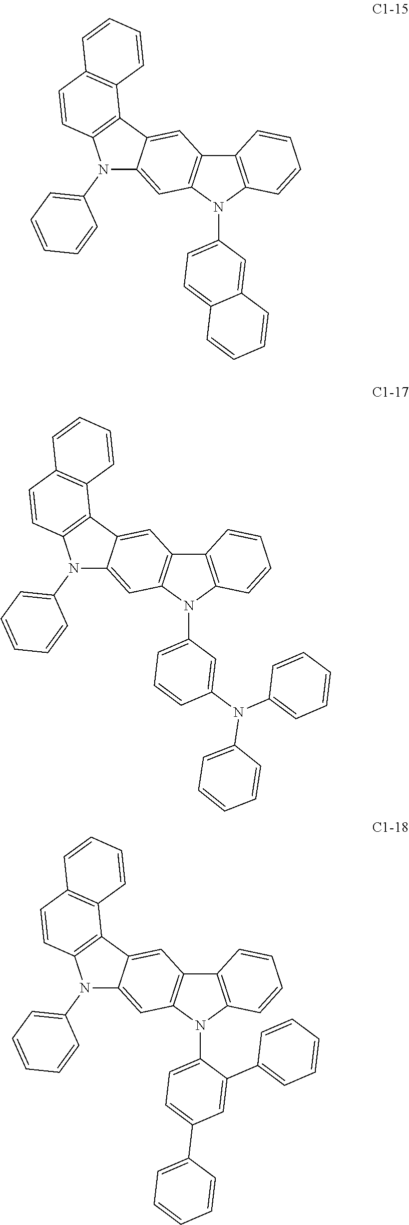 [SMILES: c1ccc(-n2c3cc4c(cc3c3c5ccccc5ccc32)c2ccccc2n4-c2ccc3ccccc3c2)cc1 ; c1ccc(N(c2ccccc2)c2cccc(-n3c4ccccc4c4cc5c6c7ccccc7ccc6n(-c6ccccc6)c5cc43)c2)cc1 ; c1ccc(-c2ccc(-n3c4ccccc4c4cc5c6c7ccccc7ccc6n(-c6ccccc6)c5cc43)c(-c3ccccc3)c2)cc1]

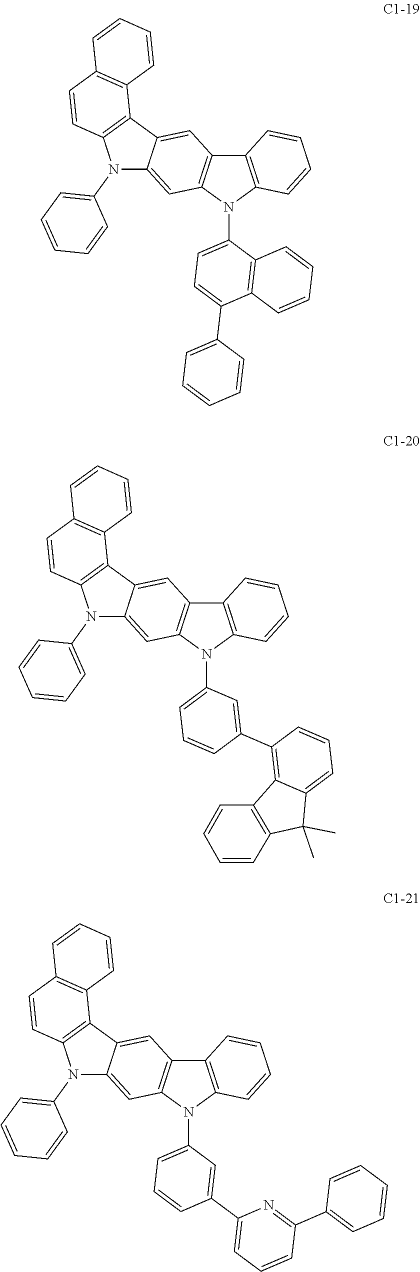 [SMILES: c1ccc(-c2ccc(-n3c4ccccc4c4cc5c6c7ccccc7ccc6n(-c6ccccc6)c5cc43)c3ccccc23)cc1 ; CC1(C)c2ccccc2-c2c(-c3cccc(-n4c5ccccc5c5cc6c7c8ccccc8ccc7n(-c7ccccc7)c6cc54)c3)cccc21 ; c1ccc(-c2cccc(-c3cccc(-n4c5ccccc5c5cc6c7c8ccccc8ccc7n(-c7ccccc7)c6cc54)c3)n2)cc1]

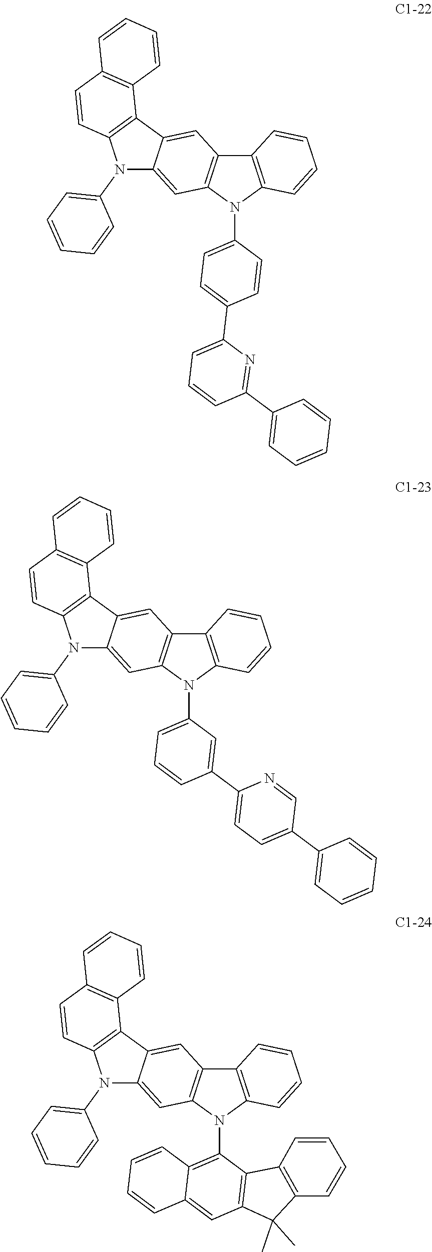 [SMILES: c1ccc(-c2cccc(-c3ccc(-n4c5ccccc5c5cc6c7c8ccccc8ccc7n(-c7ccccc7)c6cc54)cc3)n2)cc1 ; c1ccc(-c2ccc(-c3cccc(-n4c5ccccc5c5cc6c7c8ccccc8ccc7n(-c7ccccc7)c6cc54)c3)nc2)cc1 ; CC1(C)c2ccccc2-c2c1cc1ccccc1c2-n1c2ccccc2c2cc3c4c5ccccc5ccc4n(-c4ccccc4)c3cc21]

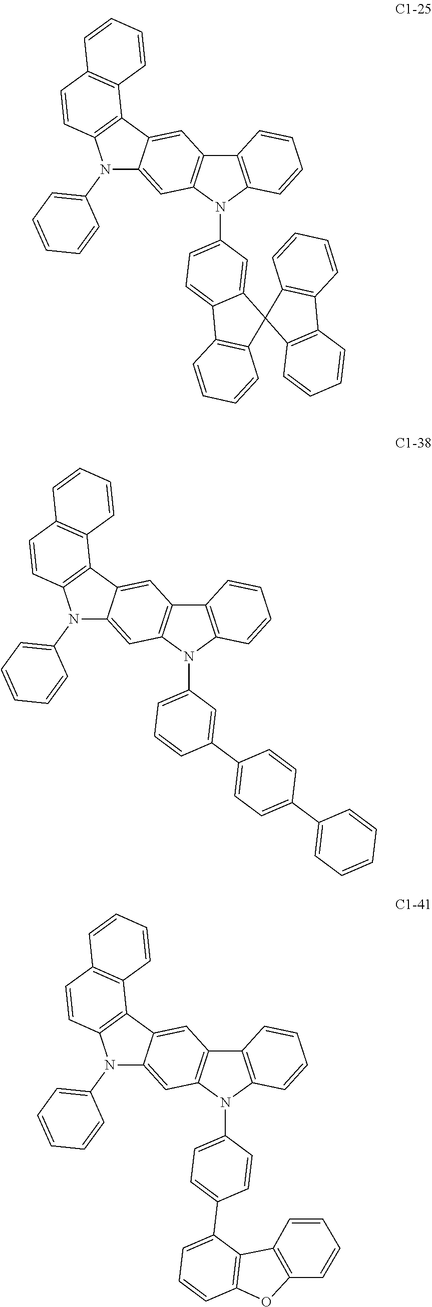 [SMILES: c1ccc(-n2c3cc4c(cc3c3c5ccccc5ccc32)c2ccccc2n4-c2ccc3c(c2)C2(c4ccccc4-c4ccccc42)c2ccccc2-3)cc1 ; c1ccc(-c2ccc(-c3cccc(-n4c5ccccc5c5cc6c7c8ccccc8ccc7n(-c7ccccc7)c6cc54)c3)cc2)cc1 ; c1ccc(-n2c3cc4c(cc3c3c5ccccc5ccc32)c2ccccc2n4-c2ccc(-c3cccc4oc5ccccc5c34)cc2)cc1]

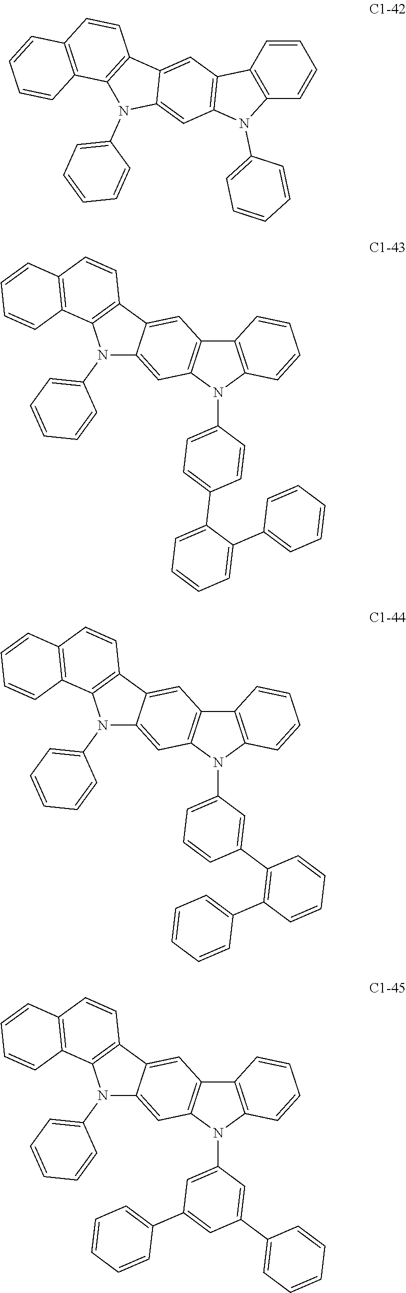 [SMILES: c1ccc(-n2c3ccccc3c3cc4c5ccc6ccccc6c5n(-c5ccccc5)c4cc32)cc1 ; c1ccc(-c2ccccc2-c2ccc(-n3c4ccccc4c4cc5c6ccc7ccccc7c6n(-c6ccccc6)c5cc43)cc2)cc1 ; c1ccc(-c2ccccc2-c2cccc(-n3c4ccccc4c4cc5c6ccc7ccccc7c6n(-c6ccccc6)c5cc43)c2)cc1 ; c1ccc(-c2cc(-c3ccccc3)cc(-n3c4ccccc4c4cc5c6ccc7ccccc7c6n(-c6ccccc6)c5cc43)c2)cc1]

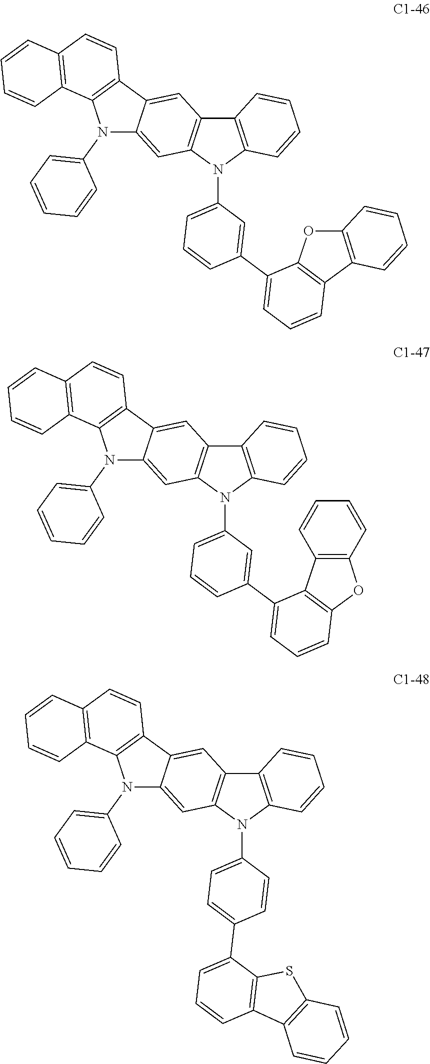 [SMILES: c1ccc(-n2c3cc4c(cc3c3ccc5ccccc5c32)c2ccccc2n4-c2cccc(-c3cccc4c3oc3ccccc34)c2)cc1 ; c1ccc(-n2c3cc4c(cc3c3ccc5ccccc5c32)c2ccccc2n4-c2cccc(-c3cccc4oc5ccccc5c34)c2)cc1 ; c1ccc(-n2c3cc4c(cc3c3ccc5ccccc5c32)c2ccccc2n4-c2ccc(-c3cccc4c3sc3ccccc34)cc2)cc1]

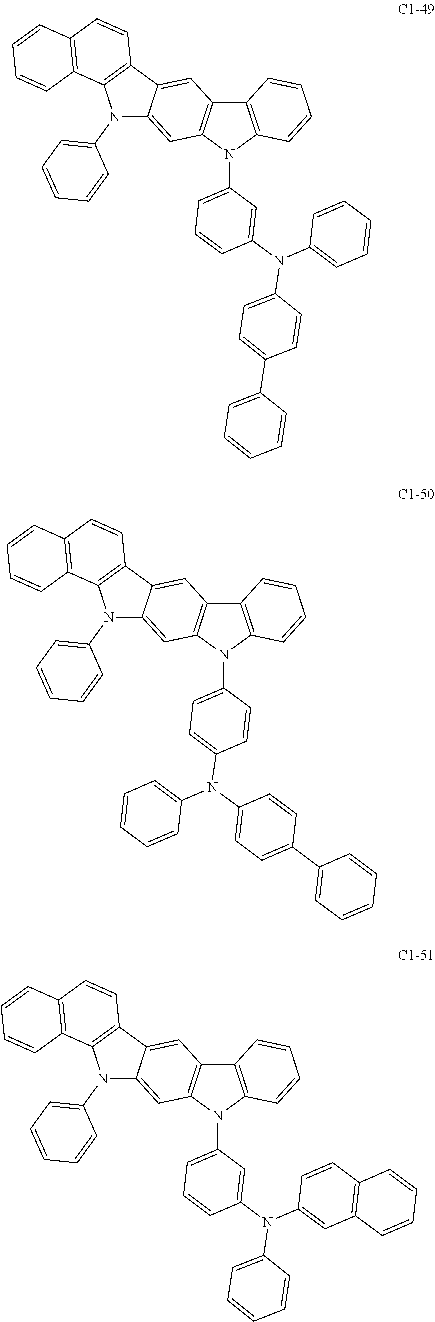 [SMILES: c1ccc(-c2ccc(N(c3ccccc3)c3cccc(-n4c5ccccc5c5cc6c7ccc8ccccc8c7n(-c7ccccc7)c6cc54)c3)cc2)cc1 ; c1ccc(-c2ccc(N(c3ccccc3)c3ccc(-n4c5ccccc5c5cc6c7ccc8ccccc8c7n(-c7ccccc7)c6cc54)cc3)cc2)cc1 ; c1ccc(N(c2cccc(-n3c4ccccc4c4cc5c6ccc7ccccc7c6n(-c6ccccc6)c5cc43)c2)c2ccc3ccccc3c2)cc1]

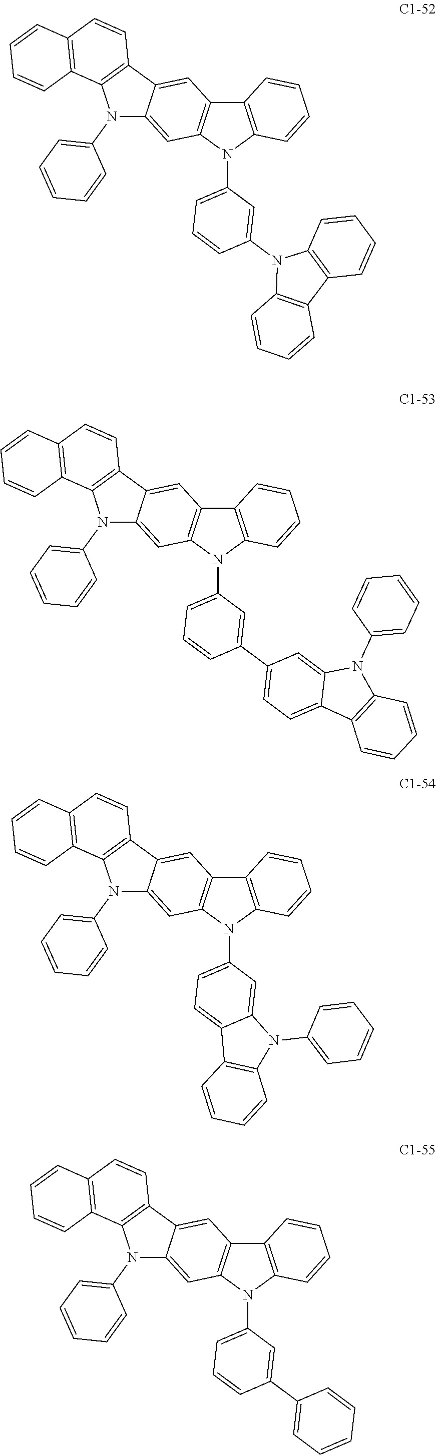 [SMILES: c1ccc(-n2c3cc4c(cc3c3ccc5ccccc5c32)c2ccccc2n4-c2cccc(-n3c4ccccc4c4ccccc43)c2)cc1 ; c1ccc(-n2c3ccccc3c3ccc(-c4cccc(-n5c6ccccc6c6cc7c8ccc9ccccc9c8n(-c8ccccc8)c7cc65)c4)cc32)cc1 ; c1ccc(-n2c3ccccc3c3ccc(-n4c5ccccc5c5cc6c7ccc8ccccc8c7n(-c7ccccc7)c6cc54)cc32)cc1 ; c1ccc(-c2cccc(-n3c4ccccc4c4cc5c6ccc7ccccc7c6n(-c6ccccc6)c5cc43)c2)cc1]

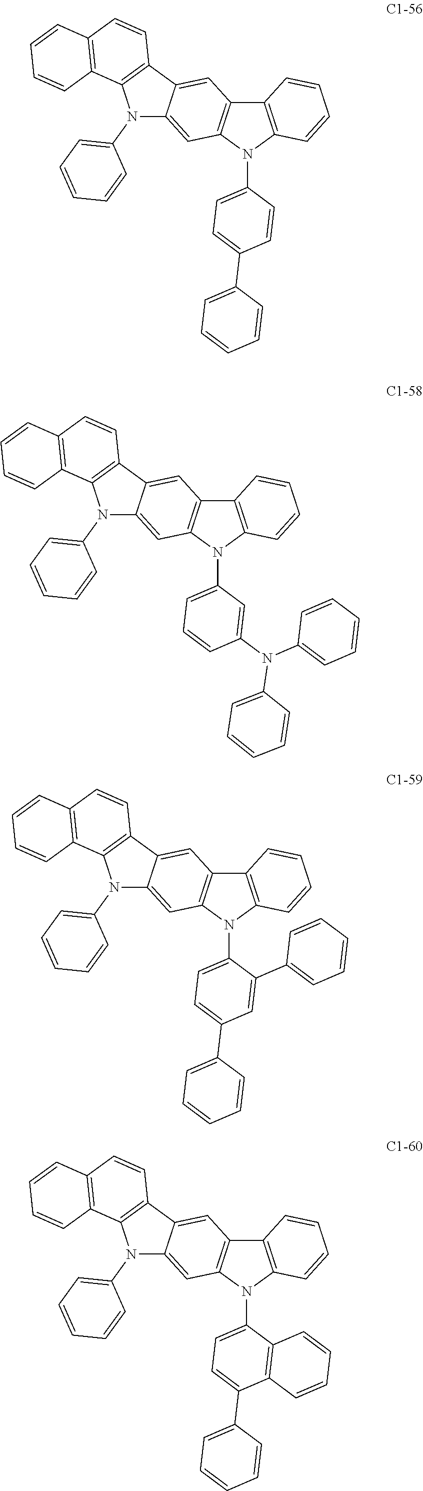 [SMILES: c1ccc(-c2ccc(-n3c4ccccc4c4cc5c6ccc7ccccc7c6n(-c6ccccc6)c5cc43)cc2)cc1 ; c1ccc(N(c2ccccc2)c2cccc(-n3c4ccccc4c4cc5c6ccc7ccccc7c6n(-c6ccccc6)c5cc43)c2)cc1 ; c1ccc(-c2ccc(-n3c4ccccc4c4cc5c6ccc7ccccc7c6n(-c6ccccc6)c5cc43)c(-c3ccccc3)c2)cc1 ; c1ccc(-c2ccc(-n3c4ccccc4c4cc5c6ccc7ccccc7c6n(-c6ccccc6)c5cc43)c3ccccc23)cc1]

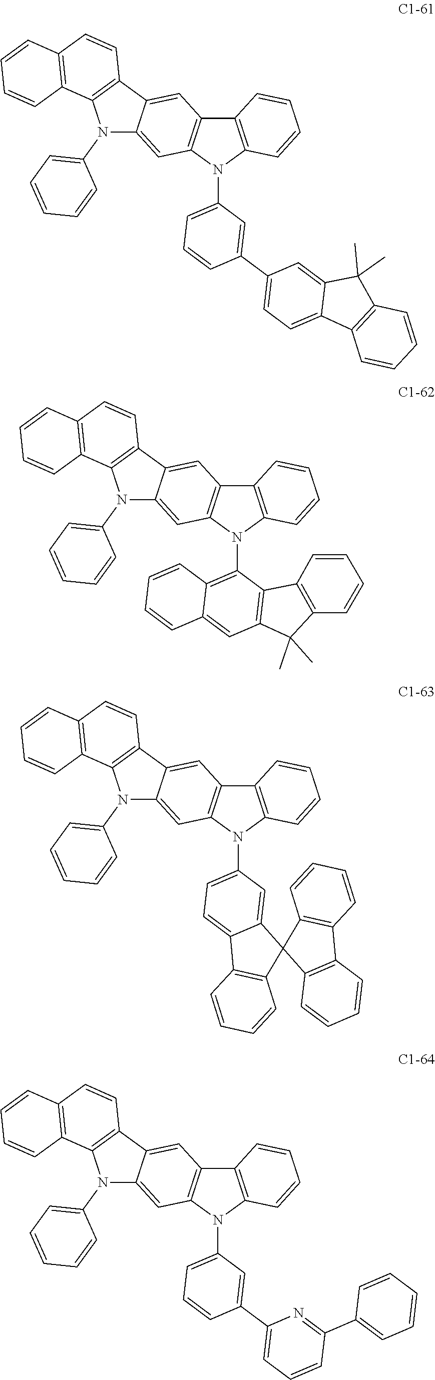 [SMILES: CC1(C)c2ccccc2-c2ccc(-c3cccc(-n4c5ccccc5c5cc6c7ccc8ccccc8c7n(-c7ccccc7)c6cc54)c3)cc21 ; CC1(C)c2ccccc2-c2c1cc1ccccc1c2-n1c2ccccc2c2cc3c4ccc5ccccc5c4n(-c4ccccc4)c3cc21 ; c1ccc(-n2c3cc4c(cc3c3ccc5ccccc5c32)c2ccccc2n4-c2ccc3c(c2)C2(c4ccccc4-c4ccccc42)c2ccccc2-3)cc1 ; c1ccc(-c2cccc(-c3cccc(-n4c5ccccc5c5cc6c7ccc8ccccc8c7n(-c7ccccc7)c6cc54)c3)n2)cc1]

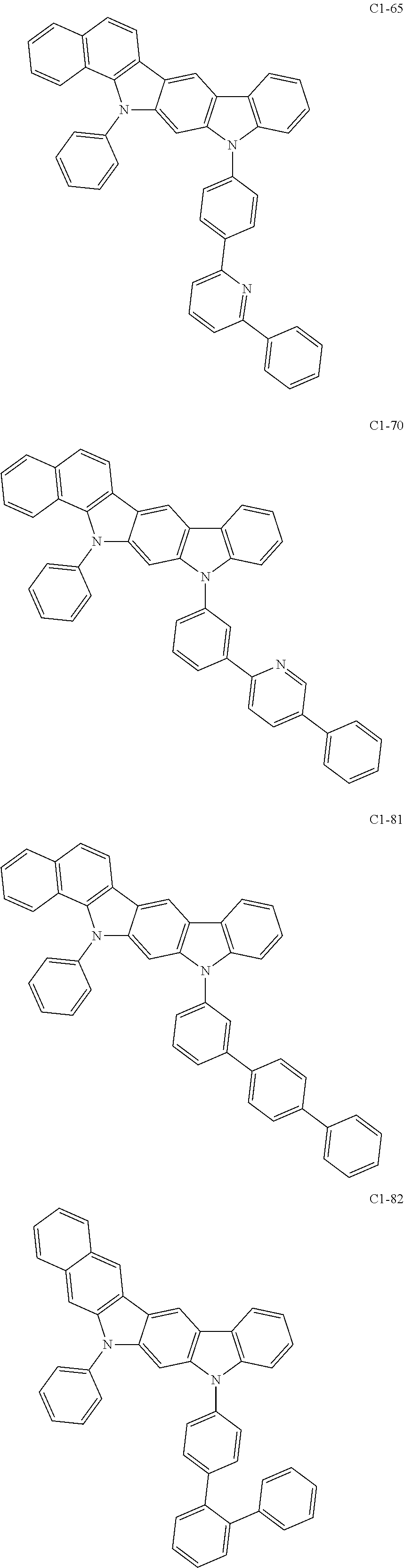 [SMILES: c1ccc(-c2cccc(-c3ccc(-n4c5ccccc5c5cc6c7ccc8ccccc8c7n(-c7ccccc7)c6cc54)cc3)n2)cc1 ; c1ccc(-c2ccc(-c3cccc(-n4c5ccccc5c5cc6c7ccc8ccccc8c7n(-c7ccccc7)c6cc54)c3)nc2)cc1 ; c1ccc(-c2ccc(-c3cccc(-n4c5ccccc5c5cc6c7ccc8ccccc8c7n(-c7ccccc7)c6cc54)c3)cc2)cc1 ; c1ccc(-c2ccccc2-c2ccc(-n3c4ccccc4c4cc5c6cc7ccccc7cc6n(-c6ccccc6)c5cc43)cc2)cc1]

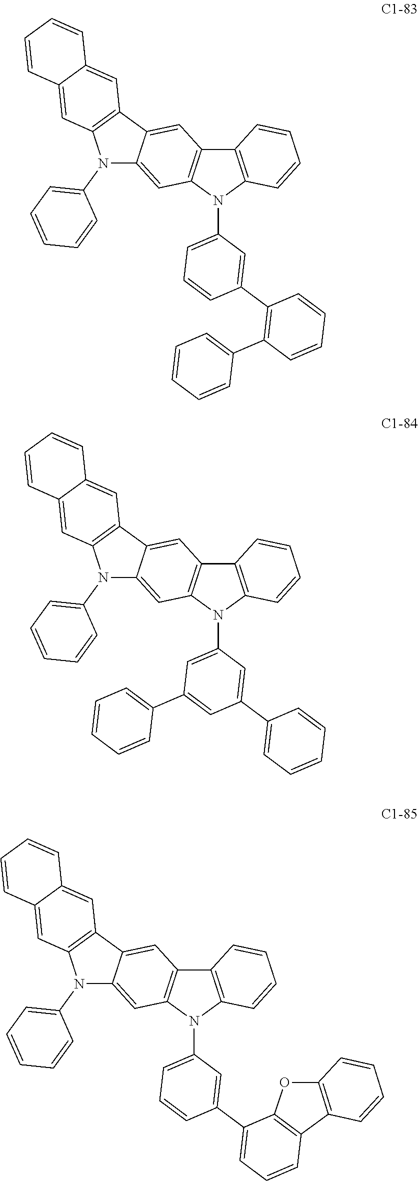 [SMILES: c1ccc(-c2ccccc2-c2cccc(-n3c4ccccc4c4cc5c6cc7ccccc7cc6n(-c6ccccc6)c5cc43)c2)cc1 ; c1ccc(-c2cc(-c3ccccc3)cc(-n3c4ccccc4c4cc5c6cc7ccccc7cc6n(-c6ccccc6)c5cc43)c2)cc1 ; c1ccc(-n2c3cc4ccccc4cc3c3cc4c5ccccc5n(-c5cccc(-c6cccc7c6oc6ccccc67)c5)c4cc32)cc1]

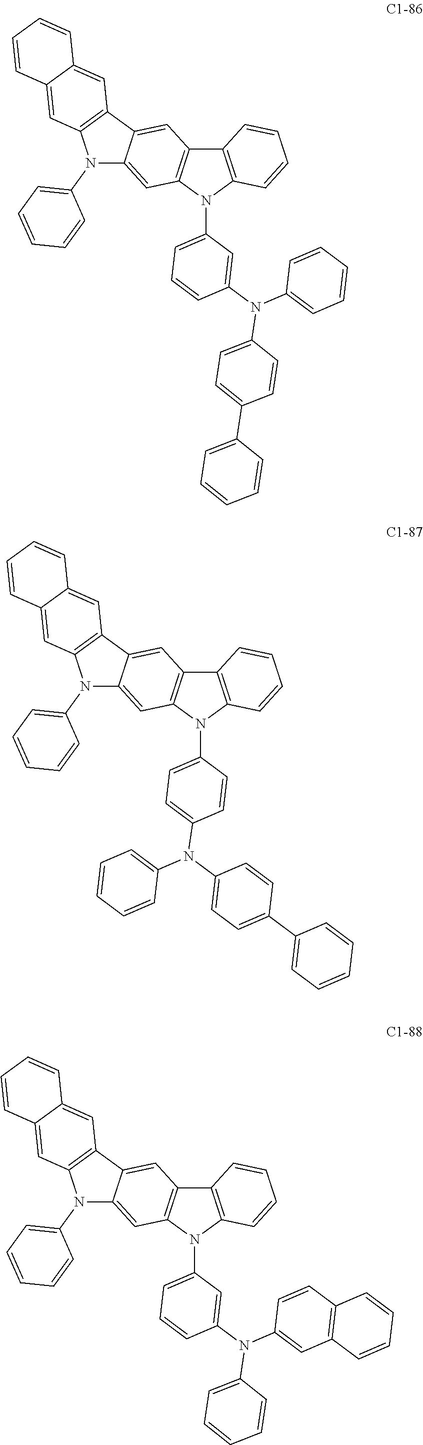 [SMILES: c1ccc(-c2ccc(N(c3ccccc3)c3cccc(-n4c5ccccc5c5cc6c7cc8ccccc8cc7n(-c7ccccc7)c6cc54)c3)cc2)cc1 ; c1ccc(-c2ccc(N(c3ccccc3)c3ccc(-n4c5ccccc5c5cc6c7cc8ccccc8cc7n(-c7ccccc7)c6cc54)cc3)cc2)cc1 ; c1ccc(N(c2cccc(-n3c4ccccc4c4cc5c6cc7ccccc7cc6n(-c6ccccc6)c5cc43)c2)c2ccc3ccccc3c2)cc1]

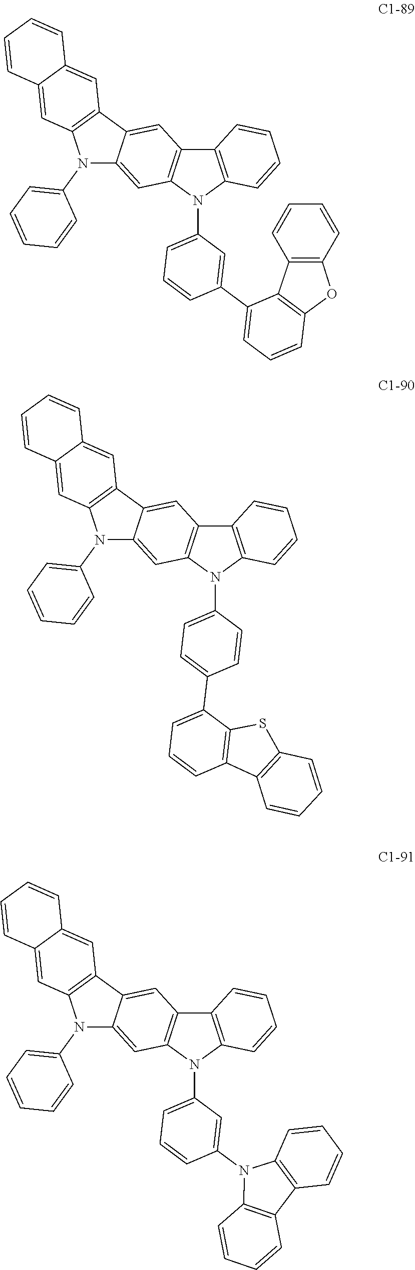 [SMILES: c1ccc(-n2c3cc4ccccc4cc3c3cc4c5ccccc5n(-c5cccc(-c6cccc7oc8ccccc8c67)c5)c4cc32)cc1 ; c1ccc(-n2c3cc4ccccc4cc3c3cc4c5ccccc5n(-c5ccc(-c6cccc7c6sc6ccccc67)cc5)c4cc32)cc1 ; c1ccc(-n2c3cc4ccccc4cc3c3cc4c5ccccc5n(-c5cccc(-n6c7ccccc7c7ccccc76)c5)c4cc32)cc1]

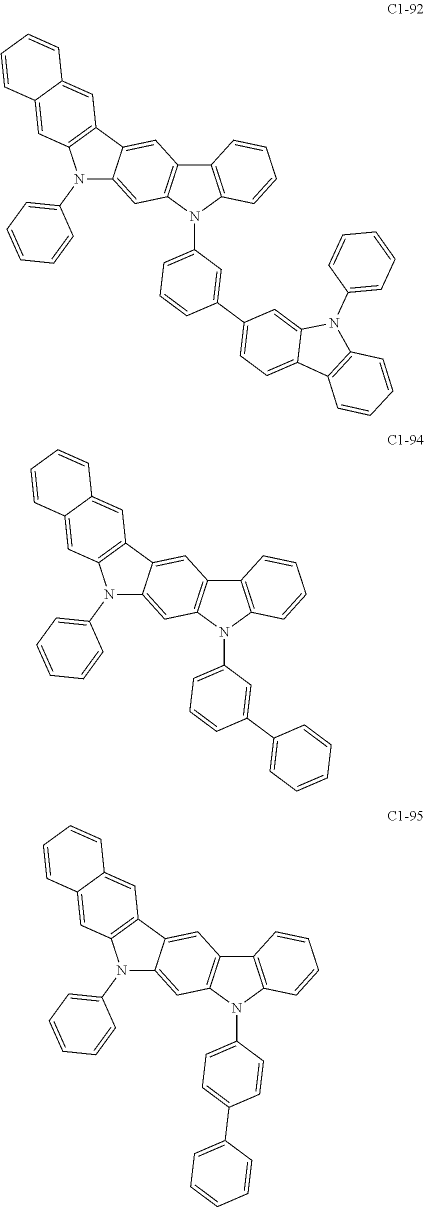 [SMILES: c1ccc(-n2c3ccccc3c3ccc(-c4cccc(-n5c6ccccc6c6cc7c8cc9ccccc9cc8n(-c8ccccc8)c7cc65)c4)cc32)cc1 ; c1ccc(-c2cccc(-n3c4ccccc4c4cc5c6cc7ccccc7cc6n(-c6ccccc6)c5cc43)c2)cc1 ; c1ccc(-c2ccc(-n3c4ccccc4c4cc5c6cc7ccccc7cc6n(-c6ccccc6)c5cc43)cc2)cc1]

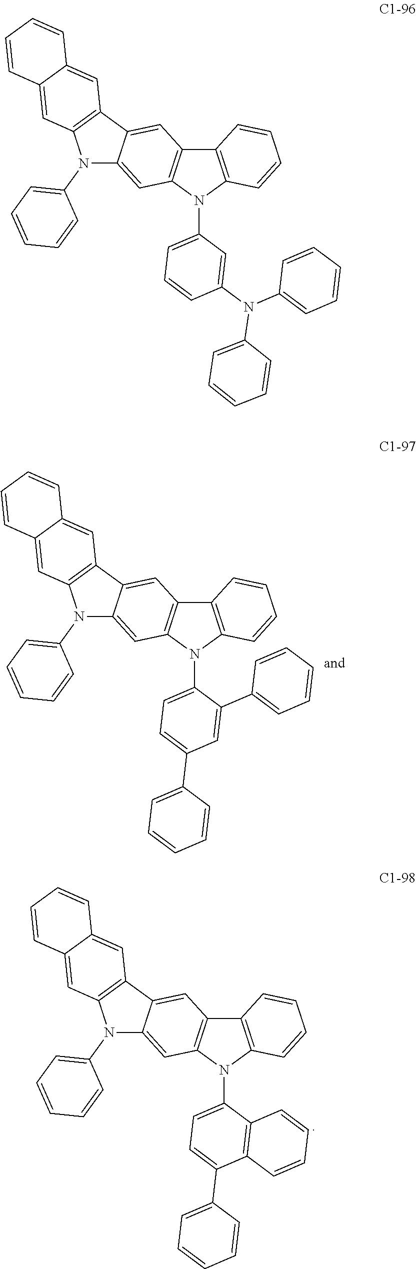 [SMILES: c1ccc(N(c2ccccc2)c2cccc(-n3c4ccccc4c4cc5c6cc7ccccc7cc6n(-c6ccccc6)c5cc43)c2)cc1 ; c1ccc(-c2ccc(-n3c4ccccc4c4cc5c6cc7ccccc7cc6n(-c6ccccc6)c5cc43)c(-c3ccccc3)c2)cc1 ; c1ccc(-c2ccc(-n3c4ccccc4c4cc5c6cc7ccccc7cc6n(-c6ccccc6)c5cc43)c3ccccc23)cc1]

6. The plurality of host materials according to claim 1, wherein the compound represented by formula 2 is at least one selected from the following compounds:

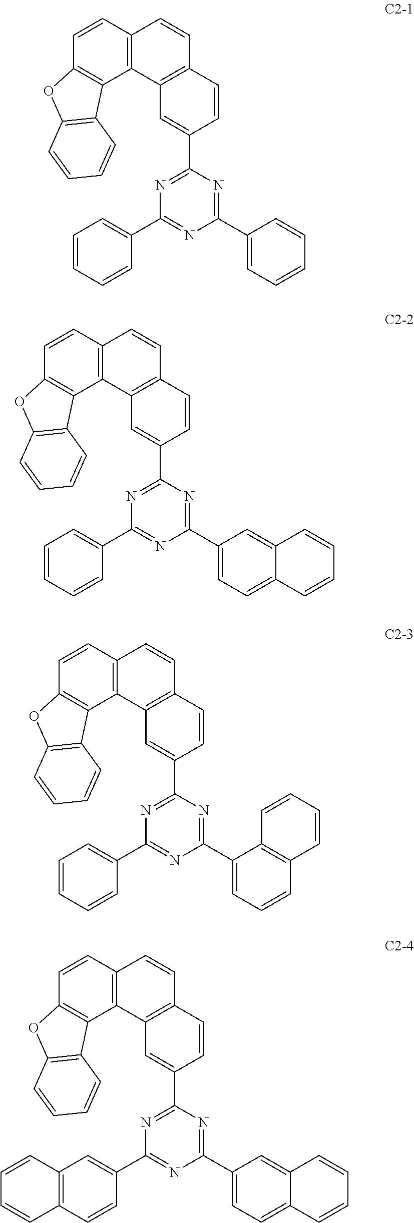

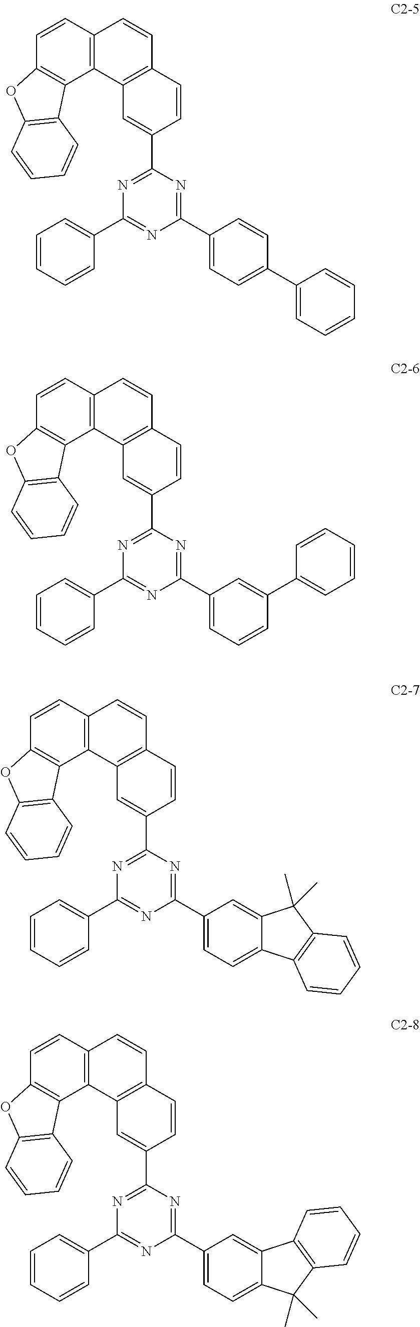 [SMILES: c1ccc(-c2ccc(-c3nc(-c4ccccc4)nc(-c4ccc5ccc6ccc7oc8ccccc8c7c6c5c4)n3)cc2)cc1 ; c1ccc(-c2cccc(-c3nc(-c4ccccc4)nc(-c4ccc5ccc6ccc7oc8ccccc8c7c6c5c4)n3)c2)cc1 ; CC1(C)c2ccccc2-c2ccc(-c3nc(-c4ccccc4)nc(-c4ccc5ccc6ccc7oc8ccccc8c7c6c5c4)n3)cc21 ; CC1(C)c2ccccc2-c2cc(-c3nc(-c4ccccc4)nc(-c4ccc5ccc6ccc7oc8ccccc8c7c6c5c4)n3)ccc21]

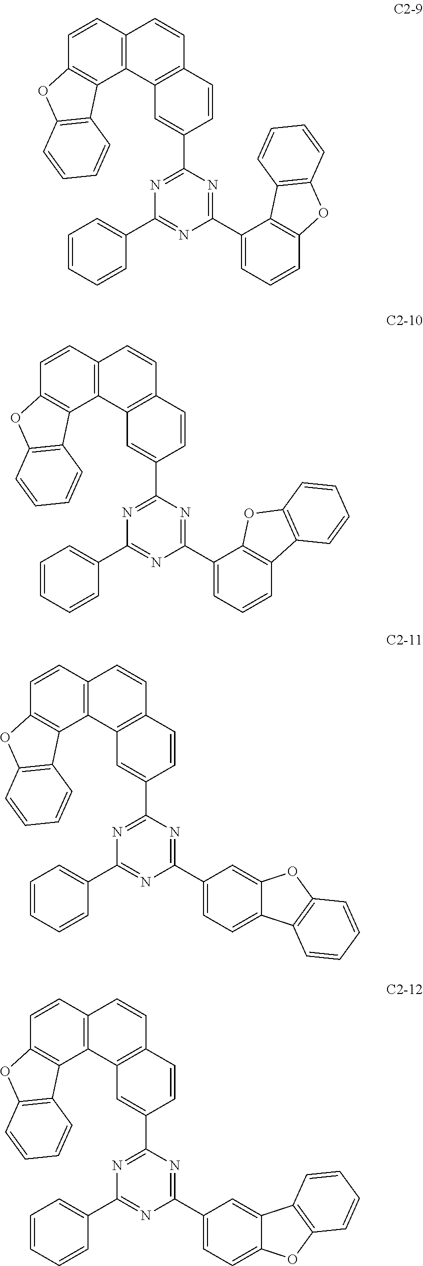 [SMILES: c1ccc(-c2nc(-c3ccc4ccc5ccc6oc7ccccc7c6c5c4c3)nc(-c3cccc4oc5ccccc5c34)n2)cc1 ; c1ccc(-c2nc(-c3ccc4ccc5ccc6oc7ccccc7c6c5c4c3)nc(-c3cccc4c3oc3ccccc34)n2)cc1 ; c1ccc(-c2nc(-c3ccc4c(c3)oc3ccccc34)nc(-c3ccc4ccc5ccc6oc7ccccc7c6c5c4c3)n2)cc1 ; c1ccc(-c2nc(-c3ccc4oc5ccccc5c4c3)nc(-c3ccc4ccc5ccc6oc7ccccc7c6c5c4c3)n2)cc1]

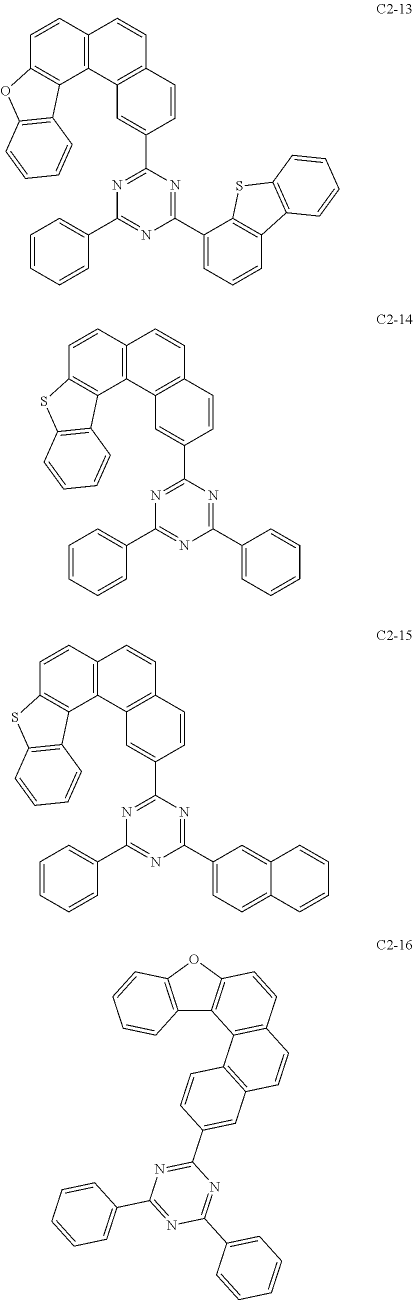 [SMILES: c1ccc(-c2nc(-c3ccc4ccc5ccc6oc7ccccc7c6c5c4c3)nc(-c3cccc4c3sc3ccccc34)n2)cc1 ; c1ccc(-c2nc(-c3ccccc3)nc(-c3ccc4ccc5ccc6sc7ccccc7c6c5c4c3)n2)cc1 ; c1ccc(-c2nc(-c3ccc4ccccc4c3)nc(-c3ccc4ccc5ccc6sc7ccccc7c6c5c4c3)n2)cc1 ; c1ccc(-c2nc(-c3ccccc3)nc(-c3ccc4c(ccc5ccc6oc7ccccc7c6c54)c3)n2)cc1]

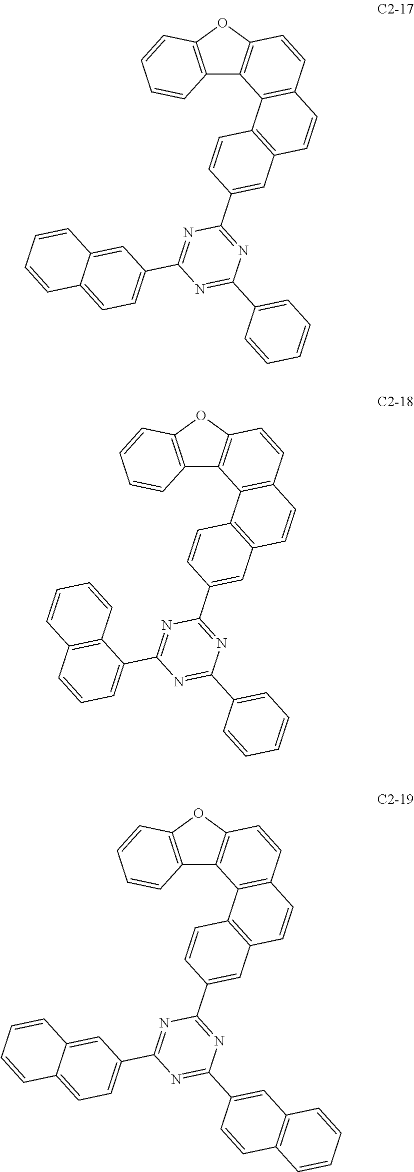 [SMILES: c1ccc(-c2nc(-c3ccc4ccccc4c3)nc(-c3ccc4c(ccc5ccc6oc7ccccc7c6c54)c3)n2)cc1 ; c1ccc(-c2nc(-c3ccc4c(ccc5ccc6oc7ccccc7c6c54)c3)nc(-c3cccc4ccccc34)n2)cc1 ; c1ccc2cc(-c3nc(-c4ccc5ccccc5c4)nc(-c4ccc5c(ccc6ccc7oc8ccccc8c7c65)c4)n3)ccc2c1]

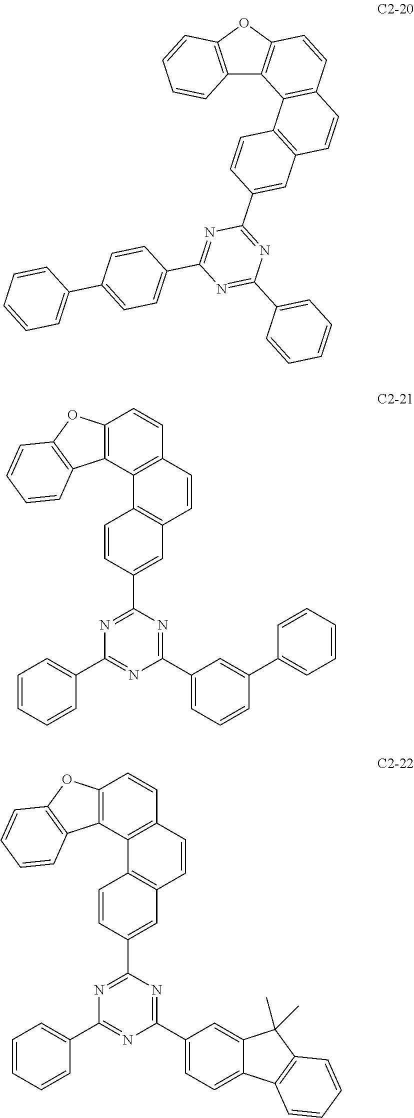 [SMILES: c1ccc(-c2ccc(-c3nc(-c4ccccc4)nc(-c4ccc5c(ccc6ccc7oc8ccccc8c7c65)c4)n3)cc2)cc1 ; c1ccc(-c2cccc(-c3nc(-c4ccccc4)nc(-c4ccc5c(ccc6ccc7oc8ccccc8c7c65)c4)n3)c2)cc1 ; CC1(C)c2ccccc2-c2ccc(-c3nc(-c4ccccc4)nc(-c4ccc5c(ccc6ccc7oc8ccccc8c7c65)c4)n3)cc21]

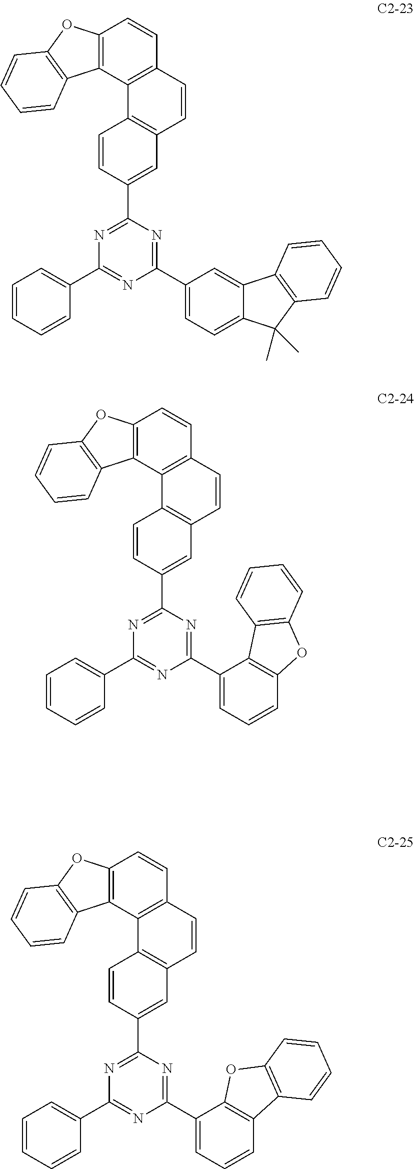 [SMILES: CC1(C)c2ccccc2-c2cc(-c3nc(-c4ccccc4)nc(-c4ccc5c(ccc6ccc7oc8ccccc8c7c65)c4)n3)ccc21 ; c1ccc(-c2nc(-c3ccc4c(ccc5ccc6oc7ccccc7c6c54)c3)nc(-c3cccc4oc5ccccc5c34)n2)cc1 ; c1ccc(-c2nc(-c3ccc4c(ccc5ccc6oc7ccccc7c6c54)c3)nc(-c3cccc4c3oc3ccccc34)n2)cc1]

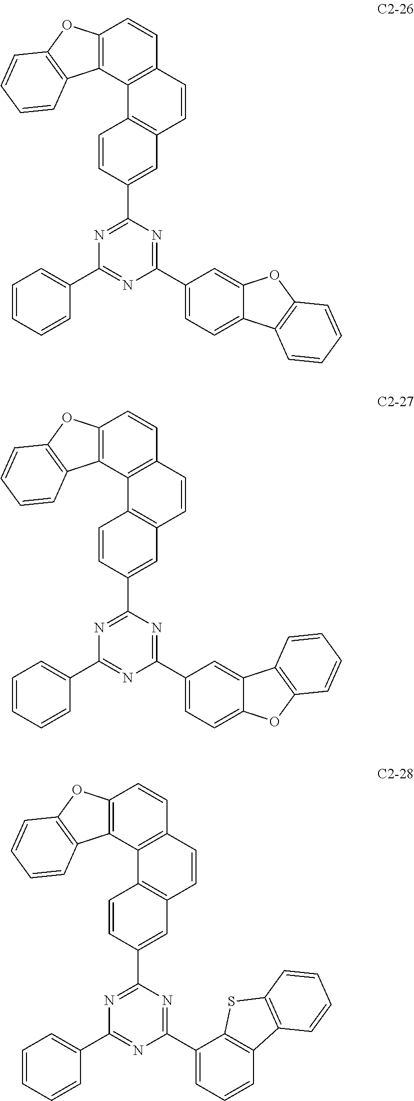 [SMILES: c1ccc(-c2nc(-c3ccc4c(ccc5ccc6oc7ccccc7c6c54)c3)nc(-c3ccc4c(c3)oc3ccccc34)n2)cc1 ; c1ccc(-c2nc(-c3ccc4c(ccc5ccc6oc7ccccc7c6c54)c3)nc(-c3ccc4oc5ccccc5c4c3)n2)cc1 ; c1ccc(-c2nc(-c3ccc4c(ccc5ccc6oc7ccccc7c6c54)c3)nc(-c3cccc4c3sc3ccccc34)n2)cc1]

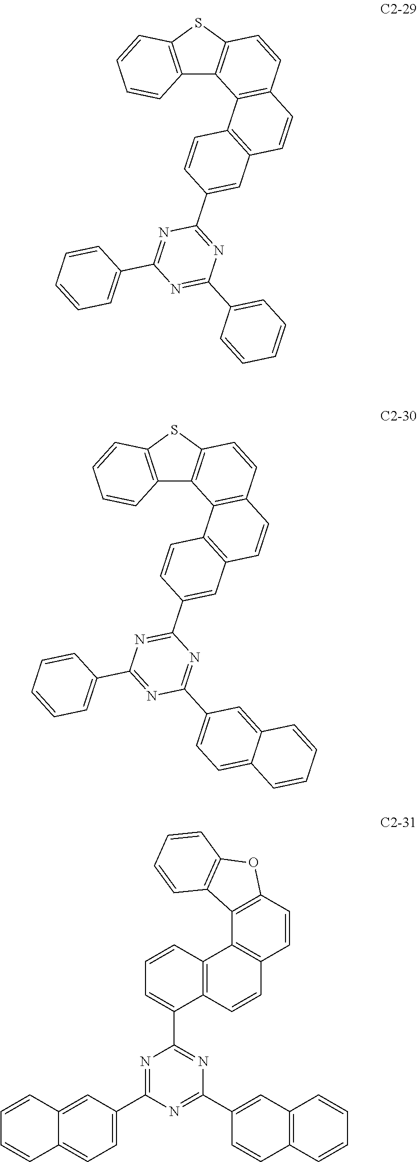 [SMILES: c1ccc(-c2nc(-c3ccccc3)nc(-c3ccc4c(ccc5ccc6sc7ccccc7c6c54)c3)n2)cc1 ; c1ccc(-c2nc(-c3ccc4ccccc4c3)nc(-c3ccc4c(ccc5ccc6sc7ccccc7c6c54)c3)n2)cc1 ; c1ccc2cc(-c3nc(-c4ccc5ccccc5c4)nc(-c4cccc5c4ccc4ccc6oc7ccccc7c6c45)n3)ccc2c1]

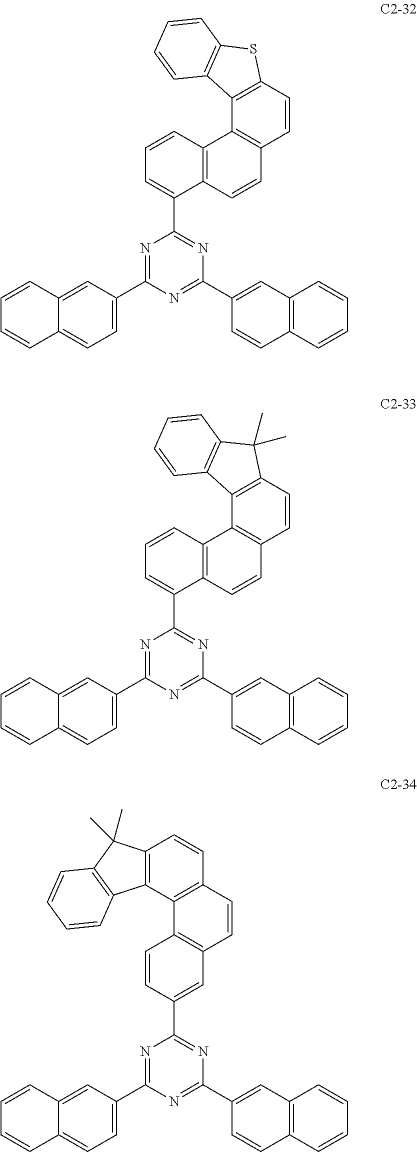 [SMILES: c1ccc2cc(-c3nc(-c4ccc5ccccc5c4)nc(-c4cccc5c4ccc4ccc6sc7ccccc7c6c45)n3)ccc2c1 ; CC1(C)c2ccccc2-c2c1ccc1ccc3c(-c4nc(-c5ccc6ccccc6c5)nc(-c5ccc6ccccc6c5)n4)cccc3c21 ; CC1(C)c2ccccc2-c2c1ccc1ccc3cc(-c4nc(-c5ccc6ccccc6c5)nc(-c5ccc6ccccc6c5)n4)ccc3c21]

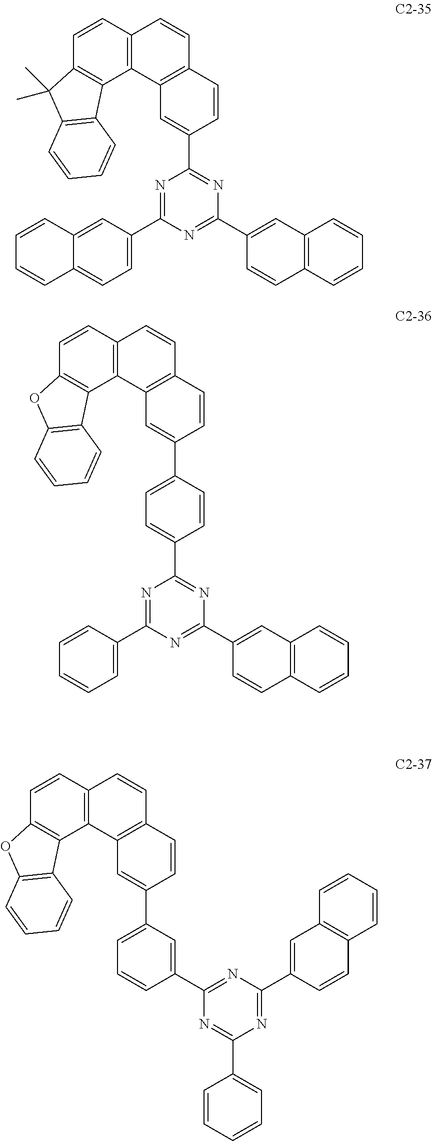

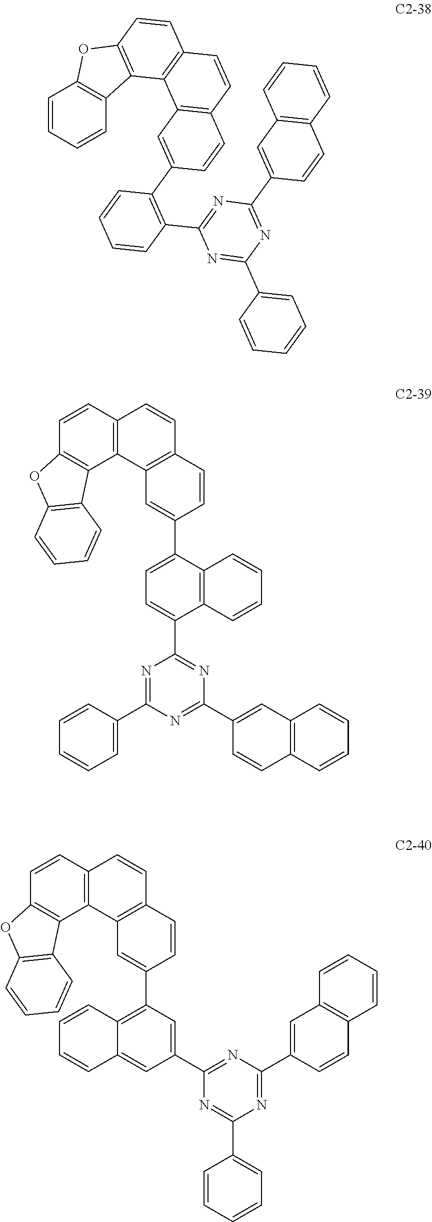 [SMILES: c1ccc(-c2nc(-c3ccc4ccccc4c3)nc(-c3ccccc3-c3ccc4ccc5ccc6oc7ccccc7c6c5c4c3)n2)cc1 ; c1ccc(-c2nc(-c3ccc4ccccc4c3)nc(-c3ccc(-c4ccc5ccc6ccc7oc8ccccc8c7c6c5c4)c4ccccc34)n2)cc1 ; c1ccc(-c2nc(-c3ccc4ccccc4c3)nc(-c3cc(-c4ccc5ccc6ccc7oc8ccccc8c7c6c5c4)c4ccccc4c3)n2)cc1]

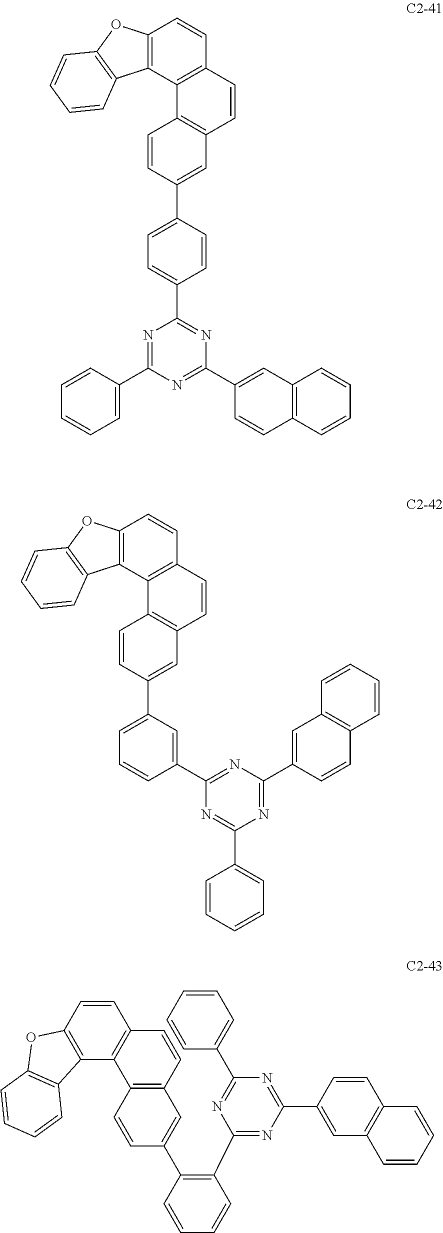 [SMILES: c1ccc(-c2nc(-c3ccc(-c4ccc5c(ccc6ccc7oc8ccccc8c7c65)c4)cc3)nc(-c3ccc4ccccc4c3)n2)cc1 ; c1ccc(-c2nc(-c3cccc(-c4ccc5c(ccc6ccc7oc8ccccc8c7c65)c4)c3)nc(-c3ccc4ccccc4c3)n2)cc1 ; c1ccc(-c2nc(-c3ccc4ccccc4c3)nc(-c3ccccc3-c3ccc4c(ccc5ccc6oc7ccccc7c6c54)c3)n2)cc1]

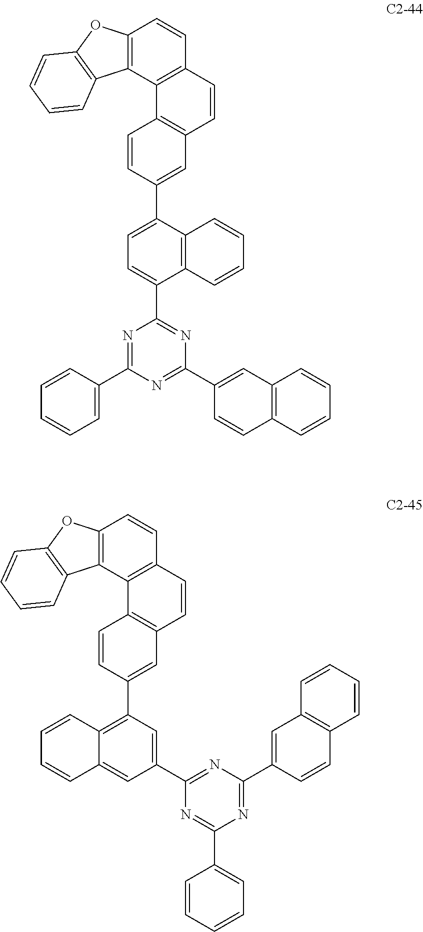 [SMILES: c1ccc(-c2nc(-c3ccc4ccccc4c3)nc(-c3ccc(-c4ccc5c(ccc6ccc7oc8ccccc8c7c65)c4)c4ccccc34)n2)cc1 ; c1ccc(-c2nc(-c3ccc4ccccc4c3)nc(-c3cc(-c4ccc5c(ccc6ccc7oc8ccccc8c7c65)c4)c4ccccc4c3)n2)cc1]

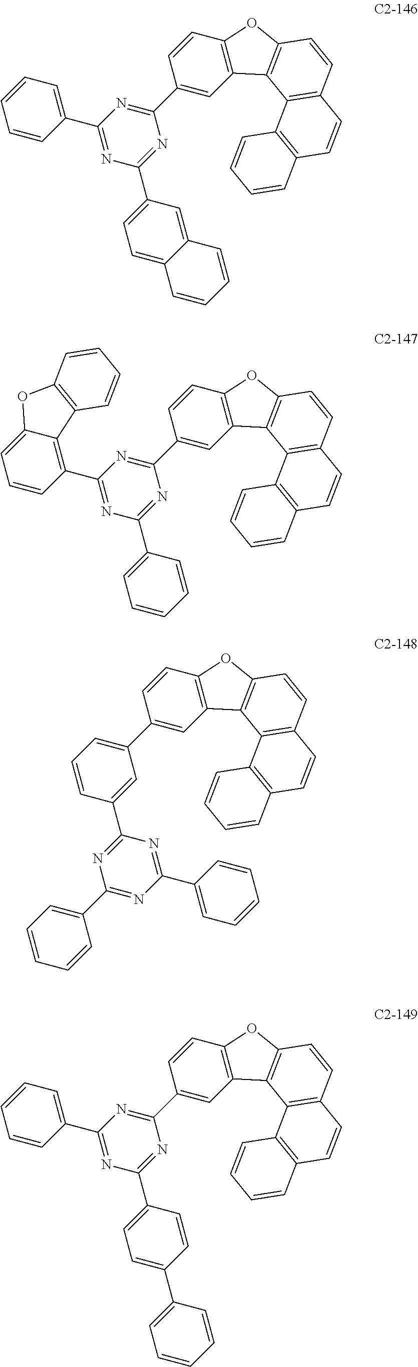 [SMILES: c1ccc(-c2nc(-c3ccc4ccccc4c3)nc(-c3ccc4oc5ccc6ccc7ccccc7c6c5c4c3)n2)cc1 ; c1ccc(-c2nc(-c3ccc4oc5ccc6ccc7ccccc7c6c5c4c3)nc(-c3cccc4oc5ccccc5c34)n2)cc1 ; c1ccc(-c2nc(-c3ccccc3)nc(-c3cccc(-c4ccc5oc6ccc7ccc8ccccc8c7c6c5c4)c3)n2)cc1 ; c1ccc(-c2ccc(-c3nc(-c4ccccc4)nc(-c4ccc5oc6ccc7ccc8ccccc8c7c6c5c4)n3)cc2)cc1]

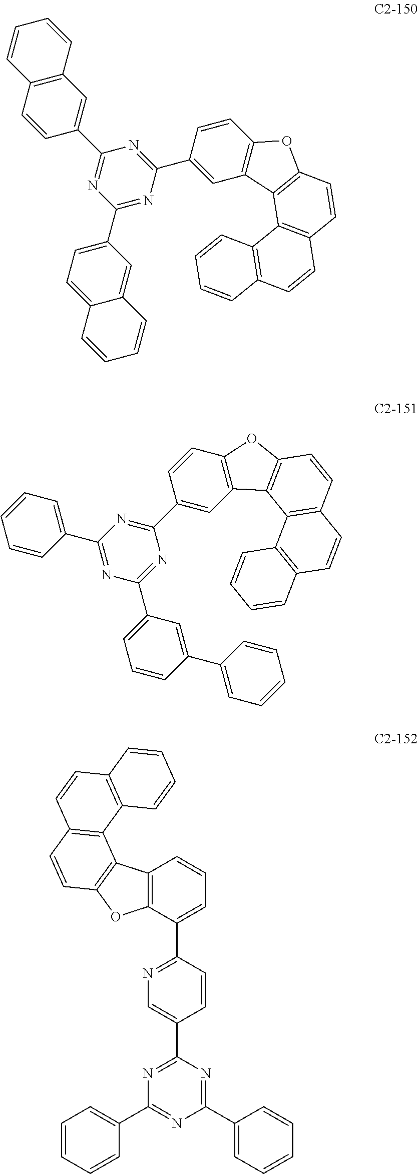 [SMILES: c1ccc2cc(-c3nc(-c4ccc5ccccc5c4)nc(-c4ccc5oc6ccc7ccc8ccccc8c7c6c5c4)n3)ccc2c1 ; c1ccc(-c2cccc(-c3nc(-c4ccccc4)nc(-c4ccc5oc6ccc7ccc8ccccc8c7c6c5c4)n3)c2)cc1 ; c1ccc(-c2nc(-c3ccccc3)nc(-c3ccc(-c4cccc5c4oc4ccc6ccc7ccccc7c6c45)nc3)n2)cc1]

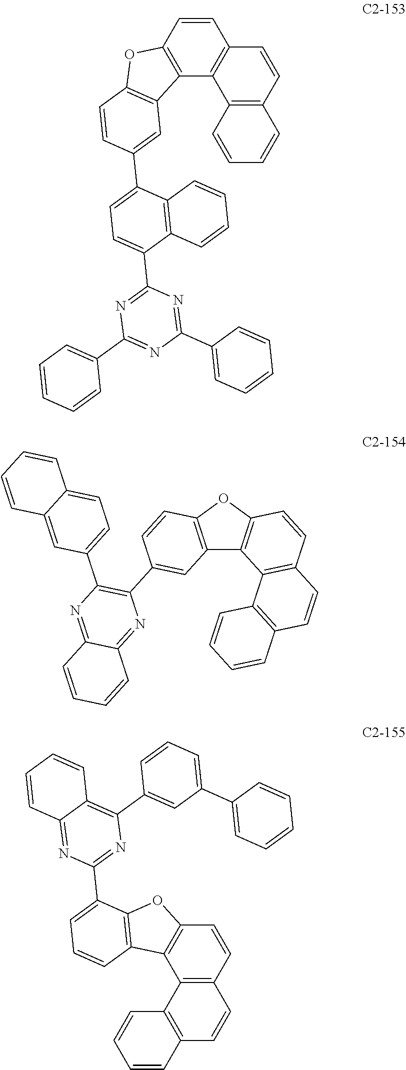 [SMILES: c1ccc(-c2nc(-c3ccccc3)nc(-c3ccc(-c4ccc5oc6ccc7ccc8ccccc8c7c6c5c4)c4ccccc34)n2)cc1 ; c1ccc2cc(-c3nc4ccccc4nc3-c3ccc4oc5ccc6ccc7ccccc7c6c5c4c3)ccc2c1 ; c1ccc(-c2cccc(-c3nc(-c4cccc5c4oc4ccc6ccc7ccccc7c6c45)nc4ccccc34)c2)cc1]

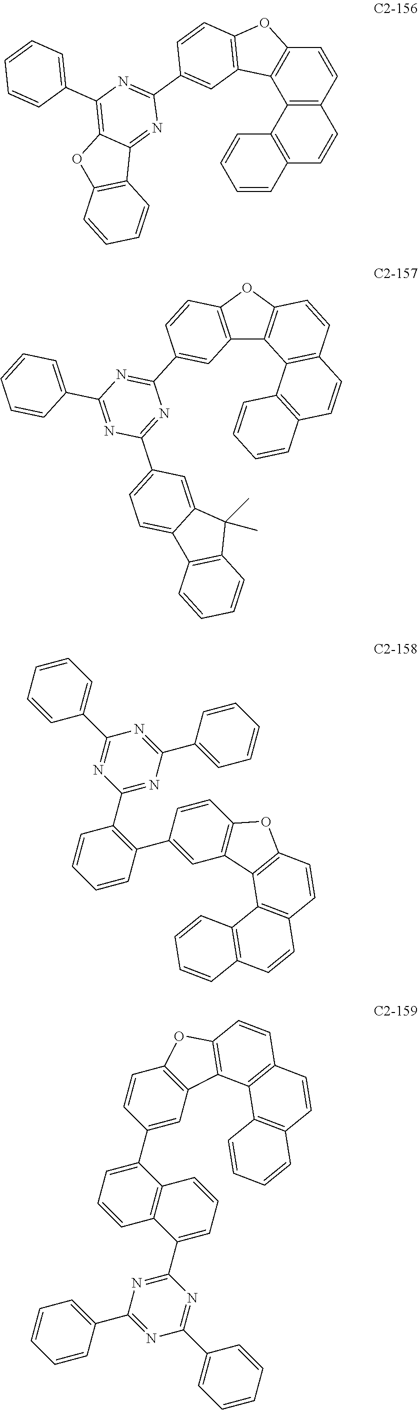 [SMILES: c1ccc(-c2nc(-c3ccc4oc5ccc6ccc7ccccc7c6c5c4c3)nc3c2oc2ccccc23)cc1 ; CC1(C)c2ccccc2-c2ccc(-c3nc(-c4ccccc4)nc(-c4ccc5oc6ccc7ccc8ccccc8c7c6c5c4)n3)cc21 ; c1ccc(-c2nc(-c3ccccc3)nc(-c3ccccc3-c3ccc4oc5ccc6ccc7ccccc7c6c5c4c3)n2)cc1 ; c1ccc(-c2nc(-c3ccccc3)nc(-c3cccc4c(-c5ccc6oc7ccc8ccc9ccccc9c8c7c6c5)cccc34)n2)cc1]

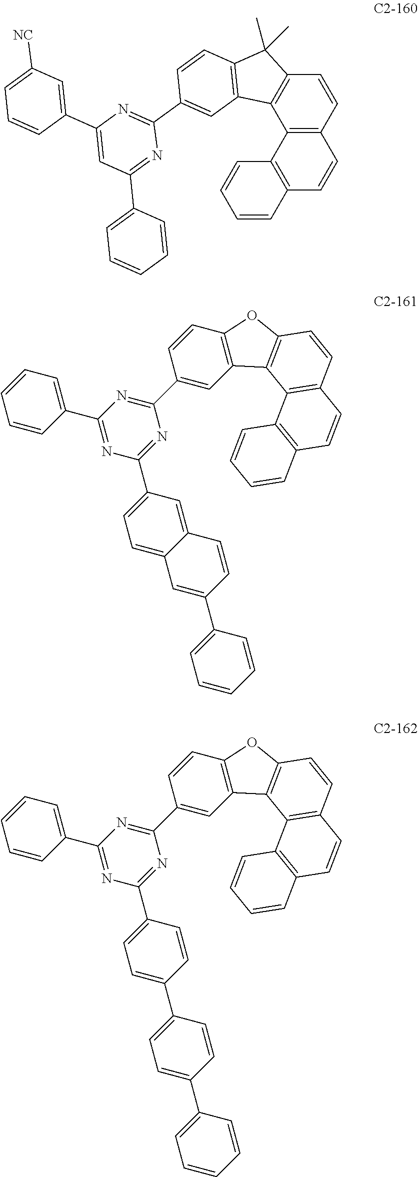 [SMILES: CC1(C)c2ccc(-c3nc(-c4ccccc4)cc(-c4cccc(C#N)c4)n3)cc2-c2c1ccc1ccc3ccccc3c21 ; c1ccc(-c2ccc3cc(-c4nc(-c5ccccc5)nc(-c5ccc6oc7ccc8ccc9ccccc9c8c7c6c5)n4)ccc3c2)cc1 ; c1ccc(-c2ccc(-c3ccc(-c4nc(-c5ccccc5)nc(-c5ccc6oc7ccc8ccc9ccccc9c8c7c6c5)n4)cc3)cc2)cc1]

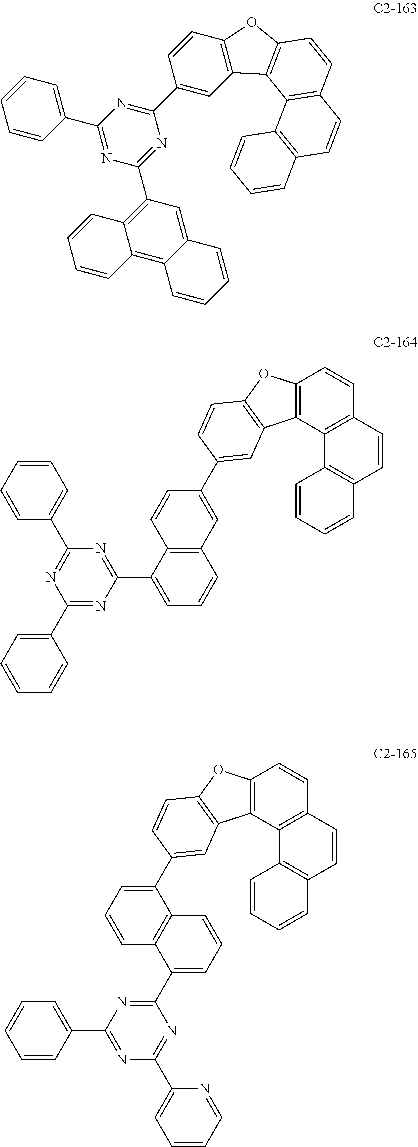 [SMILES: c1ccc(-c2nc(-c3ccc4oc5ccc6ccc7ccccc7c6c5c4c3)nc(-c3cc4ccccc4c4ccccc34)n2)cc1 ; c1ccc(-c2nc(-c3ccccc3)nc(-c3cccc4cc(-c5ccc6oc7ccc8ccc9ccccc9c8c7c6c5)ccc34)n2)cc1 ; c1ccc(-c2nc(-c3ccccn3)nc(-c3cccc4c(-c5ccc6oc7ccc8ccc9ccccc9c8c7c6c5)cccc34)n2)cc1]

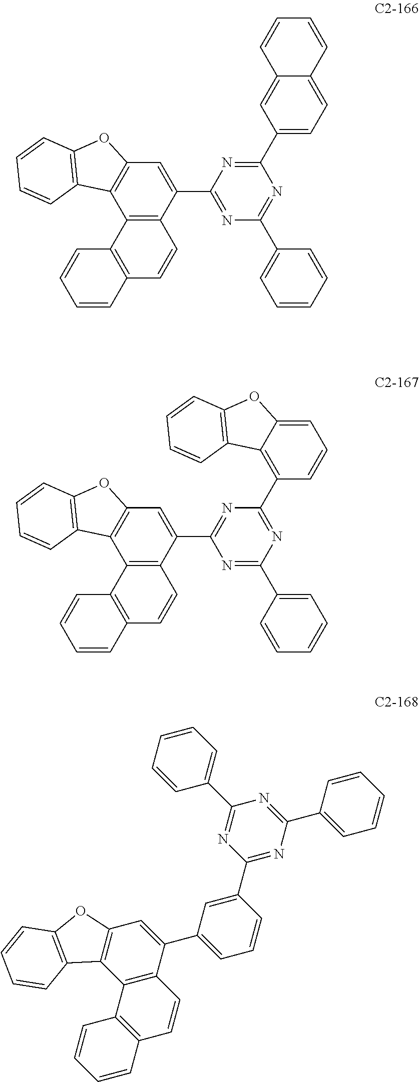 [SMILES: c1ccc(-c2nc(-c3ccc4ccccc4c3)nc(-c3cc4oc5ccccc5c4c4c3ccc3ccccc34)n2)cc1 ; c1ccc(-c2nc(-c3cc4oc5ccccc5c4c4c3ccc3ccccc34)nc(-c3cccc4oc5ccccc5c34)n2)cc1 ; c1ccc(-c2nc(-c3ccccc3)nc(-c3cccc(-c4cc5oc6ccccc6c5c5c4ccc4ccccc45)c3)n2)cc1]

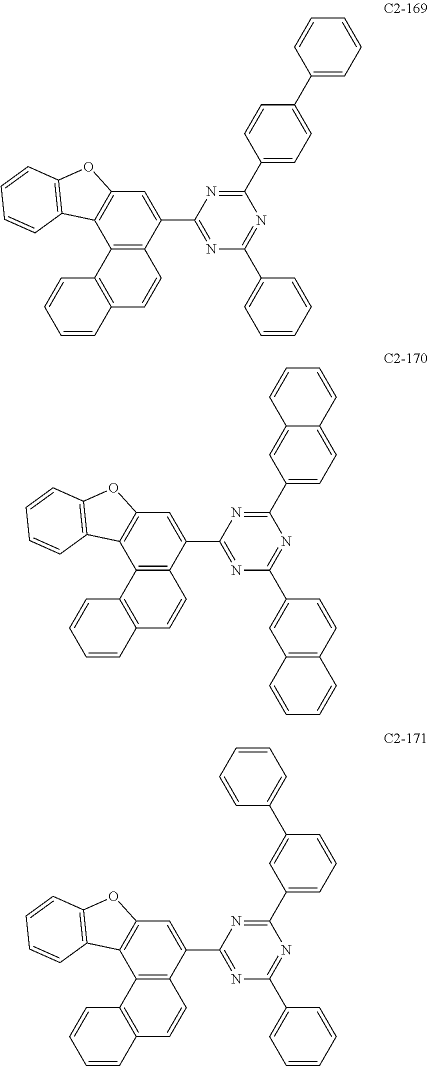

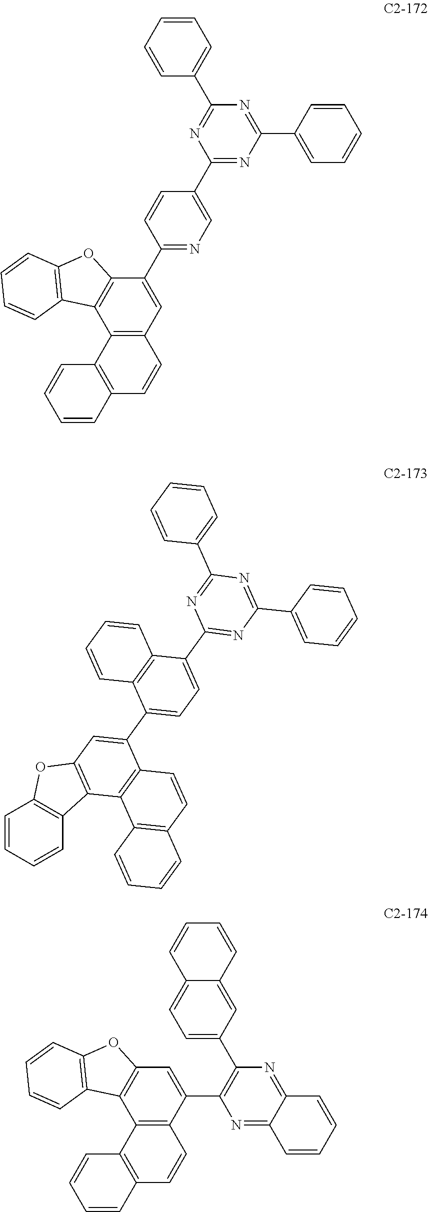

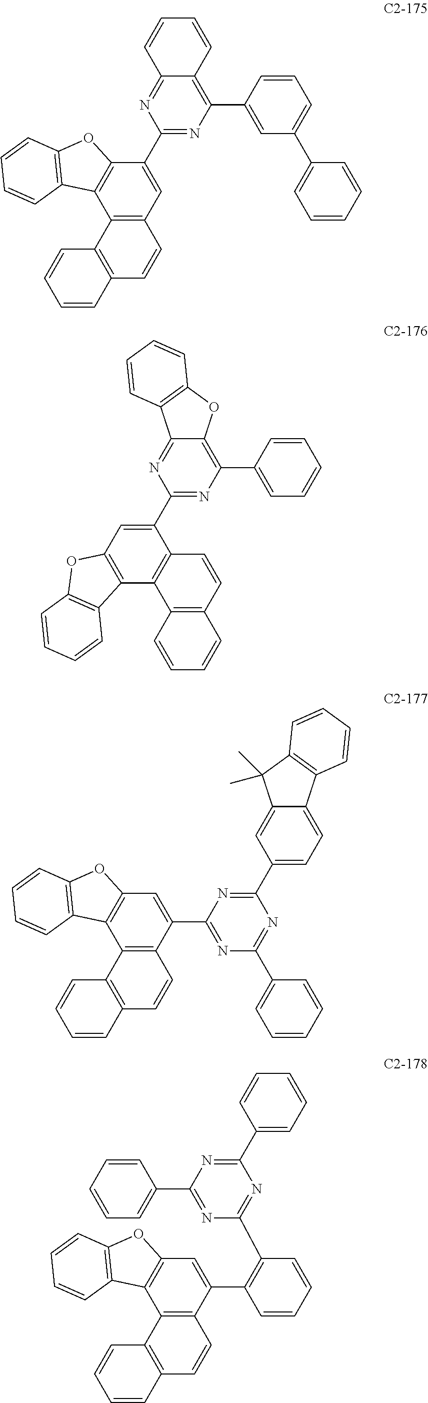

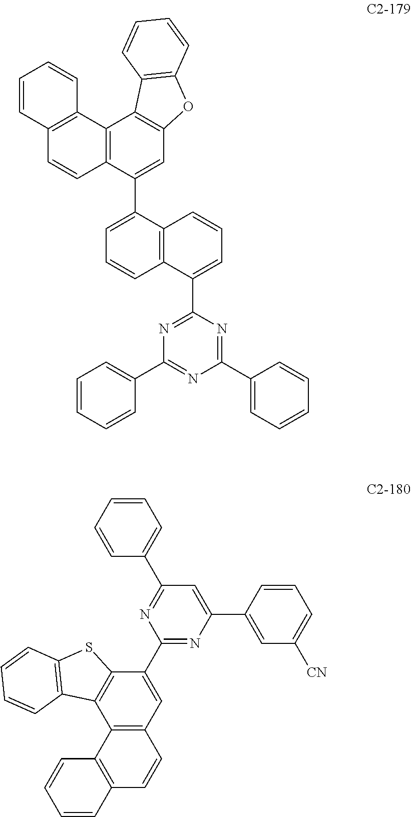 [SMILES: c1ccc(-c2nc(-c3ccccc3)nc(-c3cccc4c(-c5cc6oc7ccccc7c6c6c5ccc5ccccc56)cccc34)n2)cc1 ; N#Cc1cccc(-c2cc(-c3ccccc3)nc(-c3cc4ccc5ccccc5c4c4c3sc3ccccc34)n2)c1]

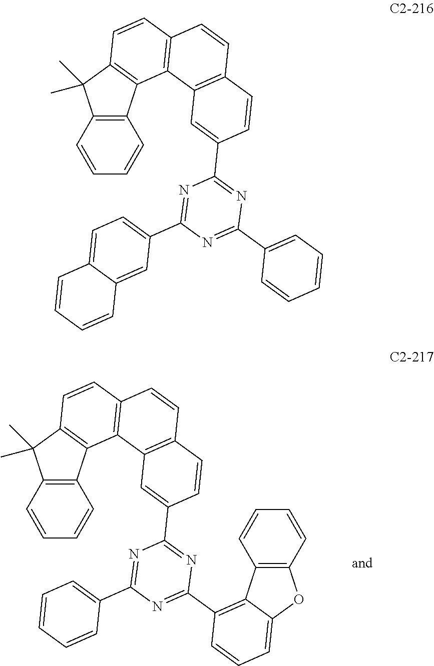 [SMILES: CC1(C)c2ccccc2-c2c1ccc1ccc3ccc(-c4nc(-c5ccccc5)nc(-c5ccc6ccccc6c5)n4)cc3c21 ; CC1(C)c2ccccc2-c2c1ccc1ccc3ccc(-c4nc(-c5ccccc5)nc(-c5cccc6oc7ccccc7c56)n4)cc3c21]

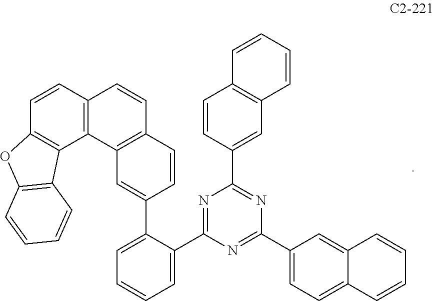 [SMILES: c1ccc(-c2nc(-c3ccc4ccccc4c3)nc(-c3ccc4ccccc4c3)n2)c(-c2ccc3ccc4ccc5oc6ccccc6c5c4c3c2)c1]

7. An organic electroluminescent device comprising an anode, a cathode, and at least one light-emitting layer between the anode and the cathode, wherein at least one layer of the light-emitting layer comprises the plurality of host materials according to claim 1.
